SEQUENCE LISTING (53 sequences)

SEQ ID NO: 1 - DNA, 139 bp, Paenibacillus polymyxa
atgtgtttttctctttttttcagaaaatttctttcttgaaaataccagttattgcattataattgtaatatacatccattgttttacattgaaagtatttcattggtttatttcaaagatgaaaagaagaggtggtagc

SEQ ID NO: 2 - DNA, 328 bp, Paenibacillus polymyxa
tataataggtaagaagaggcttgtgaaaaagcattttctcgttcaataccgatgcaggctctctatatagagggcctgttggtttgggaagggaggtgaacggcaaggctaaggatataggcgcgaagctagggaaaataaagtgacaaaaatcactcatattgtgatttttgtcacatactacactctatcattattctacaataagggtgtaagaaaggtcccgacactgagcgtaaggcatcggtgaacaggccgggctcaagaaatagctttatcgtgaaaaaaatcacaagtccggtagaccaaacaatttggagggatttac

SEQ ID NO: 3 - DNA, 194 bp, Paenibacillus polymyxa
tattgttttagttcttaggggaggtgaggcctatcgcagaagcaccggattatgctcccttgtccatgagggcaaggaaggtggatttcttattttggggaggtgagttcagcctcgttatgctctgatagggtggtagactttataaagatgacttaataagaatgtatattctgatatggaggggaaatatc

SEQ ID NO: 4 - DNA, 171 bp, Paenibacillus polymyxa
aaaatgacagagcttgtcttttcagtaatacggatgtgaatggagaaaagtggccctttatgtattcattttttcttataggttgacacctggtttccgtttcacctataataaaatttgatgaaccgtgttattgctcttggacggtctggtcggagggagggaaaactg

SEQ ID NO: 5 - DNA, 258 bp, Paenibacillus polymyxa
cgtccatttcccctttgtatcctatgttttttatctgcaacttgatcaattcaaattcattatatacctgtgcaatgagtttttggggcaagaagagggggtaaaagtggcagaattccaaactttgtgatttaattcacttcataaaataaaattttaaatagattgtgaaaaaaatcacaatatatcttgaaaagctgtgttacgatcagtgcgtgaacaagagatagtacacgagataacaggaggatgagggaa

SEQ ID NO: 6 - DNA, 183 bp, Paenibacillus polymyxa
ttttacccttgcggaattgatggttatcctgtaatataaaagttgtgttttcttaatggctttcccacagccccaattaagataatcactttaaagtaacatctaatccatcaataatgcagcaacttaacgaacgaagataaatgatgatttctgttaacgaacttaaggaggatacttttc

SEQ ID NO: 7 - DNA, 117 bp, Paenibacillus polymyxa
tcatgcaggtttttattaaacgtatatcatttaaaaaaacttgttttgctcggcaaaacgatgtgaatgttcacgaagcgagttttaccagggtaaagcttggaggaggtaatcaac

SEQ ID NO: 8 - DNA, 244 bp, Paenibacillus polymyxa
aattgtctgtggactgtattactataaaccttgttccacgcaagcagggtgccataaaacgccagtttcggcctatattgcggaggttaggtatattctatgtatatttaacgacgaaacggaaacagatgataacattatggaaggcagtacgccgcccgggttattttatacaggcgacctcatatcggtcattaacaccgataacctaaatgctggactacacttgaattgggaggtgaat

SEQ ID NO: 9 - DNA, 309 bp, Paenibacillus polymyxa
ttagacggtgggataggactagagatcggtgtcatcaatttttattaaagtgttgacacctcgattgcaccgtgataagatatttgagtatgttaattgaaaagtatggattaacaagtagaaggtccgacttctcacctgcccggtattgccggctggtcatgatccaatgatttgtgaattgaactttcatgatgaattgagtatcagacagggatgtcggctgtgtagtcgtcatcccttttttgtgcttaaacacaatacaacaataacaggttctggaggtggagggttattagtaaggaacat

SEQ ID NO: 10 - DNA, 140 bp, Paenibacillus polymyxa
ggtaagaaaataacaaatgttccatgatgctgtttcatccatttgcgcagagggcgcaactttttttgacctgttcggtatatatagacagaatcgaacgggaaaacagcacatgtatgggcgaggagggtctcactcgg

SEQ ID NO: 11 - DNA, 143 bp, Paenibacillus polymyxa
tgaccggaaacgtttacctgtgaagcattcaaagggctgctcttgaaaaagacagtaatgttagatatactatagtggttattttgtgtgcgttgattgtaatgaaaactaagtgaagattttgttggaggggaaagcataaa

SEQ ID NO: 12 - DNA, 414 bp, Paenibacillus polymyxa
atgggcgacgaaattcgcagaaatatggccttatttccaataggtattgtcatgaagctaacggacttgtcagcgcgtcagattcgttattatgaacagcataacttgatagttcctgcccgtacatcgggaaaccaacgtcttttttcttttaatgacgtagagcgtctgcttgaaattaaggcgttgatcgagaagggtgttaacattgcgggaattaaacaagtcatgaatccggttaccaaggaatcggaggaagctacggttattactgcagatacggaagttaaacgccgtgaaatgtctgatactcagcttcaccgcttgctgaaacaacaacttgttgcaggcaaaaggccaggacaggtatccctgatccaaggtgaattatcacggttcttcaataagagataa

SEQ ID NO: 13 - DNA, 414 bp, Artificial Sequence
  glnR-R99H, L114P, A116V, F133L
atgggcgacgaaattcgcagaaatatggccttatttccaataggtattgtcatgaagctaacggacttgtcagcgcgtcagattcgttattatgaacagcataacttgatagttcctgcccgtacatcgggaaaccaacgtcttttttcttttaatgacgtagagcgtctgcttgaaattaaggcgttgatcgagaagggtgttaacattgcgggaattaaacaagtcatgaatccggttaccaaggaatcggaggaagctacggttattactgcagatacggaagttaaacgccatgaaatgtctgatactcagcttcaccgcttgctgaaacaacaacctgttgtaggcaaaaggccaggacaggtatccctgatccaaggtgaattatcacggctcttcaataagagataa

SEQ ID NO: 14 - DNA, 414 bp, Artificial Sequence
  glnR-I16V, T91I, L106F, L114P, G128S
atgggcgacgaaattcgcagaaatatggccttatttccaataggcgttgtcatgaagctaacggacttgtcagcgcgtcagattcgttattatgaacagcataacttgatagttcctgcccgtacatcgggaaaccaacgtcttttttcttttaatgacgtagagcgtctgcttgaaattaaggcgttgatcgagaagggtgttaacattgcgggaattaaacaagtcatgaatccggttaccaaggaatcggaggaagctacggttattattgcagatacggaagttaaacgccgtgaaatgtctgatactcagtttcaccgcttgctgaaacaacaacctgttgcaggcaaaaggccaggacaggtatccctgatccaaagcgaattatcacggttcttcaataagagataa

SEQ ID NO: 15 - DNA, 414 bp, Artificial Sequence
  glnR-M18V, I37M, V54I, L114P, Q122R
atgggcgacgaaattcgcagaaatatggccttatttccaataggtattgtcgtgaagctaacggacttgtcagcgcgtcagattcgttattatgaacagcataacttgatggttcctgcccgtacatcgggaaaccaacgtcttttttcttttaatgacatagagcgtctgcttgaaattaaggcgttgatcgagaagggtgttaacattgcgggaattaaacaagtcatgaatccggttaccaaggaatcggaggaagctacggttattactgcagatacggaagttaaacgccgtgaaatgtctgatactcagcttcaccgcttgctgaaacaacaacctgttgcaggcaaaaggccaggacgggtatccctgatccaaggtgaattatcacggttcttcaataagagataa

SEQ ID NO: 16 - protein, 137 aa, Paenibacillus polymyxa
MGDEIRRNMALFPIGIVMKLTDLSARQIRYYEQHNLIVPARTSGNQRLFSFNDVERLLEIKALIEKGVNIAGIKQVMNPVTKESEEATVITADTEVKRREMSDTQLHRLLKQQLVAGKRPGQVSLIQGELSRFFNKR

SEQ ID NO: 17 - protein, 137 aa, Artificial Sequence
  GlnR-R99H, L114P, A116V, F133L
MGDEIRRNMALFPIGIVMKLTDLSARQIRYYEQHNLIVPARTSGNQRLFSFNDVERLLEIKALIEKGVNIAGIKQVMNPVTKESEEATVITADTEVKRHEMSDTQLHRLLKQQPVVGKRPGQVSLIQGELSRLFNKR

SEQ ID NO: 18 - protein, 137 aa, Artificial Sequence
  GlnR-I16V, T91I, L106F, L114P, G128S
MGDEIRRNMALFPIGVVMKLTDLSARQIRYYEQHNLIVPARTSGNQRLFSFNDVERLLEIKALIEKGVNIAGIKQVMNPVTKESEEATVIIADTEVKRREMSDTQFHRLLKQQPVAGKRPGQVSLIQSELSRFFNKR

SEQ ID NO: 19 - protein, 137 aa, Artificial Sequence
  GlnR-M18V, I37M, V54I, L114P, Q122R
MGDEIRRNMALFPIGIVVKLTDLSARQIRYYEQHNLMVPARTSGNQRLFSFNDIERLLEIKALIEKGVNIAGIKQVMNPVTKESEEATVITADTEVKRREMSDTQLHRLLKQQPVAGKRPGRVSLIQGELSRFFNKR

SEQ ID NO: 20 - DNA, 6316 bp, Artificial Sequence
  Reporter plasmid pPb-nifB
ttagaaatccctttgagaatgtttatatacattcaaggtaaccagccaactaatgacaatgattcctgaaaaaagtaataacaaattactatacagataagttgactgatcaacttccataggtaacaacctttgatcaagtaagggtatggataataaaccacctacaattgcaatacctgttccctctgataaaaagctggtaaagttaagcaaactcattccagcaccagcttcctgctgtttcaagctacttgaaacaattgttgatataactgttttggtgaacgaaagcccacctaaaacaaatacgattataattgtcatgaaccatgatgttgtttctaaaagaaaggaagcagttaaaaagctaacagaaagaaatgtaactccgatgtttaacacgtataaaggacctcttctatcaacaagtatcccaccaatgtagccgaaaataatgacactcattgttccagggaaaataattacacttccgatttcggcagtacttagctggtgaacatctttcatcatataaggaaccatagagacaaaccctgctactgttccaaatataattcccccacaaagaactccaatcataaaaggtatatttttccctaatccgggatcaacaaaaggatctgttactttcctgatatgttttacaaatatcaggaatgacagcacgctaacgataagaaaagaaatgctatatgatgttgtaaacaacataaaaaatacaatgcctacagacattagtataattcctttgatatcaaaatgaccttttatccttacttctttctttaataatttcataagaaacggaacagtgataattgttatcataggaatgagtagaagataggaccaatgaatataatgggctatcattccaccaatcgctggaccgactccttctcccatggctactatcgatccaataagaccaaatgctttacccctattttcctttggaatatagcgcgcaactacaaccattacgagtgctggaaatgcagctgcaccagccccttgaataaaacgagccataataagtaaggaaaagaaagaatggccaacaaacccaattaccgacccgaaacaatttattataattccaaataggagtaaccttttgatgcctaattgatcagatagctttccatatacagctgttccaatggaaaaggttaacataaaggctgtgttcacccagtttgtactcgcaggtggtttattaaaatcatttgcaatatcaggtaatgagacgttcaaaaccatttcatttaatacgctaaaaaaagataaaatgcaaagccaaattaaaatttggttgtgtcgtaaattcgattgtgaataggatgtattcacatttcaccctccaataatgagggcagacgtagtttatagggttaatgatacgcttccctcttttaattgaaccctgttacattcattattcattacacttcataattaattcctcctaaacttgattaaaacattttaccacatataaactaagttttaaattcagtatttcatcacttatacaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaagctcggtaccaaattccagaaaagaggcctcccgaaaggggggcctttttcgttttggtccaatgacggactctctttttttacgtcttgatgttattgagaatatgaaatgtaaccgcgcacatgtaaagtgtacgatatattacttgacgtaaaatttgacacatatgtgaaatgaggataaatatcagggatttcatggagaagcgaattgactgtatttgtccctgtctctaagatgtaattatattccagacaaaaacagagaattgtgtaagggaatataacgtagagaggagggaatgaatgagtaaaggagaagaacttttcactggagttgtcccaattcttgttgaattagatggtgatgttaatgggcacaaattttctgttagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatagcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggccttttgaattcgccggacggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaaatcaatctaaagtatatatgagtaaacttggtctgacagttaccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtacatcagagattttgagacacaa

SEQ ID NO: 21 - DNA, 8515 bp, Artificial Sequence
  glnR plasmid pAD-glnR
ggccttatgataccctgcataaagtgattggtaagcccggcgacggaacaggatcattgcaggattttggcgttacttatggagaaacctcactgacagcggaaggtaaagtggactgggaagcagtgaaggctgcgatccatgacaataccaaagtaatcggtattcagcgctcacgcggttatgattggagagcttcctttagtgtagcagatattgaagaaatgacagcacgtgtgaaggaaataaaacctgacgttattgtctttgttgacaattgctacggtgaatttaccgagaagctggaacctactcaagtcggtgtcgatttgatggcaggttcgttaattaaaaatcccggtggtggcattgcagaaacgggtggatatatctgcggcaaacaaaagtttgtagagctggcatcgtaccgattaactgcacctggaattggtggagaagtaggagccatgctaggtactacacgaggcatatttcagggactattccttgctccaacactggttgggcaagcggttaaagggagcgtattcgcagctgcggtctttgaagagatgggatttgagaccaaacctgcctggcatgaagagcgtacggatttgattcaggctatttcttttagtggaccagagcatctaattgcttttgtacagggcattcagcgtgcggctgccgtggatagccatgtggtgccggaaccgtgggatatgccgggttatgagcatccagttatcatggctgcaggtacgttcatacaagggggaagtttggaactatccgcagatgctcctattcgtgagccttatattggttacatgcaaggggggttaacctactctcatgttaaatttggagtgcttatggcactgcaaacgatgaaagaacgtaaattattgtgagtttttctaacatgtcattgacactttgcatcagctaaatgtacaataaggtgtataatagatcactggaaggttgatgacaaatgggcgacgaaattcgcagaaatatggccttatttccaataggtattgtcatgaagctaacggacttgtcagcgcgtcagattcgttattatgaacagcataacttgatagttcctgcccgtacatcgggaaaccaacgtcttttttcttttaatgacgtagagcgtctgcttgaaattaaggcgttgatcgagaagggtgttaacattgcgggaattaaacaagtcatgaatccggttaccaaggaatcggaggaagctacggttattactgcagatacggaagttaaacgccgtgaaatgtctgatactcagcttcaccgcttgctgaaacaacaacttgttgcaggcaaaaggccaggacaggtatccctgatccaaggtgaattatcacggttcttcaataagagataatgtcttgacttacctcttggtataaggttgccttactttaaaacctggtctcatcatttaagtaatttattgaacaactgattcgtatatagtcattacagaaagggagaggttagagtgagttatagcagagaagatatccttcggattgcgaaagaggaaaatgttcgttttattcgtttgcaattcacagatttgctgggtactattaagaacgttgaaattccggttagtcaactggaaaaagcattggataataaaatgatgtttgacggatcttctatagaaggttatgtgcgtattgaagaatctgatatgtatttatatccggatttggacacttgggttgtattcccttgggtaacctcagatcgtgtagctcgtttgatctgcgatatttacaagccggatggagtcccatttgcaggtgacccacggggcatcttgaaacgtgtacttaaggaagccgaagagctgggatacacttcgatgaatgtcggacccgagcctgaattctttttgttcaaaacggatgaaaaaggcgagccgactacagaactgaatgaccagggcgggtattttgacctggctccgatggatctgggtgaaaactgccgtcgtgaaattgtacttaagcttgaagagatgggcttcgaaattgaagcatcccatcatgaagttgcacctggtcagcatgagattgactttaaatatgcagacgcgattaaagctgcggaccagattcaaacgttcaagctcgttgttaagacgattgctcgtcagcatggtttgcatgctacctttatgccaaaacctttgtttggtgtaaatggttctggtatgcactgcaatcaatcgttgtttaaggacaatgaaaacgtattttatgatgaaacggacgaactcggattaagccagacggcccgccactatatggctggtattctgaaacatgcacgtgcaatggcagcgatcacgaatccaacagtaaattcctacatctagaagcttgagtaggacaaatccgccgagcttcgacgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaacttgtttttcgtgtgcctattttttgtgaatcgctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatacaaaacatatttcaacacaatacaaatgggttagttaaaaaagcaggccttctaaaggtctgctttttttatttgattatgtaatttttaatgccaggatgccaataagccataacctcaaatgcaccatttgcaacctcgtcatcttcttcctcaatcttgaccagatcgccgtcctccgcatcaccaagattcagctctttatgtatctccttcaaaatgccaccgtatccaattaaccttcgagctgccaacgcatcatccaagtaaagcaccgtgttcagattgtcttcagtcaccttattaccgcgcacacaatccgtatccttaaccggatatttagagatttcgcgaacagctttttgctccatcattgcgttccgcacatcgttttcaatctgttcagcgtcaatcttagctttacctttcactcgacgaatatcgacaattggagtgtaatccaatttcatcgcctttttccaaaggctcgtccactccgcctgcttaatatagtttttcccaaaataatttttccttactggtatcaacacatgaaaatgaggatgatatgtatcttcttcatgatttttggtaatctctaaagctctgaaaaatccaagaaccgaagtttttacttttttgtactggaacagtttcctaaagccttccatcatcgcagaaatttgtggcttcagccgttctccctttacatttcgaatcgtcagcgtgagaaaaatccatccgcagccgtactgtctattggcttcctctacgatcaacttattgtgataagcaatttttaacgacctgcgccacgcacacatcggacataacctcactttacaaaaatgggcttgatacagttttaacttgcccgtctccgggtctctcttaaacgaaagatactctgcacaactaattagtttttcagcctttttgccatagtaaggtgccccaatcttactctctaacgcttcgtaatgctccgccatgaggttcgtccgtctctttttccccttccaatcccgctttttacctgttgcggttttatcttcgaggatgctataatcattttcagatgaataaatcaacaaaaaaactccttctgagctagttctctagcattctattattttgattcgacaccttaataatagcagaaggagtttttacctgtcaaagaaccatcaaacccttgatacacaaggctttgacctaattttgaaaaatgatgttgtttctatatagtatcaagataagaaagaaaaggatttttcgctacgctcaaatcctttaaaaaaacacaaaagaccacattttttaatgtggtctttattcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactaacctgccccgttagtcgccattcgccagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacataccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcgccggacggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtgaattcgagctctgtttgagtcggctgaaagatcg

SEQ ID NO: 22 - DNA, 1805 bp, Paenibacillus polymyxa
taagcggagactatttcccaaaatatataatgaaatttcttgtcttaaaaggagagccgtatttacggactctctttttttacgtcttgatgttattgagaatatgaaatgtaaccgcgcacatgtaaagtgtacgatatattacttgacgtaaaatttgacacatatgtgaaatgaggataaatatcagggatttcatggagaagcgaattgactgtatttgtccctgtctctaagatgtaattatattccagacaaaaacagagaattgtgtaagggaatataacgtagagaggagggaatgaatggactctttagctgatctctcggaaacccccttagccttggaaaccctcagacgacatccctgttataacgaagaggcacatcgctattttgcgcgcattcatcttccagtagccccggcatgcaatattcagtgccattattgcaaccgcaaattcgattgcgtcaatgaaagccgtcccggcgttgttagtgaactgctcacgccggagcaggcggcgagcaagatctatggcgtagcggcacagctgatgcaactgtccgttgtcggcattgcgggacctggagatccgctggccaatgcggaggcaaccttcgataccttccgccgggtccgtgagacagttaaggatgtcatattctgtctcagcacgaatggccttaccttgatcaggcatatcgacaggattgtagagttgggtatttcgcatgtcacgatcacgatcaatgctgtagatccagtggtggggagccgcatttatggatgggtctacgatgaaggaaaacgctatgcaggagaggaggccgcgcggctgttgattgaccgccagctggcaggtttgaagatgctggcttcgagaggcgtattgtgcaaggtgaactcggtgctgattcccgaagtcaatgatgcccatctgccagaggtagccagggtggtcaaggagcacggcgcggtactgcacaacattatgccgctcatcatcgcacccggcagtcggtatgagcaggaagggatgcggcccccccgtccccgtctggtccggcagctgcaggagcaatgtgctgaggcgggagctgtcattatgcgccattgccgtcagtgcagggcggatgcgattggactgctgggcgaggatcgcaatcaggattttacatgggagaacattgcggctgctcctcccatggatgaagaggcaagggcacaatttcagaaagaactggatgagaaggtgagagtgagaatggaacgtaaggagggacaatcgcaccacaaaaaaacgtcaaccggggttggatgtagctgcccgttatcggaggataagcctgaagcaagctttacctcaaagccagtcctaatcgctgtggccagtcgtggcggagggaaggtgaatcagcatttcggtcgtgccaaggaattcatgatctatgaaagcgacgggactatcgtaaatttcataggcattcgtaaggtgcaatcctactgccatgggaaagccgattgcaatggagacaaggtcgagacgatgaaggagatcctttccatggtgcatgactgtgcattgctgctgtcgtccggcataggcgaagcccccaaagaggcattgcaggaagctggcgtgctgcctattgtatgcggcggggatattgaggaatccgttctggagtatgtaaaatttctgcgttatatgtatcctgtgcagagcaataagggaagtaagcgcaataagggagttaagggcaatcattcggatttacccattgaacattttggaggctga

SEQ ID NO: 23 - DNA, 2127 bp, Artificial Sequence
  mutant nifB gene P(pflB)-nifB_v1
taagcggagactatttcccaaaatatataatgaaatttcttgtcttaaaaggagagccgtatttacggactctctttttttacgtcttgatgttattgagaatatgaaatgtaaccgcgcacatgtaaagtgtacgatatattacttgacgtaaaatttgacacatatgtgaaatgaggataaatatcagggatttcatggagaagcgaattgactgtatttgtccctgtctctaagatgtaattatattccagacaaaaacagagaattgtgtaagggaatataacgtagagaggagggaatgaaggtaagaagaggcttgtgaaaaagcattttctcgttcaataccgatgcaggctctctatatagagggcctgttggtttgggaagggaggtgaacggcaaggctaaggatataggcgcgaagctagggaaaataaagtgacaaaaatcactcatattgtgatttttgtcacatactacactctatcattattctacaataagggtgtaagaaaggtcccgacactgagcgtaaggcatcggtgaacaggccgggctcaagaaatagctttatcgtgaaaaaaatcacaagtccggtagacgaaacaatttggagggatttacatggactctttagctgatctctcggaaacccccttagccttggaaaccctcagacgacatccctgttataacgaagaggcacatcgctattttgcgcgcattcatcttccagtagccccggcatgcaatattcagtgccattattgcaaccgcaaattcgattgcgtcaatgaaagccgtcccggcgttgttagtgaactgctcacgccggagcaggcggcgagcaagatctatggcgtagcggcacagctgatgcaactgtccgttgtcggcattgcgggacctggagatccgctggccaatgcggaggcaaccttcgataccttccgccgggtccgtgagacagttaaggatgtcatattctgtctcagcacgaatggccttaccttgatcaggcatatcgacaggattgtagagttgggtatttcgcatgtcacgatcacgatcaatgctgtagatccagtggtggggagccgcatttatggatgggtctacgatgaaggaaaacgctatgcaggagaggaggccgcgcggctgttgattgaccgccagctggcaggtttgaagatgctggcttcgagaggcgtattgtgcaaggtgaactcggtgctgattcccgaagtcaatgatgcccatctgccagaggtagccagggtggtcaaggagcacggcgcggtactgcacaacattatgccgctcatcatcgcacccggcagtcggtatgagcaggaagggatgcggcccccccgtccccgtctggtccggcagctgcaggagcaatgtgctgaggcgggagctgtcattatgcgccattgccgtcagtgcagggcggatgcgattggactgctgggcgaggatcgcaatcaggattttacatgggagaacattgcggctgctcctcccatggatgaagaggcaagggcacaatttcagaaagaactggatgagaaggtgagagtgagaatggaacgtaaggagggacaatcgcaccacaaaaaaacgtcaaccggggttggatgtagctgcccgttatcggaggataagcctgaagcaagctttacctcaaagccagtcctaatcgctgtggccagtcgtggcggagggaaggtgaatcagcatttcggtcgtgccaaggaattcatgatctatgaaagcgacgggactatcgtaaatttcataggcattcgtaaggtgcaatcctactgccatgggaaagccgattgcaatggagacaaggtcgagacgatgaaggagatcctttccatggtgcatgactgtgcattgctgctgtcgtccggcataggcgaagcccccaaagaggcattgcaggaagctggcgtgctgcctattgtatgcggcggggatattgaggaatccgttctggagtatgtaaaatttctgcgttatatgtatcctgtgcagagcaataagggaagtaagcgcaataagggagttaagggcaatcattcggatttacccattgaacattttggaggctga

SEQ ID NO: 24 - DNA, 2076 bp, Artificial Sequence
  mutant nifB gene P(pflB)-nifB_v2
taagcggagactatttcccaaaatatataatgaaatttcttgtcttaaaaggagagccgtatttacggactctctttttttacgtcttgatgttattgagaatatgaaatgtaaccgcgcacatgtaaagtgtacgatatattacttgacgtaaaatttgacacatatgtgaaatgaggataaatatcagggatttcatggagaagcgaattgactgtatttgtccctgtctctaagatgtaattatattccagaggtaagaagaggcttgtgaaaaagcattttctcgttcaataccgatgcaggctctctatatagagggcctgttggtttgggaagggaggtgaacggcaaggctaaggatataggcgcgaagctagggaaaataaagtgacaaaaatcactcatattgtgatttttgtcacatactacactctatcattattctacaataagggtgtaagaaaggtcccgacactgagcgtaaggcatcggtgaacaggccgggctcaagaaatagctttatcgtgaaaaaaatcacaagtccggtagacgaaacaatttggagggatttacatggactctttagctgatctctcggaaacccccttagccttggaaaccctcagacgacatccctgttataacgaagaggcacatcgctattttgcgcgcattcatcttccagtagccccggcatgcaatattcagtgccattattgcaaccgcaaattcgattgcgtcaatgaaagccgtcccggcgttgttagtgaactgctcacgccggagcaggcggcgagcaagatctatggcgtagcggcacagctgatgcaactgtccgttgtcggcattgcgggacctggagatccgctggccaatgcggaggcaaccttcgataccttccgccgggtccgtgagacagttaaggatgtcatattctgtctcagcacgaatggccttaccttgatcaggcatatcgacaggattgtagagttgggtatttcgcatgtcacgatcacgatcaatgctgtagatccagtggtggggagccgcatttatggatgggtctacgatgaaggaaaacgctatgcaggagaggaggccgcgcggctgttgattgaccgccagctggcaggtttgaagatgctggcttcgagaggcgtattgtgcaaggtgaactcggtgctgattcccgaagtcaatgatgcccatctgccagaggtagccagggtggtcaaggagcacggcgcggtactgcacaacattatgccgctcatcatcgcacccggcagtcggtatgagcaggaagggatgcggcccccccgtccccgtctggtccggcagctgcaggagcaatgtgctgaggcgggagctgtcattatgcgccattgccgtcagtgcagggcggatgcgattggactgctgggcgaggatcgcaatcaggattttacatgggagaacattgcggctgctcctcccatggatgaagaggcaagggcacaatttcagaaagaactggatgagaaggtgagagtgagaatggaacgtaaggagggacaatcgcaccacaaaaaaacgtcaaccggggttggatgtagctgcccgttatcggaggataagcctgaagcaagctttacctcaaagccagtcctaatcgctgtggccagtcgtggcggagggaaggtgaatcagcatttcggtcgtgccaaggaattcatgatctatgaaagcgacgggactatcgtaaatttcataggcattcgtaaggtgcaatcctactgccatgggaaagccgattgcaatggagacaaggtcgagacgatgaaggagatcctttccatggtgcatgactgtgcattgctgctgtcgtccggcataggcgaagcccccaaagaggcattgcaggaagctggcgtgctgcctattgtatgcggcggggatattgaggaatccgttctggagtatgtaaaatttctgcgttatatgtatcctgtgcagagcaataagggaagtaagcgcaataagggagttaagggcaatcattcggatttacccattgaacattttggaggctga

SEQ ID NO: 25 - DNA, 2027 bp, Artificial Sequence
  mutant nifB gene P(pflB)-nifB_v3
taagcggagactatttcccaaaatatataatgaaatttcttgtcttaaaaggagagccgtatttacggactctctttttttacgtcttgatgttattgagaatatgaaatgtaaccgcgcacatgtaaagtgtacgatatattacttgacgtaaaatttgacacatatgtgaaatgaggataaatatcagggatttcatggagaaaggtaagaagaggcttgtgaaaaagcattttctcgttcaataccgatgcaggctctctatatagagggcctgttggtttgggaagggaggtgaacggcaaggctaaggatataggcgcgaagctagggaaaataaagtgacaaaaatcactcatattgtgatttttgtcacatactacactctatcattattctacaataagggtgtaagaaaggtcccgacactgagcgtaaggcatcggtgaacaggccgggctcaagaaatagctttatcgtgaaaaaaatcacaagtccggtagacgaaacaatttggagggatttacatggactctttagctgatctctcggaaacccccttagccttggaaaccctcagacgacatccctgttataacgaagaggcacatcgctattttgcgcgcattcatcttccagtagccccggcatgcaatattcagtgccattattgcaaccgcaaattcgattgcgtcaatgaaagccgtcccggcgttgttagtgaactgctcacgccggagcaggcggcgagcaagatctatggcgtagcggcacagctgatgcaactgtccgttgtcggcattgcgggacctggagatccgctggccaatgcggaggcaaccttcgataccttccgccgggtccgtgagacagttaaggatgtcatattctgtctcagcacgaatggccttaccttgatcaggcatatcgacaggattgtagagttgggtatttcgcatgtcacgatcacgatcaatgctgtagatccagtggtggggagccgcatttatggatgggtctacgatgaaggaaaacgctatgcaggagaggaggccgcgcggctgttgattgaccgccagctggcaggtttgaagatgctggcttcgagaggcgtattgtgcaaggtgaactcggtgctgattcccgaagtcaatgatgcccatctgccagaggtagccagggtggtcaaggagcacggcgcggtactgcacaacattatgccgctcatcatcgcacccggcagtcggtatgagcaggaagggatgcggcccccccgtccccgtctggtccggcagctgcaggagcaatgtgctgaggcgggagctgtcattatgcgccattgccgtcagtgcagggcggatgcgattggactgctgggcgaggatcgcaatcaggattttacatgggagaacattgcggctgctcctcccatggatgaagaggcaagggcacaatttcagaaagaactggatgagaaggtgagagtgagaatggaacgtaaggagggacaatcgcaccacaaaaaaacgtcaaccggggttggatgtagctgcccgttatcggaggataagcctgaagcaagctttacctcaaagccagtcctaatcgctgtggccagtcgtggcggagggaaggtgaatcagcatttcggtcgtgccaaggaattcatgatctatgaaagcgacgggactatcgtaaatttcataggcattcgtaaggtgcaatcctactgccatgggaaagccgattgcaatggagacaaggtcgagacgatgaaggagatcctttccatggtgcatgactgtgcattgctgctgtcgtccggcataggcgaagcccccaaagaggcattgcaggaagctggcgtgctgcctattgtatgcggcggggatattgaggaatccgttctggagtatgtaaaatttctgcgttatatgtatcctgtgcagagcaataagggaagtaagcgcaataagggagttaagggcaatcattcggatttacccattgaacattttggaggctga

SEQ ID NO: 26 - DNA, 2063 bp, Artificial Sequence
  mutant nifB gene P(adhE)-nifB_v1
taagcggagactatttcccaaaatatataatgaaatttcttgtcttaaaaggagagccgtatttacggactctctttttttacgtcttgatgttattgagaatatgaaatgtaaccgcgcacatgtaaagtgtacgatatattacttgacgtaaaatttgacacatatgtgaaatgaggataaatatcagggatttcatggagaagcgaattgactgtatttgtccctgtctctaagatgtaattatattccagacaaaaacagagaattgtgtaagggaatataacgtagagaggagggaatgacgtccatttcccctttgtatcctatgttttttatctgcaacttgatcaattcaaattcattatatacctgtgcaatgagtttttggggcaagaagagggggtaaaagtggcagaattccaaactttgtgatttaattcacttcataaaataaaattttaaatagattgtgaaaaaaatcacaatatatcttgaaaagctgtgttacgatcagtgcgtgaacaagagatagtacacgagataacaggaggatgagggaaatggactctttagctgatctctcggaaacccccttagccttggaaaccctcagacgacatccctgttataacgaagaggcacatcgctattttgcgcgcattcatcttccagtagccccggcatgcaatattcagtgccattattgcaaccgcaaattcgattgcgtcaatgaaagccgtcccggcgttgttagtgaactgctcacgccggagcaggcggcgagcaagatctatggcgtagcggcacagctgatgcaactgtccgttgtcggcattgcgggacctggagatccgctggccaatgcggaggcaaccttcgataccttccgccgggtccgtgagacagttaaggatgtcatattctgtctcagcacgaatggccttaccttgatcaggcatatcgacaggattgtagagttgggtatttcgcatgtcacgatcacgatcaatgctgtagatccagtggtggggagccgcatttatggatgggtctacgatgaaggaaaacgctatgcaggagaggaggccgcgcggctgttgattgaccgccagctggcaggtttgaagatgctggcttcgagaggcgtattgtgcaaggtgaactcggtgctgattcccgaagtcaatgatgcccatctgccagaggtagccagggtggtcaaggagcacggcgcggtactgcacaacattatgccgctcatcatcgcacccggcagtcggtatgagcaggaagggatgcggcccccccgtccccgtctggtccggcagctgcaggagcaatgtgctgaggcgggagctgtcattatgcgccattgccgtcagtgcagggcggatgcgattggactgctgggcgaggatcgcaatcaggattttacatgggagaacattgcggctgctcctcccatggatgaagaggcaagggcacaatttcagaaagaactggatgagaaggtgagagtgagaatggaacgtaaggagggacaatcgcaccacaaaaaaacgtcaaccggggttggatgtagctgcccgttatcggaggataagcctgaagcaagctttacctcaaagccagtcctaatcgctgtggccagtcgtggcggagggaaggtgaatcagcatttcggtcgtgccaaggaattcatgatctatgaaagcgacgggactatcgtaaatttcataggcattcgtaaggtgcaatcctactgccatgggaaagccgattgcaatggagacaaggtcgagacgatgaaggagatcctttccatggtgcatgactgtgcattgctgctgtcgtccggcataggcgaagcccccaaagaggcattgcaggaagctggcgtgctgcctattgtatgcggcggggatattgaggaatccgttctggagtatgtaaaatttctgcgttatatgtatcctgtgcagagcaataagggaagtaagcgcaataagggagttaagggcaatcattcggatttacccattgaacattttggaggctga

SEQ ID NO: 27 - DNA, 2012 bp, Artificial Sequence
  mutant nifB gene P(adhE)-nifB_v2
taagcggagactatttcccaaaatatataatgaaatttcttgtcttaaaaggagagccgtatttacggactctctttttttacgtcttgatgttattgagaatatgaaatgtaaccgcgcacatgtaaagtgtacgatatattacttgacgtaaaatttgacacatatgtgaaatgaggataaatatcagggatttcatggagaagcgaattgactgtatttgtccctgtctctaagatgtaattatattccagcgtccatttcccctttgtatcctatgttttttatctgcaacttgatcaattcaaattcattatatacctgtgcaatgagtttttggggcaagaagagggggtaaaagtggcagaattccaaactttgtgatttaattcacttcataaaataaaattttaaatagattgtgaaaaaaatcacaatatatcttgaaaagctgtgttacgatcagtgcgtgaacaagagatagtacacgagataacaggaggatgagggaaatggactctttagctgatctctcggaaacccccttagccttggaaaccctcagacgacatccctgttataacgaagaggcacatcgctattttgcgcgcattcatcttccagtagccccggcatgcaatattcagtgccattattgcaaccgcaaattcgattgcgtcaatgaaagccgtcccggcgttgttagtgaactgctcacgccggagcaggcggcgagcaagatctatggcgtagcggcacagctgatgcaactgtccgttgtcggcattgcgggacctggagatccgctggccaatgcggaggcaaccttcgataccttccgccgggtccgtgagacagttaaggatgtcatattctgtctcagcacgaatggccttaccttgatcaggcatatcgacaggattgtagagttgggtatttcgcatgtcacgatcacgatcaatgctgtagatccagtggtggggagccgcatttatggatgggtctacgatgaaggaaaacgctatgcaggagaggaggccgcgcggctgttgattgaccgccagctggcaggtttgaagatgctggcttcgagaggcgtattgtgcaaggtgaactcggtgctgattcccgaagtcaatgatgcccatctgccagaggtagccagggtggtcaaggagcacggcgcggtactgcacaacattatgccgctcatcatcgcacccggcagtcggtatgagcaggaagggatgcggcccccccgtccccgtctggtccggcagctgcaggagcaatgtgctgaggcgggagctgtcattatgcgccattgccgtcagtgcagggcggatgcgattggactgctgggcgaggatcgcaatcaggattttacatgggagaacattgcggctgctcctcccatggatgaagaggcaagggcacaatttcagaaagaactggatgagaaggtgagagtgagaatggaacgtaaggagggacaatcgcaccacaaaaaaacgtcaaccggggttggatgtagctgcccgttatcggaggataagcctgaagcaagctttacctcaaagccagtcctaatcgctgtggccagtcgtggcggagggaaggtgaatcagcatttcggtcgtgccaaggaattcatgatctatgaaagcgacgggactatcgtaaatttcataggcattcgtaaggtgcaatcctactgccatgggaaagccgattgcaatggagacaaggtcgagacgatgaaggagatcctttccatggtgcatgactgtgcattgctgctgtcgtccggcataggcgaagcccccaaagaggcattgcaggaagctggcgtgctgcctattgtatgcggcggggatattgaggaatccgttctggagtatgtaaaatttctgcgttatatgtatcctgtgcagagcaataagggaagtaagcgcaataagggagttaagggcaatcattcggatttacccattgaacattttggaggctga

SEQ ID NO: 28 - DNA, 1963 bp, Artificial Sequence
  mutant nifB gene P(adhE)-nifB_v3
taagcggagactatttcccaaaatatataatgaaatttcttgtcttaaaaggagagccgtatttacggactctctttttttacgtcttgatgttattgagaatatgaaatgtaaccgcgcacatgtaaagtgtacgatatattacttgacgtaaaatttgacacatatgtgaaatgaggataaatatcagggatttcatggagaacgtccatttcccctttgtatcctatgttttttatctgcaacttgatcaattcaaattcattatatacctgtgcaatgagtttttggggcaagaagagggggtaaaagtggcagaattccaaactttgtgatttaattcacttcataaaataaaattttaaatagattgtgaaaaaaatcacaatatatcttgaaaagctgtgttacgatcagtgcgtgaacaagagatagtacacgagataacaggaggatgagggaaatggactctttagctgatctctcggaaacccccttagccttggaaaccctcagacgacatccctgttataacgaagaggcacatcgctattttgcgcgcattcatcttccagtagccccggcatgcaatattcagtgccattattgcaaccgcaaattcgattgcgtcaatgaaagccgtcccggcgttgttagtgaactgctcacgccggagcaggcggcgagcaagatctatggcgtagcggcacagctgatgcaactgtccgttgtcggcattgcgggacctggagatccgctggccaatgcggaggcaaccttcgataccttccgccgggtccgtgagacagttaaggatgtcatattctgtctcagcacgaatggccttaccttgatcaggcatatcgacaggattgtagagttgggtatttcgcatgtcacgatcacgatcaatgctgtagatccagtggtggggagccgcatttatggatgggtctacgatgaaggaaaacgctatgcaggagaggaggccgcgcggctgttgattgaccgccagctggcaggtttgaagatgctggcttcgagaggcgtattgtgcaaggtgaactcggtgctgattcccgaagtcaatgatgcccatctgccagaggtagccagggtggtcaaggagcacggcgcggtactgcacaacattatgccgctcatcatcgcacccggcagtcggtatgagcaggaagggatgcggcccccccgtccccgtctggtccggcagctgcaggagcaatgtgctgaggcgggagctgtcattatgcgccattgccgtcagtgcagggcggatgcgattggactgctgggcgaggatcgcaatcaggattttacatgggagaacattgcggctgctcctcccatggatgaagaggcaagggcacaatttcagaaagaactggatgagaaggtgagagtgagaatggaacgtaaggagggacaatcgcaccacaaaaaaacgtcaaccggggttggatgtagctgcccgttatcggaggataagcctgaagcaagctttacctcaaagccagtcctaatcgctgtggccagtcgtggcggagggaaggtgaatcagcatttcggtcgtgccaaggaattcatgatctatgaaagcgacgggactatcgtaaatttcataggcattcgtaaggtgcaatcctactgccatgggaaagccgattgcaatggagacaaggtcgagacgatgaaggagatcctttccatggtgcatgactgtgcattgctgctgtcgtccggcataggcgaagcccccaaagaggcattgcaggaagctggcgtgctgcctattgtatgcggcggggatattgaggaatccgttctggagtatgtaaaatttctgcgttatatgtatcctgtgcagagcaataagggaagtaagcgcaataagggagttaagggcaatcattcggatttacccattgaacattttggaggctga

SEQ ID NO: 29 - DNA, 1943 bp, Artificial Sequence
  mutant nifB gene P(tig)-nifB_v1
taagcggagactatttcccaaaatatataatgaaatttcttgtcttaaaaggagagccgtatttacggactctctttttttacgtcttgatgttattgagaatatgaaatgtaaccgcgcacatgtaaagtgtacgatatattacttgacgtaaaatttgacacatatgtgaaatgaggataaatatcagggatttcatggagaagcgaattgactgtatttgtccctgtctctaagatgtaattatattccagacaaaaacagagaattgtgtaagggaatataacgtagagaggagggaatgaggaaacgtttacctgtgaagcattcaaagggctgctcttgaaaaagacagtaatgttagatatactatagtggttattttgtgtgcgttgattgtaatgaaaactaagtgaagattttgttggaggggaaagcataaaatggactctttagctgatctctcggaaacccccttagccttggaaaccctcagacgacatccctgttataacgaagaggcacatcgctattttgcgcgcattcatcttccagtagccccggcatgcaatattcagtgccattattgcaaccgcaaattcgattgcgtcaatgaaagccgtcccggcgttgttagtgaactgctcacgccggagcaggcggcgagcaagatctatggcgtagcggcacagctgatgcaactgtccgttgtcggcattgcgggacctggagatccgctggccaatgcggaggcaaccttcgataccttccgccgggtccgtgagacagttaaggatgtcatattctgtctcagcacgaatggccttaccttgatcaggcatatcgacaggattgtagagttgggtatttcgcatgtcacgatcacgatcaatgctgtagatccagtggtggggagccgcatttatggatgggtctacgatgaaggaaaacgctatgcaggagaggaggccgcgcggctgttgattgaccgccagctggcaggtttgaagatgctggcttcgagaggcgtattgtgcaaggtgaactcggtgctgattcccgaagtcaatgatgcccatctgccagaggtagccagggtggtcaaggagcacggcgcggtactgcacaacattatgccgctcatcatcgcacccggcagtcggtatgagcaggaagggatgcggcccccccgtccccgtctggtccggcagctgcaggagcaatgtgctgaggcgggagctgtcattatgcgccattgccgtcagtgcagggcggatgcgattggactgctgggcgaggatcgcaatcaggattttacatgggagaacattgcggctgctcctcccatggatgaagaggcaagggcacaatttcagaaagaactggatgagaaggtgagagtgagaatggaacgtaaggagggacaatcgcaccacaaaaaaacgtcaaccggggttggatgtagctgcccgttatcggaggataagcctgaagcaagctttacctcaaagccagtcctaatcgctgtggccagtcgtggcggagggaaggtgaatcagcatttcggtcgtgccaaggaattcatgatctatgaaagcgacgggactatcgtaaatttcataggcattcgtaaggtgcaatcctactgccatgggaaagccgattgcaatggagacaaggtcgagacgatgaaggagatcctttccatggtgcatgactgtgcattgctgctgtcgtccggcataggcgaagcccccaaagaggcattgcaggaagctggcgtgctgcctattgtatgcggcggggatattgaggaatccgttctggagtatgtaaaatttctgcgttatatgtatcctgtgcagagcaataagggaagtaagcgcaataagggagttaagggcaatcattcggatttacccattgaacattttggaggctga

SEQ ID NO: 30 - DNA, 1892 bp, Artificial Sequence
  mutant nifB gene P(tig)-nifB_v2
taagcggagactatttcccaaaatatataatgaaatttcttgtcttaaaaggagagccgtatttacggactctctttttttacgtcttgatgttattgagaatatgaaatgtaaccgcgcacatgtaaagtgtacgatatattacttgacgtaaaatttgacacatatgtgaaatgaggataaatatcagggatttcatggagaagcgaattgactgtatttgtccctgtctctaagatgtaattatattccagggaaacgtttacctgtgaagcattcaaagggctgctcttgaaaaagacagtaatgttagatatactatagtggttattttgtgtgcgttgattgtaatgaaaactaagtgaagattttgttggaggggaaagcataaaatggactctttagctgatctctcggaaacccccttagccttggaaaccctcagacgacatccctgttataacgaagaggcacatcgctattttgcgcgcattcatcttccagtagccccggcatgcaatattcagtgccattattgcaaccgcaaattcgattgcgtcaatgaaagccgtcccggcgttgttagtgaactgctcacgccggagcaggcggcgagcaagatctatggcgtagcggcacagctgatgcaactgtccgttgtcggcattgcgggacctggagatccgctggccaatgcggaggcaaccttcgataccttccgccgggtccgtgagacagttaaggatgtcatattctgtctcagcacgaatggccttaccttgatcaggcatatcgacaggattgtagagttgggtatttcgcatgtcacgatcacgatcaatgctgtagatccagtggtggggagccgcatttatggatgggtctacgatgaaggaaaacgctatgcaggagaggaggccgcgcggctgttgattgaccgccagctggcaggtttgaagatgctggcttcgagaggcgtattgtgcaaggtgaactcggtgctgattcccgaagtcaatgatgcccatctgccagaggtagccagggtggtcaaggagcacggcgcggtactgcacaacattatgccgctcatcatcgcacccggcagtcggtatgagcaggaagggatgcggcccccccgtccccgtctggtccggcagctgcaggagcaatgtgctgaggcgggagctgtcattatgcgccattgccgtcagtgcagggcggatgcgattggactgctgggcgaggatcgcaatcaggattttacatgggagaacattgcggctgctcctcccatggatgaagaggcaagggcacaatttcagaaagaactggatgagaaggtgagagtgagaatggaacgtaaggagggacaatcgcaccacaaaaaaacgtcaaccggggttggatgtagctgcccgttatcggaggataagcctgaagcaagctttacctcaaagccagtcctaatcgctgtggccagtcgtggcggagggaaggtgaatcagcatttcggtcgtgccaaggaattcatgatctatgaaagcgacgggactatcgtaaatttcataggcattcgtaaggtgcaatcctactgccatgggaaagccgattgcaatggagacaaggtcgagacgatgaaggagatcctttccatggtgcatgactgtgcattgctgctgtcgtccggcataggcgaagcccccaaagaggcattgcaggaagctggcgtgctgcctattgtatgcggcggggatattgaggaatccgttctggagtatgtaaaatttctgcgttatatgtatcctgtgcagagcaataagggaagtaagcgcaataagggagttaagggcaatcattcggatttacccattgaacattttggaggctga

SEQ ID NO: 31 - DNA, 1843 bp, Artificial Sequence
  mutant nifB gene P(tig)-nifB_v3
taagcggagactatttcccaaaatatataatgaaatttcttgtcttaaaaggagagccgtatttacggactctctttttttacgtcttgatgttattgagaatatgaaatgtaaccgcgcacatgtaaagtgtacgatatattacttgacgtaaaatttgacacatatgtgaaatgaggataaatatcagggatttcatggagaaggaaacgtttacctgtgaagcattcaaagggctgctcttgaaaaagacagtaatgttagatatactatagtggttattttgtgtgcgttgattgtaatgaaaactaagtgaagattttgttggaggggaaagcataaaatggactctttagctgatctctcggaaacccccttagccttggaaaccctcagacgacatccctgttataacgaagaggcacatcgctattttgcgcgcattcatcttccagtagccccggcatgcaatattcagtgccattattgcaaccgcaaattcgattgcgtcaatgaaagccgtcccggcgttgttagtgaactgctcacgccggagcaggcggcgagcaagatctatggcgtagcggcacagctgatgcaactgtccgttgtcggcattgcgggacctggagatccgctggccaatgcggaggcaaccttcgataccttccgccgggtccgtgagacagttaaggatgtcatattctgtctcagcacgaatggccttaccttgatcaggcatatcgacaggattgtagagttgggtatttcgcatgtcacgatcacgatcaatgctgtagatccagtggtggggagccgcatttatggatgggtctacgatgaaggaaaacgctatgcaggagaggaggccgcgcggctgttgattgaccgccagctggcaggtttgaagatgctggcttcgagaggcgtattgtgcaaggtgaactcggtgctgattcccgaagtcaatgatgcccatctgccagaggtagccagggtggtcaaggagcacggcgcggtactgcacaacattatgccgctcatcatcgcacccggcagtcggtatgagcaggaagggatgcggcccccccgtccccgtctggtccggcagctgcaggagcaatgtgctgaggcgggagctgtcattatgcgccattgccgtcagtgcagggcggatgcgattggactgctgggcgaggatcgcaatcaggattttacatgggagaacattgcggctgctcctcccatggatgaagaggcaagggcacaatttcagaaagaactggatgagaaggtgagagtgagaatggaacgtaaggagggacaatcgcaccacaaaaaaacgtcaaccggggttggatgtagctgcccgttatcggaggataagcctgaagcaagctttacctcaaagccagtcctaatcgctgtggccagtcgtggcggagggaaggtgaatcagcatttcggtcgtgccaaggaattcatgatctatgaaagcgacgggactatcgtaaatttcataggcattcgtaaggtgcaatcctactgccatgggaaagccgattgcaatggagacaaggtcgagacgatgaaggagatcctttccatggtgcatgactgtgcattgctgctgtcgtccggcataggcgaagcccccaaagaggcattgcaggaagctggcgtgctgcctattgtatgcggcggggatattgaggaatccgttctggagtatgtaaaatttctgcgttatatgtatcctgtgcagagcaataagggaagtaagcgcaataagggagttaagggcaatcattcggatttacccattgaacattttggaggctga

SEQ ID NO: 32 - DNA, 1822 bp, Artificial Sequence
  mutant nifB gene P(pflB)-nifB_v0
aggtaagaagaggcttgtgaaaaagcattttctcgttcaataccgatgcaggctctctatatagagggcctgttggtttgggaagggaggtgaacggcaaggctaaggatataggcgcgaagctagggaaaataaagtgacaaaaatcactcatattgtgatttttgtcacatactacactctatcattattctacaataagggtgtaagaaaggtcccgacactgagcgtaaggcatcggtgaacaggccgggctcaagaaatagctttatcgtgaaaaaaatcacaagtccggtagacgaaacaatttggagggatttacatggactctttagctgatctctcggaaacccccttagccttggaaaccctcagacgacatccctgttataacgaagaggcacatcgctattttgcgcgcattcatcttccagtagccccggcatgcaatattcagtgccattattgcaaccgcaaattcgattgcgtcaatgaaagccgtcccggcgttgttagtgaactgctcacgccggagcaggcggcgagcaagatctatggcgtagcggcacagctgatgcaactgtccgttgtcggcattgcgggacctggagatccgctggccaatgcggaggcaaccttcgataccttccgccgggtccgtgagacagttaaggatgtcatattctgtctcagcacgaatggccttaccttgatcaggcatatcgacaggattgtagagttgggtatttcgcatgtcacgatcacgatcaatgctgtagatccagtggtggggagccgcatttatggatgggtctacgatgaaggaaaacgctatgcaggagaggaggccgcgcggctgttgattgaccgccagctggcaggtttgaagatgctggcttcgagaggcgtattgtgcaaggtgaactcggtgctgattcccgaagtcaatgatgcccatctgccagaggtagccagggtggtcaaggagcacggcgcggtactgcacaacattatgccgctcatcatcgcacccggcagtcggtatgagcaggaagggatgcggcccccccgtccccgtctggtccggcagctgcaggagcaatgtgctgaggcgggagctgtcattatgcgccattgccgtcagtgcagggcggatgcgattggactgctgggcgaggatcgcaatcaggattttacatgggagaacattgcggctgctcctcccatggatgaagaggcaagggcacaatttcagaaagaactggatgagaaggtgagagtgagaatggaacgtaaggagggacaatcgcaccacaaaaaaacgtcaaccggggttggatgtagctgcccgttatcggaggataagcctgaagcaagctttacctcaaagccagtcctaatcgctgtggccagtcgtggcggagggaaggtgaatcagcatttcggtcgtgccaaggaattcatgatctatgaaagcgacgggactatcgtaaatttcataggcattcgtaaggtgcaatcctactgccatgggaaagccgattgcaatggagacaaggtcgagacgatgaaggagatcctttccatggtgcatgactgtgcattgctgctgtcgtccggcataggcgaagcccccaaagaggcattgcaggaagctggcgtgctgcctattgtatgcggcggggatattgaggaatccgttctggagtatgtaaaatttctgcgttatatgtatcctgtgcagagcaataagggaagtaagcgcaataagggagttaagggcaatcattcggatttacccattgaacattttggaggctga

SEQ ID NO: 33 - DNA, 1758 bp, Artificial Sequence
  mutant nifB gene P(adhe)-nifB_v0
cgtccatttcccctttgtatcctatgttttttatctgcaacttgatcaattcaaattcattatatacctgtgcaatgagtttttggggcaagaagagggggtaaaagtggcagaattccaaactttgtgatttaattcacttcataaaataaaattttaaatagattgtgaaaaaaatcacaatatatcttgaaaagctgtgttacgatcagtgcgtgaacaagagatagtacacgagataacaggaggatgagggaaatggactctttagctgatctctcggaaacccccttagccttggaaaccctcagacgacatccctgttataacgaagaggcacatcgctattttgcgcgcattcatcttccagtagccccggcatgcaatattcagtgccattattgcaaccgcaaattcgattgcgtcaatgaaagccgtcccggcgttgttagtgaactgctcacgccggagcaggcggcgagcaagatctatggcgtagcggcacagctgatgcaactgtccgttgtcggcattgcgggacctggagatccgctggccaatgcggaggcaaccttcgataccttccgccgggtccgtgagacagttaaggatgtcatattctgtctcagcacgaatggccttaccttgatcaggcatatcgacaggattgtagagttgggtatttcgcatgtcacgatcacgatcaatgctgtagatccagtggtggggagccgcatttatggatgggtctacgatgaaggaaaacgctatgcaggagaggaggccgcgcggctgttgattgaccgccagctggcaggtttgaagatgctggcttcgagaggcgtattgtgcaaggtgaactcggtgctgattcccgaagtcaatgatgcccatctgccagaggtagccagggtggtcaaggagcacggcgcggtactgcacaacattatgccgctcatcatcgcacccggcagtcggtatgagcaggaagggatgcggcccccccgtccccgtctggtccggcagctgcaggagcaatgtgctgaggcgggagctgtcattatgcgccattgccgtcagtgcagggcggatgcgattggactgctgggcgaggatcgcaatcaggattttacatgggagaacattgcggctgctcctcccatggatgaagaggcaagggcacaatttcagaaagaactggatgagaaggtgagagtgagaatggaacgtaaggagggacaatcgcaccacaaaaaaacgtcaaccggggttggatgtagctgcccgttatcggaggataagcctgaagcaagctttacctcaaagccagtcctaatcgctgtggccagtcgtggcggagggaaggtgaatcagcatttcggtcgtgccaaggaattcatgatctatgaaagcgacgggactatcgtaaatttcataggcattcgtaaggtgcaatcctactgccatgggaaagccgattgcaatggagacaaggtcgagacgatgaaggagatcctttccatggtgcatgactgtgcattgctgctgtcgtccggcataggcgaagcccccaaagaggcattgcaggaagctggcgtgctgcctattgtatgcggcggggatattgaggaatccgttctggagtatgtaaaatttctgcgttatatgtatcctgtgcagagcaataagggaagtaagcgcaataagggagttaagggcaatcattcggatttacccattgaacattttggaggctga

SEQ ID NO: 34 - DNA, 1638 bp, Artificial Sequence
  mutant nifB gene P(tig)-nifB_v0
ggaaacgtttacctgtgaagcattcaaagggctgctcttgaaaaagacagtaatgttagatatactatagtggttattttgtgtgcgttgattgtaatgaaaactaagtgaagattttgttggaggggaaagcataaaatggactctttagctgatctctcggaaacccccttagccttggaaaccctcagacgacatccctgttataacgaagaggcacatcgctattttgcgcgcattcatcttccagtagccccggcatgcaatattcagtgccattattgcaaccgcaaattcgattgcgtcaatgaaagccgtcccggcgttgttagtgaactgctcacgccggagcaggcggcgagcaagatctatggcgtagcggcacagctgatgcaactgtccgttgtcggcattgcgggacctggagatccgctggccaatgcggaggcaaccttcgataccttccgccgggtccgtgagacagttaaggatgtcatattctgtctcagcacgaatggccttaccttgatcaggcatatcgacaggattgtagagttgggtatttcgcatgtcacgatcacgatcaatgctgtagatccagtggtggggagccgcatttatggatgggtctacgatgaaggaaaacgctatgcaggagaggaggccgcgcggctgttgattgaccgccagctggcaggtttgaagatgctggcttcgagaggcgtattgtgcaaggtgaactcggtgctgattcccgaagtcaatgatgcccatctgccagaggtagccagggtggtcaaggagcacggcgcggtactgcacaacattatgccgctcatcatcgcacccggcagtcggtatgagcaggaagggatgcggcccccccgtccccgtctggtccggcagctgcaggagcaatgtgctgaggcgggagctgtcattatgcgccattgccgtcagtgcagggcggatgcgattggactgctgggcgaggatcgcaatcaggattttacatgggagaacattgcggctgctcctcccatggatgaagaggcaagggcacaatttcagaaagaactggatgagaaggtgagagtgagaatggaacgtaaggagggacaatcgcaccacaaaaaaacgtcaaccggggttggatgtagctgcccgttatcggaggataagcctgaagcaagctttacctcaaagccagtcctaatcgctgtggccagtcgtggcggagggaaggtgaatcagcatttcggtcgtgccaaggaattcatgatctatgaaagcgacgggactatcgtaaatttcataggcattcgtaaggtgcaatcctactgccatgggaaagccgattgcaatggagacaaggtcgagacgatgaaggagatcctttccatggtgcatgactgtgcattgctgctgtcgtccggcataggcgaagcccccaaagaggcattgcaggaagctggcgtgctgcctattgtatgcggcggggatattgaggaatccgttctggagtatgtaaaatttctgcgttatatgtatcctgtgcagagcaataagggaagtaagcgcaataagggagttaagggcaatcattcggatttacccattgaacattttggaggctga

SEQ ID NO: 35 - protein, 137 aa, Paenibacillus polymyxa; identical to SEQ ID NO: 16

SEQ ID NO: 36 - protein, 137 aa, Paenibacillus peoriae; identical to SEQ ID NO: 16

SEQ ID NO: 37 - protein, 137 aa, Paenibacillus kribbensis; identical to SEQ ID NO: 16

SEQ ID NO: 38 - protein, 136 aa, Paenibacillus sp. FSL
MGDEIRRNMALFPIGIVMKLTDLSARQIRYYEQHSLIVPARTSGNQRLFSFNDVERLLEIKALIEKGVNIAGIKQVMNPVSKESEEATVITPDTEVRRRELSDSQLHRLLKQELVSGKRPGQVSLIQGELSRFFNK

SEQ ID NO: 39 - protein, 136 aa, Paenibacillus sabinae; identical to SEQ ID NO: 38

SEQ ID NO: 40 - protein, 136 aa, Paenibacillus durus; identical to SEQ ID NO: 38

SEQ ID NO: 41 - protein, 136 aa, Paenibacillus borealis; identical to SEQ ID NO: 38

SEQ ID NO: 42 - protein, 136 aa, Paenibacillus graminis; identical to SEQ ID NO: 38

SEQ ID NO: 43 - protein, 136 aa, Paenibacillus odorifer; identical to SEQ ID NO: 38

SEQ ID NO: 44 - protein, 136 aa, Paenibacillus stellifer; identical to SEQ ID NO: 38

SEQ ID NO: 45 - protein, 136 aa, Paenibacillus riogranensis; identical to SEQ ID NO: 38

SEQ ID NO: 46 - protein, 136 aa, Paenibacillus donghaensis
MGDEIRRNMALFPIGIVMKLTDLSARQIRYYEQHSLIVPARTSGNQRLFSFNDVERLLEIKALIEKGVNIAGIKQVMNPVTRESDEATVITPDTEVRRRELSESQLHRLLKQELVSGKRPGQVSLIQGELSRFFNK

SEQ ID NO: 47 - protein, 9 aa, Unknown
  Meganuclease peptide motif
LAGLIDADG

SEQ ID NO: 48 - DNA, 1329 bp, Paenibacillus polymyxa
gtgagttatagcagagaagatatccttcggattgcgaaagaggaaaatgttcgttttattcgtttgcaattcacagatttgctgggtactattaagaacgttgaaattccggttagtcaactggaaaaagcattggataataaaatgatgtttgacggatcttctatagaaggttatgtgcgtattgaagaatctgatatgtatttatatccggatttggacacttgggttgtattcccttgggtaacctcagatcgtgtagctcgtttgatctgcgatatttacaagccggatggagtcccatttgcaggtgacccacggggcatcttgaaacgtgtacttaaggaagccgaagagctgggatacacttcgatgaatgtcggacccgagcctgaattctttttgttcaaaacggatgaaaaaggcgagccgactacagaactgaatgaccagggcgggtattttgacctggctccgatggatctgggtgaaaactgccgtcgtgaaattgtacttaagcttgaagagatgggcttcgaaattgaagcatcccatcatgaagttgcacctggtcagcatgagattgactttaaatatgcagacgcgattaaagctgcggaccagattcaaacgttcaagctcgttgttaagacgattgctcgtcagcatggtttgcatgctacctttatgccaaaacctttgtttggtgtaaatggttctggtatgcactgcaatcaatcgttgtttaaggacaatgaaaacgtattttatgatgaaacggacgaactcggattaagccagacggcccgccactatatggctggtattctgaaacatgcacgtgcaatggcagcgatcacgaatccaacagtaaattcctacaaacgtctcgtacctggttatgaagcgccttgttatgtggcttggtctgcaagcaatcgcagtccaatgattcggattcctgcgtctcgtggtctgagcactcgcgtcgaagttcgtaaccctgacccggcggcgaatccttatttagccttggcagtgatgctgagagctggtttagatgggatcaaacgtcagttggctctcccagctccgattgatcgtaacatttacgtgatgtctgaggaagagcggattgaagaaggcattccaagtctgcctgcagacttgaaagaagctttgtctgaacttattcgtagcgaagttatctctgacgcactgggtgaccatgcattggcctacttctatgagcttaaggaaattgaatgggatatgtatcgcacacaagtccatcaatgggaacgtgatcaatatctgacgctttactaa

SEQ ID NO: 49 - DNA, 1425 bp, Paenibacillus polymyxa
atgtccgttgaaaacgtattgaaaacaattcaggaaaataatattgagtgggtagattttcgttttgtagatttgtctggtcgtgctcaccatatttcattgccagcttccgaagtagatgaagagacatttgttaacggtgttgctttcgacggttcttccattcccggctatcgtggcattgaggaatccgacatggttatgctgcccgatccagaagcggtttttatcgaccctttcactcagcatcctacactgaatatcctgtgtgacattgctacgccagacggcgaaaaatacgaccgcgatcctcgcggcatcgcaaaaaaggctgaggcatttctgcaatcggcaggagtgggcacagcagcattttttgcacctgagtccgagtttttcatctttgatgatgtacgctacgaaagcggcatgaacagctcctccttcttcgtagattccgaggaagcagcttggaacacgaaccgtaaggaagaaggcggtaacctgggctttaaagtgggagtgaagggcgggtatgtacctgtagcgccagtagacacccaacaagatatccgtagcgaaatgtgccgtttgcttgaagaagcaggtctgagaattgagcgccatcaccatgaggtagctacggcaggccaagcggaaatcaacttccgttttgatacactcaagaaaacagcagataacctgctcgtttataaatacattgtacacaacacagctcgtcaatatggtaaagtggcaacattcatgccgaaaccgatttttggggataacggaagcggtatgcacgttcaccaatctattttcgatggggacactcctttgttctacgaaaaaggtggttatgctaatctgagcgaaatggctcttcattacattggcggtattctgtaccacgcacctgcgttgatcgctttgaccaacccaagcaccaactcgttcaaacgtctcgttcctggctacgaagcaccagttaacctggtattctccaaaggtaaccgttctgcggctgtacgtattccagtagcagctgttacccctaaaggctgtcggatcgagttccgtacaccggactccacggctaacccttaccttgccttctcagcaatgctgatggcgggtctggatggcatcaagcgcaaactgaacccaatcgaattgggatatggtccacttgacagcaacatctacgagctgtctgatgctgaaaaaggtaaaatccgtagcgtacctggttcattggatgaggctctggacgctctggaagctgactacgaattcttgactgaaggcggcgtatttacgaaggactttattgataactatgtagagctcaaacgttctgaagctaaatcggtgaacatccgtgttcatccacacgaatacagcctgtattttgactgctaa

SEQ ID NO: 50 - DNA, 1410 bp, Klebsiella variicola
atgtccgctgaacacgttttgacgatgctgaacgagcacgaagtgaagtttgtcgatctgcgcttcaccgataccaaaggtaaagaacagcacgtcacgattccgtctcatcaggtaaatgccgaattcttcgaagaaggcaaaatgtttgatggctcctcgatcggtggctggaaaggtatcaacgaatctgacatggtcctgatgccggacgcttccaccgcggtcattgacccgttctacgaagaaccgaccctgatcatccgctgcgatatcctcgagccaggcaccctgcagggctatgaccgtgacccgcgctccatcgcgaaacgcgctgaagagtacctgcgcgccaccggcatcgctgacaccgtcctgttcgggccagaaccagaattcttcctgttcgacgacatccgttttggcgcctctatctccggctcccatgtcgcgatcgatgacatcgaaggtgcgtggaactcctccaccaaatacgaaggcggtaacaaaggtcaccgtccgggcgtgaaagggggttacttcccggttccgccagtagactcttctcaggatatccgttccaccatgtgtatgatcatggaagagatgggcctggttgttgaagctcaccaccacgaagtggctaccgcaggtcagaacgaagtggcaacccgcttcaacaccatgaccaaaaaagcggacgaaattcagatttacaaatacgttgttcacaacgtcgctcaccgcttcggtaaaaccgcgacctttatgccgaaaccaatgttcggcgataacggctccggcatgcactgccatatgtctctggcgaaaaacgggactaacctgttctccggcgacaaatacgcaggtctgtctgagcaggcgctgttctacatcggcggcgtaatcaaacacgctaaagctatcaacgccctggcgaacccgaccaccaactcctacaagcgtctggtcccgggttacgaagcgccggttatgctggcttactctgcgcgtaaccgctctgcctccatccgtattccggtggtgacctctccgaaagcgcgtcgtatcgaagtgcgcttcccggacccggctgccaacccgtacctgtgctttgcggcgctgctgatggctggccttgatggtatcaagaacaaaatccatccgggcgaagcaatggacaaaaacctgtatgacctgccgccggaagaagcgaaagagatcccgcaggttgcaggctctctggaagaagccctgcaggccctggatgctgaccgcgagttcctgacggctggcggcgtgttcaccaatgacgctatcgatgcttacatcgccctgcgtctggaagagaacgaccgcgtacgcatgactccgcatccggttgagttcgaactgtactacagcgtctaa

SEQ ID NO: 51 - protein, 442 aa, Paenibacillus polymyxa
MSYSREDILRIAKEENVRFIRLQFTDLLGTIKNVEIPVSQLEKALDNKMMFDGSSIEGYVRIEESDMYLYPDLDTWVVFPWVTSDRVARLICDIYKPDGVPFAGDPRGILKRVLKEAEELGYTSMNVGPEPEFFLFKTDEKGEPTTELNDQGGYFDLAPMDLGENCRREIVLKLEEMGFEIEASHHEVAPGQHEIDFKYADAIKAADQIQTFKLVVKTIARQHGLHATFMPKPLFGVNGSGMHCNQSLFKDNENVFYDETDELGLSQTARHYMAGILKHARAMAAITNPTVNSYKRLVPGYEAPCYVAWSASNRSPMIRIPASRGLSTRVEVRNPDPAANPYLALAVMLRAGLDGIKRQLALPAPIDRNIYVMSEEERIEEGIPSLPADLKEALSELIRSEVISDALGDHALAYFYELKEIEWDMYRTQVHQWERDQYLTLY

SEQ ID NO: 52 - protein, 474 aa, Paenibacillus polymyxa
MSVENVLKTIQENNIEWVDFRFVDLSGRAHHISLPASEVDEETFVNGVAFDGSSIPGYRGIEESDMVMLPDPEAVFIDPFTQHPTLNILCDIATPDGEKYDRDPRGIAKKAEAFLQSAGVGTAAFFAPESEFFIFDDVRYESGMNSSSFFVDSEEAAWNTNRKEEGGNLGFKVGVKGGYVPVAPVDTQQDIRSEMCRLLEEAGLRIERHHHEVATAGQAEINFRFDTLKKTADNLLVYKYIVHNTARQYGKVATFMPKPIFGDNGSGMHVHQSIFDGDTPLFYEKGGYANLSEMALHYIGGILYHAPALIALTNPSTNSFKRLVPGYEAPVNLVFSKGNRSAAVRIPVAAVTPKGCRIEFRTPDSTANPYLAFSAMLMAGLDGIKRKLNPIELGYGPLDSNIYELSDAEKGKIRSVPGSLDEALDALEADYEFLTEGGVFTKDFIDNYVELKRSEAKSVNIRVHPHEYSLYFDC

SEQ ID NO: 53 - protein, 469 aa, Klebsiella variicola
MSAEHVLTMLNEHEVKFVDLRFTDTKGKEQHVTIPSHQVNAEFFEEGKMFDGSSIGGWKGINESDMVLMPDASTAVIDPFYEEPTLIIRCDILEPGTLQGYDRDPRSIAKRAEEYLRATGIADTVLFGPEPEFFLFDDIRFGASISGSHVAIDDIEGAWNSSTKYEGGNKGHRPGVKGGYFPVPPVDSSQDIRSTMCMIMEEMGLVVEAHHHEVATAGQNEVATRFNTMTKKADEIQIYKYVVHNVAHRFGKTATFMPKPMFGDNGSGMHCHMSLAKNGTNLFSGDKYAGLSEQALFYIGGVIKHAKAINALANPTTNSYKRLVPGYEAPVMLAYSARNRSASIRIPVVTSPKARRIEVRFPDPAANPYLCFAALLMAGLDGIKNKIHPGEAMDKNLYDLPPEEAKEIPQVAGSLEEALQALDADREFLTAGGVFTNDAIDAYIALRLEENDRVRMTPHPVEFELYYSV

(19) United States
(12) Patent Application Publication
(10) Pub. No.: US 2023/0295559 A1
(43) Pub. Date: Sep. 21, 2023

(54) DE-REPRESSION OF NITROGEN FIXATION IN GRAM-POSITIVE MICROORGANISMS

(71) Applicant: Pivot Bio, Inc., Berkeley, CA (US)

(72) Inventors: Bilge Ozaydin Eskiyenenturk, Berkeley, CA (US); Min-Hyung Ryu, Berkeley, CA (US); Jenny Johnson, Berkeley, CA (US)

(73) Assignee: Pivot Bio, Inc., Berkeley, CA (US)

(21) Appl. No.: 17/924,916

(22) PCT Filed: May 11, 2021
(86) PCT No.: PCT/US2021/031808

§ 371 Date: Nov. 11, 2022

Related U.S. Application Data
(60) Provisional application No. 63/024,208, filed on May 13, 2020.

(51) Int. Cl. C12N 1/20; A01N 63/25

(52) U.S. Cl. CPC C12N 1/205; A01N 63/25; C12R 2001/12

(57) ABSTRACT

The present disclosure provides engineered gram-positive microbes that are able to fix atmospheric nitrogen and deliver such to plants in a targeted, efficient, and environmentally sustainable manner. The utilization of the taught microbial products will enable farmers to realize more productive and predictable crop yields without the nutrient degradation, leaching, or toxic runoff associated with traditional synthetically derived nitrogen fertilizer.

### CROSS-REFERENCE

This application claims priority to U.S. Provisional Patent Application No. 63/024,208, filed on May 13, 2020, which is entirely incorporated herein by reference.

### STATEMENT REGARDING SEQUENCE LISTING

The contents of the text file submitted electronically herewith are incorporated herein by reference in their entirety: A computer readable format copy of the Sequence Listing filename: sequencelisting.txt, date created, May 11, 2021, file size 97.1 KB.

### BACKGROUND OF THE DISCLOSURE

One of the major agricultural inputs needed to satisfy global food demand is nitrogen fertilizer. However, the current industrial standard utilized to produce nitrogen fertilizer, is an artificial nitrogen fixation method called the Haber-Bosch process, which converts atmospheric nitrogen (N₂) to ammonia (NH₃) by a reaction with hydrogen (H₂) using a metal catalyst under high temperatures and pressures. This process is resource intensive and deleterious to the environment. Furthermore, the nitrogen fertilizer produced by the industrial Haber-Bosch process is not well utilized by target crops. Rain, runoff, heat, volatilization, and the soil microbiome degrade the applied chemical fertilizer. This equates to not only wasted money, but also adds to increased pollution instead of harvested yield. To this end, the United Nations has calculated that nearly 80% of fertilizer is lost before a crop can utilize it. Consequently, modern agricultural fertilizer production and delivery is not only deleterious to the environment, but it is extremely inefficient.

In contrast to the synthetic Haber-Bosch process, certain biological systems have evolved to fix atmospheric nitrogen. These systems utilize an enzyme called nitrogenase that catalyzes the reaction between N₂ and H₂, and results in nitrogen fixation. For example, rhizobia are diazotrophic bacteria that fix nitrogen after becoming established inside root nodules of legumes. An important goal of nitrogen fixation research is the extension of this phenotype to non-leguminous plants, particularly to important agronomic grasses such as wheat, rice, and corn. However, despite the significant progress made in understanding the development of the nitrogen-fixing symbiosis between rhizobia and legumes, the path to use that knowledge to induce nitrogen-fixing nodules on non-leguminous crops is still not clear.

Currently, in order to address this urgent need, efforts have been made to engineer or remodel other microorganisms that are not rhizobia in order to extend the nitrogen fixation phenotype to non-leguminous plants. To this end, a number of gram-negative microorganisms have engineered or remodeled, but they have often been found to be less stable than gram-positive microorganisms. Diazotrophic gram-positive microorganisms are known to exist in nature; however, the nitrogen fixation pathway in said gram-positive microorganisms is tightly regulated such that the levels of fixed nitrogen often present in the environment in which non-leguminous food crops are grown is more than sufficient to repress the expression and/or activity of nitrogenase in these gram-positive microorganisms. Accordingly, provided herein are methods and compositions that allow for the provision of nitrogen to plants in the field by nitrogen fixing gram-positive microorganisms irrespective of the levels of fixed nitrogen present.

### SUMMARY OF THE DISCLOSURE

In one aspect, provided herein is an engineered gram-positive diazotrophic bacterium capable of fixing nitrogen irrespective of exogenous nitrogen levels at a rate at least equivalent to a rate of nitrogen fixation in a wild-type form of the gram-positive diazotrophic bacterium in the absence of exogenous nitrogen. In some cases, comprising a heterologous promoter operably linked to a mf operon and/or a mutant glnR gene, wherein the heterologous promoter replaces at least a portion of the nif operon endogenous promoter and promotes expression of the nif operon irrespective of nitrogen levels, and wherein the mutant glnR gene encodes a mutant GlnR protein that promotes expression of the nif operon irrespective of nitrogen levels. In some cases, the heterologous promoter completely replaces the mf operon endogenous promoter. In some cases, the heterologous promoter replaces a portion of the nif operon endogenous promoter downstream of a GlnR activator site, endogenous transcription start site and a GlnR repressor site. In some cases, the heterologous promoter replaces a portion of the nif operon endogenous promoter downstream of a GlnR activator site and endogenous transcription start site. In some cases, the heterologous promoter replaces a portion of the nif operon endogenous promoter downstream of a GlnR activator site. In some cases, the heterologous promoter is selected from a promoter for a Paenibacillus Acetolactate synthase (alsS) gene, Pyruvate formate-lyase-activating enzyme (pflB) gene, D-alanine aminotransferase (dat) gene, 30S ribosomal protein S21 (rpsU) gene, Aldehyde-alcohol dehydrogenase (adhe) gene, 50S ribosomal protein L13 (rplm) gene, 50S ribosomal protein L36 (rpmJ) gene, DNA-binding protein HU 1 (hupA) gene, Translation initiation factor IF-3 (infC) gene, ECF RNA polymerase sigma-E factor (rpoE) gene, and Trigger factor (tig) gene. In some cases, the heterologous promoter has a nucleic acid sequence selected from SEQ ID NOs: 1-11. In some cases, the engineered gram-positive diazotrophic bacterium is selected from the group consisting of strain 41-2753, 41-2755, 41-4230, 41-4231, 41-4232, 41-4233 and 41-4236.

In some cases, the mutant glnR gene comprises at least one nucleotide substitution at nucleotide position 45, 46, 52, 111, 160, 272, 296, 316, 341, 347, 365, 382, 384 or 397 of a Paenibacillus glnR gene (e.g., SEQ ID NO:12) or at a homologous nucleotide position in a homolog thereof. In some cases, the mutant glnR gene shares at least 85%, 86%, 87%, 88%, 89%, 90%, 91%, 92%, 93%, 94%, 95%, 96%, 97%, 98%, or 99% identity with the Paenibacillus glnR gene (e.g., SEQ ID NO:12) or the homolog thereof. In some cases, the mutant GlnR protein comprises at least one amino acid substitution of at amino acid position 16, 18, 37, 54, 91, 99, 106, 114, 116, 122, 128 or 133 of a Paenibacillus GlnR protein (e.g., SEQ ID NO:16) or at a homologous amino acid position in a homolog thereof. In some cases, the mutant GlnR protein comprises at least one amino acid substitution selected from the group consisting of I16V, M18V, I37M, V54I, T91I, R99H, L106F, L114P, A116V, Q122R, G128S and F133L of a Paenibacillus GlnR protein or at a homologous amino acid position in a homolog thereof. In some cases, the mutant GlnR protein shares at least 90%, 91%, 92%, 93%, 94%, 95%, 96%, 97%, 98%, or 99% identity with the Paenibacillus GlnR protein or the homolog thereof. In some cases, the mutant GlnR protein comprises an L to P mutation at position 114 of a Paenibacillus GlnR protein or at a homologous amino acid position in a homolog thereof. In some cases, the mutant GlnR protein comprises a L114P mutation and one or more of a R99H mutation, an A116V mutation, a F133L mutation, an i16V mutation, a T91I mutation, a L106F mutation, a G128S mutation, a M18V mutation, an I37M mutation, a V54I mutation, a Q122R mutation and any combination thereof of a Paenibacillus GlnR protein or at a homologous amino acid position in a homolog thereof. In some cases, the mutant GlnR protein comprises a L114P, a R99H mutation, an A116V mutation, and a F133L mutation of a Paenibacillus GlnR protein or at a homologous amino acid position in a homolog thereof. In some cases, the mutant GlnR protein comprises a L114P, an i16V mutation, a T91I mutation, a L106F mutation, and a G128S mutation of a Paenibacillus GlnR protein or at a homologous amino acid position in a homolog thereof. In some cases, the mutant GlnR protein comprises a L114P, a M18V mutation, an I37M mutation, a V54I mutation, and a Q122R mutation of a Paenibacillus GlnR protein or at a homologous amino acid position in a homolog thereof. In some cases, the Paenibacillus glnR gene comprises a nucleic acid sequence of SEQ ID NO: 12. In some cases, the mutant glnR gene comprises a nucleic acid sequence selected from the group consisting of SEQ ID NO: 13-15. In some cases, the Paenibacillus GlnR protein comprises an amino acid sequence of SEQ ID NO: 16. In some cases, the mutant GlnR protein comprises an amino acid selected from the group consisting of SEQ ID NO: 17-19. In some cases, the engineered gram-positive diazotrophic bacterium further comprises a GlnA protein with decreased activity (e.g., the GlnA protein can be truncated). The engineered gram-positive diazotrophic bacterium can include one or more mutations in a glnA gene. In some cases, the engineered gram-positive diazotrophic bacterium further comprises a deletion of a glutamine synthetase A (glnA) gene or a portion thereof. In some cases, the engineered gram-positive diazotrophic bacterium further comprises a mutated form of a glutamine synthetase A (glnA) gene, wherein the mutated form of the glnA gene encodes a mutated GlnA protein that exhibits reduced assimilation of ammonium. In some cases, the mutated GlnA comprises at least one amino acid substitution at position 67, 182, 241 or 313 of a Paenibacillus GlnA or at a homologous amino acid position in a homolog thereof. In some cases, the mutated GlnA comprises at least one amino acid substitution selected from the group consisting of M67I, E182K, G241S and N313B of a Paenibacillus GlnA or at a homologous amino acid position in a homolog thereof. In some cases, the Paenibacillus GlnA protein comprises an amino acid sequence of SEQ ID NO: 51 or 52. In some cases, the homolog thereof is a Klebsiella GlnA protein. In some cases, the homolog thereof comprises an amino acid sequence of SEQ ID NO: 53. In some cases, the engineered gram-positive diazotrophic bacterium further comprises at least one genetic variation introduced into a member selected from the group consisting of: nifB, nifH, nifD, nifK, nifE, nifN nifX, hesA, nifV genes or combinations thereof that results in increased nitrogen fixation. In some cases, said bacterium is a species from a genus selected from Paenibacillus, Bacillus and Lactobacillus. In some cases, said bacterium is selected from Paenibacillus azotofixans, Paenibacillus borealis, Paenibacillus durus, Paenibacillus macerans, Paenibacillus polymyxa, Paenibacillus alvei, Paenibacillus amylolyticus, Paenibacillus campinasensis, Paenibacillus chibensis, Paenibacillus glucanolyticus, Paenibacillus illinoisensis, Paenibacillus larvae subsp. Larvae, Paenibacillus larvae subsp. Pulvifaciens, Paenibacillus lautus, Paenibacillus macerans, Paenibacillus macquariensis, Paenibacillus graminis, Paenibacillus pabuli, Paenibacillus peoriae, Paenibacillus stellifer, Paenibacillus riograndensis, Paenibacillus donghaensis, Paenibacillus sp. FSL, and Paenibacillus odorifier. In some cases, said bacterium is a transgenic or a remodeled non-intergeneric bacterium. In some cases, the wild-type form of the gram-positive diazotrophic bacterium is Paenibacillus polymyxa strain CI41 with deposit accession number PTA-126581.

In another aspect, provided herein is an engineered gram-positive diazotrophic bacterium comprising a heterologous promoter operably linked to a nif operon and/or a mutant glnR gene, wherein the heterologous promoter replaces at least a portion of the nif operon endogenous promoter and promotes expression of the nif operon irrespective of exogenous nitrogen levels, and wherein the mutant glnR gene encodes a mutant GlnR protein promotes expression of the nif operon irrespective of exogenous nitrogen levels. In some cases, the heterologous promoter completely replaces the nif operon endogenous promoter. In some cases, the heterologous promoter replaces a portion of the nif operon endogenous promoter downstream of a GlnR activator site, endogenous transcription start site and a GlnR repressor site. In some cases, the heterologous promoter replaces a portion of the nif operon endogenous promoter downstream of a GlnR activator site and endogenous transcription start site. In some cases, the heterologous promoter replaces a portion of the nif operon endogenous promoter downstream of a GlnR activator site. In some cases, the heterologous promoter is selected from a promoter for a Paenibacillus Acetolactate synthase (alsS) gene, Pyruvate formate-lyase-activating enzyme (pflB) gene, D-alanine aminotransferase (dat) gene, 30S ribosomal protein S21 (rpsU) gene, Aldehyde-alcohol dehydrogenase (adhe) gene, 50S ribosomal protein L13 (rplm) gene, 50S ribosomal protein L36 (rpmJ) gene, DNA-binding protein HU 1 (hupA) gene, Translation initiation factor IF-3 (infC) gene, ECF RNA polymerase sigma-E factor (rpoE) gene, and Trigger factor (tig) gene. In some cases, the heterologous promoter has a nucleic acid sequence selected from SEQ ID NOs: 1-11. In some cases, the engineered gram-positive diazotrophic bacterium is selected from the group consisting of strain 41-2753, 41-2755, 41-4230, 41-4231, 41-4232, 41-4233 and 41-4236. In some cases, the mutant glnR gene comprises at least one nucleotide substitution at nucleotide position 45, 46, 52, 111, 160, 272, 296, 316, 341, 347, 365, 382, 384 or 397 of a Paenibacillus glnR gene (e.g., SEQ ID NO:12) or at a homologous nucleotide position in a homolog thereof. In some cases, the mutant glnR gene shares at least 85%, 86%, 87%, 88%, 89%, 90%, 91%, 92%, 93%, 94%, 95%, 96%, 97%, 98%, or 99% identity with the Paenibacillus glnR gene (e.g., SEQ ID NO:12) or the homolog thereof. In some cases, the mutant GlnR protein comprises at least one amino acid substitution at amino acid position 16, 18, 37, 54, 91, 99, 106, 114, 116, 122, 128 or 133 of a Paenibacillus GlnR protein or at a homologous amino acid position in a homolog thereof.

In some cases, the mutant GlnR protein comprises at least one amino acid substitution selected from the group consisting of I16V, M18V, I37M, V54I, T91I, R99H, L106F, L114P, A116V, Q122R, G128S and F133L of a Paenibacillus GlnR protein or at a homologous amino acid position in a homolog thereof. In some cases, the mutant GlnR protein shares at least 90%, 91%, 92%, 93%, 94%, 95%, 96%, 97%, 98%, or 99% identity with the Paenibacillus GlnR protein or the homolog thereof. In some cases, the mutant GlnR protein comprises an L to P mutation at position 114 of a Paenibacillus GlnR protein or at a homologous amino acid position in a homolog thereof. In some cases, the mutant GlnR protein comprises a L114P mutation and one or more of a R99H mutation, an A116V mutation, a F133L mutation, an i16V mutation, a T91I mutation, a L106F mutation, a G128S mutation, a M18V mutation, an I37M mutation, a V54I mutation, a Q122R mutation and any combination thereof of a Paenibacillus GlnR protein or at a homologous amino acid position in a homolog thereof. In some cases, the mutant GlnR protein comprises a L114P, a R99H mutation, an A116V mutation, and a F133L mutation of a Paenibacillus GlnR protein or at a homologous amino acid position in a homolog thereof. In some cases, the mutant GlnR protein comprises a L114P, an I16V mutation, a T91I mutation, a L106F mutation, and a G128S mutation of a Paenibacillus GlnR protein or at a homologous amino acid position in a homolog thereof. In some cases, the mutant GlnR protein comprises a L114P, a M18V mutation, an I37M mutation, a V54I mutation, and a Q122R mutation of a Paenibacillus GlnR protein or at a homologous amino acid position in a homolog thereof. In some cases, the Paenibacillus glnR gene comprises a nucleic acid sequence of SEQ ID NO: 12. In some cases, the mutant glnR gene comprises a nucleic acid sequence selected from the group consisting of SEQ ID NO: 13-15. In some cases, the Paenibacillus GlnR protein comprises an amino acid sequence of SEQ ID NO: 16. In some cases, the mutant GlnR protein comprises an amino acid selected from the group consisting of SEQ ID NO: 17-19. In some cases, the engineered gram-positive diazotrophic bacterium further comprises a GlnA protein with decreased activity (e.g., the GlnA protein can be truncated). The engineered gram-positive diazotrophic bacterium can include one or more mutations in a glnA gene. In some cases, the engineered gram-positive diazotrophic bacterium further comprises a deletion of a glutamine synthetase A (glnA) gene or a portion thereof. In some cases, the engineered gram-positive diazotrophic bacterium further comprises a mutated form of a glutamine synthetase A (glnA) gene, wherein the mutated form of the glnA gene encodes a mutated GlnA protein that exhibits reduced assimilation of ammonium. In some cases, the mutated GlnA comprises at least one amino acid substitution at position 67, 182, 241 or 313 of a Paenibacillus GlnA or at a homologous amino acid position in a homolog thereof. In some cases, the mutated GlnA comprises at least one amino acid substitution selected from the group consisting of M67I, E182K, G241S and N313B of a Paenibacillus GlnA or at a homologous amino acid position in a homolog thereof. In some cases, the Paenibacillus GlnA protein comprises an amino acid sequence of SEQ ID NO: 51 or 52. In some cases, the homolog thereof is a Klebsiella GlnA protein. In some cases, the homolog thereof comprises an amino acid sequence of SEQ ID NO: 53. In some cases, the engineered gram-positive diazotrophic bacterium further comprises at least one genetic variation introduced into a member selected from the group consisting of: nifB, nifH, nifD, nifK, nifE, nif, nifX, hesA, nifV genes or combinations thereof that results in increased nitrogen fixation. In some cases, said bacterium is a species from a genus selected from Paenibacillus, Bacillus and Lactobacillus. In some cases, said bacterium is selected from Paenibacillus azotofixans, Paenibacillus borealis, Paenibacillus durus, Paenibacillus macerans, Paenibacillus polymyxa, Paenibacillus alvei, Paenibacillus amylolyticus, Paenibacillus campinasensis, Paenibacillus chibensis, Paenibacillus glucanolyticus, Paenibacillus illinoisensis, Paenibacillus larvae subsp. Larvae, Paenibacillus larvae subsp. Pulvifaciens, Paenibacillus lautus, Paenibacillus macerans, Paenibacillus macquariensis, Paenibacillus graminis, Paenibacillus pabuli, Paenibacillus peoriae, Paenibacillus stellifer, Paenibacillus riograndensis, Paenibacillus donghaensis, Paenibacillus sp. FSL, and Paenibacillus odorifier. In some cases, said bacterium is a transgenic or a remodeled non-intergeneric bacterium. In some cases, the wild-type form of the gram-positive diazotrophic bacterium is Paenibacillus polymyxa strain CI41 with deposit accession number PTA-126581.

In still another aspect, provided herein is a microbial composition comprising one or more bacteria, wherein the one or more bacteria are capable of fixing nitrogen irrespective of exogenous nitrogen levels at a rate at least equivalent to a rate of nitrogen fixation in a wild-type gram-positive diazotrophic bacterium in the absence of exogenous nitrogen. In some cases, the one or more bacteria comprise one or more engineered gram-positive diazotrophic bacteria comprising a heterologous promoter operably linked to a nif operon and/or a mutant GlnR protein, wherein the heterologous promoter replaces at least a portion of the nif operon endogenous promoter and promotes expression of the nif operon irrespective of exogenous nitrogen levels, and wherein the mutant GlnR protein promotes expression of the nif operon irrespective of exogenous nitrogen levels. In some cases, the heterologous promoter completely replaces the nif operon endogenous promoter. In some cases, the heterologous promoter replaces a portion of the nif operon endogenous promoter downstream of a GlnR activator site, endogenous transcription start site and a GlnR repressor site. In some cases, the heterologous promoter replaces a portion of the nif operon endogenous promoter downstream of a GlnR activator site and endogenous transcription start site. In some cases, the heterologous promoter replaces a portion of the nif operon endogenous promoter downstream of a GlnR activator site. In some cases, the heterologous promoter is selected from a promoter for a Paenibacillus Acetolactate synthase (alsS) gene, Pyruvate formate-lyase-activating enzyme (pflB) gene, D-alanine aminotransferase (dat) gene, 30S ribosomal protein S21 (rpsU) gene, Aldehyde-alcohol dehydrogenase (adhe) gene, 50S ribosomal protein L13 (rplm) gene, 50S ribosomal protein L36 (rpmJ) gene, DNA-binding protein HU 1 (hupA) gene, Translation initiation factor IF-3 (infC) gene, ECF RNA polymerase sigma-E factor (rpoE) gene, and Trigger factor (tig) gene. In some cases, the heterologous promoter has a nucleic acid sequence selected from SEQ ID NOs: 1-11. In some cases, the one or more engineered gram-positive diazotrophic bacterium is selected from the group consisting of 41-2753, 41-2755, 41-4230, 41-4231, 41-4232, 41-4233 and 41-4236. In some cases, the mutant glnR gene comprises at least one nucleotide substitution at nucleotide position 45, 46, 52, 111, 160, 272, 296, 316, 341, 347, 365, 382, 384 or 397 of a Paenibacillus glnR gene (e.g., SEQ ID NO:12) or at a homologous nucleotide position in a homolog thereof. In some cases, the mutant glnR gene shares at least 85%, 86%, 87%, 88%, 89%, 90%, 91%, 92%, 93%, 94%, 95%, 96%, 97%, 98%, or 99% identity with the Paenibacillus glnR gene (e.g., SEQ ID NO:12) or the homolog thereof. In some cases, the mutant GlnR protein comprises at least one amino acid substitution of at amino acid position 16, 18, 37, 54, 91, 99, 106, 114, 116, 122, 128 or 133 of a Paenibacillus GlnR protein or at a homologous amino acid position in a homolog thereof. In some cases, the mutant GlnR protein comprises at least one amino acid substitution selected from the group consisting of I16V, M18V, I37M, V54I, T91I, R99H, L106F, L114P, A116V, Q122R, G128S and F133L of a Paenibacillus GlnR protein or at a homologous amino acid position in a homolog thereof. In some cases, the mutant GlnR protein shares at least 90%, 91%, 92%, 93%, 94%, 95%, 96%, 97%, 98%, or 99% identity with the Paenibacillus GlnR protein or the homolog thereof. In some cases, the mutant GlnR protein comprises an L to P mutation at position 114 of a Paenibacillus GlnR protein or at a homologous amino acid position in a homolog thereof. In some cases, the mutant GlnR protein comprises a L114P mutation and one or more of a R99H mutation, an A116V mutation, a F133L mutation, an i16V mutation, a T91I mutation, a L106F mutation, a G128S mutation, a M18V mutation, an I37M mutation, a V54I mutation, a Q122R mutation and any combination thereof of a Paenibacillus GlnR protein or at a homologous amino acid position in a homolog thereof. In some cases, the mutant GlnR protein comprises a L114P, a R99H mutation, an A116V mutation, and a F133L mutation of a Paenibacillus GlnR protein or at a homologous amino acid position in a homolog thereof. In some cases, the mutant GlnR protein comprises a L114P, an i16V mutation, a T91I mutation, a L106F mutation, and a G128S mutation of a Paenibacillus GlnR protein or at a homologous amino acid position in a homolog thereof. In some cases, the mutant GlnR protein comprises a L114P, a M18V mutation, an I37M mutation, a V54I mutation, and a Q122R mutation of a Paenibacillus GlnR protein or at a homologous amino acid position in a homolog thereof. In some cases, the Paenibacillus glnR gene comprises a nucleic acid sequence of SEQ ID NO: 12. In some cases, the mutant glnR gene comprises a nucleic acid sequence selected from the group consisting of SEQ ID NO: 13-15. In some cases, the Paenibacillus GlnR protein comprises an amino acid sequence of SEQ ID NO: 16. In some cases, the mutant GlnR protein comprises an amino acid selected from the group consisting of SEQ ID NO: 17-19. In some cases, the one or more engineered gram-positive diazotrophic bacteria further comprise a GlnA protein with decreased activity (e.g., the GlnA protein can be truncated). The one or more engineered gram-positive diazotrophic bacteria can include one or more mutations in a glnA gene. In some cases, the one or more engineered gram-positive diazotrophic bacteria further comprise a deletion of a glutamine synthetase A (glnA) gene or a portion thereof. In some cases, the one or more engineered gram-positive diazotrophic bacteria comprise a mutated form of a glutamine synthetase A (glnA) gene, wherein the mutated form of the glnA gene encodes a mutated GlnA protein that exhibits reduced assimilation of ammonium. In some cases, the mutated GlnA comprises at least one amino acid substitution at position 67, 182, 241 or 313 of a Paenibacillus GlnA or at a homologous amino acid position in a homolog thereof. In some cases, the mutated GlnA comprises at least one amino acid substitution selected from the group consisting of M67I, E182K, G241S and N313B of a Paenibacillus GlnA and homologous amino acid positions in a homolog thereof. In some cases, the Paenibacillus GlnA protein comprises an amino acid sequence of SEQ ID NO: 51 or 52. In some cases, the homolog thereof is a Klebsiella GlnA protein. In some cases, the homolog thereof comprises an amino acid sequence of SEQ ID NO: 53. In some cases, the one or more engineered gram-positive diazotrophic bacteria further comprise further comprising at least one genetic variation introduced into a member selected from the group consisting of: nifB, nifH, nifD, nifK, nifE, nifN, nifX, hesA, nifV genes and combinations thereof that results in increased nitrogen fixation. In some cases, the one or more engineered gram-positive diazotrophic bacteria comprise at least two different species of bacteria. In some cases, the one or more engineered gram-positive diazotrophic bacteria comprise at least two different strains of the same species of bacteria. In some cases, the one or more engineered gram-positive diazotrophic bacteria is a species from a genus selected from Paenibacillus, Bacillus and Lactobacillus. In some cases, the one or more engineered gram-positive diazotrophic bacteria is selected from Paenibacillus azotofixans, Paenibacillus borealis, Paenibacillus durus, Paenibacillus macerans, Paenibacillus polymyxa, Paenibacillus alvei, Paenibacillus amylolyticus, Paenibacillus campinasensis, Paenibacillus chibensis, Paenibacillus glucanolyticus, Paenibacillus illinoisensis, Paenibacillus larvae subsp. Larvae, Paenibacillus larvae subsp. Pulvifaciens, Paenibacillus lautus, Paenibacillus macerans, Paenibacillus macquariensis, Paenibacillus graminis, Paenibacillus pabuli, Paenibacillus peoriae, Paenibacillus stellifer, Paenibacillus riograndensis, Paenibacillus donghaensis, Paenibacillus sp. FSL, or Paenibacillus odorifier. In some cases, the one or more engineered gram-positive diazotrophic bacteria produce 1% or more of fixed nitrogen in a plant exposed thereto. In some cases, the composition is a solid. In some cases, the composition is a liquid. In some cases, the microbial composition is a present as a seed coat on a plant seed or other plant propagation material. In some cases, the microbial composition is present as a liquid on a plant as an in-furrow treatment. In some cases, the one or more engineered gram-positive diazotrophic bacteria are transgenic or remodeled non-intergeneric bacteria. In some cases, the wild-type gram-positive diazotrophic bacterium is Paenibacillus polymyxa strain CI41 with deposit accession number PTA-126581. In some cases, provided herein is a method of providing fixed nitrogen to a plant comprising applying the microbial composition to the plant, a plant part, or a locus in which the plant is located, or a locus in which the plant will be grown. In some cases, the applying comprises coating a seed or other plant propagation member with the microbial composition. In some cases, the one or more engineered gram-positive diazotrophic bacteria in the microbial composition has an average colonization ability per unit of plant root tissue of at least about 1.0×10⁴ colony forming unit (cfu) per gram of fresh weight of plant root tissue and produce fixed N of at least about 1×10⁻¹⁵ mmol N per bacterial cell per hour. In some cases, the applying comprises performing in-furrow treatment of the microbial composition to a locus in which the plant is present, or will be present. In some cases, the in-furrow treatment comprises applying the microbial composition at a concentration per acre of between about 1×10⁶ to about 3×10¹² cfu per acre. In some cases, the microbial composition is a liquid formulation comprising about 1×10⁶ to about 1×10¹¹ cfu of bacterial cells per milliliter.

In yet another aspect, provided herein is a glnR gene comprising at least one nucleotide substitution at nucleotide position 45, 46, 52, 111, 160, 272, 296, 316, 341, 347, 365, 382, 384 or 397 of a Paenibacillus glnR gene (e.g., SEQ ID NO:12) or at a homologous nucleotide position in a homolog thereof. In some cases, the glnR gene shares at least 85%, 86%, 87%, 88%, 89%, 90%, 91%, 92%, 93%, 94%, 95%, 96%, 97%, 98%, or 99% identity with the Paenibacillus glnR gene (e.g., SEQ ID NO:12) or the homolog thereof. In some cases, the glnR gene encodes a GlnR protein comprising at least one amino acid substitution of at amino acid position 16, 18, 37, 54, 91, 99, 106, 114, 116, 122, 128 or 133 of a Paenibacillus GlnR protein or at a homologous amino acid position in a homolog thereof. In some cases, the glnR gene encodes a GlnR protein comprising at least one amino acid substitution selected from the group consisting of I16V, M18V, I37M, V54I, T91I, R99H, L106F, L114P, A116V, Q122R, G128S and F133L of a Paenibacillus GlnR protein and homologous amino acid positions in a homolog thereof. In some cases, the GlnR protein shares at least 90%, 91%, 92%, 93%, 94%, 95%, 96%, 97%, 98%, or 99% identity with the Paenibacillus GlnR protein or the homolog thereof. In some cases, the GlnR protein comprises an L to P mutation at position 114 of the Paenibacillus GlnR protein or at a homologous amino acid position in the homolog thereof. In some cases, the GlnR protein comprises a L114P mutation and one or more of a R99H mutation, an A116V mutation, a F133L mutation, an i16V mutation, a T91I mutation, a L106F mutation, a G128S mutation, a M18V mutation, an I37M mutation, a V54I mutation, a Q122R mutation and any combination thereof of the Paenibacillus GlnR protein or at a homologous amino acid position in the homolog thereof. In some cases, the GlnR protein comprises a L114P, a R99H mutation, an A116V mutation, and a F133L mutation of the Paenibacillus GlnR protein or at a homologous amino acid position in the homolog thereof. In some cases, the GlnR protein comprises a L114P, an i16V mutation, a T91I mutation, a L106F mutation, and a G128S mutation of the Paenibacillus GlnR protein or at a homologous amino acid position in the homolog thereof. In some cases, the GlnR protein comprises a L114P, a M18V mutation, an I37M mutation, a V54I mutation, and a Q122R mutation of the Paenibacillus GlnR protein or at a homologous amino acid position in the homolog thereof. In some cases, the Paenibacillus glnR gene comprises a nucleic acid sequence of SEQ ID NO: 12. In some cases, the glnR gene comprises a nucleic acid sequence selected from the group consisting of SEQ ID NO: 13-15. In some cases, the Paenibacillus GlnR protein comprises an amino acid sequence of SEQ ID NO: 16. In some cases, the GlnR protein comprises an amino acid selected from the group consisting of SEQ ID NO: 17-19.

In one aspect, provided herein is a GlnR protein comprising at least one amino acid substitution of at amino acid position 16, 18, 37, 54, 91, 99, 106, 114, 116, 122, 128 or 133 of a Paenibacillus GlnR protein or at a homologous amino acid position in a homolog thereof. In some cases, the at least one amino acid substitution is selected from the group consisting of a I16V, M18V, I37M, V54I, T91I, R99H, L106F, L114P, A116V, Q122R, G128S and F133L of the Paenibacillus GlnR protein and homologous amino acid positions in the homolog thereof. In some cases, the GlnR protein shares at least 90%, 91%, 92%, 93%, 94%, 95%, 96%, 97%, 98%, or 99% identity with the Paenibacillus GlnR protein or the homolog thereof. In some cases, the GlnR protein comprises an L to P mutation at position 114 of the Paenibacillus GlnR protein or at a homologous amino acid position in the homolog thereof. In some cases, the GlnR protein comprises a L114P mutation and one or more of a R99H mutation, an A116V mutation, a F133L mutation, an I16V mutation, a T91I mutation, a L106F mutation, a G128S mutation, a M18V mutation, an I37M mutation, a V54I mutation, a Q122R mutation and any combination thereof of the Paenibacillus GlnR protein or at a homologous amino acid position in the homolog thereof. In some cases, the GlnR protein comprises a L114P, a R99H mutation, an A116V mutation, and a F133L mutation of the Paenibacillus GlnR protein or at a homologous amino acid position in the homolog thereof. In some cases, the GlnR protein comprises a L114P, an I16V mutation, a T91I mutation, a L106F mutation, and a G128S mutation of the Paenibacillus GlnR protein or at homologous amino acid positions in the homolog thereof. In some cases, the GlnR protein comprises a L114P, a M18V mutation, an I37M mutation, a V54I mutation, and a Q122R mutation of a Paenibacillus GlnR protein or at a homologous amino acid position in a homolog thereof. In some cases, the Paenibacillus GlnR protein comprises an amino acid sequence of SEQ ID NO: 16. In some cases, the GlnR protein comprises an amino acid selected from the group consisting of SEQ ID NO: 17-19.

In another aspect, provided herein is a method for identifying regulators of a nif operon that exhibit de-repression activity in the presence of ammonium, the method comprising: (a) introducing individual mutagenized glnR genes from a library of mutagenized glnR genes into a engineered gram-positive diazotrophic microbial host cell missing a wild-type glnR gene, wherein the gram-positive diazotrophic microbial host cell comprises a nucleic acid sequence encoding a selectable marker protein, functional fragment, and/or fusions thereof operably linked to a nifB promoter; (b) culturing the engineered gram-positive diazotrophic microbial host cell in the presence of ammonium under anaerobic conditions, wherein the engineered gram-positive diazotrophic microbial host cell expresses the selectable marker protein, functional fragment, and/or fusions thereof in the presence of ammonium if the mutagenized glnR gene introduced in step (a) encodes a GlnR protein that exhibits de-repression activity in the presence of ammonium; (c) exposing the engineered gram-positive diazotrophic microbial host cell to an agent that allows for selection of gram-positive diazotrophic microbial host cell's expressing the selectable marker protein; and (d) identifying individual mutagenized glnR genes from the library of mutagenized glnR genes as exhibiting de-repression activity in the presence of ammonium as those that result in selection of the gram-positive diazotrophic microbial host cells expressing the selectable marker protein as compared to a control. In some cases, the selectable marker protein is selected from a fluorescent marker protein, a luminescent marker protein, a chromogenic marker, an auxotrophic marker and antibiotic resistance marker protein. In some cases, the selectable marker protein is a fluorescent marker protein. In some cases, the fluorescent protein is a GFP, RFP, YFP, CFP, or functional variant or fragment thereof. In some cases, the fluorescent marker protein is GFP. In some cases, steps (b)-(d) comprise: (b) culturing the engineered gram-positive diazotrophic microbial host cell in the presence of ammonium under anaerobic conditions, wherein the engineered gram-positive diazotrophic microbial host cell expresses the fluorescent marker protein, functional fragment, and/or fusions thereof in the presence of ammonium if the mutagenized glnR gene introduced in step (a) encodes a GlnR protein that exhibits de-repression activity in the presence of ammonium; (c) exposing the engineered gram-positive diazotrophic microbial host cell to light excitation sufficient to fluoresce the fluorescent marker protein, functional fragment, and/or fusions thereof, and (d) identifying individual mutagenized glnR genes from the library of mutagenized glnR genes as exhibiting de-repression activity in the presence of ammonium as those that result in fluorescence of the fluorescent marker protein, functional fragment, and/or fusions thereof, as compared to a control. In some cases, the fluorescence is detected with a flow cytometer, a plate reader, or fluorescence-activated droplet sorting. In some cases, the control is an engineered gram-positive diazotrophic microbial host cell expressing wild-type glnR. In some cases, step (b) is performed in the presence of at least 1 mM, 2 mM, 3 mM, 4 nM, 5 mM, 6 mM, 7 mM, 8 mM, 9 mM or 10 mM ammonium. In some cases, the engineered gram-positive diazotrophic microbial host cell is selected from Paenibacillus, Bacillus and Lactobacillus. In some cases, the engineered gram-positive diazotrophic microbial host cell is selected from Paenibacillus azotofixans, Paenibacillus borealis, Paenibacillus durus, Paenibacillus macerans, Paenibacillus polymyxa, Paenibacillus alvei, Paenibacillus amylolyticus, Paenibacillus campinasensis, Paenibacillus chibensis, Paenibacillus glucanolyticus, Paenibacillus illinoisensis, Paenibacillus larvae subsp. Larvae, Paenibacillus larvae subsp. Pulvifaciens, Paenibacillus lautus, Paenibacillus macerans, Paenibacillus macquariensis, Paenibacillus graminis, Paenibacillus pabuli, Paenibacillus peoriae, Paenibacillus stellifer, Paenibacillus riograndensis, Paenibacillus donghaensis, Paenibacillus sp. FSL, and Paenibacillus odorifier. In some cases, the engineered gram-positive diazotrophic microbial host cell is a transgenic or remodeled non-intergeneric host cell. In some cases, the identified mutagenized glnR gene comprises at least one nucleotide substitution at nucleotide position 45, 46, 52, 111, 160, 272, 296, 316, 341, 347, 365, 382, 384 or 397 of a Paenibacillus glnR gene (e.g., SEQ ID NO:12) or at a homologous nucleotide position in a homolog thereof. In some cases, the mutagenized glnR gene shares at least 85%, 86%, 87%, 88%, 89%, 90%, 91%, 92%, 93%, 94%, 95%, 96%, 97%, 98%, or 99% identity with the Paenibacillus glnR gene (e.g., SEQ ID NO: 12) or the homolog thereof. In some cases, the mutagenized glnR gene encodes a GlnR protein comprising at least one amino acid substitution of at amino acid position 16, 18, 37, 54, 91, 99, 106, 114, 116, 122, 128 or 133 of a Paenibacillus GlnR protein or at a homologous amino acid position in a homolog thereof. In some cases, the mutagenized glnR gene encodes a GlnR protein comprising at least one amino acid substitution selected from the group consisting of I16V, M18V, I37M, V54I, T91I, R99H, L106F, L114P, A116V, Q122R, G128S and F133L of a Paenibacillus GlnR protein and homologous amino acid positions in a homolog thereof. In some cases, the GlnR protein shares at least 90%, 91%, 92%, 93%, 94%, 95%, 96%, 97%, 98%, or 99% identity with the Paenibacillus GlnR protein or the homolog thereof. In some cases, the GlnR protein comprises an L to P mutation at position 114 of the Paenibacillus GlnR protein or at a homologous amino acid position in the homolog thereof. In some cases, the GlnR protein comprises a L114P mutation and one or more of a R99H mutation, an A116V mutation, a F133L mutation, an I16V mutation, a T91I mutation, a L106F mutation, a G128S mutation, a M18V mutation, an I37M mutation, a V54I mutation, a Q122R mutation and any combination thereof of the Paenibacillus GlnR protein or at a homologous amino acid position in the homolog thereof. In some cases, the GlnR protein comprises a L114P, a R99H mutation, an A116V mutation, and a F133L mutation of the Paenibacillus GlnR protein or at a homologous amino acid position in the homolog thereof. In some cases, the GlnR protein comprises a L114P, an I16V mutation, a T91I mutation, a L106F mutation, and a G128S mutation of the Paenibacillus GlnR protein or at a homologous amino acid position in the homolog thereof. In some cases, the GlnR protein comprises a L114P, a M18V mutation, an I37M mutation, a V54I mutation, and a Q122R mutation of the Paenibacillus GlnR protein or at homologous amino acid positions in the homolog thereof. In some cases, the Paenibacillus glnR gene comprises a nucleic acid sequence of SEQ ID NO: 12. In some cases, the glnR gene comprises a nucleic acid sequence selected from the group consisting of SEQ ID NO: 13-15. In some cases, the Paenibacillus GlnR protein comprises an amino acid sequence of SEQ ID NO: 16. In some cases, the GlnR protein comprises an amino acid selected from the group consisting of SEQ ID NO: 17-19.

In one aspect, provided herein is a method of providing fixed nitrogen to a plant comprising applying a microbial composition to a plant, a plant part, or a locus in which the plant is located, or a locus in which the plant will be grown, wherein the microbial composition comprises one or more engineered gram-positive diazotrophic bacteria capable of fixing nitrogen irrespective of exogenous nitrogen levels. In some cases, the one or more engineered gram-positive diazotrophic bacteria comprise a heterologous promoter operably linked to a nif operon, wherein the heterologous promoter replaces at least a portion of the nif operon endogenous promoter and promotes expression of the nif operon irrespective of exogenous nitrogen levels. In some cases, the heterologous promoter completely replaces the nif operon endogenous promoter. In some cases, the heterologous promoter replaces a portion of the nif operon endogenous promoter downstream of a GlnR activator site, endogenous transcription start site and a GlnR repressor site. In some cases, the heterologous promoter replaces a portion of the nif operon endogenous promoter downstream of a GlnR activator site and endogenous transcription start site. In some cases, the heterologous promoter replaces a portion of the nif operon endogenous promoter downstream of a GlnR activator site. In some cases, the heterologous promoter is selected from a promoter for the Paenibacillus Acetolactate synthase (alsS) gene, Pyruvate formate-lyase-activating enzyme (pflB) gene, D-alanine aminotransferase (dat) gene, 30S ribosomal protein S21 (rpsU) gene, Aldehyde-alcohol dehydrogenase (adhe) gene, 50S ribosomal protein L13 (rplm) gene, 50S ribosomal protein L36 (rpmJ) gene, DNA-binding protein HU 1 (hupA) gene, Translation initiation factor IF-3 (infC) gene, ECF RNA polymerase sigma-E factor (rpoE) gene, and Trigger factor (tig) gene. In some cases, the heterologous promoter has a nucleic acid sequence selected from SEQ ID NOs: 1-11. In some cases, the one or more engineered gram-positive diazotrophic bacteria are selected from the group consisting of 41-2753, 41-2755, 41-4230, 41-4231, 41-4232, 41-4233 and 41-4236. In some cases, the one or more engineered gram-positive diazotrophic bacteria comprise a mutant glnR gene, wherein the mutant glnR gene encodes a mutant GlnR protein that promotes expression of the nif operon irrespective of exogenous nitrogen levels. In some cases, the mutant glnR gene comprises at least one nucleotide substitution at nucleotide position 45, 46, 52, 111, 160, 272, 296, 316, 341, 347, 365, 382, 384 or 397 of a Paenibacillus glnR gene (e.g., SEQ ID NO:12) or at a homologous nucleotide position in a homolog thereof. In some cases, the mutant glnR gene shares at least 85%, 86%, 87%, 88%, 89%, 90%, 91%, 92%, 93%, 94%, 95%, 96%, 97%, 98%, or 99% identity with the Paenibacillus glnR gene (e.g., SEQ ID NO:12) or the homolog thereof. In some cases, the mutant GlnR protein comprises at least one amino acid substitution of at amino acid position 16, 18, 37, 54, 91, 99, 106, 114, 116, 122, 128 or 133 of a Paenibacillus GlnR protein or at a homologous amino acid position in a homolog thereof. In some cases, the mutant GlnR protein comprises at least one amino acid substitution selected from the group consisting of I16V, M18V, I37M, V54I, T91I, R99H, L106F, L114P, A116V, Q122R, G128S and F133L of a Paenibacillus GlnR protein and homologous amino acid positions in a homolog thereof. In some cases, the mutant GlnR protein shares at least 90%, 91%, 92%, 93%, 94%, 95%, 96%, 97%, 98%, or 99% identity with the Paenibacillus GlnR protein or the homolog thereof. In some cases, the mutant GlnR protein comprises an L to P mutation at position 114 of the Paenibacillus GlnR protein or at a homologous amino acid position in the homolog thereof. In some cases, the mutant GlnR protein comprises a L114P mutation and one or more of a R99H mutation, an A116V mutation, a F133L mutation, an I16V mutation, a T91I mutation, a L106F mutation, a G128S mutation, a M18V mutation, an 137M mutation, a V54I mutation, a Q122R mutation and any combination thereof of a Paenibacillus GlnR protein or at a homologous amino acid position in a homolog thereof. In some cases, the mutant GlnR protein comprises a L114P, a R99H mutation, an A116V mutation, and a F133L mutation of a Paenibacillus GlnR protein or at a homologous amino acid position in a homolog thereof. In some cases, the mutant GlnR protein comprises a L114P, an I16V mutation, a T91I mutation, a L106F mutation, and a G128S mutation of a Paenibacillus GlnR protein or at a homologous amino acid position in a homolog thereof. In some cases, the mutant GlnR protein comprises a L114P, a M18V mutation, an I37M mutation, a V54I mutation, and a Q122R mutation of a Paenibacillus GlnR protein or at a homologous amino acid position in a homolog thereof. In some cases, the Paenibacillus glnR gene comprises a nucleic acid sequence of SEQ ID NO: 12. In some cases, the mutant glnR gene comprises a nucleic acid sequence selected from the group consisting of SEQ ID NO: 13-15. In some cases, the Paenibacillus GlnR protein comprises an amino acid sequence of SEQ ID NO: 16. In some cases, the mutant GlnR protein comprises an amino acid selected from the group consisting of SEQ ID NO: 17-19. In some cases, the one or more engineered gram-positive diazotrophic bacteria comprises a deletion of a glutamine synthetase A (glnA) gene. In some cases, the one or more engineered gram-positive diazotrophic bacteria comprises a mutated form of a glutamine synthetase A (glnA) gene, wherein the mutated form of the glnA gene encodes a mutated GlnA protein that exhibits reduced assimilation of ammonium. In some cases, the mutated GlnA protein comprises at least one amino acid substitution at position 67, 182, 241 or 313 of a Paenibacillus GlnA or at a homologous amino acid position in a homolog thereof. In some cases, the mutated GlnA protein comprises at least one amino acid substitution selected from the group consisting of M67I, E182K, G241S and N313B of a Paenibacillus GlnA and homologous amino acid positions in a homolog thereof. In some cases, the Paenibacillus GlnA protein comprises an amino acid sequence of SEQ ID NO: 51 or 52. In some cases, the homolog thereof is a Klebsiella GlnA protein. In some cases, the homolog thereof comprises an amino acid sequence of SEQ ID NO: 53. In some cases, the one or more engineered gram-positive diazotrophic bacteria comprise at least one genetic variation introduced into a member selected from the group consisting of: nifB, nifH, nifD, nifK, nifE, nifN, nifX, hesA, nifV genes and combinations thereof that results in increased nitrogen fixation. In some cases, the one or more engineered gram-positive diazotrophic bacteria comprise at least two different species of bacteria. In some cases, the one or more engineered gram-positive diazotrophic bacteria comprise at least two different strains of the same species of bacteria. In some cases, the one or more engineered gram-positive diazotrophic bacteria is a species from a genus selected from Paenibacillus, Bacillus and Lactobacillus. In some cases, the one or more engineered gram-positive diazotrophic bacteria is selected from Paenibacillus azotofixans, Paenibacillus borealis, Paenibacillus durus, Paenibacillus macerans, Paenibacillus polymyxa, Paenibacillus alvei, Paenibacillus amylolyticus, Paenibacillus campinasensis, Paenibacillus chibensis, Paenibacillus glucanolyticus, Paenibacillus illinoisensis, Paenibacillus larvae subsp. Larvae, Paenibacillus larvae subsp. Pulvifaciens, Paenibacillus lautus, Paenibacillus macerans, Paenibacillus macquariensis, Paenibacillus graminis, Paenibacillus pabuli, Paenibacillus peoriae, Paenibacillus stellifer, Paenibacillus riograndensis, Paenibacillus donghaensis, Paenibacillus sp.

FSL, or Paenibacillus odorifier. In some cases, the one or more engineered gram-positive diazotrophic bacteria produce 1% or more of fixed nitrogen in the plant. In some cases, the microbial composition is a solid. In some cases, the microbial composition is a liquid. In some cases, the one or more engineered gram-positive diazotrophic bacteria are transgenic or remodeled non-intergeneric bacteria. In some cases, the applying comprises coating a seed or other plant propagation member with the microbial composition. In some cases, the one or more engineered gram-positive diazotrophic bacteria in the microbial composition has an average colonization ability per unit of plant root tissue of at least about 1.0×10⁴ cfu per gram of fresh weight of plant root tissue and produce fixed N of at least about 1×10⁻¹⁵ mmol N per bacterial cell per hour. In some cases, the applying comprises performing in-furrow treatment of the microbial composition to a locus in which the plant is present, or will be present. In some cases, the in-furrow treatment comprises applying the microbial composition at a concentration per acre of between about 1×10⁶ to about 3×10¹² cfu per acre. In some cases, the microbial composition is a liquid formulation comprising about 1×10⁶ to about 1×10¹¹ cfu of bacterial cells per milliliter.

### BRIEF DESCRIPTION OF THE DRAWINGS

FIG. 1 illustrates nif cluster regulation in Paenibacillus. GlnR senses exogenous nitrogen levels and regulates transcription of the nif genes. The nifB promoter has two GlnR-binding operators. Under ammonium depletion GlnR binds upstream of the promoter, recruits RNA polymerase and activates transcription, whereas under ammonium excess glutamine synthetase (GS) interacts with GlnR and increases binding affinity of GlnR, which allows GlnR to bind downstream of the promoter and form a roadblock to the progress of transcribing RNA polymerase.

FIG. 2A-2B illustrates high-throughput screening system for identification of GlnR mutants. Activation of the nif cluster is judged by a reporter plasmid that encodes GFP under the nifB promoter (FIG. 2A). glnR is knocked out of the genome and complemented by randomly mutagenized glnR carried on a separate plasmid. The wild-type GlnR was complemented using the screening system and showed that the system can sense ammonium levels and regulate nif transcription (FIG. 2B).

FIG. 3 illustrates ammonium-insensitive GlnR screening. GlnR mutants that activate transcription of the nif cluster were identified on Paenibacillus minimal agar media supplemented with 10 mM ammonium chloride. The small circle indicates a GlnR mutant that led to nif gene activation visualized by GFP expression in the presence of ammonium.

FIG. 4 illustrates functional testing of a series of GlnR mutants that derepress the nifB promoter in the presence of ammonium based on the screening system. Although the deletion of the GlnR C-terminus (Δ113-137) eradicates ammonium repression, the overall nitrogenase activity was decreased by 5-fold, implying that the interaction between GlnR and glutamine synthetase is required to achieve full nitrogenase activity. A mutant (i.e., L114P) showed partial ammonium derepression. Based on this mutant, a second round of mutagenesis was applied and identified mutants that yielded complete recovery of nif gene activation in the presence of 10 mM ammonium chloride.

FIG. 5 illustrates functional testing of a series of GlnR genomic mutants that derepress the nifB promoter in the presence of ammonium. A genomic copy of GlnR was replaced with the GlnR mutants, which were identified by the screening system. Activation of the mf cluster was tested by a reporter plasmid encoding GFP under the regulation of the nifB promoter that was introduced into a series of the GlnR mutants by conjugation.

FIG. 6 illustrates nitrogenase activity in the presence and absence of ammonium. The GlnR mutant led to complete recovery of nitrogenase activity in the presence of ammonium.

FIG. 7 illustrates multiple sequence alignment of GlnR relative to CI41 across Paenibacillus. The corresponding residues that allow ammonium tolerance of GlnR are outlined.

FIG. 8 is a schematic showing the cis-elements in the nifB promoter as well as exemplary V0-V3 modifications using the pflB promoter as described in Example 2.

FIG. 9 illustrates the 13 promoters tested for potential use for nifB promoter engineering. The cold shock protein CspB promoter (i.e., cspB CDS prom) and Thioredoxin promoter (i.e., trxA CDS prom) were not carried forward.

FIG. 10 illustrates the strain ID, genotype and description of the V0 nifB promoter modifications described in Example 2. V0 strains using the cold shock protein CspB promoter (i.e., cspB CDS prom; promoter strength 6 from FIG. 9) and Thioredoxin promoter (i.e., trxA CDS prom; promoter strength 7 from FIG. 9) were not built.

FIG. 11 illustrates the results of an acetylene reduction assay (ARA) performed in nitrogen deplete (0 mM ammonium phosphate) and nitrogen rich (5 mM ammonium phosphate) media using each of the strains built for the V0 modification as described in Example 2 and depicted in FIG. 10 in graphical form.

FIG. 12 illustrates the results of an acetylene reduction assay (ARA) performed in nitrogen rich (5 mM ammonium phosphate) media using each of the strains built for the V0 modification as described in Example 2 and depicted in FIG. 10 in Table form.

FIG. 13 illustrates the strain ID, genotype and description of the strains built to test the V0-V3 modifications of the nifB promoter as described in Example 2.

FIG. 14 illustrates the results of an acetylene reduction assay (ARA) performed in nitrogen deplete (0 mM ammonium phosphate) and nitrogen rich (5 mM ammonium phosphate) media using each of the strains described in FIG. 13 in graphical form.

FIG. 15 illustrates the results of an acetylene reduction assay (ARA) performed in nitrogen rich (5 mM ammonium phosphate) media using each of the strains described in FIG. 13 in Table form.

FIG. 16 illustrates a plasmid map of the fluorescence reporter (i.e., GFP) operably linked to the nifB promoter used in the high-throughput screening system described in Example 1.

FIG. 17 illustrates an exemplary plasmid map of a glnR mutant generated from genomic DNA of Paenibacillus CI41 by error-prone PCR and assembled with into a plasmid with a rep60 origin of replication.

FIG. 18A-B illustrates an exemplary regulatory model of GlnR involved in nitrogen fixation in gram-positive diazotrophic microorganisms (e.g., Paenibacillus polymyxa WLY78) during nitrogen limitation (FIG. 18A) and excess nitrogen (FIG. 18B).

### DETAILED DESCRIPTION OF THE DISCLOSURE

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

Increased fertilizer utilization brings with it environmental concerns and is also likely not possible for many economically stressed regions of the globe. Furthermore, many industry players in the microbial arena are focused on creating intergeneric microbes. However, there is a heavy regulatory burden placed on engineered microbes that are characterized/classified as intergeneric. These intergeneric microbes face not only a higher regulatory burden, which makes widespread adoption and implementation difficult, but they also face a great deal of public perception scrutiny.

Currently, there are no engineered gram-positive microbes on the market that are capable of increasing nitrogen fixation in non-leguminous crops in a manner in which the gram-positive microorganism exhibits full or complete de-repression of nitrogenase activity regardless or irrespective of fixed exogenous nitrogen levels. This dearth of such a microbe is a missing element in helping to usher in a truly environmentally friendly and more sustainable 21^{st} century agricultural system.

The present disclosure solves the aforementioned problems and provides gram-positive microbes that have been engineered to readily fix nitrogen in crops irrespective of fixed exogenous nitrogen levels. These microbes can be characterized/classified as not being intergeneric microbes and thus will not face the steep regulatory burdens of such. Further, the taught non-intergeneric microbes will serve to help 21^{st} century farmers become less dependent upon utilizing ever increasing amounts of exogenous nitrogen fertilizer.

### Definitions

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner according to base complementarity. The complex may comprise two strands forming a duplex structure, three or more strands forming a multi-stranded complex, a single self-hybridizing strand, or any combination of these. A hybridization reaction may constitute a step in a more extensive process, such as the initiation of PCR, or the enzymatic cleavage of a polynucleotide by an endonuclease. A second sequence that is complementary to a first sequence is referred to as the “complement” of the first sequence. The term “hybridizable” as applied to a polynucleotide refers to the ability of the polynucleotide to form a complex that is stabilized via hydrogen bonding between the bases of the nucleotide residues in a hybridization reaction.

As used herein, “biofilm” or “mature biofilm” refers to associated and/or accumulated and/or aggregated microbial cells, their products (e.g. exopolymeric substances) and inorganic particles adherent to a living or inert surface.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. Sequence identity, such as for the purpose of assessing percent complementarity, may be measured by any suitable alignment algorithm, including but not limited to the Needleman-Wunsch algorithm (see e.g. the EMBOSS Needle aligner available at www.ebi.ac.uk/Tools/psa/emboss_needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/nucleotide.html, optionally with default settings).

Optimal alignment may be assessed using any suitable parameters of a chosen algorithm, including default parameters.

In general, “stringent conditions” for hybridization refer to conditions under which a nucleic acid having complementarity to a target sequence predominantly hybridizes with a target sequence, and substantially does not hybridize to non-target sequences. Stringent conditions are generally sequence-dependent and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins.

Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the term “about” is used synonymously with the term “approximately.” Illustratively, the use of the term “about” with regard to an amount indicates that values slightly outside the cited values, e.g., plus or minus 0.1% to 10%.

The term “biologically pure culture” or “substantially pure culture” refers to a culture of a bacterial species described herein containing no other bacterial species in quantities sufficient to interfere with the replication of the culture or be detected by normal bacteriological techniques.

“Plant productivity” refers generally to any aspect of growth or development of a plant that is a reason for which the plant is grown. For food crops, such as grains or vegetables, “plant productivity” can refer to the yield of grain or fruit harvested from a particular crop. As used herein, improved plant productivity refers broadly to improvements in yield of grain, fruit, flowers, or other plant parts harvested for various purposes, improvements in growth of plant parts, including stems, leaves and roots, promotion of plant growth, maintenance of high chlorophyll content in leaves, increasing fruit or seed numbers, increasing fruit or seed unit weight, reducing NO₂ emission due to reduced nitrogen fertilizer usage and similar improvements of the growth and development of plants.

Microbes in and around food crops can influence the traits of those crops. Plant traits that may be influenced by microbes include: yield (e.g., grain production, biomass generation, fruit development, flower set); nutrition (e.g., nitrogen, phosphorus, potassium, iron, micronutrient acquisition); abiotic stress management (e.g., drought tolerance, salt tolerance, heat tolerance); and biotic stress management (e.g., pest, weeds, insects, fungi, and bacteria). Strategies for altering crop traits include: increasing key metabolite concentrations; changing temporal dynamics of microbe influence on key metabolites; linking microbial metabolite production/degradation to new environmental cues; reducing negative metabolites; and improving the balance of metabolites or underlying proteins.

As used herein, a “control sequence” refers to an operator, promoter, silencer, or terminator.

As used herein, “in planta” may refer to in the plant, on the plant, or intimately associated with the plant, depending upon context of usage (e.g. endophytic, epiphytic, or rhizospheric associations). The plant may comprise plant parts, tissue, leaves, roots, root hairs, rhizomes, stems, seed, ovules, pollen, flowers, fruit, etc.

In some embodiments, native or endogenous control sequences of genes of the present disclosure are replaced with one or more intrageneric control sequences.

As used herein, “introduced” refers to the introduction by means of modern biotechnology, and not a naturally occurring introduction.

In some embodiments, the bacteria of the present disclosure have been modified such that they are not naturally occurring bacteria.

In some embodiments, the bacteria of the present disclosure are present in the plant in an amount of at least 10³ cfu, 10⁴ cfu, 10⁵ cfu, 10⁶ cfu, 10⁷ cfu, 10⁸ cfu, 10⁹ cfu, 10¹⁰ cfu, 10¹¹ cfu, 10¹² cfu, 10¹³ cfu, 10¹⁴ cfu or 10¹⁵ cfu, per gram of fresh or dry weight of the plant. In some embodiments, the bacteria of the present disclosure are present in the plant in an amount of at least about 10³ cfu, about 10⁴ cfu, about 10⁵ cfu, about 10⁶ cfu, about 10⁷ cfu, about 10⁸ cfu, about 10⁹ cfu, about 10¹⁰ cfu, about 10¹¹ cfu, about 10¹² cfu, about 10¹³ cfu, about 10¹⁴ cfu or about 10¹⁵ cfu, per gram of fresh or dry weight of the plant. In some embodiments, the bacteria of the present disclosure are present in the plant in an amount of at least 10³ to 10⁹, 10³ to 10⁷, 10³ to 10⁵, 10⁵ to 10⁹, 10⁵ to 10⁷, 10⁶ to 10¹⁰, 10⁶ to 10⁷ cfu, 10⁷ to 10¹¹ cfu, 10⁷ to 10⁸ cfu, 10⁸ to 10¹² cfu, 10⁸ to 10⁹ cfu, 10⁹ to 10¹³ cfu, 10⁹ to 10¹⁰ cfu, 10¹⁰ to 10¹⁴ cfu, 10¹⁰ to 10¹¹ cfu, 10¹¹ to 10¹¹ cfu or 10¹¹ to 10¹² cfu per gram of fresh or dry weight of the plant.

Fertilizers and exogenous nitrogen of the present disclosure may comprise the following nitrogen-containing molecules: ammonium, nitrate, nitrite, ammonia, glutamine, etc. Nitrogen sources of the present disclosure may include anhydrous ammonia, ammonia sulfate, urea, diammonium phosphate, urea-form, monoammonium phosphate, ammonium nitrate, nitrogen solutions, calcium nitrate, potassium nitrate, sodium nitrate, etc.

As used herein, “exogenous nitrogen” refers to non-atmospheric nitrogen readily available in the soil, field, or growth medium that is present under non-nitrogen limiting conditions, including ammonia, ammonium, nitrate, nitrite, urea, uric acid, ammonium acids, etc.

As used herein, “non-nitrogen limiting conditions” refers to non-atmospheric nitrogen available in the soil, field or media at concentrations greater than about 4 mM nitrogen, as disclosed by Kant et al. (2010. J. Exp. Biol. 62(4):1499-1509), which is incorporated herein by reference.

As used herein, an “intergeneric microorganism” is a microorganism that is formed by the deliberate combination of genetic material originally isolated from organisms of different taxonomic genera. An “intergeneric mutant” can be used interchangeably with “intergeneric microorganism”. An exemplary “intergeneric microorganism” includes a microorganism containing a mobile genetic element that was first identified in a microorganism in a genus different from the recipient microorganism. Further explanation can be found, inter alia, in 40 C.F.R. § 725.3.

In aspects, microbes taught herein are “non-intergeneric,” which means that the microbes are not intergeneric.

As used herein, an “intrageneric microorganism” is a microorganism that is formed by the deliberate combination of genetic material originally isolated from organisms of the same taxonomic genera. An “intrageneric mutant” can be used interchangeably with “intrageneric microorganism”.

As used herein, “introduced genetic material” means genetic material that is added to, and remains as a component of, the genome of the recipient.

As used herein, in the context of non-intergeneric microorganisms, the term “remodeled” is used synonymously with the term “engineered”. Consequently, a “non-intergeneric remodeled microorganism” has a synonymous meaning to “non-intergeneric engineered microorganism,” and will be utilized interchangeably. Further, the disclosure may refer to an “engineered strain” or “engineered derivative” or “engineered non-intergeneric microbe,” these terms are used synonymously with “remodeled strain” or “remodeled derivative” or “remodeled non-intergeneric microbe.”

In some embodiments, the nitrogen fixation and assimilation genetic regulatory network comprises polynucleotides encoding genes and non-coding sequences that direct, modulate, and/or regulate microbial nitrogen fixation and/or assimilation and can comprise polynucleotide sequences of the nif cluster (e.g., nifA, nifB, nifC, . . . nifZ), polynucleotides encoding nitrogen regulatory protein C (NitrC), polynucleotides encoding nitrogen regulatory protein B (NtrB), polynucleotide sequences of the gln cluster (e.g. glnA and glnD), glnR, draT, and ammonia transporters/permeases. In some cases, the Nif cluster may comprise NifB, NifH, NifD, NifK, NifE, NifN, NifX, hesa, and NifV. In some cases, the Nif cluster may comprise a subset of NifB, NifH, NifD, NifK, NifE, NifN, NifX, hesa, and NifV.

In some embodiments, fertilizer of the present disclosure comprises at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% nitrogen by weight.

In some embodiments, fertilizer of the present disclosure comprises at least about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% nitrogen by weight.

In some embodiments, fertilizer of the present disclosure comprises about 5% to 50%, about 5% to 75%, about 10% to 50%, about 10% to 75%, about 15% to 50%, about 15% to 75%, about 20% to 50%, about 20% to 75%, about 25% to 50%, about 25% to 75%, about 30% to 50%, about 30% to 75%, about 35% to 50%, about 35% to 75%, about 40% to 50%, about 40% to 75%, about 45% to 50%, about 45% to 75%, or about 50% to 75% nitrogen by weight.

In some embodiments, the increase of nitrogen fixation and/or the production of 1% or more of the nitrogen in the plant are measured relative to control plants, which have not been exposed to the bacteria of the present disclosure. All increases or decreases in bacteria are measured relative to control bacteria. All increases or decreases in plants are measured relative to control plants.

As used herein, a “constitutive promoter” is a promoter that is active under most conditions and/or during most development stages. There are several advantages to using constitutive promoters in expression vectors used in biotechnology, such as: high level of production of proteins used to select transgenic cells or organisms; high level of expression of reporter proteins or scoreable markers, allowing easy detection and quantification; high level of production of a transcription factor that is part of a regulatory transcription system; production of compounds that requires ubiquitous activity in the organism; and production of compounds that are required during all stages of development. Non-limiting exemplary constitutive promoters include, CaMV 35S promoter, opine promoters, ubiquitin promoter, alcohol dehydrogenase promoter, etc.

As used herein, a “non-constitutive promoter” is a promoter that is active under certain conditions, in certain types of cells, and/or during certain development stages. For example, tissue specific, tissue preferred, cell type specific, cell type preferred, inducible promoters, and promoters under development control are non-constitutive promoters. Examples of promoters under developmental control include promoters that preferentially initiate transcription in certain tissues.

As used herein, “inducible” or “repressible” promoter is a promoter that is under chemical or environmental factors control. Examples of environmental conditions that may affect transcription by inducible promoters include anaerobic conditions, certain chemicals, the presence of light, acidic or basic conditions, etc.

As used herein, a “tissue specific” promoter is a promoter that initiates transcription only in certain tissues. Unlike constitutive expression of genes, tissue-specific expression is the result of several interacting levels of gene regulation. As such, in the art sometimes it is preferable to use promoters from homologous or closely related species to achieve efficient and reliable expression of transgenes in particular tissues. This is one of the main reasons for the large amount of tissue-specific promoters isolated from particular tissues found in both scientific and patent literature.

As used herein, the term “operably linked” refers to the association of nucleic acid sequences on a single nucleic acid fragment so that the function of one is regulated by the other.

For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the disclosure can be operably linked, either directly or indirectly, 5′ to the target mRNA, or 3′ to the target mRNA, or within the target mRNA, or a first complementary region is 5′ and its complement is 3′ to the target mRNA.

In aspects, “applying to the plant one or a plurality of bacteria,” “applying to the plant one or a plurality of engineered bacteria,” or “applying to the plant one or a plurality of non-intergeneric bacteria” includes any means by which the plant (including plant parts such as a seed, root, stem, tissue, etc.) is made to come into contact (i.e. exposed) with said bacteria at any stage of the plant's life cycle. Consequently, “applying to the plant one or a plurality of bacteria,” “applying to the plant one or a plurality of engineered bacteria,” or “applying to the plant one or a plurality of non-intergeneric bacteria” includes any of the following means of exposing the plant (including plant parts such as a seed, root, stem, tissue, etc.) to said bacteria: spraying onto plant, dripping onto plant, applying as a seed coat, applying to a field that will then be planted with seed, applying to a field already planted with seed, applying to a field with adult plants, etc.

As used herein “MRTN” is an acronym for maximum return to nitrogen and is utilized as an experimental treatment in the Examples. MRTN was developed by Iowa State University and information can be found at: cnrc.agron.iastate.edu/. The MRTN is the nitrogen rate where the economic net return to nitrogen application is maximized. The approach to calculating the MRTN is a regional approach for developing corn nitrogen rate guidelines in individual states. The nitrogen rate trial data was evaluated for Illinois, Iowa, Michigan, Minnesota, Ohio, and Wisconsin where an adequate number of research trials were available for corn plantings following soybean and corn plantings following corn. The trials were conducted with spring, side dress, or split preplant/side dress applied nitrogen, and sites were not irrigated except for those that were indicated for irrigated sands in Wisconsin. MRTN was developed by Iowa State University due to apparent differences in methods for determining suggested nitrogen rates required for corn production, misperceptions pertaining to nitrogen rate guidelines, and concerns about application rates. By calculating the MRTN, practitioners can determine the following: (1) the nitrogen rate where the economic net return to nitrogen application is maximized, (2) the economic optimum nitrogen rate, which is the point where the last increment of nitrogen returns a yield increase large enough to pay for the additional nitrogen, (3) the value of corn grain increase attributed to nitrogen application, and the maximum yield, which is the yield where application of more nitrogen does not result in a corn yield increase. Thus, the MRTN calculations provide practitioners with the means to maximize corn crops in different regions while maximizing financial gains from nitrogen applications. The term mmol is an abbreviation for millimole, which is a thousandth (10⁻³) of a mole, abbreviated herein as mol.

As used herein the terms “microorganism” or “microbe” should be taken broadly. These terms, used interchangeably, include but are not limited to, the two prokaryotic domains, Bacteria and Archaea. The term may also encompass eukaryotic fungi and protists.

The term “microbial consortia” or “microbial consortium” refers to a subset of a microbial community of individual microbial species, or strains of a species, which can be described as carrying out a common function, or can be described as participating in, or leading to, or correlating with, a recognizable parameter, such as a phenotypic trait of interest.

The term “microbial community” means a group of microbes comprising two or more species or strains. Unlike microbial consortia, a microbial community does not have to be carrying out a common function, or does not have to be participating in, or leading to, or correlating with, a recognizable parameter, such as a phenotypic trait of interest.

As used herein, “isolate,” “isolated,” “isolated microbe,” and like terms, are intended to mean that the one or more microorganisms has been separated from at least one of the materials with which it is associated in a particular environment (for example soil, water, plant tissue, etc.).

Thus, an “isolated microbe” does not exist in its naturally occurring environment; rather, it is through the various techniques described herein that the microbe has been removed from its natural setting and placed into a non-naturally occurring state of existence. Thus, the isolated strain or isolated microbe may exist as, for example, a biologically pure culture, or as spores (or other forms of the strain). In aspects, the isolated microbe may be in association with an acceptable carrier, which may be an agriculturally acceptable carrier.

In certain aspects of the disclosure, the isolated microbes exist as “isolated and biologically pure cultures.” It will be appreciated by one of skill in the art that an isolated and biologically pure culture of a particular microbe, denotes that said culture is substantially free of other living organisms and contains only the individual microbe in question. The culture can contain varying concentrations of said microbe. The present disclosure notes that isolated and biologically pure microbes often “necessarily differ from less pure or impure materials.” See, e.g. In re Bergstrom, 427 F.2d 1394, (CCPA 1970) (discussing purified prostaglandins), see also, In re Bergy, 596 F.2d 952 (CCPA 1979) (discussing purified microbes), see also, Parke-Davis & Co. v. H. K. Mulford & Co., 189 F. 95 (S.D.N.Y. 1911) (Learned Hand discussing purified adrenaline), aff'd in part, rev'd in part, 196 F. 496 (2d Cir. 1912), each of which are incorporated herein by reference. Furthermore, in some aspects, the disclosure provides for certain quantitative measures of the concentration, or purity limitations, that must be found within an isolated and biologically pure microbial culture.

The presence of these purity values, in certain embodiments, is a further attribute that distinguishes the presently disclosed microbes from those microbes existing in a natural state. See, e.g., Merck & Co. v. Olin Mathieson Chemical Corp., 253 F.2d 156 (4th Cir. 1958) (discussing purity limitations for vitamin B12 produced by microbes), incorporated herein by reference.

As used herein, “individual isolates” should be taken to mean a composition, or culture, comprising a predominance of a single genera, species, or strain, of microorganism, following separation from one or more other microorganisms.

Microbes of the present disclosure may include spores and/or vegetative cells. In some embodiments, microbes of the present disclosure include microbes in a viable but non-culturable (VBNC) state. As used herein, “spore” or “spores” refer to structures produced by bacteria and fungi that are adapted for survival and dispersal. Spores are generally characterized as dormant structures; however, spores are capable of differentiation through the process of germination. Germination is the differentiation of spores into vegetative cells that are capable of metabolic activity, growth, and reproduction. The germination of a single spore results in a single fungal or bacterial vegetative cell. Fungal spores are units of asexual reproduction, and in some cases are necessary structures in fungal life cycles. Bacterial spores are structures for surviving conditions that may ordinarily be nonconducive to the survival or growth of vegetative cells.

As used herein, “microbial composition” refers to a composition comprising one or more microbes of the present disclosure. In some embodiments, a microbial composition is administered to plants (including various plant parts) and/or in agricultural fields.

As used herein, “carrier,” “acceptable carrier,” or “agriculturally acceptable carrier” refers to a diluent, adjuvant, excipient, or vehicle with which the microbe can be administered, which does not detrimentally effect the microbe.

### Regulation of Nitrogen Fixation

In some cases, nitrogen fixation pathway may act as a target for genetic engineering and optimization. One trait that may be targeted for regulation by the methods described herein is nitrogen fixation. Nitrogen fertilizer is the largest operational expense on a farm and the biggest driver of higher yields in row crops like corn and wheat. Described herein are microbial products that can deliver renewable forms of nitrogen in non-leguminous crops. While some endophytes have the genetics necessary for fixing nitrogen in pure culture, the fundamental technical challenge is that wild-type endophytes of cereals and grasses stop fixing nitrogen in fertilized fields. The application of chemical fertilizers and residual nitrogen levels in field soils signal the microbe to shut down the biochemical pathway for nitrogen fixation.

Changes to the transcriptional and post-translational levels of components of the nitrogen fixation regulatory network may be beneficial to the development of a microbe capable of fixing and transferring nitrogen to corn in the presence of fertilizer. To that end, described herein is Host-Microbe Evolution (HoME) technology to precisely evolve regulatory networks and elicit novel phenotypes. Also described herein are unique, proprietary libraries of nitrogen-fixing endophytes isolated from corn, paired with extensive omics data surrounding the interaction of microbes and host plant under different environmental conditions like nitrogen stress and excess. In some embodiments, this technology enables precision evolution of the genetic regulatory network of endophytes to produce microbes that actively fix nitrogen even in the presence of fertilizer in the field. In particular, this technology is applied to gram-positive endophytes in order to precisely evolve the genetic regulatory network of said endophytes to produce gram-positive microbes that actively fix nitrogen even in the presence of fertilizer in the field. Also described herein are evaluations of the technical potential of evolving microbes that colonize corn root tissues and produce nitrogen for fertilized plants and evaluations of the compatibility of endophytes with standard formulation practices and diverse soils to determine feasibility of integrating the microbes into modern nitrogen management strategies.

In order to utilize elemental nitrogen (N) for chemical synthesis, life forms combine nitrogen gas (N₂) available in the atmosphere with hydrogen in a process known as nitrogen fixation. Because of the energy-intensive nature of biological nitrogen fixation, diazotrophs (bacteria and archaea that fix atmospheric nitrogen gas) have evolved sophisticated and tight regulation of the nif gene cluster in response to environmental oxygen and available nitrogen. Nif genes encode enzymes involved in nitrogen fixation (such as the nitrogenase complex) and proteins that regulate nitrogen fixation. Shamseldin (2013. Global J. Biotechnol. Biochem. 8(4):84-94) discloses detailed descriptions of nif genes and their products, and is incorporated herein by reference. Described herein are methods of producing and/or identifying gram-positive microbes with the a trait that allows or enables said gram-positive microbes to fix nitrogen regardless of the level of forms of fixed nitrogen (e.g., ammonium) present. Further provided herein are methods for identifying mutations in components of the nitrogen fixation regulatory network of gram-positive bacteria that are or can be beneficial to the development of a microbe capable of fixing and transferring nitrogen to select non-leguminous crops (e.g., corn) in the presence of fertilizer. Also provided herein are compositions comprising gram-positive microbes engineered to possess full or complete de-repression of nitrogenase activity in the presence of levels of fixed nitrogen (e.g., ammonium) that would lead to repression of nitrogenase activity in control or non-engineered control microbes.

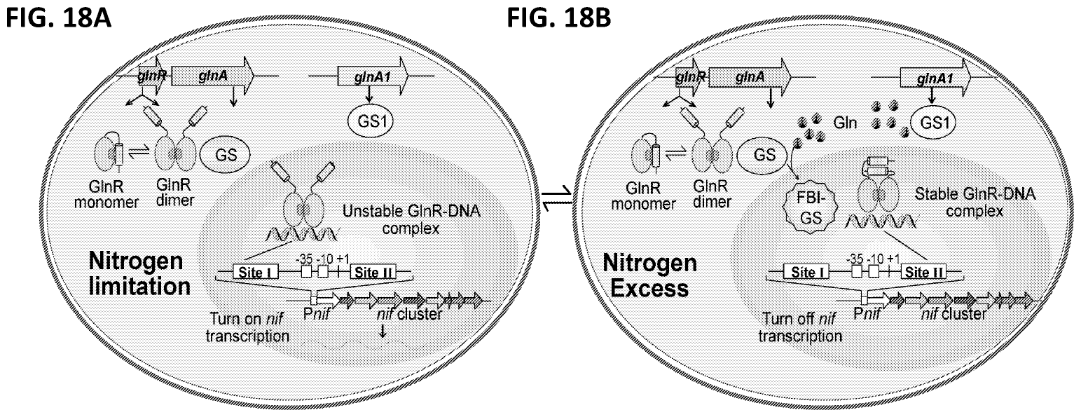

In gram-positive diazotrophic microbes (e.g., Paenibacillus, Bacillus and Lactobacillus), regulation of nitrogen fixation centers around GlnR as shown in FIGS. 18A and 18B and described in Wang T, et al. (2018) Positive and negative regulation of transferred nif genes mediated by indigenous GlnR in Gram-positive Paenibacillus polymyxa. PLOS Genetics 14(9): e1007629. GlnR protein can exists as a mixture of dimer and monomer. The monomer form of GlnR is an autoinhibitory form whose C-terminal region folds back and inhibits dimer formation. As shown in FIG. 18A, during nitrogen limitation, the dimeric form of GlnR binds to GlnR-binding site I in a weak and transient association way and activates nif transcription. Although GlnR can also sequentially or simultaneously binds to site II, binding of GlnR to this site does not repress nif transcription due to GlnR having only a weak and transient association with DNA during this condition. In addition, the large amounts of GlnR produced under this condition can enable nif transcription to carry on, since expression of glnR itself is nitrogen-dependent. As shown in FIG. 18B, during excess nitrogen, glutamine is in excess and it binds to and feedback inhibits glutamine synthetase (GS), which is encoded by glnA within the glnRA operon by forming direct interactions with the C-terminal domain of GlnR, which then controls the GlnR activity and GS1 catalyzing glutamate and NH₄⁺ production. Gln binds to and feedback inhibits GS by forming the complex feedback-inhibited (FBI)-GS (FBI-GS). The FBI-GS can interact with the C-terminal tail of GlnR and relieve its autoinhibition, shifting the monomer to the DNA-binding active form. The FBI-GS can further stabilize the binding affinity of GlnR to GlnR-binding site II and thus represses nif transcription.

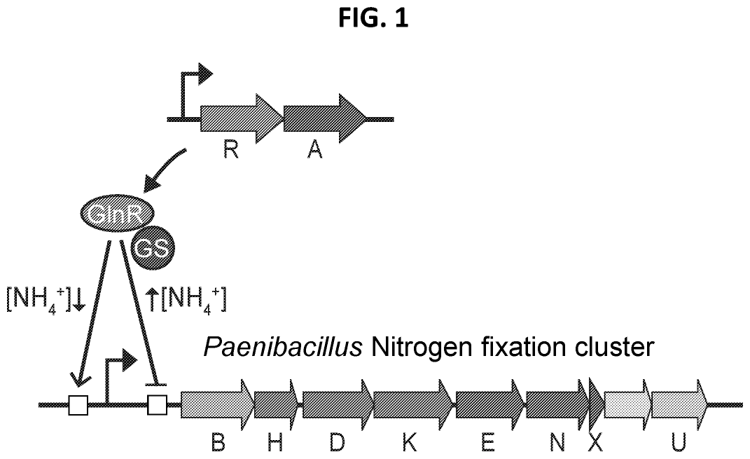

Further, the core nif cluster in these gram-positive microbes (i.e., Paenibacillus, Bacillus and Lactobacillus) is composed of nifBHDKENX-hesA-nifU and is under the control of a nifB promoter that regulates expression of the core nif cluster. The nifB promoter comprises two GlnR-binding operator sites such that under ammonium depletion (i.e., nitrogen limitation) as described herein, GlnR binds upstream of the promoter, recruits RNA polymerase and activates transcription of the nif cluster, whereas under ammonium excess (i.e., excess nitrogen), GlnR binds downstream of the promoter and inhibits transcription by impeding the binding and progression of RNA polymerase (see FIG. 1). Accordingly, in gram-positive microbes (e.g., Paenibacillus, Bacillus and Lactobacillus) multiple layers of regulation can exist that repress nitrogen fixation. This regulation can be facilitated by either cis elements in the promoter of the nif operon, or by elements that act in trans on the nif operon (e.g., transcription factors), or by elements that regulate assimilation of ammonia into the gram-positive cell.

Methods for imparting new microbial phenotypes can be performed at the transcriptional, translational, and post-translational levels. The transcriptional level includes changes at the promoter (such as changing sigma factor affinity or binding sites for transcription factors, including deletion of all or a portion of the promoter) or changing transcription terminators and attenuators. The translational level includes changes at the ribosome binding sites and changing mRNA degradation signals. The post-translational level includes mutating an enzyme's active site and changing protein-protein interactions. These changes can be achieved in a multitude of ways. Reduction of expression level (or complete abolishment) can be achieved by swapping the native ribosome binding site (RBS) or promoter with another with lower strength/efficiency. ATG start sites can be swapped to a GTG, TTG, or CTG start codon, which results in reduction in translational activity of the coding region. Complete abolishment of expression can be done by knocking out (deleting) the coding region of a gene. Frameshifting the open reading frame (ORF) likely will result in a premature stop codon along the ORF, thereby creating a non-functional truncated product. Insertion of in-frame stop codons will also similarly create a non-functional truncated product. Addition of a degradation tag at the N or C terminal can also be done to reduce the effective concentration of a particular gene.

Conversely, expression level of the genes described herein can be achieved by using a stronger promoter. To ensure high promoter activity during high nitrogen level condition (or any other condition), a transcription profile of the whole genome in a high nitrogen level condition could be obtained and active promoters with a desired transcription level can be chosen from that dataset to replace the weak promoter. Weak start codons can be swapped out with an ATG start codon for better translation initiation efficiency. Weak ribosomal binding sites (RBS) can also be swapped out with a different RBS with higher translation initiation efficiency. In addition, site-specific mutagenesis can also be performed to alter the activity of an enzyme.

In one aspect, provided herein are gram-positive microbes that possess one or more mutation(s) in the cis elements regulating expression of the core nif cluster. The mutation(s) in the cis elements of the core nif cluster can confer full or complete de-repression of expression of the nif cluster in the presence of levels of fixed nitrogen that would normally lead to repression of expression of said nif cluster. In other words, gram-positive microbes that comprise or contain the one or more mutations in the cis elements of the core mf cluster can express the mf cluster and thus possess nitrogenase activity irrespective of the levels of fixed nitrogen.

Mutations of the cis regulatory elements of the nif operon in a gram-positive microbe provided herein can comprise substitution of all or portions of the native nifB promoter controlling expression of the core nif cluster with a constitutive promoter that has been characterized to drive expression of genes under the control of said constitutive promoter in the presence of levels or concentrations of fixed nitrogen that would normally confer repression of the core nif cluster. Substitution with the constitutive promoter can be immediately upstream of the nifB gene, or can be in a region that results in deletion of 51-100 bp of the native nifB promoter region comprising the GlnR repressor-binding site or can be such that the constitutive promoter replaces the GlnR repressor-binding site along with the native promoter transcription start site. In one embodiment, the constitutive promoter completely replaces the nif operon endogenous promoter. In another embodiment, the constitutive promoter replaces a portion of the nif operon endogenous promoter downstream of a GlnR activator site, endogenous transcription start site and a GlnR repressor site. In yet another embodiment, the constitutive promoter replaces a portion of the nif operon endogenous promoter downstream of a GlnR activator site and endogenous transcription start site. In still another embodiment, the constitutive promoter replaces a portion of the nif operon endogenous promoter downstream of a GlnR activator site. The constitutive promoters can be heterologous promoters.

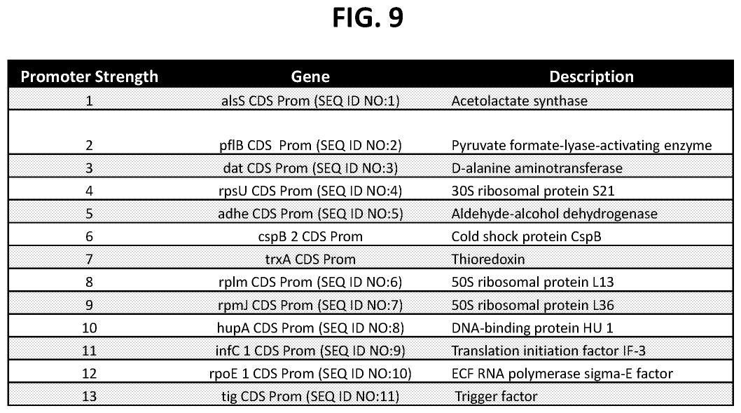

Examples of constitutive promoters suitable for use in controlling expression of the core nif cluster in gram-positive microbes provided herein can be found in FIG. 9. More specifically, the constitutive promoter suitable for use in controlling expression of the core nif cluster in gram-positive microbes provided herein can be a heterologous promoter selected from the group consisting of a promoter for the Paenibacillus Acetolactate synthase (alsS) gene, Pyruvate formate-lyase-activating enzyme (pflB) gene, D-alanine aminotransferase (dat) gene, 30S ribosomal protein S21 (rpsU) gene, Aldehyde-alcohol dehydrogenase (adhe) gene, 50S ribosomal protein L13 (rplm) gene, 50S ribosomal protein L36 (rpmJ) gene, DNA-binding protein HU 1 (hupA) gene, Translation initiation factor IF-3 (infC) gene, ECF RNA polymerase sigma-E factor (rpoE) gene, and Trigger factor (tig) gene. In one embodiment, the promoter for use in controlling expression of the core nif cluster in gram-positive microbes provided herein is selected from the group consisting of the promoter for the alsS gene, pflB gene, rpsU gene, adhe gene, rplm gene, and tig gene. In one embodiment, the promoter for use in controlling expression of the core nif cluster in gram-positive microbes provided herein is the promoter for the pflB gene. In one embodiment, the promoter for use in controlling expression of the core nif cluster in gram-positive microbes provided herein is the promoter for the adhE gene. In one embodiment, the promoter for use in controlling expression of the core nif cluster in gram-positive microbes provided herein is the promoter for the tig gene. In one embodiment, the promoter for use in controlling expression of the core nif cluster in gram-positive microbes provided herein is selected from the group consisting of the promoter with a nucleic acid sequence of SEQ ID NO: 1, 2, 3, 4, 5, 6, 7, 8, 9, 10 and 11. In one embodiment, the promoter for use in controlling expression of the core nif cluster in gram-positive microbes provided herein is selected from the group consisting of the promoter with a nucleic acid sequence of SEQ ID NO: 1, 2, 4, 5, 6 and 11. In one embodiment, the promoter for use in controlling expression of the core nif cluster in gram-positive microbes provided herein is the promoter with the nucleic acid sequence of SEQ ID NO: 2. In one embodiment, the promoter for use in controlling expression of the core nif cluster in gram-positive microbes provided herein is the promoter with the nucleic acid sequence of SEQ ID NO: 5. In one embodiment, the promoter for use in controlling expression of the core nif cluster in gram-positive microbes provided herein is the promoter with the nucleic acid sequence of SEQ ID NO: 11.

Mutations of the trans regulatory elements of the nif operon in a gram-positive microbe provided herein can comprise mutations in the GlnR and/or GlnA.

In one embodiment, mutation of a trans regulatory elements in a gram-positive microbe provided herein comprises a mutant glnR gene in said gram-positive microbe. The mutant glnR gene can comprise at least one nucleotide substitution at nucleotide position 45, 46, 52, 111, 160, 272, 296, 316, 341, 347, 365, 382, 384 or 397 of a Paenibacillus glnR gene (e.g., SEQ ID NO:12) or at a homologous nucleotide position in a homolog thereof. In some cases, the mutant glnR gene shares at least 85%, 86%, 87%, 88%, 89%, 90%, 91%, 92%, 93%, 94%, 95%, 96%, 97%, 98%, or 99% identity with the Paenibacillus glnR gene (e.g., SEQ ID NO:12) or the homolog thereof. The Paenibacillus glnR gene can comprise a nucleic acid sequence of SEQ ID NO: 12. In one embodiment, the mutant glnR gene comprises a nucleic acid sequence selected from the group consisting of SEQ ID NO: 13-15.

In one embodiment, mutation of a trans regulatory elements in a gram-positive microbe provided herein comprises a mutant glnR gene in said gram-positive microbe that encodes a mutant GlnR protein. In one embodiment, mutations of the trans regulatory elements in a gram-positive microbe provided herein comprises one or more amino acid substitutions in the GlnR protein such that said one or mutations allow for the GlnR protein to continue to work to activate the nif cluster irrespective of the levels of fixed nitrogen (e.g., ammonium). As such, the one or more mutations of the GlnR protein can remove the ability of GlnR to represses expression from the mf operon in the presence of ammonium. The mutant GlnR protein can comprise at least one amino acid substitution of at amino acid position 16, 18, 37, 54, 91, 99, 106, 114, 116, 122, 128 or 133 of a Paenibacillus GlnR protein or at a homologous amino acid position in a homolog thereof. In some cases, the mutant GlnR comprises at least one amino acid substitution selected from the group consisting of I16V, M18V, I37M, V54I, T91I, R99H, L106F, L114P, A116V, Q122R, G128S and F133L of a Paenibacillus GlnR protein or at a homologous amino acid position in a homolog thereof. The mutant GlnA protein can share at least 90%, 91%, 92%, 93%, 94%, 95%, 96%, 97%, 98%, or 99% identity with the Paenibacillus GlnR protein or the homolog thereof. In one embodiment, the GlnR protein comprises a L114P mutation. The GlnR protein can comprises a L114P mutation and one or more of a R99H mutation, an A116V mutation, a F133L mutation, an I16V mutation, a T91I mutation, a L106F mutation, a G128S mutation, a M18V mutation, an I37M mutation, a V54I mutation, a Q122R mutation and any combination thereof. In another embodiment, the GlnR protein comprises a L114P, a R99H mutation, an A116V mutation, and a F133L mutation. In yet another embodiment, the GlnR protein comprises a L114P, an I16V mutation, a T91I mutation, a L106F mutation, and a G128S mutation. In still another embodiment, the mutant GlnR protein comprises a L114P, a M18V mutation, an I37M mutation, a V54I mutation, and a Q122R mutation. The Paenibacillus glnR gene can comprise an amino acid sequence of SEQ ID NO: 16. The mutant GlnR protein present in a gram-positive microbe provided herein can comprise an amino acid sequence of SEQ ID NO. 17. The GlnR protein present in a gram-positive microbe provided herein can comprise an amino acid sequence of SEQ ID NO. 18. The GlnR protein present in a gram-positive microbe provided herein can comprise an amino acid sequence of SEQ ID NO. 19.

In one embodiment, mutation of a trans regulatory elements in a gram-positive microbe provided herein comprises a mutant glnA gene in said gram-positive microbe. In some cases, the mutant glnA gene shares at least 85%, 86%, 87%, 88%, 89%, 90%, 91%, 92%, 93%, 94%, 95%, 96%, 97%, 98%, or 99% identity with the Paenibacillus glnA gene or the homolog thereof. The Paenibacillus glnA gene can comprise a nucleic acid sequence of SEQ ID NO: 48. The Paenibacillus glnA gene can comprise a nucleic acid sequence of SEQ ID NO: 49. The homolog thereof can be a Klebsiella glnA gene. The Klebsiella glnA gene can comprise a nucleic acid sequence of SEQ ID NO: 50. In one embodiment, mutation of a trans regulatory elements in a gram-positive microbe provided herein comprises a mutant glnA gene in said gram-positive microbe that encodes a mutant GlnA protein. In one embodiment, mutations of the trans regulatory elements of the nif operon in a gram-positive microbe provided herein comprises one or more mutations in the GlnA protein such that said one or mutations allow for the GlnA protein to exhibit an increase in excretion of fixed nitrogen and/or decreased assimilation of fixed nitrogen. The GlnA protein can comprise at least one amino acid substitution of at amino acid position 67, 182, 241 or 313 of a Paenibacillus GlnA protein or at a homologous amino acid position in a homolog thereof. The homolog thereof can be a Klebsiella GlnA protein and the homologous amino acid position can be at positions 66, 208, 268 or 339. In some cases, the GlnA comprises at least one amino acid substitution selected from the group consisting of M67I, E182K, G241S and N313B of a Paenibacillus GlnA or at a homologous amino acid position in a homolog thereof. In some cases, the Paenibacillus GlnA can be a Paenibacillus polymyxa CI41 GlnA protein. In some cases, the homolog thereof can be a Klebsiella GlnA protein. In some cases, the homolog thereof can be a Klebsiella variicola CI137 GlnA protein. The homolog thereof can be a Klebsiella GlnA protein and the homologous amino acid position can be selected from the group consisting of M66I, E208K, G268S and N339D. The GlnA protein can share at least 90%, 91%, 92%, 93%, 94%, 95%, 96%, 97%, 98%, or 99% identity with the Paenibacillus GlnA protein or the homolog thereof. In some cases, the Paenibacillus GlnA can be a Paenibacillus polymyxa CI41 GlnA protein. In some cases, the homolog thereof can be a Klebsiella GlnA protein. In some cases, the homolog thereof can be a Klebsiella variicola CI137 GlnA protein. The GlnA protein can be mutated to contain one or more single nucleotide polymorphisms (SNPs) selected from the SNPs present in Table 4. The GlnA protein can comprise a substitution or any combination of substitutions that correspond to an M67I, E182K, G241S, or N313D mutation in the GlnA protein of Paenibacillus C141. The Paenibacillus GlnA protein can have the amino acid sequence of SEQ ID NO: 51. The Paenibacillus GlnA protein can have the amino acid sequence of SEQ ID NO: 52. The GlnA protein can comprise a substitution or any combination of substitutions that correspond to an M66I, E208K, G268S, or N339D mutation in the GlnA protein of K. variicola C1137. The K. variicola GlnA protein can have the amino acid sequence of SEQ ID NO: 53.

In one embodiment, a gram-positive microbe provided herein or for use in a method provided herein comprises a combination of mutations in a cis regulatory element and trans regulatory element of the nif operon as provided herein. A gram-positive microbe provided herein or for use in a method provided herein can comprise a mutation in the nifB promoter of the nif operon as provided herein in combination with a mutant GlnR as provided herein. A gram-positive microbe provided herein or for use in a method provided herein can comprise a mutation in the nifB promoter of the nif operon as provided herein in combination with a mutant GlnA as provided herein. A gram-positive microbe provided herein or for use in a method provided herein can comprise a mutant GlnA as provided herein in combination with a mutant GlnR as provided herein.

In one embodiment, a gram-positive microbe provided herein can comprise a mutant form of the nifB promoter operably linked to the nif cluster as provided herein, a mutant GlnR as provided herein, a mutant GlnA as provided herein or any combination thereof in combination with at least one genetic variation introduced into a member selected from the group consisting of: nifB, nifH, nifD, nifK, nifE, nifN, nifX, hesA, nifV genes or combinations thereof.

Increasing the level of nitrogen fixation that occurs in a plant can lead to a reduction in the amount of chemical fertilizer needed for crop production and reduce greenhouse gas emissions (e.g., nitrous oxide).

As used herein, “homolog” refers to both a protein and the DNA sequence encoding it. Homologs are identified by shared function or structure at the protein or DNA level and can be identified by protein sequence alignments, DNA sequence alignments, or comparisons of confirmed or predicted secondary or tertiary protein structure. It would be recognized by those of skill in the art, that nucleotide and protein sequence homologs may be of the same length or may contain insertions and/or deletions. In some cases, the homologous nucleotide and/or amino acid position in a homolog is identical to the nucleotide or amino acid position in a base sequence. In other cases, the homologous nucleotide and/or amino acid position in a homolog is a different nucleotide or amino acid position than in the base sequence. The homologous nucleotide or amino acid position in the homolog (the position in the homolog at which a substitution would occur based upon a substitution position disclosed herein) can be identified by aligning the homolog to a base sequence with a substitution disclosed herein and identifying the position of the nucleotide or the amino acid in the homolog that aligns with the position of the nucleotide or the amino acid in the base sequence that contains a substitution as disclosed herein. Such sequence alignment can be carried out by methods known to those of skill in the art.

### Generation of Bacterial Populations

### Isolation of Bacteria

Microbes useful in methods and compositions disclosed herein can be obtained by extracting microbes from surfaces or tissues of native plants. Microbes can be obtained by grinding seeds to isolate microbes. Microbes can be obtained by planting seeds in diverse soil samples and recovering microbes from tissues. Additionally, microbes can be obtained by inoculating plants with exogenous microbes and determining which microbes appear in plant tissues. Non-limiting examples of plant tissues may include a seed, seedling, leaf, cutting, plant, bulb, or tuber.

A method of obtaining microbes may be through the isolation of bacteria from soils. Bacteria may be collected from various soil types. In some example, the soil can be characterized by traits such as high or low fertility, levels of moisture, levels of minerals, and various cropping practices. For example, the soil may be involved in a crop rotation where different crops are planted in the same soil in successive planting seasons. The sequential growth of different crops on the same soil may prevent disproportionate depletion of certain minerals. The bacteria can be isolated from the plants growing in the selected soils. The seedling plants can be harvested at 2-6 weeks of growth. For example, at least 400 isolates can be collected in a round of harvest. Soil and plant types reveal the plant phenotype as well as the conditions, which allow for the downstream enrichment of certain phenotypes.

Microbes can be isolated from plant tissues to assess microbial traits. The parameters for processing tissue samples may be varied to isolate different types of associative microbes, such as rhizopheric bacteria, epiphytes, or endophytes. The isolates can be cultured in nitrogen-free media to enrich for bacteria that perform nitrogen fixation. Alternatively, microbes can be obtained from global strain banks.

In planta analytics are performed to assess microbial traits. In some embodiments, the plant tissue can be processed for screening by high throughput processing for DNA and RNA. Additionally, non-invasive measurements can be used to assess plant characteristics, such as colonization. Measurements on wild microbes can be obtained on a plant-by-plant basis. Measurements on wild microbes can also be obtained in the field using medium throughput methods. Measurements can be done successively over time. Model plant system can be used including, but not limited to, Setaria.

Microbes in a plant system can be screened via transcriptional profiling of a microbe in a plant system. Examples of screening through transcriptional profiling are using methods of quantitative polymerase chain reaction (qPCR), molecular barcodes for transcript detection, Next Generation Sequencing, and microbe tagging with fluorescent markers. Impact factors can be measured to assess colonization in the greenhouse including, but not limited to, microbiome, abiotic factors, soil conditions, oxygen, moisture, temperature, inoculum conditions, and root localization. Nitrogen fixation can be assessed in bacteria by measuring 15N gas/fertilizer (dilution) with IRMS or NanoSIMS as described herein NanoSIMS is high-resolution secondary ion mass spectrometry. The NanoSIMS technique is a way to investigate chemical activity from biological samples. The catalysis of reduction of oxidation reactions that drive the metabolism of microorganisms can be investigated at the cellular, subcellular, molecular and elemental level. NanoSIMS can provide high spatial resolution of greater than 0.1 μm. NanoSIMS can detect the use of isotope tracers such as ¹³C, ¹⁵N, and ¹⁸O. Therefore, NanoSIMS can be used to the chemical activity nitrogen in the cell.

Automated greenhouses can be used for in planta analytics. Plant metrics in response to microbial exposure include, but are not limited to, biomass, chloroplast analysis, CCD camera, volumetric tomography measurements.

One way of enriching a microbe population is according to genotype. For example, a polymerase chain reaction (PCR) assay with a targeted primer or specific primer. Primers designed for the nifH gene can be used to identity diazotrophs because diazotrophs express the nifH gene in the process of nitrogen fixation. A microbial population can also be enriched via single-cell culture-independent approaches and chemotaxis-guided isolation approaches. Alternatively, targeted isolation of microbes can be performed by culturing the microbes on selection media. Premeditated approaches to enriching microbial populations for desired traits can be guided by bioinformatics data and are described herein.

Enriching for Microbes with Nitrogen Fixation Capabilities Using Bioinformatics

Bioinformatics tools can be used to identify and isolate plant growth promoting rhizobacteria (PGPRs), which are selected based on their ability to perform nitrogen fixation. Microbes with high nitrogen fixing ability can promote favorable traits in plants. Bioinformatics modes of analysis for the identification of PGPRs include, but are not limited to, genomics, metagenomics, targeted isolation, gene sequencing, transcriptome sequencing, and modeling.

Genomics analysis can be used to identify PGPRs and confirm the presence of mutations with methods of Next Generation Sequencing (NGS) as described herein and microbe version control.

Metagenomics can be used to identify and isolate PGPR using a prediction algorithm for colonization. Metadata can also be used to identify the presence of an engineered strain in environmental and greenhouse samples.

Transcriptomic sequencing can be used to predict genotypes leading to PGPR phenotypes. Additionally, transcriptomic data is used to identify promoters for altering gene expression. Transcriptomic data can be analyzed in conjunction with the Whole Genome Sequence (WGS) to generate models of metabolism and gene regulatory networks.

### Domestication of Microbes

Microbes isolated from nature can undergo a domestication process wherein the microbes are converted to a form that is genetically trackable and identifiable. One way to domesticate a microbe is to engineer it with antibiotic resistance. The process of engineering antibiotic resistance can begin by determining the antibiotic sensitivity in the wild type microbial strain. If the bacteria are sensitive to the antibiotic, then the antibiotic can be a good candidate for antibiotic resistance engineering. Subsequently, an antibiotic resistant gene or a counterselectable suicide vector can be incorporated into the genome of a microbe using recombineering methods. A counterselectable suicide vector may consist of a deletion of the gene of interest, a selectable marker, and the counterselectable marker sacB. Counterselection can be used to exchange native microbial DNA sequences with antibiotic resistant genes. A medium throughput method can be used to evaluate multiple microbes simultaneously allowing for parallel domestication. Alternative methods of domestication include the use of homing nucleases to prevent the suicide vector sequences from looping out or from obtaining intervening vector sequences.

DNA vectors can be introduced into bacteria via several methods including electroporation and chemical transformations. A standard library of vectors can be used for transformations. An example of a method of gene editing is CRISPR preceded by Cas9 testing to ensure activity of Cas9 in the microbes.

### Non-Transgenic Engineering of Microbes

A microbial population with favorable traits can be obtained via directed evolution. Direct evolution is an approach wherein the process of natural selection is mimicked to evolve proteins or nucleic acids towards a user-defined goal. An example of direct evolution is when random mutations are introduced into a microbial population, the microbes with the most favorable traits are selected, and the growth of the selected microbes is continued. The most favorable traits in growth promoting rhizobacteria (PGPRs) may be in nitrogen fixation. The method of directed evolution may be iterative and adaptive based on the selection process after each iteration.

PGPRs with high capability of nitrogen fixation can be generated. The evolution of PGPRs can be carried out via the introduction of genetic variation. Genetic variation can be introduced via polymerase chain reaction mutagenesis, oligonucleotide-directed mutagenesis, saturation mutagenesis, fragment shuffling mutagenesis, homologous recombination, CRISPR/Cas9 systems, chemical mutagenesis, and combinations thereof. These approaches can introduce random mutations into the microbial population. For example, mutants can be generated using synthetic DNA or RNA via oligonucleotide-directed mutagenesis. Mutants can be generated using tools contained on plasmids, which are later cured. Genes of interest can be identified using libraries from other species with improved traits including, but not limited to, improved PGPR properties, improved colonization of cereals, increased oxygen sensitivity, increased nitrogen fixation, and increased ammonia excretion. Intrageneric genes can be designed based on these libraries using software such as Geneious or Platypus design software. Mutations can be designed with the aid of machine learning. Mutations can be designed with the aid of a metabolic model. Automated design of the mutation can be done using a la Platypus and will guide RNAs for Cas-directed mutagenesis.

The intra-generic genes can be transferred into the host microbe. Additionally, reporter systems can also be transferred to the microbe. The reporter systems characterize promoters, determine the transformation success, screen mutants, and act as negative screening tools.

The microbes carrying the mutation can be cultured via serial passaging. A microbial colony contains a single variant of the microbe. Microbial colonies are screened with the aid of an automated colony picker and liquid handler. Mutants with gene duplication and increased copy number express a higher genotype of the desired trait.

### Reporter Systems for Characterizing Mutants of Nitrogen Fixation Regulatory Network

In one aspect, provided herein is a method for identifying regulators of a mf operon that exhibit de-repression activity irrespective of the levels of fixed nitrogen and/or in the presence of ammonium. The method can comprise (a) introducing individual mutagenized glnR genes from a library of mutagenized glnR genes into a gram-positive microbial host cell missing a wild-type glnR gene such that the gram-positive microbial host cell comprises a nucleic acid sequence encoding a selectable marker protein, functional fragment, and/or fusions thereof operably linked to a nifB promoter; (b) culturing the gram-positive microbial host cell in the presence of fixed nitrogen (e.g., ammonium) under anaerobic conditions such that the gram-positive microbial host cell expresses the marker protein, functional fragment, and/or fusions thereof in the presence of fixed nitrogen (e.g., ammonium) if the mutagenized glnR gene introduced in step (a) encodes a GlnR protein that exhibits de-repression activity in the presence of or irrespective of the level or concentration of fixed nitrogen (e.g., ammonium); (c) exposing the gram-positive microbial host cell to an agent that allows for selection of gram-positive microbial host cell's expressing the selectable marker protein; and (d) identifying individual mutagenized glnR genes from the library of mutagenized glnR genes as exhibiting de-repression activity in the presence of or irrespective of the level or concentration of fixed nitrogen (e.g., ammonium) as those that result in selection of the gram-positive microbial host cell's expressing the selectable marker protein as compared to a control. The control can be a gram-positive microbial host cell expressing wild-type GlnR. The gram-positive microbial host cell and/or control can be diazotrophic. The microbial host cell and/or can be any gram-positive microbe known in the art and/or provided herein. The microbial host cell and/or control can be from the Paenibacillus, Lactobacillus or Bacillus genus. In one embodiment, the microbial host cell and/or control is a species of Paenibacillus. The gram-positive microbial host cell can be a transgenic or remodeled non-intergeneric host cell. In one embodiment, step (b) is performed in the presence of at least 1 mM, 2 mM, 3 mM, 4 nM, 5 mM, 6 mM, 7 mM, 8 mM, 9 mM or 10 mM ammonium.

The selectable marker protein for use herein can be auxotrophic markers, prototrophic markers, dominant markers, recessive markers, antibiotic resistance markers, catabolic markers, enzymatic markers, chromogenic markers, fluorescent markers, luminescent markers or combinations thereof. In one embodiment, the selectable marker protein is a fluorescent marker protein, a bioluminescent marker or photoprotein or a chemiluminescent marker protein. In some aspects, the selectable marker protein is a bioluminescent photoprotein such as aequorin, which is derived from the hydrozoan Aequorea victoria. In some aspects, the selectable marker protein can be calcium-sensitive luminescent or fluorescent molecules, such as obelin, thalassicolin, mitrocomin (halistaurin), clytin (phialidin), mnemopsin, berovin, Indo-1, Fura-2, Quin-2, Fluo-3, Rhod-2, calcium green, BAPTA, cameleons, or similar molecules. In some aspects, the selectable marker protein can be a chimeric protein that includes a Ca′ binding domain and an associated fluorescent protein. In some aspects, the selectable marker protein can be an enzyme that is adapted to produce a luminescent or fluorescent signal. In some aspects, the selectable marker protein can be an enzyme such as luciferase or alkaline phosphatase that yields a luminescent or fluorescent signal respectively. In some aspects, the selectable marker protein can also be a fluorescent protein or can include fluorescent, charged, or magnetic nanoparticles, nanodots, or quantum dots. In some aspects, the selectable marker protein can be a dye that has fluorescent, ultraviolet, or visible properties, wherein the fluorescent, ultraviolet, or visible properties undergo a detectable change.

In one embodiment, the selectable marker protein is a fluorescent marker protein. The fluorescent marker protein can be a GFP, RFP, YFP, CFP, or functional variant or fragment thereof.

In some aspects, the fluorescent marker protein is selected from the far-red class of fluorescent proteins. In some aspects, the far-red fluorescent protein is mPlum or a variant thereof. In some aspects, the fluorescent marker protein is selected from the red class of fluorescent proteins. In some aspects, the red fluorescent protein is selected from RFP, mCherry, tdTomato, mStrawberry, J-Red, DsRed-monomer, or a variant thereof. In some aspects, the fluorescent marker protein is selected from the orange class of fluorescent proteins. In some aspects, the orange fluorescent protein is selected from OFP, mOrange, mKO, or a variant thereof.

In some aspects, the fluorescent marker protein is selected from the yellow-green class of fluorescent proteins. In some aspects, the yellow-green fluorescent protein is selected from YFP, mCitrine, Venus, YPet, EYFP, or a variant thereof.

In some aspects, the fluorescent marker protein is selected from the green class of fluorescent proteins. In some aspects, the green fluorescent protein is selected from GFP, EGFP, Emerald, or a variant thereof. In some aspects, the fluorescent protein is selected from the UV-excitable green class of fluorescent proteins. In some aspects, the UV-excitable green fluorescent protein is selected from T-sapphire.

In some aspects, the fluorescent marker protein is selected from the cyan class of fluorescent proteins. In some aspects, the cyan fluorescent protein is selected from Cypet, mCFPm, Cerulean, CFP, or a variant thereof.

In some cases, the method can comprise (a) introducing individual mutagenized glnR genes from a library of mutagenized glnR genes into a gram-positive microbial host cell missing a wild-type glnR gene such that the gram-positive microbial host cell comprises a nucleic acid sequence encoding a fluorescent marker protein, functional fragment, and/or fusions thereof operably linked to a nifB promoter; (b) culturing the engineered gram-positive diazotrophic microbial host cell in the presence of fixed nitrogen (e.g., ammonium) under anaerobic conditions, such that the gram-positive microbial host cell expresses the fluorescent protein, functional fragment, and/or fusions thereof in the presence of or irrespective of the levels of fixed nitrogen (e.g., ammonium) if the mutagenized glnR gene introduced in step (a) encodes a GlnR protein that exhibits de-repression activity in the presence of or irrespective of the levels of fixed nitrogen (e.g., ammonium); (c) exposing the gram-positive microbial host cell to light excitation sufficient to fluoresce the fluorescent marker protein, functional fragment, and/or fusions thereof, and (d) identifying individual mutagenized glnR genes from the library of mutagenized glnR genes as exhibiting de-repression activity in the presence of or irrespective of the levels of fixed nitrogen (e.g., ammonium) as those that result in fluorescence of the fluorescent marker protein, functional fragment, and/or fusions thereof, as compared to a control. The fluorescence can be detected with a flow cytometer, a plate reader, or fluorescence-activated droplet sorting. The control can be a gram-positive microbial host cell expressing wild-type GlnR. The gram-positive microbial host cell and/or control can be diazotrophic. The microbial host cell and/or can be any gram-positive microbe known in the art and/or provided herein. The microbial host cell and/or control can be from the Paenibacillus, Lactobacillus or Bacillus genus. In one embodiment, the microbial host cell and/or control is a species of Paenibacillus. The gram-positive microbial host cell can be a transgenic or remodeled non-intergeneric host cell. In one embodiment, step (b) is performed in the presence of at least 1 mM, 2 mM, 3 mM, 4 nM, 5 mM, 6 mM, 7 mM, 8 mM, 9 mM or 10 mM ammonium.

### Selection of Plant Growth Promoting Microbes Based on Nitrogen Fixation

The microbial colonies can be screened using various assays to assess nitrogen fixation.

One way to measure nitrogen fixation is via a single fermentative assay, which measures nitrogen excretion. An alternative method is the acetylene reduction assay (ARA) with in-line sampling over time. ARA can be performed in high throughput plates of microtube arrays. ARA can be performed with live plants and plant tissues. The media formulation and media oxygen concentration can be varied in ARA assays. Another method of screening microbial variants is by using biosensors. The use of NanoSIMS and Raman microspectroscopy can be used to investigate the activity of the microbes. In some cases, bacteria can also be cultured and expanded using methods of fermentation in bioreactors. The bioreactors are designed to improve robustness of bacteria growth and to decrease the sensitivity of bacteria to oxygen. Medium to high TP plate-based microfermentors are used to evaluate oxygen sensitivity, nutritional needs, nitrogen fixation, and nitrogen excretion. The bacteria can also be co-cultured with competitive or beneficial microbes to elucidate cryptic pathways. Flow cytometry can be used to screen for bacteria that produce high levels of nitrogen using chemical, colorimetric, or fluorescent indicators. The bacteria may be cultured in the presence or absence of a nitrogen source. For example, the bacteria may be cultured with glutamine, ammonia, urea or nitrates.

### Guided Microbial Remodeling—An Overview

Guided microbial remodeling is a method to systematically identify and improve the role of species within the crop microbiome. In some aspects, and according to a particular methodology of grouping/categorization, the method comprises three steps: 1) selection of candidate species by mapping plant-microbe interactions and predicting regulatory networks linked to a particular phenotype, 2) pragmatic and predictable improvement of microbial phenotypes through intra-species crossing of regulatory networks and gene clusters within a microbe's genome, and 3) screening and selection of new microbial genotypes that produce desired crop phenotypes.

To systematically assess the improvement of strains, a model is created that links colonization dynamics of the microbial community to genetic activity by key species. The model is used to predict genetic targets for non-intergeneric genetic remodeling (i.e. engineering the genetic architecture of the microbe in a non-transgenic fashion).

Rational improvement of the crop microbiome may be used to increase soil biodiversity, tune impact of keystone species, and/or alter timing and expression of important metabolic pathways.

### Serial Passage

Production of bacteria to improve plant traits (e.g., nitrogen fixation) can be achieved through serial passage. The production of this bacteria can be done by selecting plants, which have a particular improved trait that is influenced by the microbial flora, in addition to identifying bacteria and/or compositions that are capable of imparting one or more improved traits to one or more plants. One method of producing a bacteria to improve a plant trait includes the steps of: (a) isolating bacteria from tissue or soil of a first plant; (b) introducing a genetic variation into one or more of the bacteria to produce one or more variant bacteria; (c) exposing a plurality of plants to the variant bacteria; (d) isolating bacteria from tissue or soil of one of the plurality of plants, wherein the plant from which the bacteria is isolated has an improved trait relative to other plants in the plurality of plants; and (e) repeating steps (b) to (d) with bacteria isolated from the plant with an improved trait (step (d)). Steps (b) to (d) can be repeated any number of times (e.g., once, twice, three times, four times, five times, ten times, or more) until the improved trait in a plant reaches a desired level. Further, the plurality of plants can be more than two plants, such as 10 to 20 plants, or 20 or more, 50 or more, 100 or more, 300 or more, 500 or more, or 1000 or more plants.

In addition to obtaining a plant with an improved trait, a bacterial population comprising bacteria comprising one or more genetic variations introduced into one or more genes (e.g., genes regulating nitrogen fixation) is obtained. By repeating the steps described above, a population of bacteria can be obtained that include the most appropriate members of the population that correlate with a plant trait of interest. The bacteria in this population can be identified and their beneficial properties determined, such as by genetic and/or phenotypic analysis. Genetic analysis may occur of isolated bacteria in step (a). Phenotypic and/or genotypic information may be obtained using techniques including: high through-put screening of chemical components of plant origin, sequencing techniques including high throughput sequencing of genetic material, differential display techniques (including DDRT-PCR, and DD-PCR), nucleic acid microarray techniques, RNA-sequencing (Whole Transcriptome Shotgun Sequencing), and qRT-PCR (quantitative real time PCR). Information gained can be used to obtain community-profiling information on the identity and activity of bacteria present, such as phylogenetic analysis or microarray-based screening of nucleic acids coding for components of rRNA operons or other taxonomically informative loci. Examples of taxonomically informative loci include 16S rRNA gene, 23S rRNA gene, 5S rRNA gene, 5.8S rRNA gene, 12S rRNA gene, 18S rRNA gene, 28S rRNA gene, gyrB gene, rpoB gene, fusA gene, recA gene, coxl gene, nifD gene. Example processes of taxonomic profiling to determine taxa present in a population are described in US20140155283. Bacterial identification may comprise characterizing activity of one or more genes or one or more signaling pathways, such as genes associated with the nitrogen fixation pathway. Synergistic interactions (where two components, by virtue of their combination, increase a desired effect by more than an additive amount) between different bacterial species may also be present in the bacterial populations.

### Genetic Variation—Locations and Sources of Genomic Alteration

The genetic variation may be a gene selected from the group consisting of: nifB, nifH, nifD, nifK, nifE, nifN, nifX, hesA and nifV. The genetic variation may be a variation in a gene encoding a protein with functionality selected from the group consisting of: glutamine synthetase, glutaminase, glutamine synthetase adenylyltransferase, transcriptional activator, anti-transcriptional activator, pyruvate flavodoxin oxidoreductase, flavodoxin, or NAD+-dinitrogen-reductase ADP-D-ribosyltransferase. The genetic variation may be a mutation that results in one or more of: decreased GlnA glutamine synthetase activity, decreased transcriptional repression of GlnR. Introducing a genetic variation may comprise insertion and/or deletion of one or more nucleotides at a target site, such as 1, 2, 3, 4, 5, 10, 25, 50, 100, 250, 500, or more nucleotides. The genetic variation introduced into one or more bacteria of the methods disclosed herein may be a knock-out mutation (e.g. deletion of a promoter, insertion or deletion to produce a premature stop codon, deletion of an entire gene, deletion of gene (e.g., GlnA)), or it may be elimination or abolishment of activity of a protein domain (e.g. point mutation affecting an active site, or deletion of a portion of a gene encoding the relevant portion of the protein product), or it may alter or abolish a regulatory sequence of a target gene (e.g., one or more portions of the nifB promoter operably linked to the nif operon). One or more regulatory sequences may also be inserted, including heterologous regulatory sequences (e.g., insertion of a promoter selected from FIG. 9) and regulatory sequences found within a genome of a bacterial species or genus corresponding to the bacteria into which the genetic variation is introduced. Moreover, regulatory sequences may be selected based on the expression level of a gene in a bacterial culture or within a plant tissue. The genetic variation may be a pre-determined genetic variation that is specifically introduced to a target site. The genetic variation may be a random mutation within the target site. The genetic variation may be an insertion or deletion of one or more nucleotides. In some cases, a plurality of different genetic variations (e.g. 2, 3, 4, 5, 10, or more) are introduced into one or more of the isolated bacteria before exposing the bacteria to plants for assessing trait improvement. For example, a gram-positive microbe provided herein can comprise a mutant form of the nifB promoter operably linked to the mf cluster as provided herein, a mutant GlnR as provided herein, a mutant GlnA as provided herein or any combination thereof. The plurality of genetic variations can be any of the above types, the same or different types, and in any combination. In some cases, a plurality of different genetic variations are introduced serially, introducing a first genetic variation after a first isolation step, a second genetic variation after a second isolation step, and so forth so as to accumulate a plurality of genetic variations in bacteria imparting progressively improved traits on the associated plants.

### Genetic Variation—Methods of Introducing Genomic Alteration

In general, the term “genetic variation” refers to any change introduced into a polynucleotide sequence relative to a reference polynucleotide, such as a reference genome or portion thereof, or reference gene or portion thereof. A genetic variation may be referred to as a “mutation,” and a sequence or organism comprising a genetic variation may be referred to as a “genetic variant” or “mutant”. Genetic variations can have any number of effects, such as the increase or decrease of some biological activity, including gene expression, metabolism, and cell signaling. Genetic variations can be specifically introduced to a target site, or introduced randomly. A variety of molecular tools and methods are available for introducing genetic variation. For example, genetic variation can be introduced via polymerase chain reaction mutagenesis, oligonucleotide-directed mutagenesis, saturation mutagenesis, fragment shuffling mutagenesis, homologous recombination, recombineering, lambda red mediated recombination, CRISPR/Cas9 systems, chemical mutagenesis, and combinations thereof. Chemical methods of introducing genetic variation include exposure of DNA to a chemical mutagen, e.g., ethyl methanesulfonate (EMS), methyl methanesulfonate (MMS), N-nitrosourea (EN U), N-methyl-N-nitro-N′-nitrosoguanidine, 4-nitroquinoline N-oxide, diethylsulfate, benzopyrene, cyclophosphamide, bleomycin, triethylmelamine, acrylamide monomer, nitrogen mustard, vincristine, diepoxyalkanes (for example, diepoxybutane), ICR-170, formaldehyde, procarbazine hydrochloride, ethylene oxide, dimethylnitrosamine, 7,12 dimethylbenz(a)anthracene, chlorambucil, hexamethylphosphoramide, bisulfan, and the like. Radiation mutation-inducing agents include ultraviolet radiation, γ-irradiation, X-rays, and fast neutron bombardment. Genetic variation can also be introduced into a nucleic acid using, e.g., trimethylpsoralen with ultraviolet light. Random or targeted insertion of a mobile DNA element, e.g., a transposable element, is another suitable method for generating genetic variation. Genetic variations can be introduced into a nucleic acid during amplification in a cell-free in vitro system, e.g., using a polymerase chain reaction (PCR) technique such as error-prone PCR. Genetic variations can be introduced into a nucleic acid in vitro using DNA shuffling techniques (e.g., exon shuffling, domain swapping, and the like). Genetic variations can also be introduced into a nucleic acid as a result of a deficiency in a DNA repair enzyme in a cell, e.g., the presence in a cell of a mutant gene encoding a mutant DNA repair enzyme is expected to generate a high frequency of mutations (i.e., about 1 mutation/100 genes-1 mutation/10,000 genes) in the genome of the cell. Examples of genes encoding DNA repair enzymes include but are not limited to Mut H, Mut S, Mut L, and Mut U, and the homologs thereof in other species (e.g., MSH 1 6, PMS 1 2, MLH 1, GTBP, ERCC-1, and the like). Example descriptions of various methods for introducing genetic variations are provided in e.g., Stemple (2004) Nature 5:1-7; Chiang et al. (1993) PCR Methods Appl 2(3): 210-217; Stemmer (1994) Proc. Natl. Acad. Sci. USA 91:10747-10751; and U.S. Pat. Nos. 6,033,861, and 6,773,900.

Genetic variations introduced into microbes may be classified as transgenic, cisgenic, intragenomic, intrageneric, intergeneric, synthetic, evolved, rearranged, or SNPs. In some cases, the engineered gram-positive microbes provided herein are non-intergeneric. In some cases, the engineered gram-positive microbes provided herein are transgenic.

Genetic variation may be introduced into numerous metabolic pathways within microbes to elicit improvements in the traits described above. Representative pathways include sulfur uptake pathways, glycogen biosynthesis, the glutamine regulation pathway, the molybdenum uptake pathway, the nitrogen fixation pathway, ammonia assimilation, ammonia excretion or secretion, nitrogen uptake, glutamine biosynthesis, annamox, phosphate solubilization, organic acid transport, organic acid production, agglutinins production, reactive oxygen radical scavenging genes, indole acetic acid biosynthesis, trehalose biosynthesis, plant cell wall degrading enzymes or pathways, root attachment genes, exopolysaccharide secretion, glutamate synthase pathway, iron uptake pathways, siderophore pathway, chitinase pathway, ACC deaminase, glutathione biosynthesis, phosphorous signaling genes, quorum quenching pathway, cytochrome pathways, hemoglobin pathway, bacterial hemoglobin-like pathway, small RNA rsmZ, rhizobitoxine biosynthesis, lapA adhesion protein, AHL quorum sensing pathway, phenazine biosynthesis, cyclic lipopeptide biosynthesis, and antibiotic production.

CRISPR/Cas9 (Clustered regularly interspaced short palindromic repeats)/CRISPR-associated (Cas) systems can be used to introduce desired mutations. CRISPR/Cas9 provide bacteria and archaea with adaptive immunity against viruses and plasmids by using CRISPR RNAs (crRNAs) to guide the silencing of invading nucleic acids. The Cas9 protein (or functional equivalent and/or variant thereof, i.e., Cas9-like protein) naturally contains DNA endonuclease activity that depends on the association of the protein with two naturally occurring or synthetic RNA molecules called crRNA and tracrRNA (also called guide RNAs). In some cases, the two molecules are covalently link to form a single molecule (also called a single guide RNA (“sgRNA”). Thus, the Cas9 or Cas9-like protein associates with a DNA-targeting RNA (which term encompasses both the two-molecule guide RNA configuration and the single-molecule guide RNA configuration), which activates the Cas9 or Cas9-like protein and guides the protein to a target nucleic acid sequence. If the Cas9 or Cas9-like protein retains its natural enzymatic function, it will cleave target DNA to create a double-stranded break, which can lead to genome alteration (i.e., editing: deletion, insertion (when a donor polynucleotide is present), replacement, etc.), thereby altering gene expression. Some variants of Cas9 (which variants are encompassed by the term Cas9-like) have been altered such that they have a decreased DNA cleaving activity (in some cases, they cleave a single strand instead of both strands of the target DNA, while in other cases, they have severely reduced to no DNA cleavage activity). Further exemplary descriptions of CRISPR systems for introducing genetic variation can be found in, e.g. U.S. Pat. No. 8,795,965.

As a cyclic amplification technique, polymerase chain reaction (PCR) mutagenesis uses mutagenic primers to introduce desired mutations. PCR is performed by cycles of denaturation, annealing, and extension. After amplification by PCR, selection of mutated DNA and removal of parental plasmid DNA can be accomplished by: 1) replacement of dCTP by hydroxymethylated-dCTP during PCR, followed by digestion with restriction enzymes to remove non-hydroxymethylated parent DNA only; 2) simultaneous mutagenesis of both an antibiotic resistance gene and the studied gene changing the plasmid to a different antibiotic resistance, the new antibiotic resistance facilitating the selection of the desired mutation thereafter; 3) after introducing a desired mutation, digestion of the parent methylated template DNA by restriction enzyme Dpnl which cleaves only methylated DNA, by which the mutagenized unmethylated chains are recovered; or 4) circularization of the mutated PCR products in an additional ligation reaction to increase the transformation efficiency of mutated DNA. Further description of exemplary methods can be found in e.g. U.S. Pat. Nos. 7,132,265, 6,713,285, 6,673,610, 6,391,548, 5,789,166, 5,780,270, 5,354,670, 5,071,743, and US20100267147.

Oligonucleotide-directed mutagenesis, also called site-directed mutagenesis, typically utilizes a synthetic DNA primer. This synthetic primer contains the desired mutation and is complementary to the template DNA around the mutation site so that it can hybridize with the DNA in the gene of interest. The mutation may be a single base change (a point mutation), multiple base changes, deletion, or insertion, or a combination of these. The single-strand primer is then extended using a DNA polymerase, which copies the rest of the gene. The gene thus copied contains the mutated site, and may then be introduced into a host cell as a vector and cloned. Finally, mutants can be selected by DNA sequencing to check that they contain the desired mutation.

Genetic variations can be introduced using error-prone PCR. In this technique, the gene of interest is amplified using a DNA polymerase under conditions that are deficient in the fidelity of replication of sequence. The result is that the amplification products contain at least one error in the sequence. When a gene is amplified and the resulting product(s) of the reaction contain one or more alterations in sequence when compared to the template molecule, the resulting products are mutagenized as compared to the template. Another means of introducing random mutations is exposing cells to a chemical mutagen, such as nitrosoguanidine or ethyl methanesulfonate (Nestmann, Mutat Res 1975 June; 28(3):323-30), and the vector containing the gene is then isolated from the host.

Saturation mutagenesis is another form of random mutagenesis, in which one tries to generate all or nearly all possible mutations at a specific site, or narrow region of a gene. In a general sense, saturation mutagenesis is comprised of mutagenizing a complete set of mutagenic cassettes (wherein each cassette is, for example, 1-500 bases in length) in defined polynucleotide sequence to be mutagenized (wherein the sequence to be mutagenized is, for example, from 15 to 100, 000 bases in length). Therefore, a group of mutations (e.g. ranging from 1 to 100 mutations) is introduced into each cassette to be mutagenized. A grouping of mutations to be introduced into one cassette can be different or the same from a second grouping of mutations to be introduced into a second cassette during the application of one round of saturation mutagenesis. Such groupings are exemplified by deletions, additions, groupings of particular codons, and groupings of particular nucleotide cassettes.

Fragment shuffling mutagenesis, also called DNA shuffling, is a way to rapidly propagate beneficial mutations. In an example of a shuffling process, DNAse is used to fragment a set of parent genes into pieces of e.g. about 50-100 bp in length. This is then followed by a polymerase chain reaction (PCR) without primers. DNA fragments with sufficient overlapping homologous sequence will anneal to each other and are then be extended by DNA polymerase. Several rounds of this PCR extension are allowed to occur, after some of the DNA molecules reach the size of the parental genes. These genes can then be amplified with another PCR, this time with the addition of primers that are designed to complement the ends of the strands. The primers may have additional sequences added to their 5′ ends, such as sequences for restriction enzyme recognition sites needed for ligation into a cloning vector. Further examples of shuffling techniques are provided in US20050266541.

Homologous recombination mutagenesis involves recombination between an exogenous DNA fragment and the targeted polynucleotide sequence. After a double-stranded break occurs, sections of DNA around the 5′ ends of the break are cut away in a process called resection. In the strand invasion step that follows, an overhanging 3′ end of the broken DNA molecule then “invades” a similar or identical DNA molecule that is not broken. The method can be used to delete a gene, remove exons, add a gene, and introduce point mutations. Homologous recombination mutagenesis can be permanent or conditional. Typically, a recombination template is also provided. A recombination template may be a component of another vector, contained in a separate vector, or provided as a separate polynucleotide. In some embodiments, a recombination template is designed to serve as a template in homologous recombination, such as within or near a target sequence nicked or cleaved by a site-specific nuclease. A template polynucleotide may be of any suitable length, such as about or more than about 10, 15, 20, 25, 50, 75, 100, 150, 200, 500, 1000, or more nucleotides in length. In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence. Non-limiting examples of site-directed nucleases useful in methods of homologous recombination include zinc finger nucleases, CRISPR nucleases, TALE nucleases, and meganuclease. For a further description of the use of such nucleases, see e.g. U.S. Pat. No. 8,795,965 and US20140301990.

Mutagens that create primarily point mutations and short deletions, insertions, transversions, and/or transitions, including chemical mutagens or radiation, may be used to create genetic variations. Mutagens include, but are not limited to, ethyl methanesulfonate, methylmethane sulfonate, N-ethyl-N-nitrosurea, triethylmelamine, N-methyl-N-nitrosourea, procarbazine, chlorambucil, cyclophosphamide, diethyl sulfate, acrylamide monomer, melphalan, nitrogen mustard, vincristine, dimethylnitrosamine, N-methyl-N′-nitro-Nitrosoguanidine, nitrosoguanidine, 2-aminopurine, 7,12 dimethyl-benz(a)anthracene, ethylene oxide, hexamethylphosphoramide, bisulfan, diepoxyalkanes (diepoxyoctane, diepoxybutane, and the like), 2-methoxy-6-chloro-9[3-(ethyl-2-chloro-ethyl)aminopropylamino]acridine dihydrochloride and formaldehyde.

Introducing genetic variation may be an incomplete process, such that some bacteria in a treated population of bacteria carry a desired mutation while others do not. In some cases, it is desirable to apply a selection pressure so as to enrich for bacteria carrying a desired genetic variation. Traditionally, selection for successful genetic variants involved selection for or against some functionality imparted or abolished by the genetic variation, such as in the case of inserting antibiotic resistance gene or abolishing a metabolic activity capable of converting a non-lethal compound into a lethal metabolite. It is also possible to apply a selection pressure based on a polynucleotide sequence itself, such that only a desired genetic variation need be introduced (e.g. without also requiring a selectable marker). In this case, the selection pressure can comprise cleaving genomes lacking the genetic variation introduced to a target site, such that selection is effectively directed against the reference sequence into which the genetic variation is sought to be introduced. Typically, cleavage occurs within 100 nucleotides of the target site (e.g. within 75, 50, 25, 10, or fewer nucleotides from the target site, including cleavage at or within the target site). Cleaving may be directed by a site-specific nuclease selected from the group consisting of a Zinc Finger nuclease, a CRISPR nuclease, a TALE nuclease (TALEN), or a meganuclease. Such a process is similar to processes for enhancing homologous recombination at a target site, except that no template for homologous recombination is provided. As a result, bacteria lacking the desired genetic variation are more likely to undergo cleavage that, left unrepaired, results in cell death. Bacteria surviving selection may then be isolated for use in exposing to plants for assessing conferral of an improved trait.

A CRISPR nuclease may be used as the site-specific nuclease to direct cleavage to a target site. An improved selection of mutated microbes can be obtained by using Cas9 to kill non-mutated cells. Plants are then inoculated with the mutated microbes to re-confirm symbiosis and create evolutionary pressure to select for efficient symbionts. Microbes can then be re-isolated from plant tissues. CRISPR nuclease systems employed for selection against non-variants can employ similar elements to those described above with respect to introducing genetic variation, except that no template for homologous recombination is provided. Cleavage directed to the target site thus enhances death of affected cells.

Other options for specifically inducing cleavage at a target site are available, such as zinc finger nucleases, TALE nuclease (TALEN) systems, and meganuclease. Zinc-finger nucleases (ZFNs) are artificial DNA endonucleases generated by fusing a zinc finger DNA binding domain to a DNA cleavage domain. ZFNs can be engineered to target desired DNA sequences and this enables zinc-finger nucleases to cleave unique target sequences. When introduced into a cell, ZFNs can be used to edit target DNA in the cell (e.g., the cell's genome) by inducing double stranded breaks. Transcription activator-like effector nucleases (TALENs) are artificial DNA endonucleases generated by fusing a TAL (Transcription activator-like) effector DNA binding domain to a DNA cleavage domain. TALENS can be quickly engineered to bind practically any desired DNA sequence and when introduced into a cell, TALENs can be used to edit target DNA in the cell (e.g., the cell's genome) by inducing double strand breaks. Meganucleases (homing endonuclease) are endodeoxyribonucleases characterized by a large recognition site (double-stranded DNA sequences of 12 to 40 base pairs. Meganucleases can be used to replace, eliminate or modify sequences in a highly targeted way. By modifying their recognition sequence through protein engineering, the targeted sequence can be changed. Meganucleases can be used to modify all genome types, whether bacterial, plant or animal and are commonly grouped into four families: the LAGLIDADG family (SEQ ID NO: 47), the GIY-YIG family, the His-Cyst box family and the HNH family. Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII.

### Genetic Variation—Methods of Identification

The microbes of the present disclosure may be identified by one or more genetic modifications or alterations, which have been introduced into said microbe. One method by which said genetic modification or alteration can be identified is via reference to a SEQ ID NO that contains a portion of the microbe's genomic sequence that is sufficient to identify the genetic modification or alteration.

Further, in the case of microbes that have not had a genetic modification or alteration (e.g. a wild type, WT) introduced into their genomes, the disclosure can utilize 16S nucleic acid sequences to identify said microbes. A 16S nucleic acid sequence is an example of a “molecular marker” or “genetic marker,” which refers to an indicator that is used in methods for visualizing differences in characteristics of nucleic acid sequences. Examples of other such indicators are restriction fragment length polymorphism (RFLP) markers, amplified fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Markers further include polynucleotide sequences encoding 16S or 18S rRNA, and internal transcribed spacer (ITS) sequences, which are sequences found between small-subunit and large-subunit rRNA genes that have proven to be especially useful in elucidating relationships or distinctions when compared against one another. Furthermore, the disclosure utilizes unique sequences found in genes of interest (e.g. nifB, nifH, nifD, nifK, nifE, nifN, nifX, hesA, nifV, etc.) to identify microbes disclosed herein.

The primary structure of major rRNA subunit 16S comprise a particular combination of conserved, variable, and hypervariable regions that evolve at different rates and enable the resolution of both very ancient lineages such as domains, and more modern lineages such as genera. The secondary structure of the 16S subunit include approximately 50 helices, which result in base pairing of about 67% of the residues. These highly conserved secondary structural features are of great functional importance and can be used to ensure positional homology in multiple sequence alignments and phylogenetic analysis. Over the previous few decades, the 16S rRNA gene has become the most sequenced taxonomic marker and is the cornerstone for the current systematic classification of bacteria and archaea (Yarza et al. 2014. Nature Rev. Micro. 12:635-45).

### Genetic Variation—Methods of Detection: Primers, Probes, and Assays

The present disclosure teaches primers, probes, and assays that are useful for detecting the microbes taught herein. In some aspects, the disclosure provides for methods of detecting the WT parental strains. In other aspects, the disclosure provides for methods of detecting the non-intergeneric engineered microbes derived from the WT strains. In aspects, the present disclosure provides methods of identifying non-intergeneric genetic alterations in a microbe.

In aspects, the genomic engineering methods of the present disclosure lead to the creation of non-natural nucleotide “junction” sequences in the derived non-intergeneric microbes. These non-naturally occurring nucleotide junctions can be used as a type of diagnostic that is indicative of the presence of a particular genetic alteration in a microbe taught herein.

The present techniques are able to detect these non-naturally occurring nucleotide junctions via the utilization of specialized quantitative PCR methods, including uniquely designed primers and probes. In some aspects, the probes of the disclosure bind to the non-naturally occurring nucleotide junction sequences. In some aspects, traditional PCR is utilized. In other aspects, real-time PCR is utilized. In some aspects, quantitative PCR (qPCR) is utilized.

Thus, the disclosure can cover the utilization of two common methods for the detection of PCR products in real-time: (1) non-specific fluorescent dyes that intercalate with any double-stranded DNA, and (2) sequence-specific DNA probes consisting of oligonucleotides that are labelled with a fluorescent reporter which permits detection only after hybridization of the probe with its complementary sequence. In some aspects, only the non-naturally occurring nucleotide junction will be amplified via the taught primers, and consequently can be detected via either a non-specific dye, or via the utilization of a specific hybridization probe. In other aspects, the primers of the disclosure are chosen such that the primers flank either side of a junction sequence, such that if an amplification reaction occurs, then said junction sequence is present.

Aspects of the disclosure involve non-naturally occurring nucleotide junction sequence molecules per se, along with other nucleotide molecules that are capable of binding to said non-naturally occurring nucleotide junction sequences under mild to stringent hybridization conditions. In some aspects, the nucleotide molecules that are capable of binding to said non-naturally occurring nucleotide junction sequences under mild to stringent hybridization conditions are termed “nucleotide probes.”

In aspects, genomic DNA can be extracted from samples and used to quantify the presence of microbes of the disclosure by using qPCR. The primers utilized in the qPCR reaction can be primers designed by Primer Blast (www.ncbi.nlm.nih.gov/tools/primer-blast/) to amplify unique regions of the wild-type genome or unique regions of the engineered non-intergeneric mutant strains. The qPCR reaction can be carried out using the SYBR GreenER qPCR SuperMix Universal (Thermo Fisher P/N 11762100) kit, using only forward and reverse amplification primers; alternatively, the Kapa Probe Force kit (Kapa Biosystems P/N KK4301) can be used with amplification primers and a TaqMan probe containing a FAM dye label at the 5′ end, an internal ZEN quencher, and a minor groove binder and fluorescent quencher at the 3′ end (Integrated DNA Technologies).

qPCR reaction efficiency can be measured using a standard curve generated from a known quantity of gDNA from the target genome. Data can be normalized to genome copies per g fresh weight using the tissue weight and extraction volume.

Quantitative polymerase chain reaction (qPCR) is a method of quantifying, in real time, the amplification of one or more nucleic acid sequences. The real time quantification of the PCR assay permits determination of the quantity of nucleic acids being generated by the PCR amplification steps by comparing the amplifying nucleic acids of interest and an appropriate control nucleic acid sequence, which may act as a calibration standard.

TaqMan probes are often utilized in qPCR assays that require an increased specificity for quantifying target nucleic acid sequences. TaqMan probes comprise an oligonucleotide probe with a fluorophore attached to the 5′ end and a quencher attached to the 3′ end of the probe. When the TaqMan probes remain as is with the 5′ and 3′ ends of the probe in close contact with each other, the quencher prevents fluorescent signal transmission from the fluorophore. TaqMan probes are designed to anneal within a nucleic acid region amplified by a specific set of primers. As the Taq polymerase extends the primer and synthesizes the nascent strand, the 5′ to 3′ exonuclease activity of the Taq polymerase degrades the probe that annealed to the template. This probe degradation releases the fluorophore, thus breaking the close proximity to the quencher and allowing fluorescence of the fluorophore. Fluorescence detected in the qPCR assay is directly proportional to the fluorophore released and the amount of DNA template present in the reaction.

The features of qPCR allow the practitioner to eliminate the labor-intensive post-amplification step of gel electrophoresis preparation, which is generally required for observation of the amplified products of traditional PCR assays. The benefits of qPCR over conventional PCR are considerable, and include increased speed, ease of use, reproducibility, and quantitative ability.

### Improvement of Traits

Methods of the present disclosure may be employed to introduce or improve one or more of a variety of desirable traits. Examples of traits that may introduced or improved include: root biomass, root length, height, shoot length, leaf number, water use efficiency, overall biomass, yield, fruit size, grain size, photosynthesis rate, tolerance to drought, heat tolerance, salt tolerance, resistance to nematode stress, resistance to a fungal pathogen, resistance to a bacterial pathogen, resistance to a viral pathogen, level of a metabolite, and proteome expression. The desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the improved traits) grown under identical conditions.

A preferred trait to be introduced or improved is nitrogen fixation, as described herein. In some cases, a plant resulting from the methods described herein exhibits a difference in the trait that is at least about 5% greater, for example at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under the same conditions in the soil. In additional examples, a plant resulting from the methods described herein exhibits a difference in the trait that is at least about 5% greater, for example at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under similar conditions in the soil.

The trait to be improved may be assessed under conditions including the application of one or more biotic or abiotic stressors. Examples of stressors include abiotic stresses (such as heat stress, salt stress, drought stress, cold stress, and low nutrient stress) and biotic stresses (such as nematode stress, insect herbivory stress, fungal pathogen stress, bacterial pathogen stress, and viral pathogen stress).

The trait improved by methods and compositions of the present disclosure may be nitrogen fixation, including in a plant not previously capable of nitrogen fixation. In some cases, bacteria isolated according to a method described herein produce 1% or more (e.g. 2%, 3%, 4%, 5%, 6%, 7%, 8%, 9%, 10%, 15%, 20%, or more) of a plant's nitrogen, which may represent an increase in nitrogen fixation capability of at least 2-fold (e.g. 3-fold, 4-fold, 5-fold, 6-fold, 7-fold, 8-fold, 9-fold, 10-fold, 20-fold, 50-fold, 100-fold, 1000-fold, or more) as compared to bacteria isolated from a plant before introducing any genetic variation. In some cases, the bacteria produce 5% or more of a plant's nitrogen. The desired level of nitrogen fixation may be achieved after repeating the steps of introducing genetic variation, exposure to a plurality of plants, and isolating bacteria from plants with an improved trait one or more times (e.g. 1, 2, 3, 4, 5, 10, 15, 25, or more times). In some cases, enhanced levels of nitrogen fixation are achieved in the presence of fertilizer supplemented with glutamine, ammonia, or other chemical source of nitrogen. Methods for assessing degree of nitrogen fixation are known, examples of which are described herein.

### Measuring Nitrogen Delivered in an Agriculturally Relevant Field Context

In the field, the amount of nitrogen delivered can be determined by the function of colonization multiplied by the activity.

$$Nitrogen delivered = {\int }_{Time \& Space} Colonization \times Activity$$

The above equation requires (1) the average colonization per unit of plant tissue, and (2) the activity as either the amount of nitrogen fixed or the amount of ammonia excreted by each microbial cell. To convert to pounds of nitrogen per acre, corn growth physiology is tracked over time, e.g., size of the plant and associated root system throughout the maturity stages.

The pounds of nitrogen delivered to a crop per acre-season can be calculated by the following equation:

Nitrogen delivered=∫Plant Tissue(t)×Colonization(t)×Activity(t)dt

The Plant Tissue(t) is the fresh weight of corn plant tissue over the growing time (t). Values for reasonably making the calculation are described in detail in the publication entitled Roots, Growth and Nutrient Uptake (Mengel. Dept. of Agronomy Pub. #AGRY-95-08 (Rev. May-95. p. 1-8.).

The Colonization (t) is the amount of the microbes of interest found within the plant tissue, per gram fresh weight of plant tissue, at any particular time, t, during the growing season. In the instance of only a single timepoint available, the single timepoint is normalized as the peak colonization rate over the season, and the colonization rate of the remaining timepoints are adjusted accordingly.

Activity(t) is the rate of which N is fixed by the microbes of interest per unit time, at any particular time, t, during the growing season. In the embodiments disclosed herein, this activity rate is approximated by in vitro acetylene reduction assay (ARA) in ARA media in the presence of 5 mM glutamine or Ammonium excretion assay in ARA media in the presence of 5 mM ammonium ions.

The nitrogen delivered amount is then calculated by numerically integrating the above function. In cases where the values of the variables described above are discretely measured at set timepoints, the values in between those timepoints are approximated by performing linear interpolation.

### Nitrogen Fixation

Described herein are methods of increasing nitrogen fixation in a plant, comprising exposing the plant to bacteria comprising one or more genetic variations introduced into one or more genes regulating nitrogen fixation, wherein the bacteria produce 1% or more (e.g. 2%, 3%, 4%, 5%, 6%, 7%, 8%, 9%, 10%, 15%, 20%, or more) of nitrogen in the plant, which may represent a nitrogen fixation capability of at least 2-fold (e.g. 3-fold, 4-fold, 5-fold, 6-fold, 7-fold, 8-fold, 9-fold, 10-fold, 20-fold, 50-fold, 100-fold, 1000-fold, or more) as compared to the plant in the absence of the bacteria. The bacteria may produce the nitrogen in the presence of fertilizer supplemented with glutamine, urea, nitrates or ammonia. Genetic variations can be any genetic variation described herein, including examples provided above, in any number and any combination. The genetic variation may be introduced into a gene selected from the group consisting of nifB, nifH, nifD, nifK, nifE, nifN, nifX, hesA, nifV, glutamine synthetase, glnA and glnR. The genetic variation may be a mutation that results in one or more of: increased expression or activity of nitrogenase; decreased expression or activity of glutamine synthetase or the repressive activity of GlnR. The genetic variation introduced into one or more bacteria of the methods disclosed herein may be a knock-out mutation or it may abolish a regulatory sequence of a target gene, or it may comprise insertion of a heterologous regulatory sequence, for example, insertion of a regulatory sequence found within the genome of the same bacterial species or genus. The regulatory sequence can be chosen based on the expression level of a gene in a bacterial culture or within plant tissue. In some cases, the engineered gram-positive microbes provided herein are non-intergeneric. In some cases, the engineered gram-positive microbes provided herein are transgenic. The genetic variation may be produced by chemical mutagenesis. The plants grown in step (c) may be exposed to biotic or abiotic stressors.

The amount of nitrogen fixation that occurs in the plants described herein may be measured in several ways, for example by an acetylene-reduction (AR) assay. An acetylene-reduction assay can be performed in vitro or in vivo. Evidence that a particular bacterium is providing fixed nitrogen to a plant can include: 1) total plant N significantly increases upon inoculation, preferably with a concomitant increase in N concentration in the plant; 2) nitrogen deficiency symptoms are relieved under N-limiting conditions upon inoculation (which should include an increase in dry matter); 3) N₂ fixation is documented through the use of an ¹⁵N approach (which can be isotope dilution experiments, ¹⁵N₂ reduction assays, or ¹⁵N natural abundance assays); 4) fixed N is incorporated into a plant protein or metabolite; and 5) all of these effects are not be seen in non-inoculated plants or in plants inoculated with a mutant of the inoculum strain.

The wild-type nitrogen fixation regulatory cascade can be represented as a digital logic circuit where the inputs O₂ and NH₄⁺ pass through a NOR gate, the output of which enters an AND gate in addition to ATP. In some embodiments, the methods disclosed herein disrupt the influence of NH₄⁺ on this circuit, at multiple points in the regulatory cascade, so that microbes can produce nitrogen even in fertilized fields. However, the methods disclosed herein also envision altering the impact of ATP or O₂ on the circuitry, or replacing the circuitry with other regulatory cascades in the cell, or altering genetic circuits other than nitrogen fixation. Gene clusters can be re-engineered to generate functional products under the control of a heterologous regulatory system.

By eliminating native regulatory elements outside of, and within, coding sequences of gene clusters, and replacing them with alternative regulatory systems, the functional products of complex genetic operons and other gene clusters can be controlled and/or moved to heterologous cells, including cells of different species other than the species from which the native genes were derived. Once re-engineered, the synthetic gene clusters can be controlled by genetic circuits or other inducible regulatory systems, thereby controlling the products' expression as desired. The expression cassettes can be designed to act as logic gates, pulse generators, oscillators, switches, or memory devices. The controlling expression cassette can be linked to a promoter such that the expression cassette functions as an environmental sensor, such as an oxygen, temperature, touch, osmotic stress, membrane stress, or redox sensor.

Synthetic genes can be designed by codon randomizing the DNA encoding each amino acid sequence. Codon selection is performed, specifying that codon usage be as divergent as possible from the codon usage in the native gene. Proposed sequences are scanned for any undesired features, such as restriction enzyme recognition sites, transposon recognition sites, repetitive sequences, sigma 54 and sigma 70 promoters, cryptic ribosome binding sites, and rho independent terminators. Synthetic ribosome binding sites are chosen to match the strength of each corresponding native ribosome binding site, such as by constructing a fluorescent reporter plasmid in which the 150 bp surrounding a gene's start codon (from −60 to +90) is fused to a fluorescent gene. This chimera can be expressed under control of the Ptac promoter, and fluorescence measured via flow cytometry. To generate synthetic ribosome binding sites, a library of reporter plasmids using 150 bp (−60 to +90) of a synthetic expression cassette is generated. Briefly, a synthetic expression cassette can consist of a random DNA spacer, a degenerate sequence encoding an RBS library, and the coding sequence for each synthetic gene. Multiple clones are screened to identify the synthetic ribosome binding site that best matched the native ribosome binding site. Synthetic operons that consist of the same genes as the native operons are thus constructed and tested for functional complementation. A further exemplary description of synthetic operons is provided in US20140329326.

### Bacterial Species

Microbes useful in the methods and compositions disclosed herein may be obtained from any source. In some cases, microbes may be bacteria. The microbes of this disclosure may be nitrogen fixing microbes, for example a nitrogen fixing bacteria, nitrogen fixing archaea, nitrogen fixing fungi, nitrogen fixing yeast, or nitrogen fixing protozoa. Microbes useful in the methods and compositions disclosed herein may be spore forming microbes, for example spore forming bacteria. In some cases, bacteria useful in the methods and compositions disclosed herein may be Gram-positive bacteria. In some cases, the bacteria may be an endospore forming bacteria of the Firmicute phylum. In some cases, the bacteria may be a diazotroph. In some cases, the bacteria may not be a diazotroph. In one embodiment, the bacteria useful in the methods and compositions disclosed herein are gram-positive bacteria. In another embodiment, the bacteria useful in the methods and compositions disclosed herein are gram-positive diazotrophic bacteria. In some cases, the gram-positive microbes provided herein are non-intergeneric. In some cases, the engineered gram-positive microbes provided herein are transgenic.

In some cases, bacteria which may be useful include, but are not limited to Bacillus acidocaldarius, Bacillus acidoterrestris, Bacillus agri, Bacillus aizawai, Bacillus albolactis, Bacillus alcalophilus, Bacillus alvei, Bacillus aminoglucosidicus, Bacillus aminovorans, Bacillus amylolyticus (also known as Paenibacillus amylolyticus) Bacillus amyloliquefaciens, Bacillus aneurinolyticus, Bacillus atrophaeus, Bacillus azotoformans, Bacillus badius, Bacillus cereus (synonyms: Bacillus endorhythmos, Bacillus medusa), Bacillus chitinosporus, Bacillus circulans, Bacillus coagulans, Bacillus endoparasiticus Bacillus fastidiosus, Bacillus firmus, Bacillus kurstaki, Bacillus lacticola, Bacillus lactimorbus, Bacillus lactis, Bacillus laterosporus (also known as Brevibacillus laterosporus), Bacillus lautus, Bacillus lentimorbus, Bacillus lentus, Bacillus lichenformis, Bacillus maroccanus, Bacillus megaterium, Bacillus metiens, Bacillus mycoides, Bacillus natto, Bacillus nematocida, Bacillus nigrificans, Bacillus nigrum, Bacillus pantothenticus, Bacillus popillae, Bacillus psychrosaccharolyticus, Bacillus pumilus, Bacillus siamensis, Bacillus smithii, Bacillus sphaericus, Bacillus subtilis, Bacillus thuringiensis, Bacillus uniflagellatus, Brevibacillus brevis Brevibacillus laterosporus (formerly Bacillus laterosporus), Lactobacillus acidophilus, Paenibacillus alvei, Paenibacillus polymyxa, Paenibacillus popilliae (formerly Bacillus popilliae), Pasteuria penetrans (formerly Bacillus penetrans), Pasteuria usgae, Bacillus sp. AQ175 (ATCC Accession No. 55608), Bacillus sp. AQ 177 (ATCC Accession No. 55609) and Bacillus sp. AQ178 (ATCC Accession No. 53522). In some cases, the bacterium may be Paenibacillus, Bacillus or Lactobacillus.

Microbes useful in the methods and compositions disclosed herein can be obtained by extracting microbes from surfaces or tissues of native plants; grinding seeds to isolate microbes; planting seeds in diverse soil samples and recovering microbes from tissues; or inoculating plants with exogenous microbes and determining which microbes appear in plant tissues. Non-limiting examples of plant tissues include a seed, seedling, leaf, cutting, plant, bulb, tuber, root, and rhizomes. In some cases, bacteria are isolated from a seed. The parameters for processing samples may be varied to isolate different types of associative microbes, such as rhizospheric, epiphytes, or endophytes. Bacteria may also be sourced from a repository, such as environmental strain collections, instead of initially isolating from a first plant. The microbes can be genotyped and phenotyped, via sequencing the genomes of isolated microbes; profiling the composition of communities in planta; characterizing the transcriptomic functionality of communities or isolated microbes; or screening microbial features using selective or phenotypic media (e.g., nitrogen fixation or phosphate solubilization phenotypes). Selected candidate strains or populations can be obtained via sequence data; phenotype data; plant data (e.g., genome, phenotype, and/or yield data); soil data (e.g., pH, N/P/K content, and/or bulk soil biotic communities); or any combination of these.

The bacteria and methods of producing bacteria described herein may apply to bacteria able to self-propagate efficiently on the leaf surface, root surface, or inside plant tissues without inducing a damaging plant defense reaction, or bacteria that are resistant to plant defense responses. The bacteria described herein may be isolated by culturing a plant tissue extract or leaf surface wash in a medium with no added nitrogen. However, the bacteria may be unculturable, that is, not known to be culturable or difficult to culture using standard methods known in the art. The bacteria described herein may be an endophyte or an epiphyte or a bacterium inhabiting the plant rhizosphere (rhizospheric bacteria). The bacteria obtained after repeating the steps of introducing genetic variation, exposure to a plurality of plants, and isolating bacteria from plants with an improved trait one or more times (e.g. 1, 2, 3, 4, 5, 10, 15, 25, or more times) may be endophytic, epiphytic, or rhizospheric. Endophytes are organisms that enter the interior of plants without causing disease symptoms or eliciting the formation of symbiotic structures, and are of agronomic interest because they can enhance plant growth and improve the nutrition of plants (e.g., through nitrogen fixation). The bacteria can be a seed-borne endophyte. Seed-borne endophytes include bacteria associated with or derived from the seed of a grass or plant, such as a seed-borne bacterial endophyte found in mature, dry, undamaged (e.g., no cracks, visible fungal infection, or prematurely germinated) seeds. The seed-borne bacterial endophyte can be associated with or derived from the surface of the seed; alternatively, or in addition, it can be associated with or derived from the interior seed compartment (e.g., of a surface-sterilized seed). In some cases, a seed-borne bacterial endophyte is capable of replicating within the plant tissue, for example, the interior of the seed. Also, in some cases, the seed-borne bacterial endophyte is capable of surviving desiccation.

The bacterial isolated according to methods of the disclosure, or used in methods or compositions of the disclosure, can comprise a plurality of different bacterial taxa in combination. By way of example, the bacteria may include Firmicutes (such as Bacillus, Paenibacillus, Lactobacillus, Mycoplasma, and Acetabacterium) and Actinobacteria (such as Streptomyces, Rhodacoccus, Microbacterium, and Curtobacterium). The bacteria used in methods and compositions of this disclosure may include nitrogen fixing bacterial consortia of two or more species. In some cases, one or more bacterial species of the bacterial consortia may be capable of fixing nitrogen. In some cases, one or more species of the bacterial consortia may facilitate or enhance the ability of other bacteria to fix nitrogen. The bacteria which fix nitrogen and the bacteria which enhance the ability of other bacteria to fix nitrogen may be the same or different. In some examples, a bacterial strain may be able to fix nitrogen when in combination with a different bacterial strain, or in a certain bacterial consortia, but may be unable to fix nitrogen in a monoculture. Examples of bacterial genera which may be found in a nitrogen fixing bacterial consortia include, but are not limited to, Paenibacillus, Lactobacillus, and Bacillus.

Bacteria that can be produced by the methods disclosed herein include Paenibacillus sp., Bacillus sp., or Lactobacillus sp. In some cases, the bacteria may be of the genus Paenibacillus, for example Paenibacillus azotofixans, Paenibacillus borealhs, Paenibacillus durus, Paenibacillus macerans, Paenibacillus polymyxa, Paenibacillus alvei, Paenibacillus amylolyticus, Paenibacillus campinasensis, Paenibacillus chibensis, Paenibacillus glucanolyticus, Paenibacillus illinoisensis, Paenibacillus larvae subsp. Larvae, Paenibacillus larvae subsp. Pulvifaciens, Paenibacillus lautus, Paenibacillus macerans, Paenibacillus macquariensis, Paenibacillus macquariensis, Paenibacillus pabuli, Paenibacillus peoriae, or Paenibacillus polymyxa. In some examples, bacteria isolated according to methods of the disclosure can be a member of one or more of the following taxa: Bacillus, Lactobacillus, and Paenibacillus,

In some cases, a Gram-positive microbe may have a molybdenum-iron nitrogenase system comprising: nifH, nifD, nifK, nifB, nifE, nifN, nifX, hesA, nifV, nifW, nifU, nifS, nifl1, and nifl2. In some cases, a Gram-positive microbe may have a vanadium nitrogenase system comprising: vnfDG, vnfK, vnfE, vnfN, vupC, vupB, vupA, vnfV, vnfR1, vnfH, vnfR2, vnfA (transcriptional regulator). In some cases, a Gram-positive microbe may have an iron-only nitrogenase system comprising: anfK, anfG, anfD, anfH, anfA (transcriptional regulator). In some cases, a Gram-positive microbe may have a nitrogenase system comprising glnB, and glnK (nitrogen signaling proteins). Some examples of enzymes involved in nitrogen metabolism in Gram-positive microbes include glnA (glutamine synthetase), gdh (glutamate dehydrogenase), bdh (3-hydroxybutyrate dehydrogenase), glutaminase, gltAB gltB gltS (glutamate synthase), asnA asnB (aspartate-ammonia ligase/asparagine synthetase), and ansA ansZ (asparaginase). Some examples of proteins involved in nitrogen transport in Gram-positive microbes include amtB (ammonium transporter), glnK (regulator of ammonium transport), glnPHQ glnQHMP (ATP-dependent glutamine/glutamate transporters), glnT/alsTyrbD/yflA (glutamine-like proton symport transporters), and gltP/gltT/yhcl/nqt (glutamate-like proton symport transporters).

Examples of Gram-positive microbes which may be of particular interest include Paenibacillus polymyxa, Paenibacillus riograndensis, Paenibacillus sp., Frankia sp., Heliobacterium sp., Heliobacterium chlorum, Heliobacillus sp., Heliophilum sp., Heliorestis sp., Clostridium acetobutylicum, Clostridium sp., Mycobacterium flaum, Mycobacterium sp., Arthrobacter sp., Agromyces sp., Corynebacterium autitrophicum, Corynebacterium sp., Micromonspora sp., Propionibacteria sp., Streptomyces sp., and Microbacterium sp.

Some examples of genetic alterations which may be made in Gram-positive microbes include: deleting glnR to remove negative regulation of BNF in the presence of environmental nitrogen, mutating glnR to remove repressive activity in the presence of fixed nitrogen (e.g., ammonium), inserting different promoters directly upstream of the nif cluster to eliminate regulation by GlnR in response to environmental nitrogen, eliminating portions of the promoters directly upstream of the nif cluster to eliminate regulation by GlnR in response to environmental nitrogen, eliminating portions of the promoters directly upstream of the nif cluster and inserting different promoters directly upstream of the nif cluster to eliminate regulation by GlnR in response to environmental nitrogen, mutating glnA to reduce the rate of ammonium assimilation by the GS-GOGAT pathway, deleting amtB to reduce uptake of ammonium from the media, mutating glnA so it is constitutively in the feedback-inhibited (FBI-GS) state, to reduce ammonium assimilation by the GS-GOGAT pathway. In some cases, the Gram-positive microbes have reduced GlnA protein activity (e.g., the GlnA protein is truncated) or the GlnA protein expressed from the glnRA operon is eliminated, allowing the microbes to fix nitrogen continuously and secrete ammonium.

In some cases, glnR is the main regulator of N metabolism and fixation in Paenibacillus species. In some cases, the genome of a Paenibacillus species may not contain a gene to produce glnR. In some cases, the genome of a Paenibacillus species may contain a gene to produce a mutant glnR that does not show any or substantially any repressive activity in the presence of fixed nitrogen (e.g., ammonium). In some cases, the genome of a Paenibacillus species may not contain a gene to produce glnE or glnD. In some cases, the genome of a Paenibacillus species may contain a gene to produce glnB or glnK. For example, Paenibacillus sp. WLY78 does not contain a gene for glnB, or its homologs found in the archaeon Methanococcus maripaludis, nifl1 and nifl2. In some cases, the genomes of Paenibacillus species may be variable. For example, Paenibacillus polymixa E681 lacks glnK and gdh, has several nitrogen compound transporters, but only amtB appears to be controlled by GlnR. In another example, Paenibacillus sp. JDR2 has glnK, gdh and most other central nitrogen metabolism genes, has many fewer nitrogen compound transporters, but does have glnPHQ controlled by GlnR. Paenibacillus riograndensis SBR5 contains a standard glnRA operon, anfdx gene, a main nif operon, a secondary nif operon, and an anf operon (encoding iron-only nitrogenase). Putative GlnR/TnrA sites were found upstream of each of these operons. GlnR may regulate all of the above operons, except the anf operon. GlnR may bind to each of these regulatory sequences as a dimer.

Paenibacillus N-fixing strains may fall into two subgroups: Subgroup I, which contains only a minimal nif gene cluster and subgroup II, which contains a minimal cluster, plus an uncharacterized gene between nifX and hesA, and often other clusters duplicating some of the nif genes, such as nifH, nifHDK, nifBEN, or clusters encoding vanadium nitrogenase (vnf) or iron-only nitrogenase (anf) genes.

In some cases, the genome of a Paenibacillus species may not contain a gene to produce GlnB or GlnK. In some cases, the genome of a Paenibacillus species may contain a minimal nif cluster with nine genes transcribed from a sigma-70 promoter. In some cases, a Paenibacillus nif cluster may be negatively regulated by nitrogen or oxygen. In some cases, the genome of a Paenibacillus species may not contain a gene to produce sigma-54. For example, Paenibacillus sp. WLY78 does not contain a gene for sigma-54. In some cases, a nif cluster may be regulated by GlnR, and/or TnrA. In some cases, activity of a nif cluster may be altered by altering activity of GlnR, and/or TnrA.

In Bacilli, glutamine synthetase (GS) is feedback-inhibited by high concentrations of intracellular glutamine, causing a shift in confirmation (referred to as FBI-GS). Nif clusters contain distinct binding sites for the regulators GlnR and TnrA in several Bacilli species. GlnR binds and represses gene expression in the presence of excess intracellular glutamine and AMP.

A role of GlnR may be to prevent the influx and intracellular production of glutamine and ammonium under conditions of high nitrogen availability. TnrA may bind and/or activate (or repress) gene expression in the presence of limiting intracellular glutamine, and/or in the presence of FBI-GS. In some cases, the activity of a Bacilli nif cluster may be altered by altering the activity of GlnR.

Feedback-inhibited glutamine synthetase (FBI-GS) may bind GlnR and stabilize binding of GlnR to recognition sequences. Several bacterial species have a GlnR/TnrA binding site upstream of the mf cluster. Altering the binding of FBI-GS and GlnR may alter the activity of the nif pathway.

### Budapest Treaty on the International Recognition of the Deposit of Microorganisms for the Purpose of Patent Procedures

The microbial deposits of the present disclosure were made under the provisions of the Budapest Treaty on the International Recognition of the Deposit of Microorganisms for the Purpose of Patent Procedure (Budapest Treaty). See Table 1. The disclosure contemplates embodiments comprising any one of the microbes listed in Table 1, as well as derivatives, variants, and/or mutants thereof. Further, the disclosure contemplates agricultural compositions comprising any one of the microbes listed in Table 1, as well as derivatives, variants, and/or mutants thereof. Further, the disclosure contemplates methods of utilizing any one of the microbes listed in Table 1, as well as derivatives, variants, and/or mutants thereof. Methods of the disclosure may comprise applying said microbe to a plant or plant part (such as a seed), or to an area in which said plant or plant part is to be grown, in order to supply fixed atmospheric nitrogen to said plant.

Applicants state that pursuant to 37 C.F.R. § 1.808(a)(2) “all restrictions imposed by the depositor on the availability to the public of the deposited material will be irrevocably removed upon the granting of the patent.” This statement is subject to paragraph (b) of this section (i.e. 37 C.F.R. § 1.808(b)).

In some aspects, the bacteria is selected from Table 1. In some aspects, the bacteria is selected from Table 1 and was deposited with the American Type Culture Collection (ATCC), located at 10801 University Boulevard, Manassas, Virginia 20110-2209, USA with the name designation, taxonomy, accession number and date of deposit as found in Table 1.

TABLE 1
Microorganisms Deposited under the Budapest Treaty

| | Name | | Accession | Date of |
|---|---|---|---|---|
| Depository | Designation | Taxonomy | Number | Deposit |
| ATCC | 41, CI-41 | Paenibacillus polymyxa | PTA-126581 | Dec. 26, 2019 |

### Sources of Microbes

The bacteria (or any microbe according to the disclosure) may be obtained from any general terrestrial environment, including its soils, plants, fungi, animals (including invertebrates) and other biota, including the sediments, water and biota of lakes and rivers; from the marine environment, its biota and sediments (for example, sea water, marine muds, marine plants, marine invertebrates (for example, sponges), marine vertebrates (for example, fish)); the terrestrial and marine geosphere (regolith and rock, for example, crushed subterranean rocks, sand and clays); the cryosphere and its meltwater; the atmosphere (for example, filtered aerial dusts, cloud and rain droplets); urban, industrial and other man-made environments (for example, accumulated organic and mineral matter on concrete, roadside gutters, roof surfaces, and road surfaces).

The plants from which the bacteria (or any microbe according to the disclosure) are obtained may be a plant having one or more desirable traits, for example a plant which naturally grows in a particular environment or under certain conditions of interest. By way of example, a certain plant may naturally grow in sandy soil or sand of high salinity, or under extreme temperatures, or with little water, or it may be resistant to certain pests or disease present in the environment, and it may be desirable for a commercial crop to be grown in such conditions, particularly if they are, for example, the only conditions available in a particular geographic location. By way of further example, the bacteria may be collected from commercial crops grown in such environments, or more specifically from individual crop plants best displaying a trait of interest amongst a crop grown in any specific environment: for example the fastest-growing plants amongst a crop grown in saline-limiting soils, or the least damaged plants in crops exposed to severe insect damage or disease epidemic, or plants having desired quantities of certain metabolites and other compounds, including fiber content, oil content, and the like, or plants displaying desirable colors, taste or smell. The bacteria may be collected from a plant of interest or any material occurring in the environment of interest, including fungi and other animal and plant biota, soil, water, sediments, and other elements of the environment as referred to previously.

The bacteria (or any microbe according to the disclosure) may be isolated from plant tissue. This isolation can occur from any appropriate tissue in the plant, including for example root, stem and leaves, and plant reproductive tissues. By way of example, conventional methods for isolation from plants typically include the sterile excision of the plant material of interest (e.g. root or stem lengths, leaves), surface sterilization with an appropriate solution (e.g. 2% sodium hypochlorite), after which the plant material is placed on nutrient medium for microbial growth. Alternatively, the surface-sterilized plant material can be crushed in a sterile liquid (usually water) and the liquid suspension, including small pieces of the crushed plant material spread over the surface of a suitable solid agar medium, or media, which may or may not be selective (e.g. contain only phytic acid as a source of phosphorus). This approach is especially useful for bacteria that form isolated colonies and can be picked off individually to separate plates of nutrient medium, and further purified to a single species by well-known methods. Alternatively, the plant root or foliage samples may not be surface sterilized but only washed gently thus including surface-dwelling epiphytic microorganisms in the isolation process, or the epiphytic microbes can be isolated separately, by imprinting and lifting off pieces of plant roots, stem or leaves onto the surface of an agar medium and then isolating individual colonies as above. This approach is especially useful for bacteria, for example. Alternatively, the roots may be processed without washing off small quantities of soil attached to the roots, thus including microbes that colonize the plant rhizosphere. Otherwise, soil adhering to the roots can be removed, diluted and spread out onto agar of suitable selective and non-selective media to isolate individual colonies of rhizospheric bacteria.

### Isolated and Biologically Pure Microorganisms

The present disclosure, in certain embodiments, provides isolated and biologically pure microorganisms that have applications, inter alia, in agriculture. The disclosed microorganisms can be utilized in their isolated and biologically pure states, as well as being formulated into compositions (see below section for exemplary composition descriptions). Furthermore, the disclosure provides microbial compositions containing at least two members of the disclosed isolated and biologically pure microorganisms, as well as methods of utilizing said microbial compositions. Furthermore, the disclosure provides for methods of modulating nitrogen fixation in plants via the utilization of the disclosed isolated and biologically pure microbes. In some aspects, the isolated and biologically pure microorganisms of the disclosure are gram-positive microbes provided herein that comprise a nif operon with an altered or mutated promoter operably linked thereto, a mutated GlnR protein that allows for expression of the nif operon irrespective of the presence of levels of fixed nitrogen (e.g., ammonium), a mutated GlnA protein that exhibits decreased assimilation of fixed nitrogen or a combination thereof. In other aspects, the isolated and biologically pure microorganisms of the disclosure are a microorganism of Table 1 from PCT/US2020/012564 (e.g., one or more of NCMA Accession No. 201701001, 201701003, 201701002, 201708004, 201708003, 201708002, 201708001, 201712001, or 201712002, ATCC Accession No. PTA-126575 PTA-126576, PTA-126577, PTA-126578, PTA-126579, PTA-126580, PTA-126581, PTA-126582, PTA-126583, PTA-126584, PTA-126585, PTA-126586, PTA-126587, or PTA-126588) in combination with one or more gram-positive microbes provided herein that comprise a mf operon with an altered or mutated promoter operably linked thereto, a mutated GlnR protein that allows for expression of the nif operon irrespective of the presence of levels of fixed nitrogen (e.g., ammonium), and/or a mutated GlnA protein that exhibits decreased assimilation of fixed nitrogen. For example, a strain, child, mutant, or derivative, of an engineered gram-positive microbes are provided herein. The disclosure contemplates all possible combinations of engineered gram-positive microbes provided herein. The engineered gram-positive microbes can comprise one or any combination of a nif operon operably linked to a nifB promoter altered or mutated as described herein, a GlnR comprising one, all or any combination of mutations provided herein and a GlnA comprising one, all or any combination of SNPs provided herein. The disclosure further contemplates all possible combinations of microbes listed in Table 1 from PCT/US2020/012564 with the engineered gram-positive microbes provided herein, said combinations sometimes forming a microbial consortia. The microbes from provided herein, either individually or in any combination, can be combined with any plant, active molecule (synthetic, organic, etc.), adjuvant, carrier, supplement, or biological, mentioned in the disclosure. In some cases, the gram-positive microbes provided herein are non-intrageneric. In some cases, the gram-positive microbes provided herein are transgenic.

### Agricultural Compositions

Compositions comprising bacteria or bacterial populations produced according to methods described herein and/or having characteristics as described herein can be in the form of a liquid, a foam, or a dry product. Compositions comprising bacteria or bacterial populations produced according to methods described herein and/or having characteristics as described herein may also be used to improve plant traits. Compositions comprising bacteria or bacterial populations can comprise engineered gram-positive microbes that comprise one or any combination of a mf operon operably linked to a nifB promoter altered or mutated as described herein, a GlnR comprising one, all or any combination of mutations provided herein and a GlnA comprising one, all or any combination of SNPs provided herein. Any composition provided herein comprising one or more engineered gram-positive microbes as provided herein can further comprise one or more microbes from Table 1 from PCT/US2020/012564. In some cases, the gram-positive microbes provided herein are non-intrageneric. In some cases, the gram-positive microbes provided herein are transgenic.

In some examples, a composition comprising bacterial populations may be in the form of a dry powder, a slurry of powder and water, or a flowable seed treatment. The compositions comprising bacterial populations may be coated on a surface of a seed, and may be in liquid form.

The composition can be fabricated in bioreactors such as continuous stirred tank reactors, batch reactors, and on the farm. In some examples, compositions can be stored in a container, such as a jug or in mini bulk. In some examples, compositions may be stored within an object selected from the group consisting of a bottle, jar, ampule, package, vessel, bag, box, bin, envelope, carton, container, silo, shipping container, truck bed, and/or case.

Compositions may also be used to improve plant traits. In some examples, one or more compositions may be coated onto a seed. In some examples, one or more compositions may be coated onto a seedling. In some examples, one or more compositions may be coated onto a surface of a seed. In some examples, one or more compositions may be coated as a layer above a surface of a seed. In some examples, a composition that is coated onto a seed may be in liquid form, in dry product form, in foam form, in a form of a slurry of powder and water, or in a flowable seed treatment. In some examples, one or more compositions may be applied to a seed and/or seedling by spraying, immersing, coating, encapsulating, and/or dusting the seed and/or seedling with the one or more compositions. In some examples, multiple bacteria or bacterial populations can be coated onto a seed and/or a seedling of the plant. In some examples, at least two, at least three, at least four, at least five, at least six, at least seven, at least eight, at least nine, at least ten, or more than ten bacteria of a bacterial combination can be selected from one of the following genera: Bacillus, Curtobacterium, Paenibacillus, Saccharibacillus, and Lactobacillus.

Examples of compositions may include seed coatings for commercially important agricultural crops, for example, sorghum, canola, tomato, strawberry, barley, rice, maize, and wheat. Examples of compositions can also include seed coatings for corn, soybean, canola, sorghum, potato, rice, vegetables, cereals, and oilseeds. Seeds as provided herein can be genetically modified organisms (GMO), non-GMO, organic, or conventional. In some examples, compositions may be sprayed on the plant aerial parts, or applied to the roots by inserting into furrows in which the plant seeds are planted, watering to the soil, or dipping the roots in a suspension of the composition. In some examples, compositions may be dehydrated in a suitable manner that maintains cell viability and the ability to artificially inoculate and colonize host plants. The bacterial species may be present in compositions at a concentration of between 10⁸ to 10¹⁰ CFU/ml. In some examples, compositions may be supplemented with trace metal ions, such as molybdenum ions, iron ions, manganese ions, or combinations of these ions. The concentration of ions in examples of compositions as described herein may between about 0.1 mM and about 50 mM. Some examples of compositions may also be formulated with a carrier, such as beta-glucan, carboxylmethyl cellulose (CMC), bacterial extracellular polymeric substance (EPS), sugar, animal milk, or other suitable carriers. In some examples, peat or planting materials can be used as a carrier, or biopolymers in which a composition is entrapped in the biopolymer can be used as a carrier. The compositions comprising the bacterial populations described herein can improve plant traits, such as promoting plant growth, maintaining high chlorophyll content in leaves, increasing fruit or seed numbers, and increasing fruit or seed unit weight.

The compositions comprising the bacterial populations described herein may be coated onto the surface of a seed. As such, compositions comprising a seed coated with one or more bacteria described herein are also contemplated. The seed coating can be formed by mixing the bacterial population with a porous, chemically inert granular carrier. Alternatively, the compositions may be inserted directly into the furrows into which the seed is planted or sprayed onto the plant leaves or applied by dipping the roots into a suspension of the composition. An effective amount of the composition can be used to populate the sub-soil region adjacent to the roots of the plant with viable bacterial growth, or populate the leaves of the plant with viable bacterial growth. In general, an effective amount is an amount sufficient to result in plants with improved traits (e.g. a desired level of nitrogen fixation).

Bacterial compositions described herein can be formulated using an agriculturally acceptable carrier. The formulation useful for these embodiments may include at least one member selected from the group consisting of a tackifier, a microbial stabilizer, a fungicide, an antibacterial agent, a preservative, a stabilizer, a surfactant, an anti-complex agent, an herbicide, a nematicide, an insecticide, a plant growth regulator, a fertilizer, a rodenticide, a dessicant, a bactericide, a nutrient, a hormone, or any combination thereof. In some examples, compositions may be shelf-stable. For example, any of the compositions described herein can include an agriculturally acceptable carrier (e.g., one or more of a fertilizer such as a non-naturally occurring fertilizer, an adhesion agent such as a non-naturally occurring adhesion agent, and a pesticide such as a non-naturally occurring pesticide). A non-naturally occurring adhesion agent can be, for example, a polymer, copolymer, or synthetic wax. For example, any of the coated seeds, seedlings, or plants described herein can contain such an agriculturally acceptable carrier in the seed coating. In any of the compositions or methods described herein, an agriculturally acceptable carrier can be or can include a non-naturally occurring compound (e.g., a non-naturally occurring fertilizer, a non-naturally occurring adhesion agent such as a polymer, copolymer, or synthetic wax, or a non-naturally occurring pesticide). Non-limiting examples of agriculturally acceptable carriers are described below. Additional examples of agriculturally acceptable carriers are known in the art.

In some cases, bacteria are mixed with an agriculturally acceptable carrier. The carrier can be a solid carrier or liquid carrier, and in various forms including microspheres, powders, emulsions and the like. The carrier may be any one or more of a number of carriers that confer a variety of properties, such as increased stability, wettability, or dispersability. Wetting agents such as natural or synthetic surfactants, which can be nonionic or ionic surfactants, or a combination thereof can be included in the composition. Water-in-oil emulsions can also be used to formulate a composition that includes the isolated bacteria (see, for example, U.S. Pat. No. 7,485,451). Suitable formulations that may be prepared include wettable powders, granules, gels, agar strips or pellets, thickeners, and the like, microencapsulated particles, and the like, liquids such as aqueous flowables, aqueous suspensions, water-in-oil emulsions, etc. The formulation may include grain or legume products, for example, ground grain or beans, broth or flour derived from grain or beans, starch, sugar, or oil.

In some embodiments, the agricultural carrier may be soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the bacteria, such as barley, rice, or other biological materials such as seed, plant parts, sugar cane bagasse, hulls or stalks from grain processing, ground plant material or wood from building site refuse, sawdust or small fibers from recycling of paper, fabric, or wood.

For example, a fertilizer can be used to help promote the growth or provide nutrients to a seed, seedling, or plant. Non-limiting examples of fertilizers include nitrogen, phosphorous, potassium, calcium, sulfur, magnesium, boron, chloride, manganese, iron, zinc, copper, molybdenum, and selenium (or a salt thereof). Additional examples of fertilizers include one or more amino acids, salts, carbohydrates, vitamins, glucose, NaCl, yeast extract, NH₄H₂PO₄, (NH₄)₂SO₄, glycerol, valine, L-leucine, lactic acid, propionic acid, succinic acid, malic acid, citric acid, KH tartrate, xylose, lyxose, and lecithin. In one embodiment, the formulation can include a tackifier or adherent (referred to as an adhesive agent) to help bind other active agents to a substance (e.g., a surface of a seed). Such agents are useful for combining bacteria with carriers that can contain other compounds (e.g., control agents that are not biologic), to yield a coating composition. Such compositions help create coatings around the plant or seed to maintain contact between the microbe and other agents with the plant or plant part. In one embodiment, adhesives are selected from the group consisting of: alginate, gums, starches, lecithins, formononetin, polyvinyl alcohol, alkali formononetinate, hesperetin, polyvinyl acetate, cephalins, gum arabic, xanthan gum, mineral oil, polyethylene glycol (PEG), polyvinyl pyrrolidone (PVP), arabino-galactan, methyl cellulose, PEG 400, chitosan, polyacrylamide, polyacrylate, polyacrylonitrile, glycerol, triethylene glycol, vinyl acetate, gellan gum, polystyrene, polyvinyl, carboxymethyl cellulose, gum ghatti, and polyoxyethylene-polyoxybutylene block copolymers.

In some embodiments, the adhesives can be, e.g. a wax such as carnauba wax, beeswax, Chinese wax, shellac wax, spermaceti wax, candelilla wax, castor wax, ouricury wax, and rice bran wax, a polysaccharide (e.g., starch, dextrins, maltodextrins, alginate, and chitosans), a fat, oil, a protein (e.g., gelatin and zeins), gum arables, and shellacs. Adhesive agents can be non-naturally occurring compounds, e.g., polymers, copolymers, and waxes. For example, non-limiting examples of polymers that can be used as an adhesive agent include: polyvinyl acetates, polyvinyl acetate copolymers, ethylene vinyl acetate (EVA) copolymers, polyvinyl alcohols, polyvinyl alcohol copolymers, celluloses (e.g., ethylcelluloses, methylcelluloses, hydroxymethylcelluloses, hydroxypropylcelluloses, and carboxymethylcelluloses), polyvinylpyrolidones, vinyl chloride, vinylidene chloride copolymers, calcium lignosulfonates, acrylic copolymers, polyvinylacrylates, polyethylene oxide, acylamide polymers and copolymers, polyhydroxyethyl acrylate, methylacrylamide monomers, and polychloroprene.

In some examples, one or more of the adhesion agents, anti-fungal agents, growth regulation agents, and pesticides (e.g., insecticide) are non-naturally occurring compounds (e.g., in any combination). Additional examples of agriculturally acceptable carriers include dispersants (e.g., polyvinylpyrrolidone/vinyl acetate PVPIVA S-630), surfactants, binders, and filler agents.

The formulation can also contain a surfactant. Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

In certain cases, the formulation includes a microbial stabilizer. Such an agent can include a desiccant, which can include any compound or mixture of compounds that can be classified as a desiccant regardless of whether the compound or compounds are used in such concentrations that they in fact have a desiccating effect on a liquid inoculant. Such desiccants are ideally compatible with the bacterial population used, and should promote the ability of the microbial population to survive application on the seeds and to survive desiccation. Examples of suitable desiccants include one or more of trehalose, sucrose, glycerol, and methylene glycol. Other suitable desiccants include, but are not limited to, non reducing sugars and sugar alcohols (e.g., mannitol or sorbitol). The amount of desiccant introduced into the formulation can range from about 5% to about 50% by weight/volume, for example, between about 10% to about 40%, between about 15% to about 35%, or between about 20% to about 30%. In some cases, it is advantageous for the formulation to contain agents such as a fungicide, an antibacterial agent, an herbicide, a nematicide, an insecticide, a plant growth regulator, a rodenticide, bactericide, or a nutrient. In some examples, agents may include protectants that provide protection against seed surface-borne pathogens. In some examples, protectants may provide some level of control of soil-borne pathogens. In some examples, protectants may be effective predominantly on a seed surface.

In some examples, a fungicide may include a compound or agent, whether chemical or biological, that can inhibit the growth of a fungus or kill a fungus. In some examples, a fungicide may include compounds that may be fungistatic or fungicidal. In some examples, fungicide can be a protectant, or agents that are effective predominantly on the seed surface, providing protection against seed surface-borne pathogens and providing some level of control of soil-borne pathogens. Non-limiting examples of protectant fungicides include captan, maneb, thiram, or fludioxonil.

In some examples, fungicide can be a systemic fungicide, which can be absorbed into the emerging seedling and inhibit or kill the fungus inside host plant tissues. Systemic fungicides used for seed treatment include, but are not limited to the following: azoxystrobin, carboxin, mefenoxam, metalaxyl, thiabendazole, trifloxystrobin, and various triazole fungicides, including difenoconazole, ipconazole, tebuconazole, and triticonazole. Mefenoxam and metalaxyl are primarily used to target the water mold fungi Pythium and Phytophthora. Some fungicides are preferred over others, depending on the plant species, either because of subtle differences in sensitivity of the pathogenic fungal species, or because of the differences in the fungicide distribution or sensitivity of the plants. In some examples, fungicide can be a biological control agent, such as a bacterium or fungus. Such organisms may be parasitic to the pathogenic fungi, or secrete toxins or other substances that can kill or otherwise prevent the growth of fungi. Any type of fungicide, particularly ones that are commonly used on plants, can be used as a control agent in a seed composition.

In some examples, the seed coating composition comprises a control agent that has antibacterial properties. In one embodiment, the control agent with antibacterial properties is selected from the compounds described herein elsewhere. In another embodiment, the compound is streptomycin, oxytetracycline, oxolinic acid, or gentamicin. Other examples of antibacterial compounds which can be used as part of a seed coating composition include those based on dichlorophene and benzylalcohol hemi formal (Proxel® from ICI or Acticide® RS from Thor Chemie and Kathon® MK 25 from Rohm & Haas) and isothiazolinone derivatives such as alkylisothiazolinones and benzisothiazolinones (Acticide® MBS from Thor Chemie). In some examples, growth regulator is selected from the group consisting of: abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione phosphorotrithioate, 2,3,5-tri-iodobenzoic acid, trinexapac-ethyl and uniconazole. Additional non-limiting examples of growth regulators include brassinosteroids, cytokinines (e.g., kinetin and zeatin), auxins (e.g., indolylacetic acid and indolylacetyl aspartate), flavonoids and isoflavanoids (e.g., formononetin and diosmetin), phytoaixins (e.g., glyceolline), and phytoalexin-inducing oligosaccharides (e.g., pectin, chitin, chitosan, polygalacuronic acid, and oligogalacturonic acid), and gibellerins. Such agents are ideally compatible with the agricultural seed or seedling onto which the formulation is applied (e.g., it should not be deleterious to the growth or health of the plant). Furthermore, the agent is ideally one, which does not cause safety concerns for human, animal or industrial use (e.g., no safety issues or the compound is sufficiently labile that the commodity plant product derived from the plant contains negligible amounts of the compound).

Some examples of nematode-antagonistic biocontrol agents include ARF18; 30 Arthrobotrys spp.; Chaetomium spp.; Cylindrocarpon spp.; Exophilia spp.; Fusarium spp.; Gliocladium spp.; Hirsutella spp.; Lecanicillium spp.; Monacrosporium spp.; Myrothecium spp.; Neocosmospora spp.; Paecilomyces spp.; Pochonia spp.; Stagonospora spp.; vesicular-arbuscular mycorrhizal fungi, Burkholderia spp.; Pasteuria spp., Brevibacillus spp.; Pseudomonas spp.; and Rhizobacteria. Particularly preferred nematode-antagonistic biocontrol agents include ARF18, Arthrobotrys oligospora, Arthrobotrys dactyloides, Chaetomium globosum, Cylindrocarpon heteronema, Exophilia jeanselmei, Exophilia pisciphila, Fusarium aspergilus, Fusarium solani, Gliocladium catenulatum, Gliocladium roseum, Gliocladium vixens, Hirsutella rhossiliensis, Hirsutella minnesotensis, Lecanicillium lecanii, Monacrosporium drechsleri, Monacrosporium gephyropagum, Myrotehcium verrucaria, Neocosmospora vasinfecta, Paecilomyces lilacinus, Pochonia chlamydosporia, Stagonospora heteroderae, Stagonospora phaseoli, vesicular-arbuscular mycorrhizal fungi, Burkholderia cepacia, Pasteuria penetrans, Pasteuria thornei, Pasteuria nishizawae, Pasteuria ramosa, Pastrueia usage, Brevibacillus laterosporus strain G4, Pseudomonas fluorescens and Rhizobacteria.

Some examples of nutrients can be selected from the group consisting of a nitrogen fertilizer including, but not limited to urea, ammonium nitrate, ammonium sulfate, non-pressure nitrogen solutions, aqua ammonia, anhydrous ammonia, ammonium thiosulfate, sulfur-coated urea, urea-formaldehydes, IBDU, polymer-coated urea, calcium nitrate, ureaform, and methylene urea, phosphorous fertilizers such as diammonium phosphate, monoammonium phosphate, ammonium polyphosphate, concentrated superphosphate and triple superphosphate, and potassium fertilizers such as potassium chloride, potassium sulfate, potassium-magnesium sulfate, potassium nitrate. Such compositions can exist as free salts or ions within the seed coat composition. Alternatively, nutrients/fertilizers can be complexed or chelated to provide sustained release over time.

Some examples of rodenticides may include selected from the group of substances consisting of 2-isovalerylindan-1,3-dione, 4-(quinoxalin-2-ylamino) benzenesulfonamide, alpha-chlorohydrin, aluminum phosphide, antu, arsenous oxide, barium carbonate, bisthiosemi, brodifacoum, bromadiolone, bromethalin, calcium cyanide, chloralose, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, hydrogen cyanide, iodomethane, lindane, magnesium phosphide, methyl bromide, norbormide, phosacetim, phosphine, phosphorus, pindone, potassium arsenite, pyrinuron, scilliroside, sodium arsenite, sodium cyanide, sodium fluoroacetate, strychnine, thallium sulfate, warfarin and zinc phosphide.

In the liquid form, for example, solutions or suspensions, bacterial populations can be mixed or suspended in water or in aqueous solutions. Suitable liquid diluents or carriers include water, aqueous solutions, petroleum distillates, or other liquid carriers.

Solid compositions can be prepared by dispersing the bacterial populations in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

The solid carriers used upon formulation include, for example, mineral carriers such as kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran may be used. The liquid carriers include vegetable oils such as soybean oil and cottonseed oil, glycerol, ethylene glycol, polyethylene glycol, propylene glycol, polypropylene glycol, etc.

### Pesticidal Compositions Comprising a Pesticide and Microbe of the Disclosure

As aforementioned, agricultural compositions of the disclosure, which may comprise any microbe taught herein, are sometimes combined with one or more pesticides. Pesticides can include herbicides, insecticides, fungicides, nematicides, etc.

In some embodiments, the pesticides/microbial combinations can be applied in the form of compositions and can be applied to the crop area or plant to be treated, simultaneously or in succession, with other compounds. These compounds can be fertilizers, weed killers, cryoprotectants, surfactants, detergents, pesticidal soaps, dormant oils, polymers, and/or time release or biodegradable carrier formulations that permit long term dosing of a target area following a single application of the formulation. They can also be selective herbicides, chemical insecticides, virucides, microbicides, amoebicides, pesticides, fungicides, bacteriocides, nematicides, molluscicides or mixtures of several of these preparations, if desired, together with further agriculturally acceptable carriers, surfactants or application promoting adjuvants customarily employed in the art of formulation. Suitable carriers (i.e. agriculturally acceptable carriers) and adjuvants can be solid or liquid and correspond to the substances ordinarily employed in formulation technology, e.g. natural or regenerated mineral substances, solvents, dispersants, wetting agents, sticking agents, tackifiers, binders or fertilizers. Likewise, the formulations may be prepared into edible baits or fashioned into pest traps to permit feeding or ingestion by a target pest of the pesticidal formulation.

### Herbicides

As aforementioned, agricultural compositions of the disclosure, which may comprise any microbe taught herein, are sometimes combined with one or more herbicides.

Compositions comprising bacteria or bacterial populations produced according to methods described herein and/or having characteristics as described herein may further include one or more herbicides. In some embodiments, herbicidal compositions are applied to the plants and/or plant parts. In some embodiments, herbicidal compositions may be included in the compositions set forth herein, and can be applied to a plant(s) or a part(s) thereof simultaneously or in succession, with other compounds.

Herbicides include 2,4-D, 2,4-DB, acetochlor, acifluorfen, alachlor, ametryn, atrazine, aminopyralid, benefin, bensulfuron, bensulide, bentazon, bicyclopyrone, bromacil, bromoxynil, butylate, carfentrazone, chlorimuron, chlorsulfuron, clethodim, clomazone, clopyralid, cloransulam, cycloate, DCPA, desmedipham, dicamba, dichlobenil, diclofop, diclosulam, diflufenzopyr, dimethenamid, diquat, diuron, DSMA, endothall, EPTC, ethalfluralin, ethofumesate, fenoxaprop, fluazifop-P, flucarbzone, flufenacet, flumetsulam, flumiclorac, flumioxazin, fluometuron, fluroxypyr, fomesafen, foramsulfuron, glufosinate, glyphosate, halosulfuron, hexazinone, imazamethabenz, imazamox, imazapic, imazaquin, imazethapyr, isoxaflutole, lactofen, linuron, MCPA, MCPB, mesotrione, metolachlor-s, metribuzin, indaziflam, metsulfuron, molinate, MSMA, napropamide, naptalam, nicosulfuron, norflurazon, oryzalin, oxadiazon, oxyfluorfen, paraquat, pelargonic acid, pendimethalin, phenmedipham, picloram, primisulfuron, prodiamine, prometryn, pronamide, propanil, prosulfuron, pyrazon, pyrithioac, quinclorac, quizalofop, rimsulfuron, S-metolachlor, sethoxydim, siduron, simazine, sulfentrazone, sulfometuron, sulfosulfuron, tebuthiuron, tembotrione, terbacil, thiazopyr, thifensulfuron, thiobencarb, topramezone, tralkoxydim, triallate, triasulfuron, tribenuron, triclopyr, trifluralin, and triflusulfuron.

In some embodiments, any one or more of the herbicides set forth herein may be utilized with any one or more of the plants or parts thereof set forth herein.

Herbicidal products may include CORVUS®, BALANCE® FLEXX, CAPRENO®, DIFLEXX, LIBERTY®, LAUDIS, AUTUMN SUPER, and DIFLEXX DUO®.

In some embodiments, any one or more of the herbicides set forth in the below Table 2 may be utilized with any one or more of the microbes taught herein, and can be applied to any one or more of the plants or parts thereof set forth herein.

TABLE 2
List of exemplary herbicides, which can be combined with microbes of the disclosure

| | Herbicide | | |
|---|---|---|---|
| | Group | | |
| Site of Action | Number | Chemical Family | Herbicide |
| ACCase | 1 | Cyclohexanediones | Sethoxydim (Poast, |
| inhibitors | | | Poast Plus) |
| | | | Clethodim (Select, |
| | | | Select Max, Arrow) |
| | | Aryloxyphenoxypropionates | Fluazifop (Fusilade DX, |
| | | | component in Fusion) |
| | | | Fenoxaprop (Puma, |
| | | | component in Fusion) |
| | | | Quizalofop (Assure II, |
| | | | Targa) |
| | | Phenylpyrazolins | Pinoxaden (Axial XL) |
| ALS inhibitors | 2 | Imidazolinones | Imazethapyr (Pursuit) |
| | | | Imazamox (Raptor) |
| | | Sulfonylureas | Chlorimuron (Classic) |
| | | | Halosulfuron (Permit, |
| | | | Sandea) |
| | | | Iodosulfuron |
| | | | (component in Autumn |
| | | | Super) |
| | | | Mesosulfuron (Osprey) |
| | | | Nicosulfuron (Accent Q) |
| | | | Primisulfuron (Beacon) |
| | | | Prosulfuron (Peak) |
| | | | Rimsulfuron (Matrix, |
| | | | Resolve) |
| | | | Thifensulfuron |
| | | | (Harmony) |
| | | | Tribenuron (Express) |
| | | | Triflusulfuron (UpBeet) |
| | | Triazolopyrimidine | Flumetsulam (Python) |
| | | | Cloransulam-methyl |
| | | | (FirstRate) |
| | | | Pyroxsulam (PowerFlex |
| | | | HL) |
| | | | Florasulam (component |
| | | | in Quelex) |
| | | Sulfonylaminocarbonyltriazolinones | Propoxycarbazone |
| | | | (Olympus) |
| | | | Thiencarbazone-methyl |
| | | | (component in |
| | | | Capreno) |
| Microtubule | 3 | Dinitroanilines | Trifluralin (many |
| inhibitors (root | | | names) |
| inhibitors) | | | Ethalfluralin (Sonalan) |
| | | | Pendimethalin |
| | | | (Prowl/Prowl H₂O) |
| | | Benzamide | Pronamide (Kerb) |
| Synthetic auxins | 4 | Arylpicolinate | Halauxifen (Elevore, |
| | | | component in Quelex) |
| | | Phenoxy acetic acids | 2,4-D (Enlist One, |
| | | | others) |
| | | | 2,4-DB (Butyrac 200, |
| | | | Butoxone 200) |
| | | | MCPA |
| | | Benzoic acids | Dicamba (Banvel, |
| | | | Clarity, DiFlexx, |
| | | | Eugenia, XtendiMax; |
| | | | component in Status) |
| | | Pyridines | Clopyralid (Stinger) |
| | | | Fluroxypyr (Starane |
| | | | Ultra) |
| Photosystem II | 5 | Triazines | Atrazine |
| inhibitors | | | Simazine (Princep, Sim- |
| | | | Trol) |
| | | Triazinone | Metribuzin (Metribuzin, |
| | | | others) |
| | | | Hexazinone (Velpar) |
| | | Phenyl-carbamates | Desmedipham (Betenex) |
| | | | Phenmedipham |
| | | | (component in Betamix) |
| | | Uracils | Terbacil (Sinbar) |
| | 6 | Benzothiadiazoles | Bentazon (Basagran, |
| | | | others) |
| | | Nitriles | Bromoxynil (Buctril, |
| | | | Moxy, others) |
| | 7 | Phenylureas | Linuron (Lorox, Linex) |
| Lipid synthesis | 8 | Thiocarbamates | EPTC (Eptam) |
| inhibitor | | | |
| EPSPS inhibitor | 9 | Organophosphorus | Glyphosate |
| Glutamine | 10 | Organophosphorus | Glufosinate (Liberty, |
| synthetase | | | Rely) |
| inhibitor | | | |
| Diterpene | 13 | Isoxazolidinone | Clomazone (Command) |
| biosynthesis | | | |
| inhibitor | | | |
| (bleaching) | | | |
| Protoporphyrinogen | 14 | Diphenylether | Acifluorfen (Ultra |
| oxidase | | | Blazer) |
| inhibitors (PPO) | | | Fomesafen (Flexstar, |
| | | | Reflex) |
| | | | Lactofen (Cobra, |
| | | | Phoenix) |
| | | N-phenylphthalimide | Flumiclorac (Resource) |
| | | | Flumioxazin (Valor, |
| | | | Valor EZ, Rowel) |
| | | Aryl triazolinone | Sulfentrazone |
| | | | (Authority, Spartan) |
| | | | Carfentrazone (Aim) |
| | | | Fluthiacet-methyl |
| | | | (Cadet) |
| | | Pyrazoles | Pyraflufen-ethyl (Vida) |
| | | Pyrimidinedione | Saflufenacil (Sharpen) |
| Long-chain fatty | 15 | Acetamides | Acetochlor (Harness, |
| acid inhibitors | | | Surpass NXT, |
| | | | Breakfree NXT, |
| | | | Warrant) |
| | | | Dimethenamid-P |
| | | | (Outlook) |
| | | | Metolachlor (Parallel) |
| | | | Pyroxasulfone (Zidua, |
| | | | Zidua SC) |
| | | | s-metolachlor (Dual |
| | | | Magnum, Dual II |
| | | | Magnum, Cinch) |
| | | | Flufenacet (Define) |
| Specific site | 16 | Benzofuranes | Ethofumesate (Nortron) |
| unknown | | | |
| Auxin transport | 19 | Semicarbazone | diflufenzopyr |
| inhibitor | | | (component in Status) |
| Photosystem I | 22 | Bipyridiliums | Paraquat (Gramoxone, |
| inhibitors | | | Parazone) |
| | | | Diquat (Reglone) |
| 4-HPPD | 27 | Isoxazole | Isoxaflutole (Balance |
| inhibitors | | Pyrazole | Flexx) |
| (bleaching) | | Pyrazolone | Pyrasulfotole |
| | | Triketone | (component in Huskie) |
| | | | Topramezone |
| | | | (Armezon/Impact) |
| | | | Bicyclopyrone |
| | | | (component in Acuron) |
| | | | Mesotrione (Callisto) |
| | | | Tembotrione (Laudis) |

### Fungicides

As aforementioned, agricultural compositions of the disclosure, which may comprise any microbe taught herein, are sometimes combined with one or more fungicides.

Compositions comprising bacteria or bacterial populations produced according to methods described herein and/or having characteristics as described herein may further include one or more fungicides. In some embodiments, fungicidal compositions may be included in the compositions set forth herein, and can be applied to a plant(s) or a part(s) thereof simultaneously or in succession, with other compounds. The fungicides include azoxystrobin, captan, carboxin, ethaboxam, fludioxonil, mefenoxam, fludioxonil, thiabendazole, thiabendaz, ipconazole, mancozeb, cyazofamid, zoxamide, metalaxyl, PCNB, metaconazole, pyraclostrobin, Bacillus subtilis strain QST 713, sedaxane, thiamethoxam, fludioxonil, thiram, tolclofos-methyl, trifloxystrobin, Bacillus subtilis strain MBI 600, pyraclostrobin, fluoxastrobin, Bacillus pumilus strain QST 2808, chlorothalonil, copper, flutriafol, fluxapyroxad, mancozeb, gludioxonil, penthiopyrad, triazole, propiconaozole, prothioconazole, tebuconazole, fluoxastrobin, pyraclostrobin, picoxystrobin, qols, tetraconazole, trifloxystrobin, cyproconazole, flutriafol, SDHI, EBDCs, sedaxane, MAXIM QUATTRO (gludioxonil, mefenoxam, azoxystrobin, and thiabendaz), RAXIL (tebuconazole, prothioconazole, metalaxyl, and ethoxylated tallow alkyl amines), and benzovindiflupyr.

In some embodiments, any one or more of the fungicides set forth herein may be utilized with any one or more of the plants or parts thereof set forth herein.

### Hormones

As aforementioned, agricultural compositions of the disclosure, which may comprise any microbe taught herein, are sometimes combined with one or more hormones.

Compositions comprising bacteria or bacterial populations produced according to methods described herein and/or having characteristics as described herein may further include one or more hormones. In some embodiments, hormone compositions are applied to the plants and/or plant parts. In some embodiments, hormone compositions may be included in the compositions set forth herein, and can be applied to a plant(s) or a part(s) thereof simultaneously or in succession, with other compounds.

Hormones include, but are not limited to, auxins, cytokinins, gibberellins, abscisic acid, ethylene, brassinosteroids, jasmonic acid, strigolactones, and chemical mimics of strigolactone.

In some embodiments, any one or more of the hormones set forth herein may be utilized with any one or more of the plants or parts thereof set forth herein.

### Strigolactones

As aforementioned, agricultural compositions of the disclosure, which may comprise any microbe taught herein, are sometimes combined with one or more strigolactone or chemical mimics of strigolactone. Such compounds are described in PCT/US2016/029080, filed Apr. 23, 2016, and entitled: Methods for Hydraulic Enhancement of Crops, which is hereby incorporated by reference. They are further described in U.S. Pat. No. 9,994,557, issued on Jun. 12, 2018, and entitled: Strigolactone Formulations and Uses Thereof, which is hereby incorporated by reference.

### Nematicides

As aforementioned, agricultural compositions of the disclosure, which may comprise any microbe taught herein, are sometimes combined with one or more nematicides.

### Fertilizers, Nitrogen Stabilizers, and Urease Inhibitors

As aforementioned, agricultural compositions of the disclosure, which may comprise any microbe taught herein, are sometimes combined with one or more of a: fertilizer, nitrogen stabilizer, or urease inhibitor.

In some embodiments, fertilizers are used in combination with the methods and bacteria of the present disclosure. Fertilizers include anhydrous ammonia, urea, ammonium nitrate, and urea-ammonium nitrate (UAN) compositions, among many others. In some embodiments, pop-up fertilization and/or starter fertilization is used in combination with the methods and bacteria of the present disclosure.

In some embodiments, nitrogen stabilizers are used in combination with the methods and bacteria of the present disclosure. Nitrogen stabilizers include nitrapyrin, 2-chloro-6-(trichloromethyl) pyridine, N-SERVE 24, INSTINCT, dicyandiamide (DCD).

In some embodiments, urease inhibitors are used in combination with the methods and bacteria of the present disclosure. Urease inhibitors include N-(n-butyl)-thiophosphoric triamide (NBPT), AGROTAIN, AGROTAIN PLUS, and AGROTAIN PLUS SC. Further, the disclosure contemplates utilization of AGROTAIN ADVANCED 1.0, AGROTAIN DRI-MAXX, and AGROTAIN ULTRA.

Further, stabilized forms of fertilizer can be used. For example, a stabilized form of fertilizer is SUPER U, containing 46% nitrogen in a stabilized, urea-based granule, SUPER U contains urease and nitrification inhibitors to guard from denitrification, leaching, and volatilization. Stabilized and targeted foliar fertilizer such as NITAMIN may also be used herein.

Pop-up fertilizers are commonly used in corn fields. Pop-up fertilization comprises applying a few pounds of nutrients with the seed at planting. Pop-up fertilization is used to increase seedling vigor.

Slow- or controlled-release fertilizer that may be used herein entails: A fertilizer containing a plant nutrient in a form which delays its availability for plant uptake and use after application, or which extends its availability to the plant significantly longer than a reference ‘rapidly available nutrient fertilizer’ such as ammonium nitrate or urea, ammonium phosphate or potassium chloride. Such delay of initial availability or extended time of continued availability may occur by a variety of mechanisms. These include controlled water solubility of the material by semi-permeable coatings, occlusion, protein materials, or other chemical forms, by slow hydrolysis of water-soluble low molecular weight compounds, or by other unknown means.

Stabilized nitrogen fertilizer that may be used herein entails: A fertilizer to which a nitrogen stabilizer has been added. A nitrogen stabilizer is a substance added to a fertilizer that extends the time the nitrogen component of the fertilizer remains in the soil in the urea-N or ammoniacal-N form.

Nitrification inhibitor that may be used herein entails: A substance that inhibits the biological oxidation of ammoniacal-N to nitrate-N. Some examples include: (1) 2-chloro-6-(trichloromethyl-pyridine), common name Nitrapyrin, manufactured by Dow Chemical; (2) 4-amino-1,2,4-6-triazole-HCl, common name ATC, manufactured by Ishihada Industries; (3) 2,4-diamino-6-trichloro-methyltriazine, common name CI-1580, manufactured by American Cyanamid; (4) Dicyandiamide, common name DCD, manufactured by Showa Denko; (5) Thiourea, common name TU, manufactured by Nitto Ryuso; (6) 1-mercapto-1,2,4-triazole, common name MT, manufactured by Nippon; (7) 2-amino-4-chloro-6-methyl-pyramidine, common name AM, manufactured by Mitsui Toatsu; (8) 3,4-dimethylpyrazole phosphate (DMPP), from BASF; (9) 1-amide-2-thiourea (ASU), from Nitto Chemical Ind.; (10) Ammoniumthiosulphate (ATS); (11) 1H-1,2,4-triazole (HPLC); (12) 5-ethylene oxide-3-trichloro-methlyl,2,4-thiodiazole (Terrazole), from Olin Mathieson; (13) 3-methylpyrazole (3-MP); (14) 1-carbamoyle-3-methyl-pyrazole (CMP); (15) Neem; and (16) DMPP.

Urease inhibitor that may be used herein entails: A substance that inhibits hydrolytic action on urea by the enzyme urease. Thousands of chemicals have been evaluated as soil urease inhibitors (Kiss and Simihaian, 2002). However, only a few of the many compounds tested meet the necessary requirements of being nontoxic, effective at low concentration, stable, and compatible with urea (solid and solutions), degradable in the soil and inexpensive. They can be classified according to their structures and their assumed interaction with the enzyme urease (Watson, 2000, 2005). Four main classes of urease inhibitors have been proposed: (a) reagents, which interact with the sulphydryl groups (sulphydryl reagents), (b) hydroxamates, (c) agricultural crop protection chemicals, and (d) structural analogues of urea and related compounds. N-(n-Butyl) thiophosphoric triamide (NBPT), phenylphosphorodiamidate (PPD/PPDA), and hydroquinone are probably the most thoroughly studied urease inhibitors (Kiss and Simihaian, 2002). Research and practical testing has also been carried out with N-(2-nitrophenyl) phosphoric acid triamide (2-NPT) and ammonium thiosulphate (ATS). The organo-phosphorus compounds are structural analogues of urea and are some of the most effective inhibitors of urease activity, blocking the active site of the enzyme (Watson, 2005).

### Insecticidal Seed Treatments (ISTs) for Corn

Corn seed treatments normally target three spectrums of pests: nematodes, fungal seedling diseases, and insects.

Insecticide seed treatments are usually the main component of a seed treatment package. Most corn seed available today comes with a base package that includes a fungicide and insecticide. In some aspects, the insecticide options for seed treatments include PONCHO (clothianidin), CRUISER/CRUISER EXTREME (thiamethoxam) and GAUCHO (Imidacloprid). All three of these products are neonicotinoid chemistries. CRUISER and PONCHO at the 250 (0.25 mg AI/seed) rate are some of the most common base options available for corn. In some aspects, the insecticide options for treatments include CRUISER 250 thiamethoxam, CRUISER 250 (thiamethoxam) plus LUMIVIA (chlorantraniliprole), CRUISER 500 (thiamethoxam), and PONCHO VOTIVO 1250 (Clothianidin & Bacillus firmus I-1582).

Pioneer's base insecticide seed treatment package consists of CRUISER 250 with PONCHO/VOTIVO 1250 also available. VOTIVO is a biological agent that protects against nematodes.

Monsanto's products including corn, soybeans, and cotton fall under the ACCELERON treatment umbrella. Dekalb corn seed comes standard with PONCHO 250. Producers also have the option to upgrade to PONCHO/VOTIVO, with PONCHO applied at the 500 rate.

Agrisure, Golden Harvest and Garst have a base package with a fungicide and CRUISER 250. AVICTA complete corn is also available; this includes CRUISER 500, fungicide, and nematode protection. CRUISER EXTREME is another option available as a seed treatment package, however; the amounts of CRUISER are the same as the conventional CRUISER seed treatment, i.e. 250, 500, or 1250.

Another option is to buy the minimum insecticide treatment available, and have a dealer treat the seed downstream.

Commercially available ISTs for corn are listed in the below Table 3 and can be combined with one or more of the microbes taught herein.

TABLE 3
List of exemplary seed treatments, including ISTs, which
can be combined with microbes of the disclosure

| Treatment Type | Active Ingredient(s) | Product Trade Name | Crop |
|---|---|---|---|
| F | azoxystrobin | DYNASTY | Corn, Soybean |
| | | PROTÉGÉ FL | Corn |
| F | Bacillus pumilus | YIELD SHIELD | Corn, Soybean |
| F | Bacillus subtilis | HISTICK N/T | Soybean |
| | | VAULT HP | Corn, Soybean |
| F | Captan | CAPTAN 400 | Corn, Soybean |
| | | CAPTAN 400-C | Corny Soybean |
| F | Fludioxonil | MAXIM 4FS | Corn, Soybean |
| F | Hydrogen peroxide | OXIDATE | Soybean |
| | | STOROX | Soybean |
| F | ipconazole | ACCELERON DC-509 | Corn |
| | | RANCONA 3.8 FS | Corn, Soybean |
| | | VORTEX | Corn |
| F | mancozeb | BONIDE MANCOZEB w/Zinc | Corn |
| | | Concentrate | |
| | | DITHANE 75DF RAINSHIELD | Corn |
| | | DITHANE DF RAINSHIELD | Corn |
| | | DITHANE F45 RAINSHIELD | Corn |
| | | DITHANE M45 | Corn |
| | | LESCO 4 FLOWABLE | Corn |
| | | MANCOZEB | |
| | | PENNCOZEB 4FL FLOWABLE | |
| | | PENNCOZEB 75DF DRY | Corn |
| | | FLOWABLE | Corn |
| | | PENNCOZEB 80WP | Corn |
| F | mefenoxam | APRON XL | Corn, Soybean |
| F | metalaxyl | ACCELERON DC-309 | Corn |
| | | ACCELERON DX-309 | Corn, Soybean |
| | | ACQUIRE | Corn, Soybean |
| | | AGRI STAR METALAXYL 265 | Corn, Soybean |
| | | ST | |
| | | ALLEGIANCE DRY | Corn, Soybean |
| | | ALLEGIANCE FL | Corn, Soybean |
| | | BELMONT 2.7 FS | Corn, Soybean |
| | | DYNA-SHIELD METALAXYL | Corn, Soybean |
| | | SEBRING 2.65 ST | Corn, Soybean |
| | | SEBRING 318 FS | Corn, Soybean |
| | | SEBRING 480 FS | Corn, Soybean |
| | | VIREO MEC | Soybean |
| F | pyraclostrobin | ACCELERON DX-109 | Soybean |
| | | STAMINA | Corn |
| F | Streptomyces | MYCOSTOP | Corn, Soybean |
| | griseoviridis | | |
| F | Streptomyces lydicus | ACTINOGROW ST | Corn, Soybean |
| F | tebuconazole | AMTIDE TEBU 3.6F | Corn |
| | | SATIVA 309 FS | Corn |
| | | SATIVA 318 FS | Corn |
| | | TEBUSHA 3.6FL | Corn |
| | | TEBUZOL 3.6F | Corn |
| F | thiabendazole | MERTECT 340-F | Soybean |
| F | thiram | 42-S THIRAM | Corn, Soybean |
| | | FLOWSAN | Corn, Soybean |
| | | SIGNET 480 FS | Corn, Soybean |
| F | Trichoderma | T-22 HC | Corn, Soybean |
| | harzianum Rifai | | |
| F | trifloxystrobin | ACCELERON DX-709 | Corn |
| | | TRILEX FLOWABLE | Corn, soybean |
| I | chlorpyrifos | LORSBAN 50W in water soluble | Corn |
| | | packets | |
| I | clothianidin | ACCELERON IC-609 | Corn |
| | | NIPSIT INSIDE | Corn, Soybean |
| | | PONCHO 600 | Corn |
| I | imidacloprid | ACCELERON IX-409 | Corn |
| | | AGRI STAR MACHO 600 ST | Corn, Soybean |
| | | AGRISOLUTIONS NITRO | Corn, Soybean |
| | | SHIELD | |
| | | ATTENDANT 600 | Corn, Soybean |
| | | AXCESS | Corn, Soybean |
| | | COURAZE 2F | Soybean |
| | | DYNA-SHIELD | Corn, Soybean |
| | | IMIDACLOPRID 5 | |
| | | GAUCHO 480 FLOWABLE | Corn, Soybean |
| | | GAUCHO 600 FLOWABLE | Corn, Soybean |
| | | GAUCHO SB FLOWABLE | Corn, Soybean |
| | | NUPRID 4.6F PRO | Soybean |
| | | SENATOR 600 FS | Corn, Soybean |
| I | thiamethoxam | CRUISER 5FS | Corn, Soybean |
| N | abamectin | AVICTA 500 FS | Corn, Soybean |
| N | Bacillus firmus | VOTIVO FS | Soybean |
| P | cytokinin | SOIL X-CYTO | Soybean |
| | | X-CYTE | Soybean |
| P | harpin alpha beta | ACCELERON HX-209 | Corn, Soybean |
| | protein | N-HIBIT GOLD CST | Corn, Soybean |
| | | N-HIBIT HX-209 | Corn, Soybean |
| P | indole butyric acid | KICKSTAND PGR | Corn, Soybean |
| I, N | thiamethoxam, | AVICTA DUO CORN | Corn |
| | abamectin | AVICTA DUO 250 | |
| I, F | clothianidin, | PONCHO VOTIVO | Corn, Soybean |
| | Bacillus firmus | | |
| F, F | carboxin, captan | ENHANCE | Soybean |
| I, F | permethrin, carboxin | KERNEL GUARD SUPREME | Corn, Soybean |
| F, F | carboxin, thiram | VITAFLO 280 | Corn, Soybean |
| F, F | mefenoxam, fludioxonil | MAXIM XL | Corn, Soybean |
| | | WARDEN RTA | Soybean |
| | | APRON MAXX RFC | |
| | | APRON MAXX RTA + MOLY | |
| | | APRON MANX RTA | |
| I, F | imidacloprid, metalaxyl | AGRISOLUTIONS CONCUR | Corn |
| F, F | metalaxyl, ipconazole | RANCONA SUMMIT | Soybean |
| | | RANCONA XXTRA | |
| F, F | thiram, metalaxyl | PROTECTOR-L-ALLEGIANCE | Soybean |
| F, F | trifloxystrobin, | TRILEX AL | Soybean |
| | metalaxyl | TRILEX 2000 | |
| P, P, P | cytokinin, gibberellic | STIMULATE YIELD | Corn, Soybean |
| | acid, indole butyric acid | ENHANCER ASCEND | |
| F, F, I | mefenoxam, | CRUISERMAXX PLUS | Soybean |
| | fludioxonil, | | |
| | thiamethoxam | | |
| F, F, F | captan, carboxin, | BEAN GUARD/ALLEGIANCE | Soybean |
| | metalaxyl | | |
| F, F, I | captan, carboxin, | ENHANCE AW | Soybean |
| | imidacloprid | | |
| F, F, I | carboxin, | LATITUDE | Corn, Soybean |
| | metalaxyl, imidacloprid | | |
| F, F, F | metalaxyl, | STAMINA F3 HL | Corn |
| | pyraclostrobin, | | |
| | triticonazole | | |
| F, F, F, I | azoxystrobin, | CRUISER EXTREME | Corn |
| | fludioxonil, | | |
| | mefenoxam, | | |
| | thiamethoxam | | |
| F, F, F, F, F | azoxystrobin, | MAXIM QUATTRO | Corn |
| | fludioxonil, | | |
| | mefenoxam, | | |
| | thiabendazole | | |
| I | Chlorantraniliprole | LUMIVIA | Corn |

F = Fungicide; I = Insecticide; N = Nematicide; P = Plant Growth Regulator

### Application of Bacterial Populations on Crops

The composition of the bacteria or bacterial population described herein can be applied in furrow, in talc, or as seed treatment. The composition can be applied to a seed package in bulk, mini bulk, in a bag, or in talc.

The planter can plant the treated seed and grows the crop according to conventional ways, twin row, or ways that do not require tilling. The seeds can be distributed using a control hopper or an individual hopper. Seeds can also be distributed using pressurized air or manually. Seed placement can be performed using variable rate technologies. Additionally, application of the bacteria or bacterial population described herein may be applied using variable rate technologies. In some examples, the bacteria can be applied to seeds of corn, soybean, canola, sorghum, potato, rice, vegetables, cereals, pseudocereals, and oilseeds. Examples of cereals may include barley, fonio, oats, palmer's grass, rye, pearl millet, sorghum, spelt, teff, triticale, and wheat. Examples of pseudocereals may include breadnut, buckwheat, cattail, chia, flax, grain amaranth, hanza, quinoa, and sesame. In some examples, seeds can be genetically modified organisms (GMO), non-GMO, organic or conventional.

Additives such as micro-fertilizer, PGR, herbicide, insecticide, and fungicide can be used additionally to treat the crops. Examples of additives include crop protectants such as insecticides, nematicides, fungicide, enhancement agents such as colorants, polymers, pelleting, priming, and disinfectants, and other agents such as inoculant, PGR, softener, and micronutrients. PGRs can be natural or synthetic plant hormones that affect root growth, flowering, or stem elongation. PGRs can include auxins, gibberellins, cytokinins, ethylene, and abscisic acid (ABA).

The composition can be applied in furrow in combination with liquid fertilizer. In some examples, the liquid fertilizer may be held in tanks. NPK fertilizers contain macronutrients of sodium, phosphorous, and potassium.

The composition may improve plant traits, such as promoting plant growth, maintaining high chlorophyll content in leaves, increasing fruit or seed numbers, and increasing fruit or seed unit weight. Methods of the present disclosure may be employed to introduce or improve one or more of a variety of desirable traits. Examples of traits that may introduced or improved include: root biomass, root length, height, shoot length, leaf number, water use efficiency, overall biomass, yield, fruit size, grain size, photosynthesis rate, tolerance to drought, heat tolerance, salt tolerance, tolerance to low nitrogen stress, nitrogen use efficiency, resistance to nematode stress, resistance to a fungal pathogen, resistance to a bacterial pathogen, resistance to a viral pathogen, level of a metabolite, modulation in level of a metabolite, proteome expression. The desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the introduced and/or improved traits) grown under identical conditions. In some examples, the desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the introduced and/or improved traits) grown under similar conditions.

An agronomic trait to a host plant may include, but is not limited to, the following: altered oil content, altered protein content, altered seed carbohydrate composition, altered seed oil composition, and altered seed protein composition, chemical tolerance, cold tolerance, delayed senescence, disease resistance, drought tolerance, ear weight, growth improvement, health enhancement, heat tolerance, herbicide tolerance, herbivore resistance improved nitrogen fixation, improved nitrogen utilization, improved root architecture, improved water use efficiency, increased biomass, increased root length, increased seed weight, increased shoot length, increased yield, increased yield under water-limited conditions, kernel mass, kernel moisture content, metal tolerance, number of ears, number of kernels per ear, number of pods, nutrition enhancement, pathogen resistance, pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a metabolite, a detectable modulation in the level of a transcript, and a detectable modulation in the proteome, compared to an isoline plant grown from a seed without said seed treatment formulation.

In some cases, plants are inoculated with bacteria or bacterial populations that are isolated from the same species of plant as the plant element of the inoculated plant. For example, a bacteria or bacterial population that is normally found in one variety of Zea mays (corn) is associated with a plant element of a plant of another variety of Zea mays that in its natural state lacks said bacteria and bacterial populations. In one embodiment, the bacteria and bacterial populations is derived from a plant of a related species of plant as the plant element of the inoculated plant. For example, an bacteria and bacterial populations that is normally found in Zea diploperennis Iltis et al., (diploperennial teosinte) is applied to a Zea mays (corn), or vice versa. In some cases, plants are inoculated with bacteria and bacterial populations that are heterologous to the plant element of the inoculated plant. In one embodiment, the bacteria and bacterial populations is derived from a plant of another species. For example, a bacteria and bacterial populations that is normally found in dicots is applied to a monocot plant (e.g., inoculating corn with a soybean-derived bacteria and bacterial populations), or vice versa. In other cases, the bacteria and bacterial populations to be inoculated onto a plant is derived from a related species of the plant that is being inoculated. In one embodiment, the bacteria and bacterial populations is derived from a related taxon, for example, from a related species. The plant of another species can be an agricultural plant. In another embodiment, the bacteria and bacterial populations is part of a designed composition inoculated into any host plant element.

In some examples, the bacteria or bacterial population is exogenous wherein the bacteria and bacterial population is isolated from a different plant than the inoculated plant. For example, in one embodiment, the bacteria or bacterial population can be isolated from a different plant of the same species as the inoculated plant. In some cases, the bacteria or bacterial population can be isolated from a species related to the inoculated plant.

In some examples, the bacteria and bacterial populations described herein are capable of moving from one tissue type to another. For example, the present disclosure's detection and isolation of bacteria and bacterial populations within the mature tissues of plants after coating on the exterior of a seed demonstrates their ability to move from seed exterior into the vegetative tissues of a maturing plant. Therefore, in one embodiment, the population of bacteria and bacterial populations is capable of moving from the seed exterior into the vegetative tissues of a plant. In one embodiment, the bacteria and bacterial populations that is coated onto the seed of a plant is capable, upon germination of the seed into a vegetative state, of localizing to a different tissue of the plant. For example, bacteria and bacterial populations can be capable of localizing to any one of the tissues in the plant, including: the root, adventitious root, seminal 5 root, root hair, shoot, leaf, flower, bud, tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In one embodiment, the bacteria and bacterial populations is capable of localizing to the root and/or the root hair of the plant. In another embodiment, the bacteria and bacterial populations is capable of localizing to the photosynthetic tissues, for example, leaves and shoots of the plant. In other cases, the bacteria and bacterial populations is localized to the vascular tissues of the plant, for example, in the xylem and phloem. In still another embodiment, the bacteria and bacterial populations is capable of localizing to the reproductive tissues (flower, pollen, pistil, ovaries, stamen, fruit) of the plant. In another embodiment, the bacteria and bacterial populations is capable of localizing to the root, shoots, leaves and reproductive tissues of the plant. In still another embodiment, the bacteria and bacterial populations colonizes a fruit or seed tissue of the plant. In still another embodiment, the bacteria and bacterial populations is able to colonize the plant such that it is present in the surface of the plant (i.e., its presence is detectably present on the plant exterior, or the episphere of the plant). In still other embodiments, the bacteria and bacterial populations is capable of localizing to substantially all, or all, tissues of the plant. In certain embodiments, the bacteria and bacterial populations is not localized to the root of a plant. In other cases, the bacteria and bacterial populations is not localized to the photosynthetic tissues of the plant.

The effectiveness of the compositions can also be assessed by measuring the relative maturity of the crop or the crop heating unit (CHU). For example, the bacterial population can be applied to corn, and corn growth can be assessed according to the relative maturity of the corn kernel or the time at which the corn kernel is at maximum weight. The crop heating unit (CHU) can also be used to predict the maturation of the corn crop. The CHU determines the amount of heat accumulation by measuring the daily maximum temperatures on crop growth.

In examples, bacterial may localize to any one of the tissues in the plant, including: the root, adventitious root, seminal root, root hair, shoot, leaf, flower, bud tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In another embodiment, the bacteria or bacterial population is capable of localizing to the photosynthetic tissues, for example, leaves and shoots of the plant. In other cases, the bacteria and bacterial populations is localized to the vascular tissues of the plant, for example, in the xylem and phloem. In another embodiment, the bacteria or bacterial population is capable of localizing to reproductive tissues (flower, pollen, pistil, ovaries, stamen, or fruit) of the plant. In another embodiment, the bacteria and bacterial populations is capable of localizing to the root, shoots, leaves and reproductive tissues of the plant. In another embodiment, the bacteria or bacterial population colonizes a fruit or seed tissue of the plant. In still another embodiment, the bacteria or bacterial population is able to colonize the plant such that it is present in the surface of the plant. In another embodiment, the bacteria or bacterial population is capable of localizing to substantially all, or all, tissues of the plant. In certain embodiments, the bacteria or bacterial population is not localized to the root of a plant. In other cases, the bacteria and bacterial populations is not localized to the photosynthetic tissues of the plant.

The effectiveness of the bacterial compositions applied to crops can be assessed by measuring various features of crop growth including, but not limited to, planting rate, seeding vigor, root strength, drought tolerance, plant height, dry down, and test weight.

### Plant Species

The methods and bacteria described herein are suitable for any of a variety of plants, such as plants in the genera Hordeum, Oryza, Zea, and Triticeae. Other non-limiting examples of suitable plants include mosses, lichens, and algae. In some cases, the plants have economic, social and/or environmental value, such as food crops, fiber crops, oil crops, plants in the forestry or pulp and paper industries, feedstock for biofuel production and/or ornamental plants. In some examples, plants may be used to produce economically valuable products such as a grain, a flour, a starch, a syrup, a meal, an oil, a film, a packaging, a nutraceutical product, a pulp, an animal feed, a fish fodder, a bulk material for industrial chemicals, a cereal product, a processed human-food product, a sugar, an alcohol, and/or a protein. Non-limiting examples of crop plants include maize, rice, wheat, barley, sorghum, millet, oats, rye triticale, buckwheat, sweet corn, sugar cane, onions, tomatoes, strawberries, and asparagus. In some embodiments, the methods and bacteria described herein are suitable for any of a variety of transgenic plants, non-transgenic plants, and hybrid plants thereof.

In some examples, plants that may be obtained or improved using the methods and composition disclosed herein may include plants that are important or interesting for agriculture, horticulture, biomass for the production of biofuel molecules and other chemicals, and/or forestry. Some examples of these plants may include pineapple, banana, coconut, lily, grasspeas and grass; and dicotyledonous plants, such as, for example, peas, alfalfa, tomatillo, melon, chickpea, chicory, clover, kale, lentil, soybean, tobacco, potato, sweet potato, radish, cabbage, rape, apple trees, grape, cotton, sunflower, thale cress, canola, citrus (including orange, mandarin, kumquat, lemon, lime, grapefruit, tangerine, tangelo, citron, and pomelo), pepper, bean, lettuce, Panicum virgatum (switch), Sorghum bicolor (sorghum, sudan), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), Pennisetum glaucum (pearl millet), Panicum spp. Sorghum spp., Miscanthus spp., Saccharum spp., Erianthus spp., Populus spp., Secale cereale (rye), Salix spp. (willow), Eucalyptus spp. (eucalyptus), Triticosecale spp. (triticum-25 wheat X rye), Bamboo, Carthamus tinctorius (safflower), Jatropha curcas (Jatropha), Ricinus communis (castor), Elaeis guineensis (oil palm), Phoenix dactylfera (date palm), Archontophoenix cunninghamiana (king palm), Syagrus romanzoffiana (queen palm), Linum usitatissimum (flax), Brassica juncea, Manihot esculenta (cassaya), Lycopersicon esculentum (tomato), Lactuca saliva (lettuce), Musa paradisiaca (banana), Solanum tuberosum (potato), Brassica oleracea (broccoli, cauliflower, brussel sprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Papaver somniferum (opium poppy), Papaver orientale, Taxus baccata, Taxus brevifolia, Artemisia annua, Cannabis saliva, Camptotheca acuminate, Catharanthus roseus, Vinca rosea, Cinchona officinalis, Coichicum autumnale, Veratrum calfornica, Digitalis lanata, Digitalis purpurea, Dioscorea 5 spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana, Alstroemeria spp., Rosa spp. (rose), Dianthus caryophyllus (carnation), Petunia spp. (petunia), Poinsettia pulcherrima (poinsettia), Nicotiana tabacum (tobacco), Lupinus albus (lupin), Uniola paniculata (oats), Hordeum vulgare (barley), and Lolium spp. (rye).

In some examples, a monocotyledonous plant may be used. Monocotyledonous plants belong to the orders of the Alismatales, Arales, Arecales, Bromeliales, Commelinales, Cyclanthales, Cyperales, Eriocaulales, Hydrocharitales, Juncales, Lilliales, Najadales, Orchidales, Pandanales, Poales, Restionales, Triuridales, Typhales, and Zingiberales. Plants belonging to the class of the Gymnospermae are Cycadales, Ginkgoales, Gnetales, and Pinales. In some examples, the monocotyledonous plant can be selected from the group consisting of a maize, rice, wheat, barley, and sugarcane.

In some examples, a dicotyledonous plant may be used, including those belonging to the orders of the Aristochiales, Asterales, Batales, Campanulales, Capparales, Caryophyllales, Casuarinales, Celastrales, Cornales, Diapensales, Dilleniales, Dipsacales, Ebenales, Ericales, Eucomiales, Euphorbiales, Fabales, Fagales, Gentianales, Geraniales, Haloragales, Hamamelidales, Middles, Juglandales, Lamiales, Laurales, Lecythidales, Leitneriales, Magniolales, Malvales, Myricales, Myrtales, Nymphaeales, Papeverales, Piperales, Plantaginales, Plumb aginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Theales, Trochodendrales, Umbellales, Urticales, and Violates. In some examples, the dicotyledonous plant can be selected from the group consisting of cotton, soybean, pepper, and tomato.

In some cases, the plant to be improved is not readily amenable to experimental conditions. For example, a crop plant may take too long to grow enough to practically assess an improved trait serially over multiple iterations. Accordingly, a first plant from which bacteria are initially isolated, and/or the plurality of plants to which genetically manipulated bacteria are applied may be a model plant, such as a plant more amenable to evaluation under desired conditions. Non-limiting examples of model plants include Setaria, Brachypodium, and Arabidopsis. Ability of bacteria isolated according to a method of the disclosure using a model plant may then be applied to a plant of another type (e.g. a crop plant) to confirm conferral of the improved trait.

Traits that may be improved by the methods disclosed herein include any observable characteristic of the plant, including, for example, growth rate, height, weight, color, taste, smell, changes in the production of one or more compounds by the plant (including for example, metabolites, proteins, drugs, carbohydrates, oils, and any other compounds). Selecting plants based on genotypic information is also envisaged (for example, including the pattern of plant gene expression in response to the bacteria, or identifying the presence of genetic markers, such as those associated with increased nitrogen fixation). Plants may also be selected based on the absence, suppression or inhibition of a certain feature or trait (such as an undesirable feature or trait) as opposed to the presence of a certain feature or trait (such as a desirable feature or trait).

### Non-Genetically Modified Maize

The methods and bacteria described herein are suitable for any of a variety of non-genetically modified maize plants or part thereof. And in some aspects the corn is organic. Furthermore, the methods and bacteria described herein are suitable for any of the following non-genetically modified hybrids, varities, lineages, etc. In some embodiments, corn varieties generally fall under six categories: sweet corn, flint corn, popcorn, dent corn, pod corn, and flour corn.

### Sweet Corn

Yellow su varieties include Earlivee, Early Sunglow, Sundance, Early Golden Bantam, Iochief, Merit, Jubilee, and Golden Cross Bantam. White su varieties include True Platinum, Country Gentleman, Silver Queen, and Stowell's Evergreen. Bicolor su varieties include Sugar & Gold, Quickie, Double Standard, Butter & Sugar, Sugar Dots, Honey & Cream. Multicolor su varieties include Hookers, Triple Play, Painted Hill, Black Mexican/Aztec.

Yellow se varieties include Buttergold, Precocious, Spring Treat, Sugar Buns, Colorow, Kandy King, Bodacious R/M, Tuxedo, Incredible, Merlin, Miracle, and Kandy Korn EH. White se varieties include Spring Snow, Sugar Pearl, Whiteout, Cloud Nine, Alpine, Silver King, and Argent. Bicolor se varieties include Sugar Baby, Fleet, Bon Jour, Trinity, Bi-Licious, Temptation, Luscious, Ambrosia, Accord, Brocade, Lancelot, Precious Gem, Peaches and Cream Mid EH, and Delectable R/M. Multicolor se varieties include Ruby Queen.

Yellow sh2 varieties include Extra Early Super Sweet, Takeoff, Early Xtra Sweet, Raveline, Summer Sweet Yellow, Krispy King, Garrison, Illini Gold, Challenger, Passion, Excel, Jubilee SuperSweet, Illini Xtra Sweet, and Crisp 'N Sweet. White sh2 varieties include Summer Sweet White, Tahoe, Aspen, Treasure, How Sweet It Is, and Camelot. Bicolor sh2 varieties include Summer Sweet Bicolor, Radiance, Honey 'N Pearl, Aloha, Dazzle, Hudson, and Phenomenal.

Yellow sy varieties include Applause, Inferno, Honeytreat, and Honey Select. White sy varieties include Silver Duchess, Cinderella, Mattapoisett, Avalon, and Captivate. Bicolor sy varieties include Pay Dirt, Revelation, Renaissance, Charisma, Synergy, Montauk, Kristine, Serendipity/Providence, and Cameo.

Yellow augmented supersweet varieties include Xtra-Tender 1ddA, Xtra-Tender 11dd, Mirai 131Y, Mirai 130Y, Vision, and Mirai 002. White augmented supersweet varieties include Xtra-Tender 3dda, Xtra-Tender 31dd, Mirai 421W, XTH 3673, and Devotion. Bicolor augmented supersweet varieties include Xtra-Tender 2dda, Xtra-Tender 21dd, Kickoff XR, Mirai 308BC, Anthem XR, Mirai 336BC, Fantastic XR, Triumph, Mirai 301BC, Stellar, American Dream, Mirai 350BC, and Obsession.

### Flint Corn

Flint corn varieties include Bronze-Orange, Candy Red Flint, Floriani Red Flint, Glass Gem, Indian Ornamental (Rainbow), Mandan Red Flour, Painted Mountain, Petmecky, Cherokee White Flour,

### Popcorn

Popcorn varieties include Monarch Butterfly, Yellow Butterfly, Midnight Blue, Ruby Red, Mixed Baby Rice, Queen Mauve, Mushroom Flake, Japanese Hull-less, Strawberry, Blue Shaman, Miniature Colored, Miniature Pink, Pennsylvania Dutch Butter Flavor, and Red Strawberry.

### Dent Corn

Dent corn varieties include Bloody Butcher, Blue Clarage, Ohio Blue Clarage, Cherokee White Eagle, Hickory Cane, Hickory King, Jellicorse Twin, Kentucky Rainbow, Daymon Morgan's Knt. Butcher, Leaming, Leaming's Yellow, McCormack's Blue Giant, Neal Paymaster, Pungo Creek Butcher, Reid's Yellow Dent, Rotten Clarage, and Tennessee Red Cob.

In some embodiments, corn varieties include P1618W, P1306W, P1345, P1151, P1197, P0574, P0589, and P0157. W=white corn. In some embodiments, the methods and bacteria described herein are suitable for any hybrid of the maize varieties set forth herein.

### Genetically Modified Maize

The methods and bacteria described herein are suitable for any of a hybrid, variety, lineage, etc. of genetically modified maize plants or part thereof.

Furthermore, the methods and bacteria described herein are suitable for any of the following genetically modified maize events, which have been approved in one or more countries: 32138 (32138 SPT Maintainer), 3272 (ENOGEN), 3272×Bt11, 3272×bt11×GA21, 3272×Bt11×MIR604, 3272×Bt11×MIR604×GA21, 3272×Bt11×MIR604×TC1507×5307×GA21, 3272×GA21, 3272×MIR604, 3272×MIR604×GA21, 4114, 5307 (AGRISURE Duracade), 5307×GA21, 5307×MIR604×Btl1×TC1507×GA21 (AGRISURE Duracade 5122), 5307×MIR604×Bt11×TC1507×GA21×MIR162 (AGRISURE Duracade 5222), 59122 (HERCULEX RW), 59122×DAS40278, 59122×GA21, 59122×MIR604, 59122×MIR604×GA21, 59122×MIR604×TC1507, 59122×MIR604×TC1507×GA21, 59122×MON810, 59122×MON810×MIR604, 59122×MON810×NK603, 59122×MON810×NK603×MIR604, 59122×MON88017, 59122×MON88017×DAS40278, 59122×NK603 (Herculex RW ROUNDUP READY 2), 59122×NK603×MIR604, 59122×TC1507×GA21, 676, 678, 680, 3751 IR, 98140, 98140×59122, 98140×TC1507, 98140×TC1507×59122, Bt10 (Bt10), Bt11[X4334CBR, X4734CBR] (AGRISURE CB/LL), Btl1×5307, Btl1×5307×GA21, Btl1×59122×MIR604, Br11×59122×MIR604×GA21, Btl1×59122×MIR604×TC1507, M53, M56, DAS-59122-7, Bt11×59122×MIR604×TC1507×GA21, Bt11×59122×TC1507, TC1507×DAS-59122-7, Bt11×59122×TC1507×GA21, Bt11×GA21 (AGRISURE GT/CB/LL), Bt11×MIR162 (AGRISURE Viptera 2100), BT11×MIR162×5307, Bt11×MIR162×5307×GA21, Bt11×MIR162×GA21 (AGRISURE Viptera 3110), Btl1×MIR162×MIR604 (AGRISURE Viptera 3100), Bt11×MIR162×MIR604×5307, Bt11×MIR162×MIR604×5307×GA21, Bt11×MIR162×MIR604×GA21 (AGRISURE Viptera 3111/AGRISURE Viptera 4), Bt11, MIR162×MIR604×MON89034×5307×GA21, Bt11×MIR162×MIR604×TC1507, Bt11×MIR162×MIR604×TC1507×5307, Bt11×MIR162×MIR604×TC1507×GA21, Bt11×MIR162×MON89034, Bt11×MIR162×MON89034×GA21, Bt11×MIR162×TC1507, Bt11×MIR162×TC1507×5307, Bt11×MIR162×TC1507×5307×GA21, Bt11×MR162×TC1507×GA21 (AGRISURE Viptera 3220), BT11×MIR604 (Agrisure BC/LL/RW), Btl1×MIR604×5307, Bt11×MIR604×5307×GA21, Bt11×MIR604×GA21, Bt11×MIR604×TC1507, Bt11×MIR604×TC1507×5307, Bt11×MIR604×TC1507×GA21, Bt11×MON89034×GA21, Btl1×TC1507, Btl1×TC1507×5307, Btl1×TC1507×GA21, Bt176 [176] (NaturGard KnockOut/Maximizer), BVLA430101, CBH-351 (STARLINK Maize), DAS40278 (ENLIST Maize), DAS40278×NK603, DBT418 (Bt Xtra Maize), DLL25 [B16], GA21 (ROUNDUP READY Maize/AGRISURE GT), GA21×MON810 (ROUNDUP READY Yieldgard Maize), GA21×T25, HCEM485, LY038 (MAVERA Maize), LY038×MON810 (MAVERA Yieldgard Maize), MIR162 (AGRISURE Viptera), MIR162×5307, MIR162×5307×GA21, MIR162×GA21, MIR162×MIR604, MIR162×MIR604×5307, MIR162×MIR604×5307×GA21, MIR162×MIR604×GA21, MIR162×MIR604×TC1507×5307, MIR162×MIR604×TC1507×5307×GA21, MIR162×MIR604×TC1507×GA21, MIR162×MON89034, MIR162×NK603, MIR162×TC1507, MIR162×TC1507×5307, MIR162×TC1507×5307×GA21, MIR162×TC1507×GA21, MIR604 (AGRISURE RW), MIR604×5307, MIR604×5307×GA21, MIR604×GA21 (AGRISURE GT/RW), MIR604×NK603, MIR604×TC1507, MIR604×TC1507×5307, MIR604×TC1507×5307 xGA21, MIR604×TC1507×GA21, MON801 [MON80100], MON802, MON809, MON810 (YIELDGARD, MAIZEGARD), MON810×MIR162, MON810×MIR162×NK603, MON810×MIR604, MON810×MON88017 (YIELDGARD VT Triple), MON810×NK603×MIR604, MON832 (ROUNDUP READY Maize), MON863 (YIELDGARD Rootworm RW, MAXGARD), MON863×MON810 (YIELDGARD Plus), MON863×MON810×NK603 (YIELDGARD Plus with RR), MON863×NK603 (YIELDGARD RW+RR), MON87403, MON87411, MON87419, MON87427 (ROUNDUP READY Maize), MON87427×59122, MON87427×MON88017, MON87427×MON88017×59122, MON87427×MON89034, MON87427×MON89034×59122, MON87427×MON89034×MIR162×MON87411, MON87427×MON89034×MON88017, MON87427×MON89034×MON88017×59122, MON87427×MON89034×NK603, MON87427×MON89034×TC1507, MON87427×MON89034×TC1507×59122, MON87427×MON89034×TC1507×MON87411×59122, MON87427×MON89034×TC1507×MON87411×59122×DAS40278, MON87427×MON89034×TC1507×MON88017, MON87427×MON89034×MIR162×NK603, MON87427×MON89034×TC1507×MON88017×59122, MON87427×TC1507, MON87427×TC1507×59122, MON87427×TC1507×MON88017, MON87427×TC1507×MON88017×59122, MON87460 (GENUITY DROUGHTGARD), MON87460×MON88017, MON87460×MON89034×MON88017, MON87460×MON89034×NK603, MON87460×NK603, MON88017, MON88017×DAS40278, MON89034, MON89034×59122, MON89034×59122×DAS40278, MON89034×59122×MON88017, MON89034×59122×MON88017×DAS40278, MON89034×DAS40278, MON89034×MON87460, MON89034×MON88017 (GENUITY VT Triple Pro), MON89034×MON88017×DAS40278, MON89034×NK603 (GENUITY VT Double Pro), MON89034×NK603×DAS40278, MON89034×TC1507, MON89034×TC1507×59122, MON89034×TC1507×59122×DAS40278, MON89034×TC1507×DAS40278, MON89034×TC1507×MON88017, MON89034×TC1507×MON88017×59122 (GENUITY SMARTSTAX), MON89034×TC1507×MON88017×59122×DAS40278, MON89034×TC1507×MON88017×DAS40278, MON89034×TC1507×NK603 (POWER CORE), MON89034×TC1507×NK603×DAS40278, MON89034×TC1507×NK603×MIR162, MON89034×TC1507×NK603×MIR162×DAS40278, MON89034×GA21, MS3 (INVIGOR Maize), MS6 (INVIGOR Maize), MZHGOJG, MZIR098, NK603 (ROUNDUP READY 2 Maize), NK603×MON810×4114×MIR604, NK603×MON810 (YIELDGARD CB+RR), NK603×T25 (ROUNDUP READY LIBERTY LINK Maize), T14 (LIBERTY LINK Maize), T25 (LIBERTY LINK Maize), T25×MON810 (LIBERTY LINK YIELDGARD Maize), TC1507 (HERCULEX I, HERCULEX CB), TC1507×59122×MON810×MIR604×NK603 (OPTIMUM INTRASECT XTREME), TC1507×MON810×MIR604×NK603, TC1507×5307, TC1507×5307×GA21, TC1507×59122 (HERCULEX XTRA), TC1507×59122×DAS40278, TC1507×59122×MON810, TC1507×59122×MON810×MIR604, TC1507×59122×MON810×NK603 (OPTIMUM INTRASECT XTRA), TC1507×59122×MON88017, TC1507×59122×MON88017×DAS40278, TC1507×59122×NK603 (HERCULEX XTRA RR), TC1507×59122×NK603×MIR604, TC1507×DAS40278, TC1507×GA21, TC1507×MIR162×NK603, TC1507×MIR604×NK603 (OPTIMUM TRISECT), TC1507×MON810, TC1507×MON810×MIR162, TC1507×MON810×MIR162×NK603, TC1507×MON810×MIR604, TC1507×MON810×NK603 (OPTIMUM INTRASECT), TC1507×MON810×NK603×MIR604, TC1507×MON88017, TC1507×MON88017×DAS40278, TC1507×NK603 (HERCULEX I RR), TC1507×NK603×DAS40278, TC6275, and VCO-01981-5.

### Concentrations and Rates of Application of Agricultural Compositions

As aforementioned, the agricultural compositions of the present disclosure, which comprise a taught microbe, can be applied to plants in a multitude of ways. In two particular aspects, the disclosure contemplates an in-furrow treatment or a seed treatment

For seed treatment embodiments, the microbes of the disclosure can be present on the seed in a variety of concentrations. For example, the microbes can be found in a seed treatment at a cfu concentration, per seed of: 1×10¹, 1×10², 1×10³, 1×10⁴, 1×10⁵, 1×10⁶, 1×10⁷, 1×10⁸, 1×10⁹, 1×10¹⁰, or more. In particular aspects, the seed treatment compositions comprise about 1×10⁴ to about 1×10⁸ cfu per seed. In other particular aspects, the seed treatment compositions comprise about 1×10⁵ to about 1×10⁷ cfu per seed. In other aspects, the seed treatment compositions comprise about 1×10⁶ cfu per seed. In general, the one or more engineered gram-positive diazotrophic bacteria present in an agricultural or microbial composition provided herein can have an average colonization ability per unit of plant root tissue of at least about 1.0×10⁴ bacterial cells per gram of fresh weight of plant root tissue and can produce fixed N of at least about 1×10⁻¹⁷ mmol N per bacterial cell per hour.

In the United States, about 10% of corn acreage is planted at a seed density of above about 36,000 seeds per acre; ⅓ of the corn acreage is planted at a seed density of between about 33,000 to 36,000 seeds per acre; ⅓ of the corn acreage is planted at a seed density of between about 30,000 to 33,000 seeds per acre, and the remainder of the acreage is variable. See, “Corn Seeding Rate Considerations,” written by Steve Butzen, available at: www.pioneer.com/home/site/us/agronomy/library/corn-seeding-rate-considerations/

Table 4 below utilizes various cfu concentrations per seed in a contemplated seed treatment embodiment (rows across) and various seed acreage planting densities (1^{st} column: 15K-41K) to calculate the total amount of cfu per acre, which would be utilized in various agricultural scenarios (i.e. seed treatment concentration per seed×seed density planted per acre). Thus, if one were to utilize a seed treatment with 1×10⁶ cfu per seed and plant 30,000 seeds per acre, then the total cfu content per acre would be 3×10¹⁰ (i.e. 30K*1×10⁶).

TABLE 4
Total CFU Per Acre Calculation for Seed Treatment Embodiments

| Corn Population | | | | | | | | |
|---|---|---|---|---|---|---|---|---|
| (i.e. seeds per | | | | | | | | |
| acre) | 1.00E+02 | 1.00E+03 | 1.00E+04 | 1.00E+05 | 1.00E+06 | 1.00E+07 | 1.00E+08 | 1.00E+09 |
| 15,000 | 1.50E+06 | 1.50E+07 | 1.50E+08 | 1.50E+09 | 1.50E+10 | 1.50E+11 | 1.50E+12 | 1.50E+13 |
| 16,000 | 1.60E+06 | 1.60E+07 | 1.60E+08 | 1.60E+09 | 1.60E+10 | 1.60E+11 | 1.60E+12 | 1.60E+13 |
| 17,000 | 1.70E+06 | 1.70E+07 | 1.70E+08 | 1.70E+09 | 1.70E+10 | 1.70E+11 | 1.70E+12 | 1.70E+13 |
| 18,000 | 1.80E+06 | 1.80E+07 | 1.80E+08 | 1.80E+09 | 1.80E+10 | 1.80E+11 | 1.80E+12 | 1.80E+13 |
| 19,000 | 1.90E+06 | 1.90E+07 | 1.90E+08 | 1.90E+09 | 1.90E+10 | 1.90E+11 | 1.90E+12 | 1.90E+13 |
| 20,000 | 2.00E+06 | 2.00E+07 | 2.00E+08 | 2.00E+09 | 2.00E+10 | 2.00E+11 | 2.00E+12 | 2.00E+13 |
| 21,000 | 2.10E+06 | 2.10E+07 | 2.10E+08 | 2.10E+09 | 2.10E+10 | 2.10E+11 | 2.10E+12 | 2.10E+13 |
| 22,000 | 2.20E+06 | 2.20E+07 | 2.20E+08 | 2.20E+09 | 2.20E+10 | 2.20E+11 | 2.20E+12 | 2.20E+13 |
| 23,000 | 2.30E+06 | 2.30E+07 | 2.30E+08 | 2.30E+09 | 2.30E+10 | 2.30E+11 | 2.30E+12 | 2.30E+13 |
| 24,000 | 2.40E+06 | 2.40E+07 | 2.40E+08 | 2.40E+09 | 2.40E+10 | 2.40E+11 | 2.40E+12 | 2.40E+13 |
| 25,000 | 2.50E+06 | 2.50E+07 | 2.50E+08 | 2.50E+09 | 2.50E+10 | 2.50E+11 | 2.50E+12 | 2.50E+13 |
| 26,000 | 2.60E+06 | 2.60E+07 | 2.60E+08 | 2.60E+09 | 2.60E+10 | 2.60E+11 | 2.60E+12 | 2.60E+13 |
| 27,000 | 2.70E+06 | 2.70E+07 | 2.70E+08 | 2.70E+09 | 2.70E+10 | 2.70E+11 | 2.70E+12 | 2.70E+13 |
| 28,000 | 2.80E+06 | 2.80E+07 | 2.80E+08 | 2.80E+09 | 2.80E+10 | 2.80E+11 | 2.80E+12 | 2.80E+13 |
| 29,000 | 2.90E+06 | 2.90E+07 | 2.90E+08 | 2.90E+09 | 2.90E+10 | 2.90E+11 | 2.90E+12 | 2.90E+13 |
| 30,000 | 3.00E+06 | 3.00E+07 | 3.00E+08 | 3.00E+09 | 3.00E+10 | 3.00E+11 | 3.00E+12 | 3.00E+13 |
| 31,000 | 3.10E+06 | 3.10E+07 | 3.10E+08 | 3.10E+09 | 3.10E+10 | 3.10E+11 | 3.10E+12 | 3.10E+13 |
| 32,000 | 3.20E+06 | 3.20E+07 | 3.20E+08 | 3.20E+09 | 3.20E+10 | 3.20E+11 | 3.20E+12 | 3.20E+13 |
| 33,000 | 3.30E+06 | 3.30E+07 | 3.30E+08 | 3.30E+09 | 3.30E+10 | 3.30E+11 | 3.30E+12 | 3.30E+13 |
| 34,000 | 3.40E+06 | 3.40E+07 | 3.40E+08 | 3.40E+09 | 3.40E+10 | 3.40E+11 | 3.40E+12 | 3.40E+13 |
| 35,000 | 3.50E+06 | 3.50E+07 | 3.50E+08 | 3.50E+09 | 3.50E+10 | 3.50E+11 | 3.50E+12 | 3.50E+13 |
| 36,000 | 3.60E+06 | 3.60E+07 | 3.60E+08 | 3.60E+09 | 3.60E+10 | 3.60E+11 | 3.60E+12 | 3.60E+13 |
| 37,000 | 3.70E+06 | 3.70E+07 | 3.70E+08 | 3.70E+09 | 3.70E+10 | 3.70E+11 | 3.70E+12 | 3.70E+13 |
| 38,000 | 3.80E+06 | 3.80E+07 | 3.80E+08 | 3.80E+09 | 3.80E+10 | 3.80E+11 | 3.80E+12 | 3.80E+13 |
| 39,000 | 3.90E+06 | 3.90E+07 | 3.90E+08 | 3.90E+09 | 3.90E+10 | 3.90E+11 | 3.90E+12 | 3.90E+13 |
| 40,000 | 4.00E+06 | 4.00E+07 | 4.00E+08 | 4.00E+09 | 4.00E+10 | 4.00E+11 | 4.00E+12 | 4.00E+13 |
| 41,000 | 4.10E+06 | 4.10E+07 | 4.10E+08 | 4.10E+09 | 4.10E+10 | 4.10E+11 | 4.10E+12 | 4.10E+13 |

For in-furrow embodiments, the microbes of the disclosure can be applied at a cfu concentration per acre of: 1×10⁶, 3.20×10¹⁰, 1.60×10¹¹, 3.20×10¹¹, 8.0×10¹¹, 1.6×10¹², 3.20×10¹², or more. Therefore, in aspects, the liquid in-furrow compositions can be applied at a concentration of between about 1×10⁶ to about 3×10¹² cfu per acre.

In some aspects, the in-furrow compositions are contained in a liquid formulation. In the liquid in-furrow embodiments, the microbes can be present at a cfu concentration per milliliter of: 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, or more. In certain aspects, the liquid in-furrow compositions comprise microbes at a concentration of about 1×10⁶ to about 1×10¹¹ cfu per milliliter. In other aspects, the liquid in-furrow compositions comprise microbes at a concentration of about 1×10⁷ to about 1×10¹⁰ cfu per milliliter. In other aspects, the liquid in-furrow compositions comprise microbes at a concentration of about 1×10⁸ to about 1×10⁹ cfu per milliliter. In other aspects, the liquid in-furrow compositions comprise microbes at a concentration of up to about 1×10¹³ cfu per milliliter.

### Transcriptomic Profiling of Candidate Microbes

Transcriptomic profiling of a gram-positive diazotrophic microbe (e.g., Paenibacillus polymyxa strain CI41) can be performed in order to identify promoters that are active in the presence of environmental nitrogen. Said identified promoters can serve as promoters for potential use in altering the promoter of the mf operon of said gram-positive diazotrophic microorganism in order to facilitate expression of the mf operon in the presence of environmental fixed nitrogen as described herein. The transcriptomic profiling can entail culturing the gram-positive diazotrophic microbe (e.g., Paenibacillus polymyxa strain CI41) in a defined, nitrogen-free media supplemented with glutamine (e.g., 10 mM glutamine). Total RNA can then be extracted from these cultures (QIAGEN RNeasy kit) and subjected to RNAseq sequencing (e.g., via Illumina HiSeq (SeqMatic, Fremont CA)). Sequencing reads can then be mapped to the gram-positive diazotrophic microbe (e.g., CI41) host cell's genome data (e.g., using Geneious), and highly expressed genes under control of proximal transcriptional promoters can be identified. In one embodiment, transcriptomic profiling of Paenibacillus polymyxa strain CI41 in the presence of 10 mM glutamine identified a number of candidate promoters (see FIG. 9) for use in altering the nifB promoter to confer expression of the mf operon irrespective of the levels of environmental fixed nitrogen.

### EXAMPLES

The following examples are given for the purpose of illustrating various embodiments of the disclosure and are not meant to limit the present disclosure in any fashion. Changes therein and other uses which are encompassed within the spirit of the disclosure, as defined by the scope of the claims, will be recognized by those skilled in the art.

### Example 1: Complete Ammonium De-Repression in Paenibacillus sp. Enabled by GlnR Engineering

Paenibacilli are gram-positive diazotrophs that can fix nitrogen through nitrogenase whose activity is under the tight control of ammonium. These strains stop fixing nitrogen in the presence of available nitrogen. In Paenibacilli, GlnR works as a master regulator with dual function for the nitrogen fixation pathway. GlnR activates nif gene expression at low or no fixed nitrogen and represses nif gene expression at high fixed nitrogen via the interaction of the glutamine synthetase GlnA that senses high glutamine levels. The nifB promoter that regulates expression of the core nif cluster composed of nifBHDKENX-hesA-nifU has two GlnR-binding operator sites. Under ammonium depletion, GlnR binds upstream of the promoter, recruits RNA polymerase and activates transcription of the nif cluster, whereas under ammonium excess GlnR binds downstream of the promoter and inhibits transcription by impeding the binding and progression of RNA polymerase (see FIG. 1).

Previous efforts to remove ammonium repression of nitrogenase activity in Paenibacilli have seen limited success. Deleting glnR and/or glnA led to ammonium derepression but their nitrogenase activities decreased 6 to 30-fold compared to that of the wild type even in the absence of ammonium. In addition, removing the GlnR binding sites in the nifB promoter did not completely remove ammonium derepression from the previous work (Wang, Tianshu, et al. PLoS genetics 14.9 (2018): e1007629).

In this Example, a strategy to isolate GlnR mutant microbes that continue to fix nitrogen in the presence of ammonium without compromising nitrogenase activity was developed and tested. Further, residues in GlnR, a master regulator of nitrogen pathways, were identified via protein engineering and high-throughput screening.

### Methods/Results

### Bacterial Strains and Growth Media

E. coli DH10-beta (New England Biolabs) was used for cloning. For rich media, LB medium were used for E. coli and BHI medium was used for Paenibacillus. For minimal media, Paenibacillus minimal medium (10.4 g/L Na₂HPO₄, 3.4 g/L KH₂PO₄, 4 g/L glucose, 26 mg/L CaCl₂·2H₂O, 30 mg/L MgSO₄, 3 mg/L MnSO₄, 7.6 mg/L Na₂MoO₄·2H₂O, 18 mg/L Fe-citrate) was used for Paenibacillus. Antibiotics were used at the following concentrations: kanamycin, 30 μg/ml; ampicillin, 100 μg/ml; chloramphenicol, 5 μg/ml; tetracycline, 0.2 μg/ml; erythromycin, 1 μg/ml; polymyxin B, 40 μg/ml.

### Development of a High-Throughput Screening System for GlnR Mutants for Ammonium Resistance

Unlike in Bacillus in which TnrA and GlnR oppositely regulate transcription of the nitrogen pathway by nitrogen availability, Paenibacillus species lack TnrA. GlnR senses nitrogen levels through GlnA and solely controls transcription of the nif genes by nitrogen availability. To identify GlnR mutants that can induce the nif expression in the presence of ammonium, a high-throughput system was developed that allows for screening large mutant libraries of GlnR with respect to their ability to activate the nif genes in the presence of ammonium.

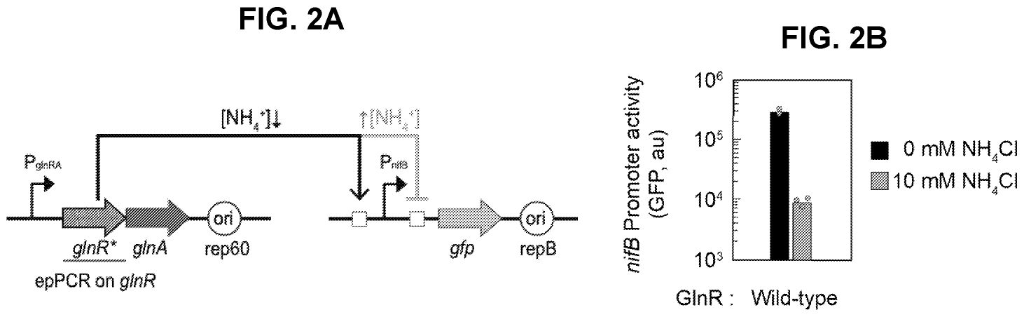

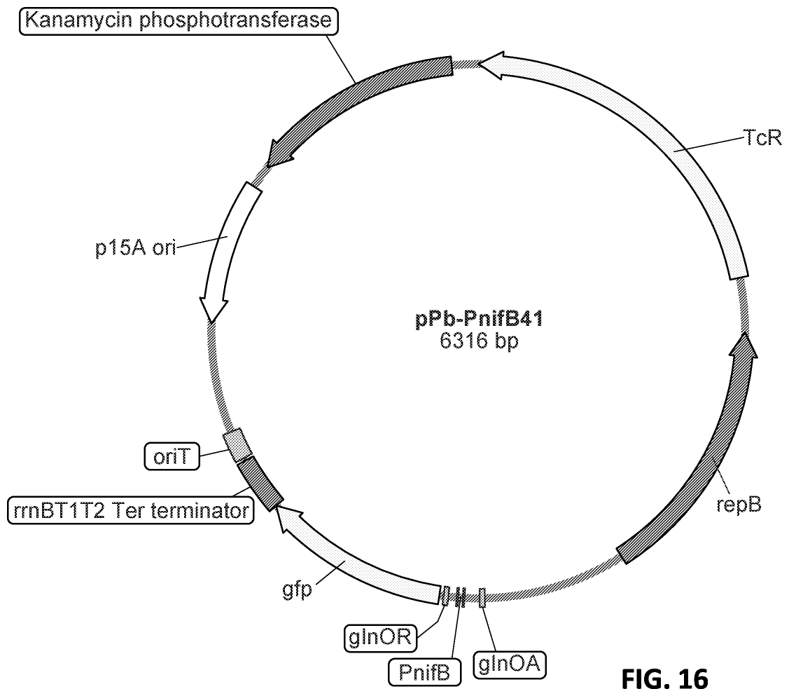

To construct the high-throughput screening system, the genomic copy of glnR was deleted from a strain of Paenibacillus. Moreover, a reporter plasmid based on a repB origin in which a fluorescence reporter (i.e., GFP) is operably linked to the nifB promoter was generated (see FIG. 16) and then introduced into this strain. More specifically, the reporter plasmid was constructed by amplifying the nifB promoter from genomic DNA of Paenibacillus polymyxa CI41 and placed upstream of GFP in a plasmid based on a repB origin. The reporter plasmid also contained the RK2 origin of transfer (oriT) in order to enable conjugative transfer from E. coli to Paenibacillus. Triparental mating was then used to transfer DNA from E. coli to the Paenibacillus strain lacking glnR. An aliquot of 80 μl of late-log phase donor cells and 80 μl of late-log phase helper cells containing a helper plasmid that allowed conjugative delivery of the reporter plasmid in donor cells were mixed with 200 μl of late-log phase recipient Paenibacillus cells lacking glnR and washed with 200 μl of BHI medium. Mating was initiated by spotting 20 μl of the mixed cells on BHI plates and incubated at 30° C. for 16 hr. The mating mixtures were plated on BHI medium supplemented with polymyxin B to kill E. coli and appropriate antibiotics to select plasmid transfer. Additionally, the glnRA operon with its own promoter was cloned on a rep60 origin plasmid to complement glnR deletion (see FIG. 2A and FIG. 16). The rep60 origin plasmid also contained the RK2 origin of transfer (oriT) (see FIG. 16; nucleic acid sequence of SEQ ID NO: 20) and were introduced into the Paenibacillus cells lacking glnR in the manner described for the reporter plasmid.

Subsequently, the ability of the system to recapitulate the native regulation of the nifB promoter with GlnR complementation was tested by introducing wild-type glnR on a rep60-origin plasmid. The wild-type glnR on a rep60-origin plasmid also contained the RK2 origin of transfer (oriT) and was introduced into the Paenibacillus cells lacking glnR in the manner described for the reporter plasmid.

To test control of the nif cluster by the mutated glnR in response to ammonium, the nifB promoter activity was analyzed using flow cytometry. Single colonies were inoculated into 0.5 ml BHI medium supplemented with antibiotics in 96-deep-well plates and incubated overnight at 30° C. and 900 r.p.m. Aliquots (1 μl) of the overnight cultures were diluted in 100 μl Paenibacillus minimal medium containing antibiotics in 96-well plates, and incubated for 15 hr at 30° C. and 800 r.p.m in the anaerobic chamber. Aliquots (8 l) of these cultures were then diluted in 150 μl PBS with 2 mg/ml kanamycin for flow cytometry analysis. Cultures with fluorescence proteins were analyzed by flow cytometry using an Attune NxT Flow Cytometer with a 488 nm laser and 510/20-nm band-pass filter for GFP. The cells were collected over 10,000 events, which were gated using forward and side scatter to remove background events using FlowJo (TreeStar Inc.). The median fluorescence from the cytometry histograms was calculated for all samples. The median autofluorescence was subtracted from the median fluorescence and reported as the fluorescence value in arbitrary units. As shown in FIG. 2B, the induction of the nifB promoters were abolished in the presence of 10 mM ammonium chloride when incubated anaerobically, indicating the system can be used to select GlnR mutants that do not repress the mf cluster in the presence of ammonium while maintaining their activity regardless of ammonium availability.

### Generation of GlnR Mutants for Use in High-Throughput Screening Assay for Ammonium Resistance

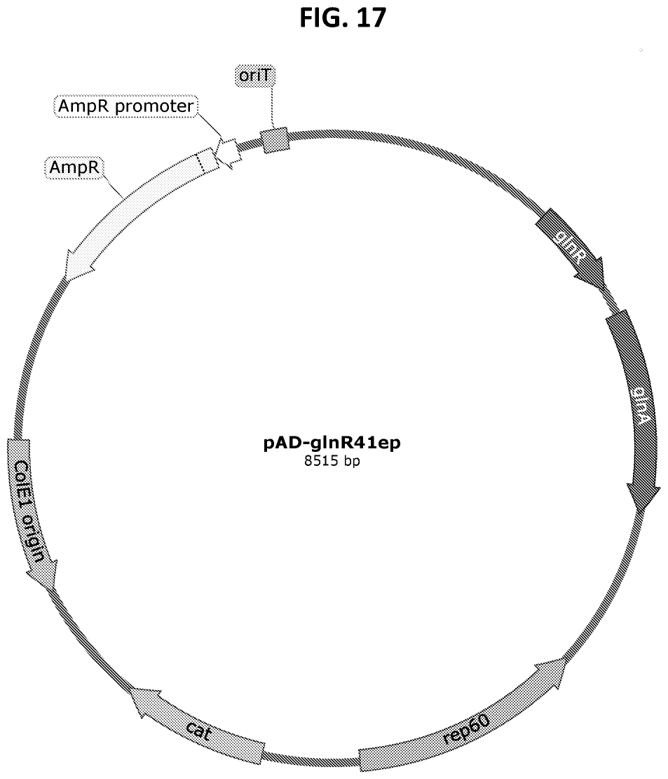

In order to generate potentially ammonium resistant glnR mutants, glnR was amplified from genomic DNA of Paenibacillus polymyxa CI41 by error-prone PCR and assembled with a plasmid based on a rep60 origin as shown FIG. 17 with the nucleic acid sequence of SEQ ID NO. 21. The error-prone PCR utilized PCR reactions with 1×PCR buffer supplemented with 7 mM MgSO₄, 0.4 mM MnSO₄, 1 mM dNTP and 0.05 U Go Taq DNA polymerase (Promega). Further, the plasmids the glnR mutants generated from the error-prone PCR were cloned into also contained the RK2 origin of transfer (oriT) to enable the conjugative transfer from E. coli to Paenibacillus. Like for the reporter plasmid, triparental mating was used to transfer DNA from E. coli to Paenibacillus. An aliquot of 80 μl of late-log phase donor cells and 80 μl of late-log phase helper cells containing a helper plasmid that allowed conjugative delivery of a glnR mutant containing plasmid in donor cells were mixed with 200 μl of late-log phase recipient Paenibacillus cells and washed with 200 μl of BHI medium. Mating was initiated by spotting 20 ml of the mixed cells on BHI plates and incubated at 30° C. for 16 hr. The mating mixtures were plated on BHI medium supplemented with polymyxin B to kill E. coli and appropriate antibiotics to select glnR mutant containing plasmid transfer.

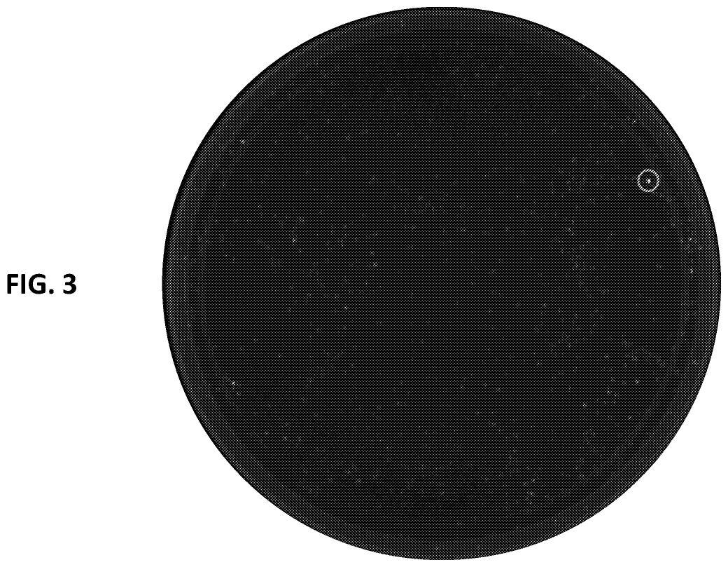

In order to test the ammonium resistance of the glnR mutants in the high-throughput screening assay, the reporter plasmid was introduced into Paenibacillus as described above, and donor cells containing glnR mutant libraries (library size of 10⁸ recombinants) were transferred and selected on Paenibacillus medium supplemented with 10 mM NH₄Cl and appropriate antibiotics. The plates were incubated at 30° C. for 5 days under anaerobic conditions. Derepression of the mf cluster was visualized by GFP expression and colonies showing induction of the nifB promoter arose with at a frequency of ˜10⁵ (see FIG. 3).

Isolation of GlnR Mutants that Fully Recovers Nitrogenase Activity in the Presence of Ammonium

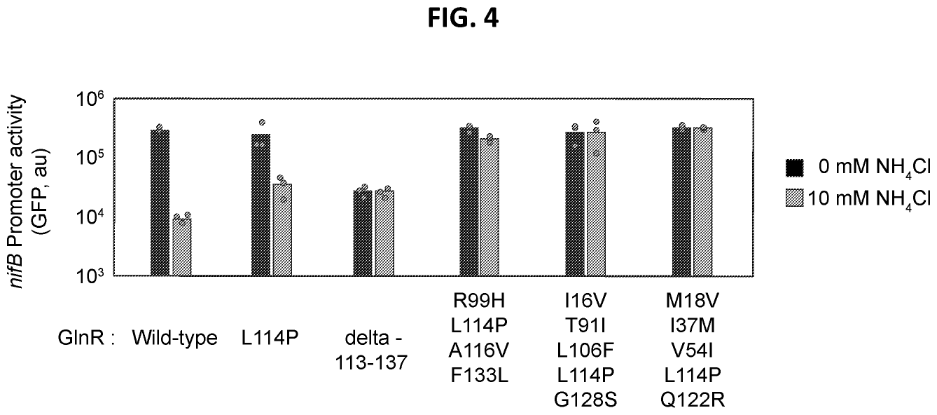

After isolation of the GFP expressing colonies, the nifB promoter induction with glnR mutants to the one with the wild-type GlnR were compared and a residue (L114P) in one of the GlnR mutants that enabled partial derepression of the nifB promoter activity in the presence of ammonium was identified using flow cytometry as described previously herein and shown in FIG. 4. The C-terminal domain (113-137) of GlnR was predicted to function as an ammonium-sensor by interacting with glutamine synthetase, GlnA, whose interaction with GlnR is regulated by glutamine levels. Thus, the glnR with C-terminal deletion (D113-137) was also tested in the GFP reporter assay described above to evaluate the extent to which the deletion affects ammonium repression. As shown in FIG. 4, this C-terminal deletion mutant yielded partial induction of the nif cluster but also lowered overall nitrogenase activity when tested in the nitrogenase assay described in this example, which is in agreement with the lowered nitrogenase activity as reported previously for this type of mutant (Wang, Tianshu, et al. PLoS genetics 14.9 (2018): e1007629).

In order to completely recover the expression of the nif cluster in the presence of ammonium, a second-round of mutagenesis was performed using error-prone PCR on the GlnR mutant L114P that showed partial ammonium derepression. Colonies were isolated as described above and tested for the nifB promoter activity in the presence and absence of ammonium via the GFP reporter assay described in this Example. Three of them resulted in full induction of the nifB promoter with or without the addition of 10 mM ammonium chloride, and additional SNPs were identified throughout the GlnR protein as shown in FIG. 4.

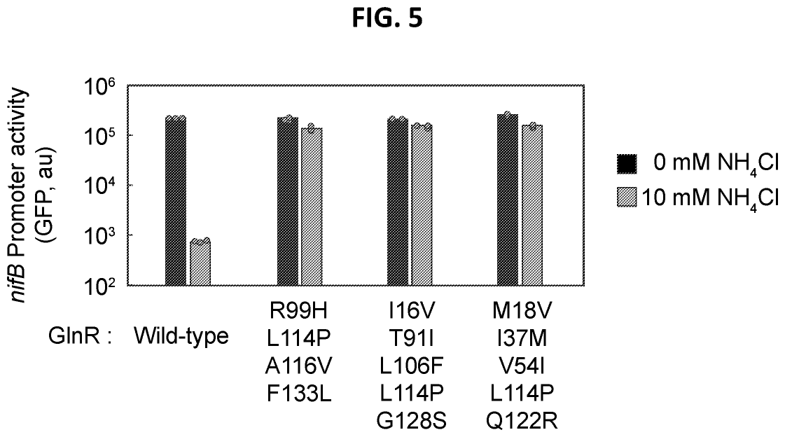

Once the glnR mutants from the screening system were isolated, the genomic glnR gene in the wild-type Paenibacillus was replaced with the mutant glnR. Ammonium derepression was then assessed by the reporter plasmid that encodes GFP driven by the nifB promoter in the Paenibacillus strain. The wild-type Paenibacillus showed 289-fold reduction in the nifB promoter activity by the addition of ammonium, while there was no repression of the promoter activity in the glnR mutants (see FIG. 5).

Finally, the strains with glnR mutations were grown and evaluated for nitrogenase activity using an acetylene reduction assay (ARA). In summary, the acetylene reduction assay was as follows: cultures were initiated by inoculating a single colony into 5 ml BHI in 15 ml culture tubes and grown overnight at 30° C. and 250 rpm. 1 ml of overnight cultures were diluted into 25 ml of Paenibacillus minimal medium supplemented with 10 mM glutamine in 125 ml flasks and incubated overnight at 30° C. and 250 rpm. Cultures were collected by centrifugation and resuspended in 5 ml of Paenibacillus minimal medium. 1 ml of resuspended culture was diluted into 4 ml of Paenibacillus minimal medium in hungate tubes with septa screw caps. Headspace in the vials was replaced with 95% nitrogen and 5% hydrogen in a COY chamber and acetylene freshly generated from CaC₂ in a Burris bottle was injected to 10% (vol/vol) into each culture tube to begin the reaction. The acetylene reduction was carried out for 16 hr at 30° C. Ethylene production was analyzed by gas chromatography equipped with an autosampler and flame ionization detector.

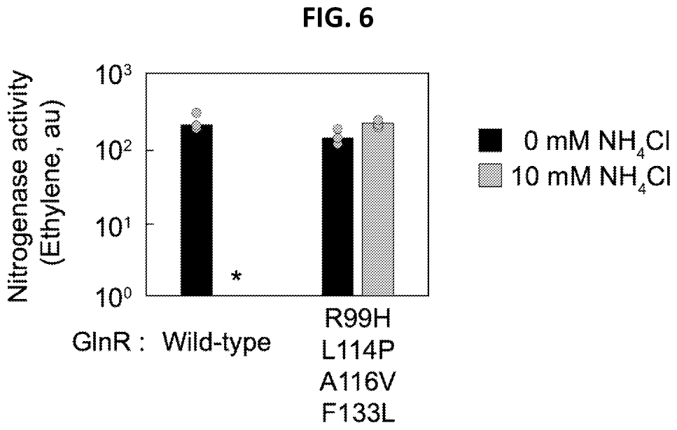

As previously reported, activity from the wild-type Paenibacillus was eliminated in the presence of ammonium. In contrast, the engineered Paenibacillus strain with a mutant glnR identified from the GFP screen described above fully recovered nitrogenase activity regardless of ammonium addition as shown in FIG. 6.

In summary, Gram-positive Paenibacilli that contain the nif cluster in their genome can fix nitrogen through nitrogenase whose activity is under the tight control of ammonium. In Paenibacilli, GlnR works as a master regulator for the nitrogen fixation pathway that activates nif gene expression at low fixed nitrogen and represses nif gene expression in the presence of ammonium. As described in this Example, a strategy was developed that can identify mutants in GlnR that lead to nitrogen fixation in the presence of ammonium. The results described in this Example revealed that multiple residues are required to fully recover the expression of the nif cluster as well as nitrogenase activity in the presence of ammonium.

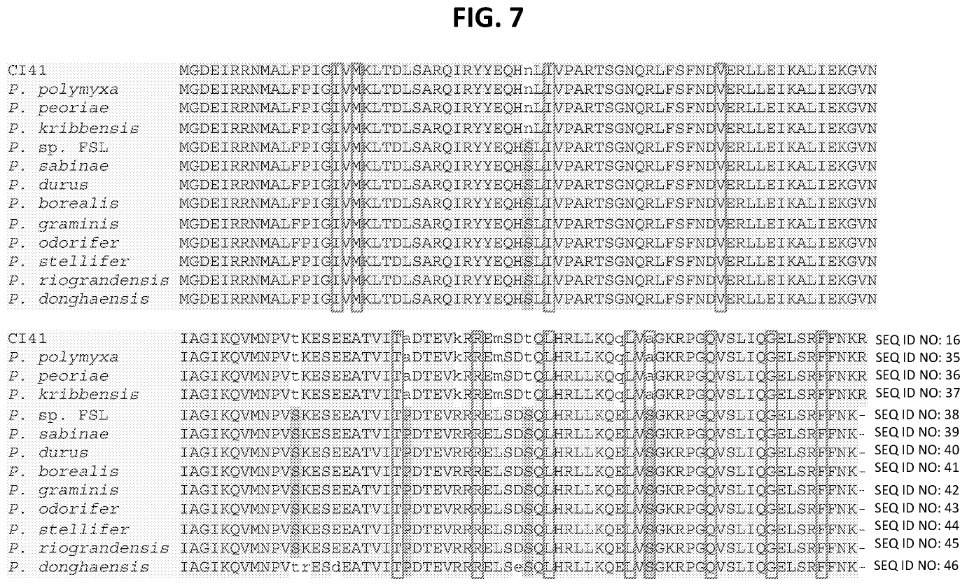

Interestingly, a sequence comparison of the nif cluster in all Paenibacillus species revealed that the corresponding residues for ammonium tolerance identified in this Example were found to be conserved across all Paenibacillus species that contain the nif cluster (see FIG. 7). Accordingly, the high-throughput screening system described in this Example is widely applicable for identifying mutations in a master regulator of nitrogen fixation that overcome ammonium repression in diverse Gram-positive species, and the glnR mutants identified here can be adapted to remove ammonium repression in nitrogen fixing Paenibacillus species.

### Example 2: Engineering nifB Promoter for Constitutive Expression Under High Levels of Fixed Nitrogen

In paenibacillus, the core genes essential for nitrogen fixation are clustered in a single operon comprising nifB, nifH, nifD, nifK, nifE, nifN, nifX, hesA and nifV genes, collectively known as the mf cluster. Expression of the mf cluster is controlled by a 670 promoter located upstream of nifB. This promoter contains multiple GlnR-interacting cis elements that regulate the transcription of the nif cluster in a nitrogen dependent manner. GlnR is a trans-acting regulatory protein with both activating and inhibiting roles for nitrogen metabolism in gram-positive bacteria. Under nitrogen-limited conditions, GlnR binds to the activation site located in the nifB promoter, 157 bps upstream of the start codon, allowing for transcription of the nif cluster. Upon the availability of external nitrogen, GlnR binds the repressor site located in the nifB promoter, 21 bps upstream of the start codon, leading to inhibition of the transcription.

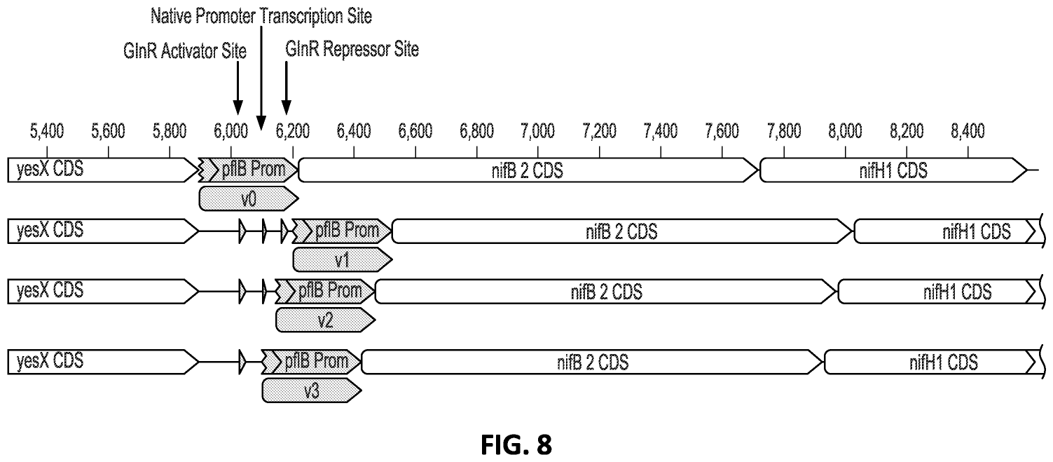

To overcome GlnR regulation and maintain the nif regulon being constitutively expressed independent of exogenous nitrogen levels, four types of modifications within the native promoter were designed and paired with strong endogenous promoters. A schematic of the native promoter showing the GlnR activator and repressor sites as well as the native promoter transcription site is shown in FIG. 8.

In summary, the four modifications were:

Modification V0: deletion of all the GlnR-interacting cis elements of the native promoter. For this modification, 13 strong constitutive native promoters of Paenibacillus polymyxa C141 as characterized via RNA-seq analysis were selected and inserted upstream of nifB gene resulting in the deletion of the 305 bp sequence upstream of the start codon of the nifB gene. The 13 promoters are shown in FIG. 9. More specifically, Paenibacillus polymyxa CI41 cultures were grown anaerobically in both ARA minimal media with no nitrogen source and ARA minimal media with 5 mM glutamine to characterize the expression levels of its genes. Total RNA was isolated from said cultures and subjected to RNA-seq analysis in order to ascertain expression levels of genes expressed in Paenibacillus when grown in nitrogen excess and limited environments. The expression levels of the Paenibacillus genes in the two conditions were ranked and 13 genes with consistent levels of expression in both conditions were characterized. The predicted promoter regions of these 13 genes were amplified using PCR and were subsequently introduced upstream of the nifB gene in a manner that deleted all of the GlnR-interacting cis elements of the native promoter as explained under “Strain Engineering” below.

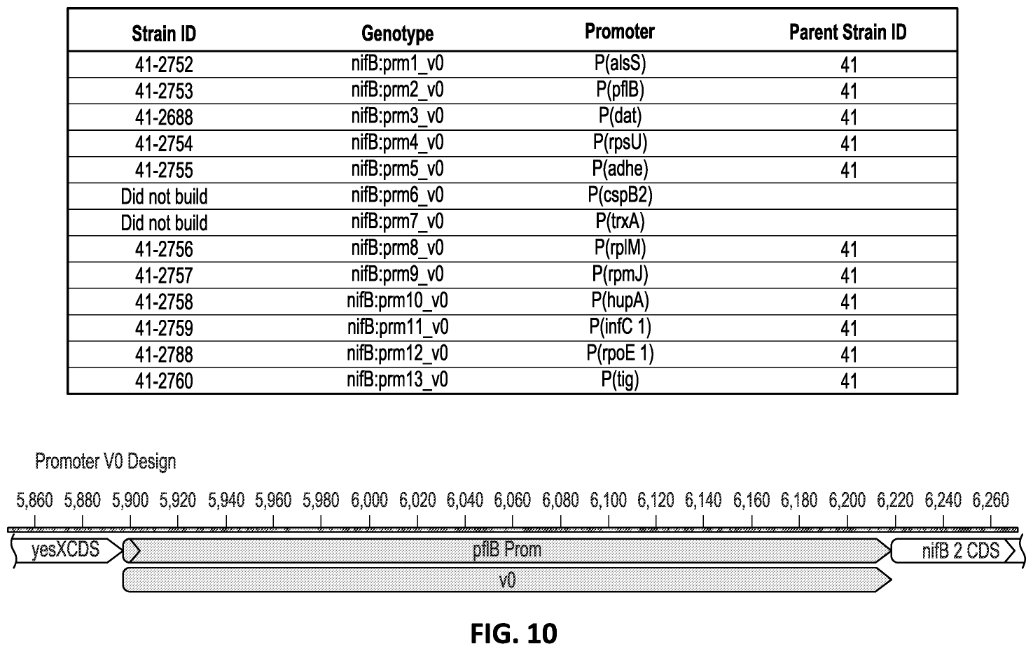

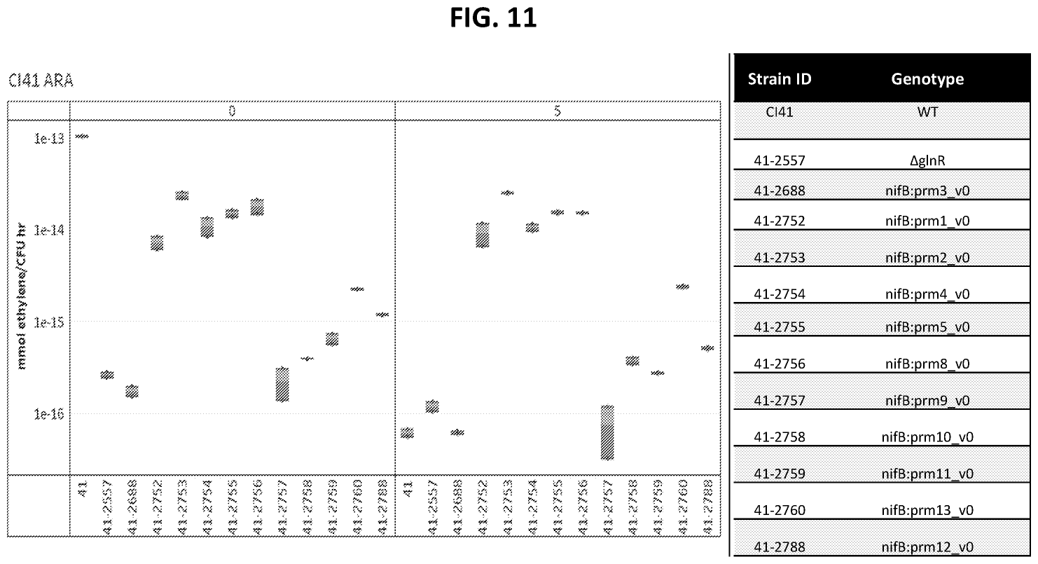

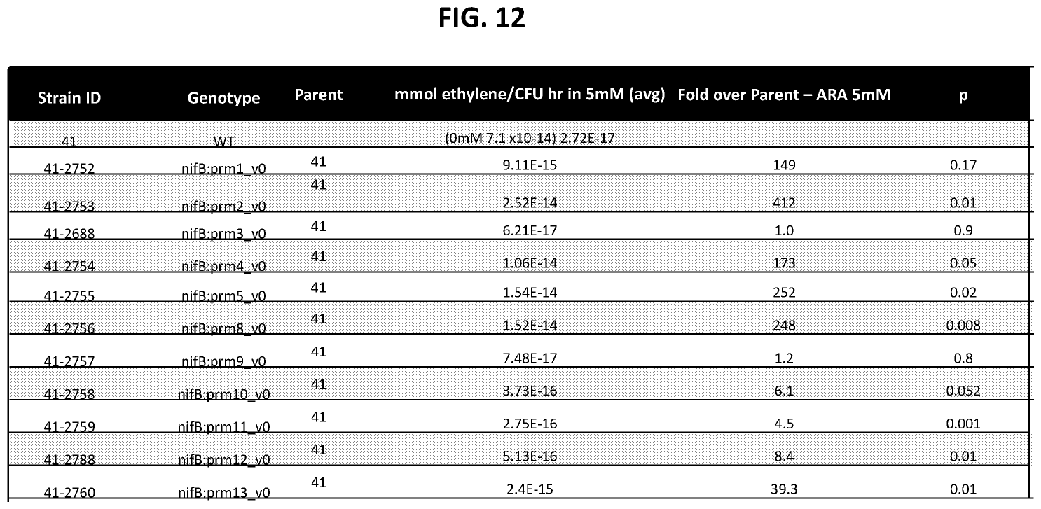

The strains built and tested for modification V0 are shown in FIG. 10. From this initial analysis of 13 promoters, insertions of promoters 1, 2, 4, 5, 8 and 13 upstream of nifB resulted in de-repression of nitrogen fixation (see FIGS. 11 and 12). As indicated in FIG. 10, V0 strains using the cold shock protein CspB promoter (i.e., cspB CDS prom; promoter strength 6 from FIG. 9) and Thioredoxin promoter (i.e., trxA CDS prom; promoter strength 7 from FIG. 9) were not built.

Modification V1: addition of constitutive promoter in front nifB gene with retention of GlnR-interacting cis elements. For this modification, three endogenous constitutive promoters, pflB, adhE and tig (promoters 2, 5, and 13, respectively in the attached slide deck) that showed highest derepression in the first design were inserted in front of the nitrogenase cluster (upstream of nifB gene). FIG. 8 shows an exemplary V1 modification using the pflB promoter.

Modification V2: deletion of the GlnR repressor binding site. For this modification, the 51 bp sequence upstream of the start codon of nifB gene was deleted and three endogenous constitutive promoters (i.e., pflB, adhE and tig from the second modification) were inserted in front of the nitrogenase cluster (upstream of nifB gene). Without the GlnR repressor binding site, GlnR should be unable to repress transcription under nitrogen excess conditions. FIG. 8 shows an exemplary V2 modification using the pflB promoter.

Modification V3: deletion of the GlnR repressor binding site and the native promoter transcription site. For this modification, the 100 bp sequence upstream of the start codon was deleted and three endogenous constitutive promoters (i.e., pflB, adhE and tig from the second modification) were inserted in front of the nitrogenase cluster (upstream of nifB gene). The deleted 100 bp includes the GlnR repressor binding site and the native promoter transcription site. Therefore, with this design, GlnR should be unable to repress the cluster under nitrogen excess conditions and the remaining native promoter sequence should be unable to initiate transcription so transcription is only initiated through the transcription start site of the introduced constitutive promoter. FIG. 8 shows an exemplary V3 modification using the pflB promoter.

### Plasmid Design and Strain Engineering

To construct the promoter insertions upstream (i.e., modifications V0-V3) of nifB, the integration vector, pKBT, was used containing a promoter of interest as described above (i.e., promoters 1-5 and 8-13 in FIG. 9) and homology arms. The promoter of interest and approximately 600 bp of DNA sequence homologous to upstream and downstream regions of the promoter insertion site from the CI41 Paenibacillus genome were amplified using high-fidelity polymerase, KOD. The promoter of interest was cloned between the up and down homology arms into the pKBT vector, using the Gibson DNA assembly protocol. Each assembled plasmid was transformed into E. coli strain St18, which was used for conjugation to the CI41 strain. pAD43-OriT-SceI was used to cut the vector, pKBT, and induce its loop-out from the Paenibacillus genome.

For rich media, SOB medium was used for E. coli and BHI medium was used for Paenibacillus. ARA minimal medium was used for Paenibacillus containing in a 10×Sugar buffer: 20×MoFe Solution, 500 mL; Di H20, 500 mL; Sucrose, 200 g; NaCl, 10 g; CaCl₂)×2H2O, 1 g; MgSO4×7H2O, 2.5 g; and in a 1×Salt Solution: Di H2O, 900 mL; Na2HlPO4, 25 g; KH₂PO4, 3 g; pH to 7.5 with HCl. Antibiotics were at the following concentrations: 100 mg/ml; Carbenicillin, 15 mg/ml; chloramphenicol, 3 mg/ml; erythromycin, 50 mg/ml 5-aminolevulinic acid.

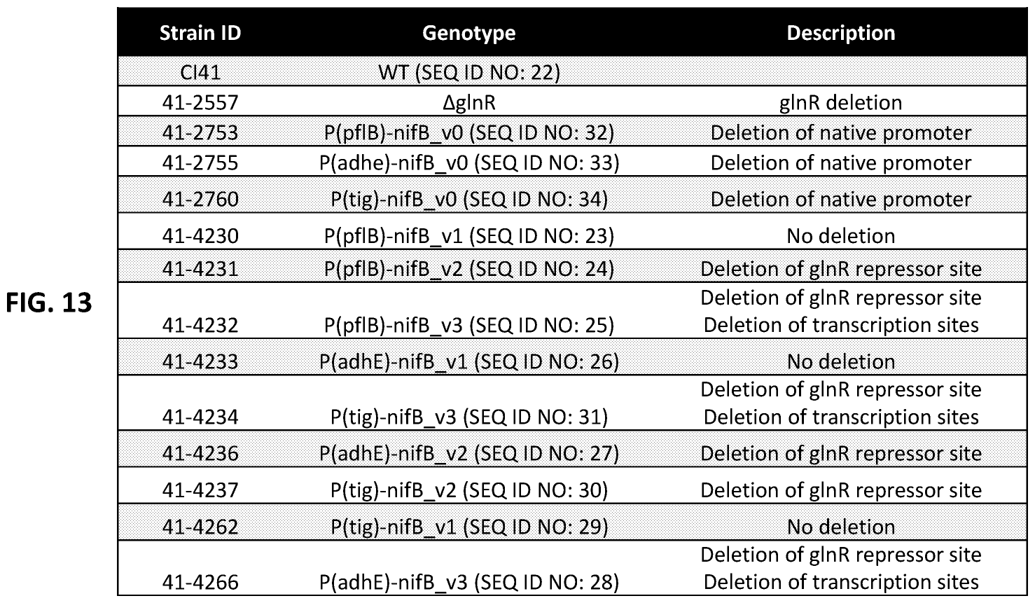

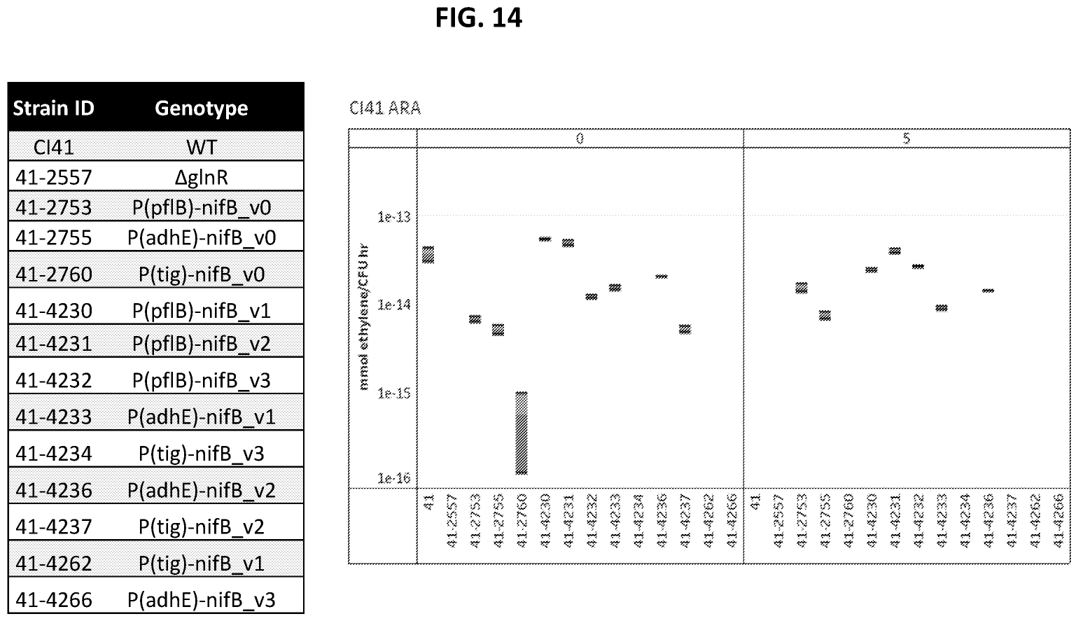

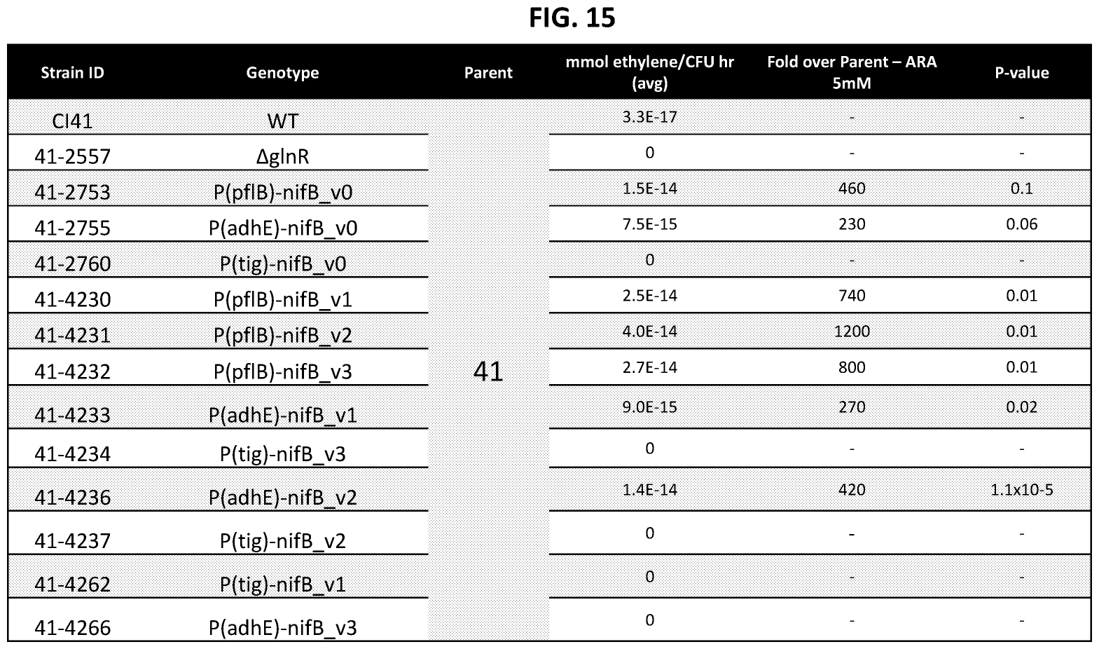

Strains comprising modifications V0-V3 as well as a wild-type CI41 strain and a strain lacking GlnR (delta GlnR) were subjected to the acetylene reduction assay (ARA) as described in Example 1. The strain ID, genotype (including the SEQ ID NO of the promoter-nifB gene present in each respective strain) and description of the strains are shown FIG. 13 and the results of the testing are shown in FIGS. 14 and 15. It should be noted that the Paenibacillus polymyxa CI41 nifB gene with its native promoter is denoted as WT in FIGS. 11-15 and has the nucleic acid sequence associated with SEQ ID NO: 22.

As shown in FIGS. 14 and 15, deletion of the repressor site and insertion of the pflB promoter provided the greatest level of de-repression, while designs using the tig promoter provided no de-repression.

### Example 3: Increased Ammonium Excretion in Paenibacillus sp. Enabled by GlnA Engineering

In this example, ammonium excretion was increased by mutagenizing glutamine synthetase (GS) glnA in Paenibacillus sp.

Mutants of Paenibacillus sp. strain CI41 were allowed to arise spontaneously. Three Paenibacillus CI41 mutants were selected for further study and ammonium excretion of the mutants was measured as follows. Cultures were initiated by inoculating a single colony into 0.4 ml BHI+1% sucrose in 96-deep-well plates and incubated overnight at 30° C. and 900 r.p.m. Aliquots (4 μl) of the overnight cultures were diluted in 200 μl Paenibacillus minimal medium with 10 mM NH₄Cl in 96-deep-well plates, and incubated for 24 h at 30° C. and 900 r.p.m. Aliquots (60 μl) of the cultures were diluted in 540 μl Paenibacillus minimal medium without a nitrogen source and transferred into an anaerobic chamber. The reaction was carried out for 48 h at 30° C. with shaking at 800 r.p.m. and ammonium excreted into the supernatant was analyzed by a Megazyme ammonia assay kit (Megazyme) according to the manufacturer's instructions.

Ammonium excretion was detected from the Paenibacillus CI41 mutants in which the GlnA protein in the glnRA operon was truncated through mutations resulting in either a premature stop codon or a frame shift (see Table 5), but was not detected in wild-type Paenibacillus CI41 with an intact glnA gene. This indicated that eliminating the GlnA protein expressed from the glnRA operon allowed the Paenibacillus mutants to fix nitrogen continuously and secrete ammonium.

The addition of ammonium analogues such as methylammonium inhibited the diazotrophic growth of Paenibacillus CI41 while producing toxic intermediate by the glutamine synthase (GS) activity. Mutations that arose spontaneously in the genomic regions that caused a decrease in GS activity (see Table 5) allowed the Paenibacillus CI41 mutants to survive in the presence of 25 mM methylammonium.

The mutants with low GS activity continuously fixed nitrogen in the presence of a high level of ammonium while keeping intracellular glutamine levels low likely because high levels of glutamine inhibit nitrogenase expression. Simultaneously, ammonium excretion likely increased in the mutants due to impaired ammonium assimilation.

TABLE 5
Ammonium excretion of the mutants from which GlnA
protein was disrupted through truncation.

| | Base | | |
|---|---|---|---|
| | substitution | Consequence | GAmmonium excretion (mM) |
| Strain | in glnA | of a mutation | (±, standard deviation) |
| 41-5877 | T insertion | Frameshift | 0.71 ± 0.07 |
| | between | | |
| | 283-284 bp | | |
| 41-5879 | C276A | Stop codon | 0.41 ± 0.02 |
| 41-5880 | G775T | Stop codon | 0.65 ± 0.04 |

### Numbered Embodiments of the Disclosure

Other subject matter contemplated by the present disclosure is set out in the following numbered embodiments:
1. An engineered gram-positive diazotrophic bacterium capable of fixing nitrogen irrespective of exogenous nitrogen levels at a rate at least equivalent to a rate of nitrogen fixation in a wild-type form of the gram-positive diazotrophic bacterium in the absence of exogenous nitrogen.
2. The engineered gram-positive diazotrophic bacterium of embodiment 1, comprising a heterologous promoter operably linked to a nif operon and/or a mutant glnR gene, wherein the heterologous promoter replaces at least a portion of the nif operon endogenous promoter and promotes expression of the mf operon irrespective of nitrogen levels, and wherein the mutant glnR gene encodes a mutant GlnR protein that promotes expression of the nif operon irrespective of nitrogen levels.
3. An engineered gram-positive diazotrophic bacterium comprising a heterologous promoter operably linked to a nif operon and/or a mutant glnR gene, wherein the heterologous promoter replaces at least a portion of the nif operon endogenous promoter and promotes expression of the nif operon irrespective of exogenous nitrogen levels, and wherein the mutant glnR gene encodes a mutant GlnR protein promotes expression of the nif operon irrespective of exogenous nitrogen levels.
4. The engineered gram-positive diazotrophic bacterium of embodiment 2 or 3, wherein the heterologous promoter completely replaces the nif operon endogenous promoter.
5. The engineered gram-positive diazotrophic bacterium of embodiment 2 or 3, wherein the heterologous promoter replaces a portion of the nif operon endogenous promoter downstream of a GlnR activator site, endogenous transcription start site and a GlnR repressor site.
6. The engineered gram-positive diazotrophic bacterium of embodiment 2 or 3, wherein the heterologous promoter replaces a portion of the nif operon endogenous promoter downstream of a GlnR activator site and endogenous transcription start site.
7. The engineered gram-positive diazotrophic bacterium of embodiment 2 or 3, wherein the heterologous promoter replaces a portion of the nif operon endogenous promoter downstream of a GlnR activator site.
8. The engineered gram-positive diazotrophic bacterium of any one of embodiments 2-7, wherein the heterologous promoter is selected from a promoter for a Paenibacillus Acetolactate synthase (alsS) gene, Pyruvate formate-lyase-activating enzyme (pflB) gene, D-alanine aminotransferase (dat) gene, 30S ribosomal protein S21 (rpsU) gene, Aldehyde-alcohol dehydrogenase (adhe) gene, 50S ribosomal protein L13 (rplm) gene, 50S ribosomal protein L36 (rpmJ) gene, DNA-binding protein HU 1 (hupA) gene, Translation initiation factor IF-3 (infC) gene, ECF RNA polymerase sigma-E factor (rpoE) gene, and Trigger factor (tig) gene.
9. The engineered gram-positive diazotrophic bacterium of any one of embodiments 2-8, wherein the heterologous promoter has a nucleic acid sequence selected from SEQ ID NOs: 1-11.
10. The engineered gram-positive diazotrophic bacterium of any one of the above embodiments, wherein the engineered gram-positive diazotrophic bacterium is selected from the group consisting of 41-2753, 41-2755, 41-4230, 41-4231, 41-4232, 41-4233 and 41-4236.
11. The engineered gram-positive diazotrophic bacterium of any one of embodiments 2-10, wherein the mutant glnR gene comprises at least one nucleotide substitution at nucleotide position 45, 46, 52, 111, 160, 272, 296, 316, 341, 347, 365, 382, 384 or 397 of a Paenibacillus glnR gene or at a homologous nucleotide position in a homolog thereof.
12. The engineered gram-positive diazotrophic bacterium of embodiment 11, wherein the mutant glnR gene shares at least 85%, 86%, 87%, 88%, 89%, 90%, 91%, 92%, 93%, 94%, 95%, 96%, 97%, 98%, or 99% identity with the Paenibacillus glnR gene or the homolog thereof.
13. The engineered gram-positive diazotrophic bacterium of any one of embodiments 2-12, wherein the mutant GlnR protein comprises at least one amino acid substitution of at amino acid position 16, 18, 37, 54, 91, 99, 106, 114, 116, 122, 128 or 133 of a Paenibacillus GlnR protein or at a homologous amino acid position in a homolog thereof.
14. The engineered gram-positive diazotrophic bacterium of any one of embodiments 2-12, wherein the mutant GlnR protein comprises at least one amino acid substitution selected from the group consisting of a I16V, M18V, I37M, V54I, T91I, R99H, L106F, L114P, A116V, Q122R, G128S and F133L of a Paenibacillus GlnR protein or at a homologous amino acid position in a homolog thereof.
15. The engineered gram-positive diazotrophic bacterium of any one of embodiments 2-14, wherein the mutant GlnR protein shares at least 90%, 91%, 92%, 93%, 94%, 95%, 96%, 97%, 98%, or 99% identity with the Paenibacillus GlnR protein or the homolog thereof.
16. The engineered gram-positive diazotrophic bacterium of any one of embodiments 2-15, wherein the mutant GlnR protein comprises an L to P mutation at position 114 of a Paenibacillus GlnR protein or at a homologous amino acid position in a homolog thereof.
17. The engineered gram-positive diazotrophic bacterium of any one of embodiments 2-16, wherein the mutant GlnR protein comprises a L114P mutation and one or more of a R99H mutation, an A116V mutation, a F133L mutation, an I16V mutation, a T91I mutation, a L106F mutation, a G128S mutation, a M18V mutation, an I37M mutation, a V54I mutation, a Q122R mutation and any combination thereof of a Paenibacillus GlnR protein or at a homologous amino acid position in a homolog thereof.
18. The engineered gram-positive diazotrophic bacterium of any one of embodiments 2-17, wherein the mutant GlnR protein comprises a L114P, a R99H mutation, an A116V mutation, and a F133L mutation of a Paenibacillus GlnR protein or at a homologous amino acid position in a homolog thereof.
19. The engineered gram-positive diazotrophic bacterium of any one of embodiments 2-17, wherein the mutant GlnR protein comprises a L114P, an I16V mutation, a T91I mutation, a L106F mutation, and a G128S mutation of a Paenibacillus GlnR protein or at a homologous amino acid position in a homolog thereof.
20. The engineered gram-positive diazotrophic bacterium of any one of embodiments 2-17, wherein the mutant GlnR protein comprises a L114P, a M18V mutation, an I37M mutation, a V54I mutation, and a Q122R mutation of a Paenibacillus GlnR protein or at a homologous amino acid position in a homolog thereof.
21. The engineered gram-positive diazotrophic bacterium of any one of embodiments 2-12, wherein the Paenibacillus glnR gene comprises a nucleic acid sequence of SEQ ID NO: 12.
22. The engineered gram-positive diazotrophic bacterium of any one of embodiments 2-21, wherein the mutant glnR gene comprises a nucleic acid sequence selected from the group consisting of SEQ ID NO: 13-15.
23. The engineered gram-positive diazotrophic bacterium of any one of embodiments 13-20, wherein the Paenibacillus GlnR protein comprises an amino acid sequence of SEQ ID NO: 16.
24. The engineered gram-positive diazotrophic bacterium of any one of embodiments 2-23, wherein the mutant GlnR protein comprises an amino acid selected from the group consisting of SEQ ID NO: 17-19.
25. The engineered gram-positive diazotrophic bacterium of any one of the above embodiments, further comprising a deletion of a glutamine synthetase A (glnA) gene.
26. The engineered gram-positive diazotrophic bacterium of any one of embodiments 1-24, further comprising a mutated form of a glutamine synthetase A (glnA) gene, wherein the mutated form of the glnA gene encodes a mutated GlnA protein that exhibits reduced assimilation of ammonium.
27. The engineered gram-positive diazotrophic bacterium of embodiment 26, wherein the mutated GlnA comprises at least one amino acid substitution at position 67, 182, 241 or 313 of a Paenibacillus GlnA or at a homologous amino acid position in a homolog thereof.
28. The engineered gram-positive diazotrophic bacterium of embodiment 26, wherein the mutated GlnA comprises at least one amino acid substitution selected from the group consisting of M67I, E182K, G241S and N313B of a Paenibacillus GlnA or at a homologous amino acid position in a homolog thereof.
29. The engineered gram-positive diazotrophic bacterium of any one of the above embodiments, further comprising at least one genetic variation introduced into a member selected from the group consisting of: nifB, nifH, nifD, nifK, nifE, nifN, nifX, hesA, nifV genes or combinations thereof that results in increased nitrogen fixation.
30. The engineered gram-positive diazotrophic bacterium of any one of the above embodiments, wherein said bacterium is a species from a genus selected from Paenibacillus, Bacillus and Lactobacillus.
31. The engineered gram-positive diazotrophic bacterium of any one of the above embodiments, wherein said bacterium is selected from Paenibacillus azotofixans, Paenibacillus borealis, Paenibacillus durus, Paenibacillus macerans, Paenibacillus polymyxa, Paenibacillus alvei, Paenibacillus amylolyticus, Paenibacillus campinasensis, Paenibacillus chibensis, Paenibacillus glucanolyticus, Paenibacillus illinoisensis, Paenibacillus larvae subsp. Larvae, Paenibacillus larvae subsp. Pulvifaciens, Paenibacillus lautus, Paenibacillus macerans, Paenibacillus macquariensis, Paenibacillus graminis, Paenibacillus pabuli, Paenibacillus peoriae, Paenibacillus stellifer, Paenibacillus riograndensis, Paenibacillus donghaensis, Paenibacillus sp. FSL, and Paenibacillus odorifier.
32. The engineered gram-positive diazotrophic bacterium of any one of the above embodiments, wherein said bacterium is a transgenic or a remodeled non-intergeneric bacterium.
33. The engineered gram-positive diazotrophic bacterium of any one of the above embodiments, wherein the wild-type form of the gram-positive diazotrophic bacterium is Paenibacillus polymyxa strain CI41 with deposit accession number PTA-126581.
34. A microbial composition comprising one or more bacteria, wherein the one or more bacteria are capable of fixing nitrogen irrespective of exogenous nitrogen levels at a rate at least equivalent to a rate of nitrogen fixation in a wild-type gram-positive diazotrophic bacterium in the absence of exogenous nitrogen.
35. The microbial composition of embodiment 34, wherein the one or more bacteria comprise one or more engineered gram-positive diazotrophic bacteria comprising a heterologous promoter operably linked to a nif operon and/or a mutant GlnR protein, wherein the heterologous promoter replaces at least a portion of the nif operon endogenous promoter and promotes expression of the nif operon irrespective of exogenous nitrogen levels, and wherein the mutant GlnR protein promotes expression of the nif operon irrespective of exogenous nitrogen levels.
36. The microbial composition of embodiment 35, wherein the heterologous promoter completely replaces the nif operon endogenous promoter.
37. The microbial composition of embodiment 35, wherein the heterologous promoter replaces a portion of the nif operon endogenous promoter downstream of a GlnR activator site, endogenous transcription start site and a GlnR repressor site.
38. The microbial composition of embodiment 35, wherein the heterologous promoter replaces a portion of the nif operon endogenous promoter downstream of a GlnR activator site and endogenous transcription start site.
39. The microbial composition of embodiment 35, wherein the heterologous promoter replaces a portion of the nif operon endogenous promoter downstream of a GlnR activator site.
40. The microbial composition of any one of embodiments 35-39, wherein the heterologous promoter is selected from a promoter for a Paenibacillus Acetolactate synthase (alsS) gene, Pyruvate formate-lyase-activating enzyme (pflB) gene, D-alanine aminotransferase (dat) gene, 30S ribosomal protein S21 (rpsU) gene, Aldehyde-alcohol dehydrogenase (adhe) gene, 50S ribosomal protein L13 (rplm) gene, 50S ribosomal protein L36 (rpmJ) gene, DNA-binding protein HU 1 (hupA) gene, Translation initiation factor IF-3 (infC) gene, ECF RNA polymerase sigma-E factor (rpoE) gene, and Trigger factor (tig) gene.
41. The microbial composition of any one of embodiments 35-40, wherein the heterologous promoter has a nucleic acid sequence selected from SEQ ID NOs: 1-11.
42. The microbial composition of any one of embodiments 35-41, wherein the one or more engineered gram-positive diazotrophic bacterium is selected from the group consisting of 41-2753, 41-2755, 41-4230, 41-4231, 41-4232, 41-4233 and 41-4236.
43. The microbial composition of any one of embodiments 35-42, wherein the mutant glnR gene comprises at least one nucleotide substitution at nucleotide position 45, 46, 52, 111, 160, 272, 296, 316, 341, 347, 365, 382, 384 or 397 of a Paenibacillus glnR gene or at a homologous nucleotide position in a homolog thereof.
44. The microbial composition of embodiment 43, wherein the mutant glnR gene shares at least 85%, 86%, 87%, 88%, 89%, 90%, 91%, 92%, 93%, 94%, 95%, 96%, 97%, 98%, or 99% identity with the Paenibacillus glnR gene or the homolog thereof.
45. The microbial composition of any one of embodiments 35-44, wherein the mutant GlnR protein comprises at least one amino acid substitution of at amino acid position 16, 18, 37, 54, 91, 99, 106, 114, 116, 122, 128 or 133 of a Paenibacillus GlnR protein or at a homologous amino acid position in a homolog thereof.
46. The microbial composition of any one of embodiments 35-44, wherein the mutant GlnR protein comprises at least one amino acid substitution selected from the group consisting of a I16V, M18V, I37M, V54I, T91I, R99H, L106F, L114P, A116V, Q122R, G128S and F133L of a Paenibacillus GlnR protein or at a homologous amino acid position in a homolog thereof.
47. The microbial composition of any one of embodiments 35-46, wherein the mutant GlnR protein shares at least 90%, 91%, 92%, 93%, 94%, 95%, 96%, 97%, 98%, or 99% identity with the Paenibacillus GlnR protein or the homolog thereof.
48. The microbial composition of any one of embodiments 35-47, wherein the mutant GlnR protein comprises an L to P mutation at position 114 of a Paenibacillus GlnR protein or at a homologous amino acid position in a homolog thereof.
49. The microbial composition of any one of embodiments 35-48, wherein the mutant GlnR protein comprises a L114P mutation and one or more of a R99H mutation, an A116V mutation, a F133L mutation, an I16V mutation, a T91I mutation, a L106F mutation, a G128S mutation, a M18V mutation, an I37M mutation, a V54I mutation, a Q122R mutation and any combination thereof of a Paenibacillus GlnR protein or at a homologous amino acid position in a homolog thereof.
50. The microbial composition of any one of embodiments 35-49, wherein the mutant GlnR protein comprises a L114P, a R99H mutation, an A116V mutation, and a F133L mutation of a Paenibacillus GlnR protein or at a homologous amino acid position in a homolog thereof.
51. The microbial composition of any one of embodiments 35-49, wherein the mutant GlnR protein comprises a L114P, an i16V mutation, a T91I mutation, a L106F mutation, and a G128S mutation of a Paenibacillus GlnR protein or at a homologous amino acid position in a homolog thereof.
52. The microbial composition of any one of embodiments 35-49, wherein the mutant GlnR protein comprises a L114P, a M18V mutation, an I37M mutation, a V54I mutation, and a Q122R mutation of a Paenibacillus GlnR protein or at a homologous amino acid position in a homolog thereof.
53. The microbial composition of any one of embodiments 35-44, wherein the Paenibacillus glnR gene comprises a nucleic acid sequence of SEQ ID NO: 12.
54. The microbial composition of any one of embodiments 35-53, wherein the mutant glnR gene comprises a nucleic acid sequence selected from the group consisting of SEQ ID NO: 13-15.
55. The microbial composition of any one of embodiments 45-52, wherein the Paenibacillus GlnR protein comprises an amino acid sequence of SEQ ID NO: 16.
56. The microbial composition of any one of embodiments 35-56, wherein the mutant GlnR protein comprises an amino acid selected from the group consisting of SEQ ID NO: 17-19.
57. The microbial composition of any one of embodiments 35-56, wherein the one or more engineered gram-positive diazotrophic bacteria comprise deletion of a glutamine synthetase A (glnA) gene.
58. The microbial composition of any one of embodiments 35-56, wherein the one or more engineered gram-positive diazotrophic bacteria comprise a mutated form of a glutamine synthetase A (glnA) gene, wherein the mutated form of the glnA gene encodes a mutated GlnA protein that exhibits reduced assimilation of ammonium.
59. The microbial composition of embodiment 58, wherein the mutated GlnA comprises at least one amino acid substitution at position 67, 182, 241 or 313 of a Paenibacillus GlnA or at a homologous amino acid position in a homolog thereof.
60. The microbial composition of embodiment 58, wherein the mutated GlnA comprises at least one amino acid substitution selected from the group consisting of M67I, E182K, G241S and N313B of a Paenibacillus GlnA and homologous amino acid positions in a homolog thereof.
61. The microbial composition of any one of embodiments 35-60, wherein the one or more engineered gram-positive diazotrophic bacteria further comprise at least one genetic variation introduced into a member selected from the group consisting of: nifB, nifH, nifD, nifK, nifE, nifN, nifX, hesA, nifV genes and combinations thereof that results in increased nitrogen fixation.
62. The microbial composition of any one of embodiments 35-61, wherein the one or more engineered gram-positive diazotrophic bacteria comprise at least two different species of bacteria.
63. The microbial composition of any one of embodiments 35-61, wherein the one or more engineered gram-positive diazotrophic bacteria comprise at least two different strains of the same species of bacteria.
64. The microbial composition of any one of embodiments 35-63, wherein the one or more engineered gram-positive diazotrophic bacteria is a species from a genus selected from Paenibacillus, Bacillus and Lactobacillus.
65. The microbial composition of any one of embodiments 35-64, wherein the one or more engineered gram-positive diazotrophic bacteria is selected from Paenibacillus azotofixans, Paenibacillus borealis, Paenibacillus durus, Paenibacillus macerans, Paenibacillus polymyxa, Paenibacillus alvei, Paenibacillus amylolyticus, Paenibacillus campinasensis, Paenibacillus chibensis, Paenibacillus glucanolyticus, Paenibacillus illinoisensis, Paenibacillus larvae subsp. Larvae, Paenibacillus larvae subsp. Pulvifaciens, Paenibacillus lautus, Paenibacillus macerans, Paenibacillus macquariensis, Paenibacillus graminis, Paenibacillus pabuli, Paenibacillus peoriae, Paenibacillus stellifer, Paenibacillus riograndensis, Paenibacillus donghaensis, Paenibacillus sp. FSL, or Paenibacillus odorifier.
66. The microbial composition of any one of embodiments 35-65, wherein the one or more engineered gram-positive diazotrophic bacteria produce 1% or more of fixed nitrogen in a plant exposed thereto.
67. The microbial composition of any one of embodiments 35-66, wherein the composition is a solid.
68. The microbial composition of any one of embodiments 35-66, wherein the composition is a liquid.
69. The microbial composition of any one of embodiments 35-66, wherein the microbial composition is a present as a seed coat on a plant seed or other plant propagation material.
70. The microbial composition of embodiment 68, wherein the microbial composition is present as a liquid on a plant as an in-furrow treatment.
71. The microbial composition of any one of embodiments 35-70, wherein the one or more engineered gram-positive diazotrophic bacteria are transgenic or remodeled non-intergeneric bacteria.
72. The microbial composition of any one of embodiments 34-71, wherein the wild-type gram-positive diazotrophic bacterium is Paenibacillus polymyxa strain CI41 with deposit accession number PTA-126581.
73. A method of providing fixed nitrogen to a plant comprising applying the microbial composition of any one of embodiments 34-72 to the plant, a plant part, or a locus in which the plant is located, or a locus in which the plant will be grown.
74. The method of embodiment 73, wherein the applying comprises coating a seed or other plant propagation member with the microbial composition.
75. The method of embodiment 74, wherein the one or more engineered gram-positive diazotrophic bacteria in the microbial composition has an average colonization ability per unit of plant root tissue of at least about 1.0×10⁴ colony forming unit (cfu) per gram of fresh weight of plant root tissue and produce fixed N of at least about 1×10⁻¹⁵ mmol N per bacterial cell per hour.
76. The method of embodiment 73, wherein the applying comprises performing in-furrow treatment of the microbial composition to a locus in which the plant is present, or will be present.
77. The method of embodiment 76, wherein the in-furrow treatment comprises applying the microbial composition at a concentration per acre of between about 1×10⁶ to about 3×10¹² cfu per acre.
78. The method of embodiment 76 or 77, wherein the microbial composition is a liquid formulation comprising about 1×10⁶ to about 1×10¹¹ cfu of bacterial cells per milliliter.
79. A glnR gene comprising at least one nucleotide substitution at nucleotide position 45, 46, 52, 111, 160, 272, 296, 316, 341, 347, 365, 382, 384 or 397 of a Paenibacillus glnR gene or at a homologous nucleotide position in a homolog thereof.
80. The glnR gene of embodiment 79, wherein the glnR gene shares at least 85%, 86%, 87%, 88%, 89%, 90%, 91%, 92%, 93%, 94%, 95%, 96%, 97%, 98%, or 99% identity with the Paenibacillus glnR gene or the homolog thereof.
81. The glnR gene of embodiment 79 or 80, wherein the glnR gene encodes a GlnR protein comprising at least one amino acid substitution of at amino acid position 16, 18, 37, 54, 91, 99, 106, 114, 116, 122, 128 or 133 of a Paenibacillus GlnR protein or at a homologous amino acid position in a homolog thereof.
82. The glnR gene of embodiment 79 or 80, wherein the glnR gene encodes a GlnR protein comprising at least one amino acid substitution selected from the group consisting of a I16V, M18V, I37M, V54I, T91I, R99H, L106F, L114P, A116V, Q122R, G128S and F133L of a Paenibacillus GlnR protein and homologous amino acid positions in a homolog thereof.
83. The glnR gene of embodiment 81 or 82, wherein the GlnR protein shares at least 90%, 91%, 92%, 93%, 94%, 95%, 96%, 97%, 98%, or 99% identity with the Paenibacillus GlnR protein or the homolog thereof.
84. The glnR gene of any one of embodiments 81-83, wherein the GlnR protein comprises an L to P mutation at position 114 of the Paenibacillus GlnR protein or at a homologous amino acid position in the homolog thereof.
85. The glnR gene of any one of embodiments 81-84, wherein the GlnR protein comprises a L114P mutation and one or more of a R99H mutation, an A116V mutation, a F133L mutation, an I16V mutation, a T91I mutation, a L106F mutation, a G128S mutation, a M18V mutation, an I37M mutation, a V54I mutation, a Q122R mutation and any combination thereof of the Paenibacillus GlnR protein or at a homologous amino acid position in the homolog thereof.
86. The glnR gene of any one of embodiments 81-85, wherein the GlnR protein comprises a L114P, a R99H mutation, an A116V mutation, and a F133L mutation of the Paenibacillus GlnR protein or at a homologous amino acid position in the homolog thereof.
87. The glnR gene of any one of embodiments 81-85, wherein the GlnR protein comprises a L114P, an I16V mutation, a T91I mutation, a L106F mutation, and a G128S mutation of the Paenibacillus GlnR protein or at a homologous amino acid position in the homolog thereof.
88. The glnR gene of any one of embodiments 81-85, wherein the GlnR protein comprises a L114P, a M18V mutation, an 137M mutation, a V54I mutation, and a Q122R mutation of the Paenibacillus GlnR protein or at a homologous amino acid position in the homolog thereof.
89. The glnR gene of any one of embodiments 79-88, wherein the Paenibacillus glnR gene comprises a nucleic acid sequence of SEQ ID NO: 12.
90. The glnR gene of any one of embodiments 79-89, wherein the glnR gene comprises a nucleic acid sequence selected from the group consisting of SEQ ID NO: 13-15.
91. The glnR gene of any one of embodiments 81-90, wherein the Paenibacillus GlnR protein comprises an amino acid sequence of SEQ ID NO: 16.
92. The glnR gene of any one of embodiments 81-91, wherein the GlnR protein comprises an amino acid selected from the group consisting of SEQ ID NO: 17-19.
93. A GlnR protein comprising at least one amino acid substitution of at amino acid position 16, 18, 37, 54, 91, 99, 106, 114, 116, 122, 128 or 133 of a Paenibacillus GlnR protein or at a homologous amino acid position in a homolog thereof.
94. The GlnR protein of embodiment 93, wherein the at least one amino acid substitution is selected from the group consisting of a i16V, M18V, 137M, V54I, T91I, R99H, L106F, L114P, A116V, Q122R, G128S and F133L of the Paenibacillus GlnR protein and homologous amino acid positions in the homolog thereof.
95. The GlnR protein of embodiment 93 or 94, wherein the GlnR protein shares at least 90%, 91%, 92%, 93%, 94%, 95%, 96%, 97%, 98%, or 99% identity with the Paenibacillus GlnR protein or the homolog thereof.
96. The GlnR protein of any one of embodiments 93-95, wherein the GlnR protein comprises an L to P mutation at position 114 of the Paenibacillus GlnR protein or at a homologous amino acid position in the homolog thereof.
97. The GlnR protein of any one of embodiments 93-96, wherein the GlnR protein comprises a L114P mutation and one or more of a R99H mutation, an A116V mutation, a F133L mutation, an I16V mutation, a T91I mutation, a L106F mutation, a G128S mutation, a M18V mutation, an I37M mutation, a V54I mutation, a Q122R mutation and any combination thereof of the Paenibacillus GlnR protein or at a homologous amino acid position in the homolog thereof.
98. The GlnR protein of any one of embodiments 93-96, wherein the GlnR protein comprises a L114P, a R99H mutation, an A116V mutation, and a F133L mutation of the Paenibacillus GlnR protein or at a homologous amino acid position in the homolog thereof.
99. The GlnR protein of any one of embodiments 93-96, wherein the GlnR protein comprises a L114P, an i16V mutation, a T91I mutation, a L106F mutation, and a G128S mutation of the Paenibacillus GlnR protein or at homologous amino acid positions in the homolog thereof.
100. The GlnR protein of any one of embodiments 93-96, wherein the GlnR protein comprises a L114P, a M18V mutation, an I37M mutation, a V54I mutation, and a Q122R mutation of a Paenibacillus GlnR protein or at a homologous amino acid position in a homolog thereof.
101. The GlnR protein of any one of embodiments 93-100, wherein the Paenibacillus GlnR protein comprises an amino acid sequence of SEQ ID NO: 16.
102. The GlnR protein of any one of embodiments 93-101, wherein the GlnR protein comprises an amino acid selected from the group consisting of SEQ ID NO: 17-19.
103. A method for identifying regulators of a nif operon that exhibit de-repression activity in the presence of ammonium, the method comprising:
(a) introducing individual mutagenized glnR genes from a library of mutagenized glnR genes into a engineered gram-positive diazotrophic microbial host cell missing a wild-type glnR gene, wherein the gram-positive diazotrophic microbial host cell comprises a nucleic acid sequence encoding a selectable marker protein, functional fragment, and/or fusions thereof operably linked to a nifB promoter;
(b) culturing the engineered gram-positive diazotrophic microbial host cell in the presence of ammonium under anaerobic conditions, wherein the engineered gram-positive diazotrophic microbial host cell expresses the selectable marker protein, functional fragment, and/or fusions thereof in the presence of ammonium if the mutagenized glnR gene introduced in step (a) encodes a GlnR protein that exhibits de-repression activity in the presence of ammonium;
(c) exposing the engineered gram-positive diazotrophic microbial host cell to an agent that allows for selection of gram-positive diazotrophic microbial host cell's expressing the selectable marker protein; and
(d) identifying individual mutagenized glnR genes from the library of mutagenized glnR genes as exhibiting de-repression activity in the presence of ammonium as those that result in selection of the gram-positive diazotrophic microbial host cells expressing the selectable marker protein as compared to a control.
104. The method of embodiment 103, wherein the selectable marker protein is selected from a fluorescent marker protein, a luminescent marker protein, a chromogenic marker, an auxotrophic marker and antibiotic resistance marker protein.
105. The method of embodiment 104, wherein the selectable marker protein is a fluorescent marker protein.
106. The method of embodiment 105, wherein the fluorescent protein is a GFP, RFP, YFP, CFP, or functional variant or fragment thereof.
107. The method of embodiment 105 or 106, wherein the fluorescent marker protein is GFP.
108. The method of any one of embodiments 105-107, wherein steps (b)-(d) comprise:
(b) culturing the engineered gram-positive diazotrophic microbial host cell in the presence of ammonium under anaerobic conditions, wherein the engineered gram-positive diazotrophic microbial host cell expresses the fluorescent marker protein, functional fragment, and/or fusions thereof in the presence of ammonium if the mutagenized glnR gene introduced in step (a) encodes a GlnR protein that exhibits de-repression activity in the presence of ammonium;
(c) exposing the engineered gram-positive diazotrophic microbial host cell to light excitation sufficient to fluoresce the fluorescent marker protein, functional fragment, and/or fusions thereof; and
(d) identifying individual mutagenized glnR genes from the library of mutagenized glnR genes as exhibiting de-repression activity in the presence of ammonium as those that result in fluorescence of the fluorescent marker protein, functional fragment, and/or fusions thereof, as compared to a control.
109. The method of any one of embodiments 105-108, wherein the fluorescence is detected with a flow cytometer, a plate reader, or fluorescence-activated droplet sorting.
110. The method of any one of embodiments 103-109, wherein the control is an engineered gram-positive diazotrophic microbial host cell expressing wild-type glnR.
111. The method of any one of embodiments 103-110, wherein step (b) is performed in the presence of at least 1 mM, 2 mM, 3 mM, 4 nM, 5 mM, 6 mM, 7 mM, 8 mM, 9 mM or 10 mM ammonium.
112. The method of any one of embodiments 103-111, wherein the engineered gram-positive diazotrophic microbial host cell is selected from Paenibacillus, Bacillus and Lactobacillus.
113. The method of any one of embodiments 103-112, wherein the engineered gram-positive diazotrophic microbial host cell is selected from Paenibacillus azotofixans, Paenibacillus borealis, Paenibacillus durus, Paenibacillus macerans, Paenibacillus polymyxa, Paenibacillus alvei, Paenibacillus amylolyticus, Paenibacillus campinasensis, Paenibacillus chibensis, Paenibacillus glucanolyticus, Paenibacillus illinoisensis, Paenibacillus larvae subsp. Larvae, Paenibacillus larvae subsp. Pulvifaciens, Paenibacillus lautus, Paenibacillus macerans, Paenibacillus macquariensis, Paenibacillus graminis, Paenibacillus pabuli, Paenibacillus peoriae, Paenibacillus stellifer, Paenibacillus riograndensis, Paenibacillus donghaensis, Paenibacillus sp. FSL, and Paenibacillus odorifier.
114. The method of any one of embodiments 103-113, wherein the engineered gram-positive diazotrophic microbial host cell is a transgenic or remodeled non-intergeneric host cell.
115. The method of any one of embodiments 103-114, wherein the identified mutagenized glnR gene comprises at least one nucleotide substitution at nucleotide position 45, 46, 52, 111, 160, 272, 296, 316, 341, 347, 365, 382, 384 or 397 of a Paenibacillus glnR gene or at a homologous nucleotide position in a homolog thereof.
116. The method of embodiment 115, wherein the mutagenized glnR gene shares at least 85%, 86%, 87%, 88%, 89%, 90%, 91%, 92%, 93%, 94%, 95%, 96%, 97%, 98%, or 99% identity with the Paenibacillus glnR gene or the homolog thereof.
117. The method of embodiment 115 or 116, wherein the mutagenized glnR gene encodes a GlnR protein comprising at least one amino acid substitution of at amino acid position 16, 18, 37, 54, 91, 99, 106, 114, 116, 122, 128 or 133 of a Paenibacillus GlnR protein or at a homologous amino acid position in a homolog thereof.
118. The method of embodiment 115 or 116, wherein the mutagenized glnR gene encodes a GlnR protein comprising at least one amino acid substitution selected from the group consisting of a I16V, M18V, I37M, V54I, T91I, R99H, L106F, L114P, A116V, Q122R, G128S and F133L of a Paenibacillus GlnR protein and homologous amino acid positions in a homolog thereof.
119. The method of embodiment 117 or 118, wherein the GlnR protein shares at least 90%, 91%, 92%, 93%, 94%, 95%, 96%, 97%, 98%, or 99% identity with the Paenibacillus GlnR protein or the homolog thereof.
120. The method of any one of embodiments 117-119, wherein the GlnR protein comprises an L to P mutation at position 114 of the Paenibacillus GlnR protein or at a homologous amino acid position in the homolog thereof.
121. The method of any one of embodiments 117-120, wherein the GlnR protein comprises a L114P mutation and one or more of a R99H mutation, an A116V mutation, a F133L mutation, an I16V mutation, a T91I mutation, a L106F mutation, a G128S mutation, a M18V mutation, an I37M mutation, a V54I mutation, a Q122R mutation and any combination thereof of the Paenibacillus GlnR protein or at a homologous amino acid position in the homolog thereof.
122. The method of any one of embodiments 117-121, wherein the GlnR protein comprises a L114P, a R99H mutation, an A116V mutation, and a F133L mutation of the Paenibacillus GlnR protein or at a homologous amino acid position in the homolog thereof.
123. The method of any one of embodiments 117-121, wherein the GlnR protein comprises a L114P, an I16V mutation, a T91I mutation, a L106F mutation, and a G128S mutation of the Paenibacillus GlnR protein or at a homologous amino acid position in the homolog thereof.
124. The method of any one of embodiments 117-121, wherein the GlnR protein comprises a L114P, a M18V mutation, an 137M mutation, a V54I mutation, and a Q122R mutation of the Paenibacillus GlnR protein or at homologous amino acid positions in the homolog thereof.
125. The method of any one of embodiments 117-121, wherein the Paenibacillus glnR gene comprises a nucleic acid sequence of SEQ ID NO: 12.
126. The method of any one of embodiments 115-125, wherein the glnR gene comprises a nucleic acid sequence selected from the group consisting of SEQ ID NO: 13-15.
127. The method of any one of embodiments 117-126, wherein the Paenibacillus GlnR protein comprises an amino acid sequence of SEQ ID NO: 16.
128. The method of any one of embodiments 117-127, wherein the GlnR protein comprises an amino acid selected from the group consisting of SEQ ID NO: 17-19.
129. A method of providing fixed nitrogen to a plant comprising applying a microbial composition to a plant, a plant part, or a locus in which the plant is located, or a locus in which the plant will be grown, wherein the microbial composition comprises one or more engineered gram-positive diazotrophic bacteria capable of fixing nitrogen irrespective of exogenous nitrogen levels.
130. The method of embodiment 129, wherein the one or more engineered gram-positive diazotrophic bacteria comprise a heterologous promoter operably linked to a nif operon, wherein the heterologous promoter replaces at least a portion of the nif operon endogenous promoter and promotes expression of the nif operon irrespective of exogenous nitrogen levels.
131. The method of embodiment 130, wherein the heterologous promoter completely replaces the nif operon endogenous promoter.
132. The method of embodiment 130, wherein the heterologous promoter replaces a portion of the nif operon endogenous promoter downstream of a GlnR activator site, endogenous transcription start site and a GlnR repressor site.
133. The method of embodiment 130, wherein the heterologous promoter replaces a portion of the nif operon endogenous promoter downstream of a GlnR activator site and endogenous transcription start site.
134. The method of embodiment 130, wherein the heterologous promoter replaces a portion of the nif operon endogenous promoter downstream of a GlnR activator site.
135. The method of any one of embodiments 130-134, wherein the heterologous promoter is selected from a promoter for the Paenibacillus Acetolactate synthase (alsS) gene, Pyruvate formate-lyase-activating enzyme (pflB) gene, D-alanine aminotransferase (dat) gene, 30S ribosomal protein S21 (rpsU) gene, Aldehyde-alcohol dehydrogenase (adhe) gene, 50S ribosomal protein L13 (rplm) gene, 50S ribosomal protein L36 (rpmJ) gene, DNA-binding protein HU 1 (hupA) gene, Translation initiation factor IF-3 (infC) gene, ECF RNA polymerase sigma-E factor (rpoE) gene, and Trigger factor (tig) gene.
136. The method of any one of embodiments 130-135, wherein the heterologous promoter has a nucleic acid sequence selected from SEQ ID NOs: 1-11.
137. The method of any one of embodiments 129-136, wherein the one or more engineered gram-positive diazotrophic bacteria are selected from the group consisting of 41-2753, 41-2755, 41-4230, 41-4231, 41-4232, 41-4233 and 41-4236.
138. The method of any one of embodiments 129-136, wherein the one or more engineered gram-positive diazotrophic bacteria comprise a mutant glnR gene, wherein the mutant glnR gene encodes a mutant GlnR protein that promotes expression of the nif operon irrespective of exogenous nitrogen levels.
139. The method of embodiment 138, wherein the mutant glnR gene comprises at least one nucleotide substitution at nucleotide position 45, 46, 52, 111, 160, 272, 296, 316, 341, 347, 365, 382, 384 or 397 of a Paenibacillus glnR gene or at a homologous nucleotide position in a homolog thereof.
140. The method of embodiment 138 or 139, wherein the mutant glnR gene shares at least 85%, 86%, 87%, 88%, 89%, 90%, 91%, 92%, 93%, 94%, 95%, 96%, 97%, 98%, or 99% identity with the Paenibacillus glnR gene or the homolog thereof.
141. The method of any one of embodiments 138-140, wherein the mutant GlnR protein comprises at least one amino acid substitution of at amino acid position 16, 18, 37, 54, 91, 99, 106, 114, 116, 122, 128 or 133 of a Paenibacillus GlnR protein or at a homologous amino acid position in a homolog thereof.
142. The method of any one of embodiments 138-140, wherein the mutant GlnR protein comprises at least one amino acid substitution selected from the group consisting of a I16V, M18V, I37M, V54I, T91I, R99H, L106F, L114P, A116V, Q122R, G128S and F133L of a Paenibacillus GlnR protein and homologous amino acid positions in a homolog thereof.
143. The method of any one of embodiments 138-142, wherein the mutant GlnR protein shares at least 90%, 91%, 92%, 93%, 94%, 95%, 96%, 97%, 98%, or 99% identity with the Paenibacillus GlnR protein or the homolog thereof.
144. The method of any one of embodiments 138-143, wherein the mutant GlnR protein comprises an L to P mutation at position 114 of the Paenibacillus GlnR protein or at a homologous amino acid position in the homolog thereof.
145. The method of any one of embodiments 138-144, wherein the mutant GlnR protein comprises a L114P mutation and one or more of a R99H mutation, an A116V mutation, a F133L mutation, an I16V mutation, a T91I mutation, a L106F mutation, a G128S mutation, a M18V mutation, an 137M mutation, a V54I mutation, a Q122R mutation and any combination thereof of a Paenibacillus GlnR protein or at a homologous amino acid position in a homolog thereof.
146. The method of any one of embodiments 138-145, wherein the mutant GlnR protein comprises a L114P, a R99H mutation, an A116V mutation, and a F133L mutation of a Paenibacillus GlnR protein or at a homologous amino acid position in a homolog thereof.
147. The method of any one of embodiments 138-145, wherein the mutant GlnR protein comprises a L114P, an I16V mutation, a T91I mutation, a L106F mutation, and a G128S mutation of a Paenibacillus GlnR protein or at a homologous amino acid position in a homolog thereof.
148. The method of any one of embodiments 138-145, wherein the mutant GlnR protein comprises a L114P, a M18V mutation, an I37M mutation, a V54I mutation, and a Q122R mutation of a Paenibacillus GlnR protein or at a homologous amino acid position in a homolog thereof.
149. The method of embodiment 139 or 140, wherein the Paenibacillus glnR gene comprises a nucleic acid sequence of SEQ ID NO: 12.
150. The method of any one of embodiments 138-140 or 149, wherein the mutant glnR gene comprises a nucleic acid sequence selected from the group consisting of SEQ ID NO: 13-15.
151. The method of any one of embodiments 141-148, wherein the Paenibacillus GlnR protein comprises an amino acid sequence of SEQ ID NO: 16.
152. The method of any one of embodiments 138-148 or 151, wherein the mutant GlnR protein comprises an amino acid selected from the group consisting of SEQ ID NO: 17-19.
153. The method of any one of embodiments 129-152, wherein the one or more engineered gram-positive diazotrophic bacteria comprises a deletion of a glutamine synthetase A (glnA) gene.
154. The method of any one of embodiments 129-152, wherein the one or more engineered gram-positive diazotrophic bacteria comprises a mutated form of a glutamine synthetase A (glnA) gene, wherein the mutated form of the glnA gene encodes a mutated GlnA protein that exhibits reduced assimilation of ammonium.
155. The method of embodiment 154, wherein the mutated GlnA protein comprises at least one amino acid substitution at position 67, 182, 241 or 313 of a Paenibacillus GlnA or at a homologous amino acid position in a homolog thereof.
156. The method of embodiment 154, wherein the mutated GlnA protein comprises at least one amino acid substitution selected from the group consisting of M67I, E182K, G241S and N313B of a Paenibacillus GlnA and homologous amino acid positions in a homolog thereof.
157. The method of any one of embodiments 129-156, wherein the one or more engineered gram-positive diazotrophic bacteria comprise at least one genetic variation introduced into a member selected from the group consisting of: nifB, nifH, nifD, nifK, nifE, nifN, nifX, hesA, nifV genes and combinations thereof that results in increased nitrogen fixation.
158. The method of any one of embodiments 129-157, wherein the one or more engineered gram-positive diazotrophic bacteria comprise at least two different species of bacteria.
159. The method of any one of embodiments 129-157, wherein the one or more engineered gram-positive diazotrophic bacteria comprise at least two different strains of the same species of bacteria.
160. The method of any one of embodiments 129-159, wherein the one or more engineered gram-positive diazotrophic bacteria is a species from a genus selected from Paenibacillus, Bacillus and Lactobacillus.
161. The method of any one of embodiments 129-160, wherein the one or more engineered gram-positive diazotrophic bacteria is selected from Paenibacillus azotofixans, Paenibacillus borealis, Paenibacillus durus, Paenibacillus macerans, Paenibacillus polymyxa, Paenibacillus alvei, Paenibacillus amylolyticus, Paenibacillus campinasensis, Paenibacillus chibensis, Paenibacillus glucanolyticus, Paenibacillus illinoisensis, Paenibacillus larvae subsp. Larvae, Paenibacillus larvae subsp. Pulvifaciens, Paenibacillus lautus, Paenibacillus macerans, Paenibacillus macquariensis, Paenibacillus graminis, Paenibacillus pabuli, Paenibacillus peoriae, Paenibacillus stellifer, Paenibacillus riograndensis, Paenibacillus donghaensis, Paenibacillus sp. FSL, or Paenibacillus odorifier.
162. The method of any one of embodiments 129-161, wherein the one or more engineered gram-positive diazotrophic bacteria produce 1% or more of fixed nitrogen in the plant.
163. The method of any one of embodiments 129-162, wherein the microbial composition is a solid.
164. The method of any one of embodiments 129-162, wherein the microbial composition is a liquid.
165. The method of any one of embodiments 129-164, wherein the one or more engineered gram-positive diazotrophic bacteria are transgenic or remodeled non-intergeneric bacteria.
166. The method of any one of embodiments 129-165, wherein the applying comprises coating a seed or other plant propagation member with the microbial composition.
167. The method of embodiment 166, wherein the one or more engineered gram-positive diazotrophic bacteria in the microbial composition has an average colonization ability per unit of plant root tissue of at least about 1.0×10⁴ cfu per gram of fresh weight of plant root tissue and produce fixed N of at least about 1×10⁻¹⁵ mmol N per bacterial cell per hour.
168. The method of embodiment 166, wherein the applying comprises performing in-furrow treatment of the microbial composition to a locus in which the plant is present, or will be present.
169. The method of embodiment 168, wherein the in-furrow treatment comprises applying the microbial composition at a concentration per acre of between about 1×10⁶ to about 3×10² cfu per acre.
170. The method of embodiment 168 or 169, wherein the microbial composition is a liquid formulation comprising about 1×10⁶ to about 1×10¹¹ cfu of bacterial cells per milliliter.
171. The engineered gram-positive diazotrophic bacterium of embodiment 27 or 28, wherein the Paenibacillus GlnA protein comprises an amino acid sequence of SEQ ID NO: 51 or 52.
172. The engineered gram-positive diazotrophic bacterium of embodiment 171, wherein the homolog thereof is a Klebsiella GlnA protein.
173. The engineered gram-positive diazotrophic bacterium of embodiment 172, wherein the homolog thereof comprises an amino acid sequence of SEQ ID NO: 53.
174. The microbial composition of embodiment 59 or 60, wherein the Paenibacillus GlnA protein comprises an amino acid sequence of SEQ ID NO: 51 or 52.
175. The microbial composition of embodiment 174, wherein the homolog thereof is a Klebsiella GlnA protein.
176. The microbial composition of embodiment 175, wherein the homolog thereof comprises an amino acid sequence of SEQ TD NO: 53.
177. The method of embodiment 155 or 156, wherein the Paenibacillus GlnA protein comprises an amino acid sequence of SEQ TD NO: 51 or 52.
178. The method of embodiment 177, wherein the homolog thereof is a Klebsiella GlnA protein.
179. The method of embodiment 178, wherein the homolog thereof comprises an amino acid sequence of SEQ TD NO: 53.

SEQUENCES OF THE DISCLOSURE WITH SEQ ID NO IDENTIFIERS

| | Nucleic Acid | Amino Acid | |
|---|---|---|---|
| Gene Name | SEQ ID NO. | SEQ ID NO: | Genus/Species/Strain |
| Acetolactate synthase (alsS) | 1 | | Paenibacillus polymyxa CI41 |
| gene promoter (p(alsS)) | | | |
| Pyruvate formate-lyase- | 2 | | P. polymyxa CI41 |
| activating enzyme (pflB) | | | |
| promoter (p(pflB)) | | | |
| D-alanine aminotransferase | 3 | | P. polymyxa CI41 |
| (dat) gene promoter (p(dat)) | | | |
| 30S ribosomal protein S21 | 4 | | P. polymyxa CI41 |
| (rpsU) gene promoter | | | |
| (p(rpsU)) | | | |
| Aldehyde-alcohol | 5 | | P. polymyxa CI41 |
| dehydrogenase (adhe) gene | | | |
| promoter (p(adhE)) | | | |
| 50S ribosomal protein L13 | 6 | | P. polymyxa CI41 |
| (rplm) gene promoter | | | |
| (p(rplM)) | | | |
| 50S ribosomal protein L36 | 7 | | P. polymyxa CI41 |
| (rpmJ) gene promoter | | | |
| (p(rpmJ)) | | | |
| DNA-binding protein HU 1 | 8 | | P. polymyxa CI41 |
| (hupA) gene promoter | | | |
| (p(hupA)) | | | |
| Translation initiation factor | 9 | | P. polymyxa CI41 |
| IF-3 (infC1) gene promoter | | | |
| (p(infC)) | | | |
| ECF RNA polymerase | 10 | | P. polymyxa CI41 |
| sigma-E factor (rpoE1) gene | | | |
| promoter (p(rpoE1)) | | | |
| Trigger factor (tig) gene | 11 | | P. polymyxa CI41 |
| promoter (p(tig)) | | | |
| glnR | 12 | | P. polymyxa CI41 |
| glnR-R99H, L114P, A116V, | 13 | | P. polymyxa |
| F133L | | | |
| glnR-I16V, T91I, L106F, | 14 | | P. polymyxa |
| L114P, G128S | | | |
| glnR-M18V, I37M, V54I, | 15 | | P. polymyxa |
| L114P, Q122R | | | |
| GlnR | | 16 | P. polymyxa CI41 |
| GlnR-R99H, L114P, A116V, | | 17 | P. polymyxa |
| F133L | | | |
| GlnR-I16V, T91I, L106F, | | 18 | P. polymyxa |
| L114P, G128S | | | |
| GlnR-M18V, I37M, V54I, | | 19 | P. polymyxa |
| L114P, Q122R | | | |
| pPb-nifB | 20 | | Reporter plasmid |
| pAD-glnR | 21 | | glnR plasmid |
| nifB | 22 | | P. polymyxa CI41 |
| p(pflB)_nifB_v1 | 23 | | P. polymyxa 41-4230 |
| p(pflB)_nifB_v2 | 24 | | P. polymyxa 41-4231 |
| p(pflB)_nifB_v3 | 25 | | P. polymyxa 41-4232 |
| p(adhE) nifB v1 | 26 | | P. polymyxa 41-4233 |
| p(adhE) nifB v2 | 27 | | P. polymyxa 41-4236 |
| p(adhE) nifB v3 | 28 | | P. polymyxa 41-4266 |
| p(tig)_nifB_v1 | 29 | | P. polymyxa 41-4262 |
| p(tig) nifB v2 | 30 | | P. polymyxa 41-4237 |
| p(tig) nifB v3 | 31 | | P. polymyxa 41-4234 |
| p(pflB)_nifB_v0 | 32 | | P. polymyxa 41-2753 |
| p(adhE)_nifB_v0 | 33 | | P. polymyxa 41-2755 |
| p(tig)_nifB_v0 | 34 | | P. polymyxa 41-2760 |
| | | 35 | P. polymyxa |
| | | 36 | Paenibacillus peoriae |
| | | 37 | Paenibacillus kribbensis |
| | | 38 | Paenibacillus sp. FSL |
| | | 39 | Paenibacillus sabinae |
| | | 40 | Paenibacillus durus |
| | | 41 | Paenibacillus borealis |
| | | 42 | Paenibacillus graminis |
| | | 43 | Paenibacillus odorifier |
| | | 44 | Paenibacillus stellifer |
| | | 45 | Paenibacillus riograndensis |
| | | 46 | Paenibacillus donghaensis |
| Meganuclease peptide motif | | 47 | |
| glnA | 48 | | P. polymyxa CI41 |
| glnA1 | 49 | | P. polymyxa CI41 |
| glnA | 50 | | Klebsiella variicola CI137 |
| GlnA | | 51 | P. polymyxa CI41 |
| GlnA1 | | 52 | P. polymyxa CI41 |
| GlnA | | 53 | K. variicola CI137 |

### INCORPORATION BY REFERENCE

All references, articles, publications, patents, patent publications, and patent applications cited herein are incorporated by reference in their entireties for all purposes.

However, mention of any reference, article, publication, patent, patent publication, and patent application cited herein is not, and should not be taken as, an acknowledgment or any form of suggestion that they constitute valid prior art or form part of the common general knowledge in any country in the world.

1.-36. (canceled)

37. A microbial composition comprising one or more bacteria, wherein the one or more bacteria are capable of fixing nitrogen irrespective of exogenous nitrogen levels at a rate at least equivalent to a rate of nitrogen fixation in a wild-type gram-positive diazotrophic bacterium in the absence of exogenous nitrogen.

38. The microbial composition of claim 37, wherein the one or more bacteria comprise one or more engineered gram-positive diazotrophic bacteria comprising a heterologous promoter operably linked to a nif operon and/or a mutant GlnR protein, wherein the heterologous promoter replaces at least a portion of the nif operon endogenous promoter and promotes expression of the nif operon irrespective of exogenous nitrogen levels, and wherein the mutant GlnR protein promotes expression of the nif operon irrespective of exogenous nitrogen levels.

39.-44. (canceled)

45. The microbial composition of claim 38, wherein the one or more engineered gram-positive diazotrophic bacterium is selected from the group consisting of 41-2753, 41-2755, 41-4230, 41-4231, 41-4232, 41-4233 and 41-4236.

46.-79. (canceled)

80. A method of providing fixed nitrogen to a plant comprising applying the microbial composition of claim 37 to the plant, a plant part, or a locus in which the plant is located, or a locus in which the plant will be grown.

81.-85. (canceled)

86. A glnR gene comprising at least one nucleotide substitution at nucleotide position 341, 382, 384, 45, 46, 52, 111, 160, 272, 296, 316, 347, 365, or 397 of a Paenibacillus glnR gene or at a homologous nucleotide position in a homolog thereof.

87. (canceled)

88. The glnR gene of claim 86, wherein the glnR gene encodes a GlnR protein comprising at least one amino acid substitution at amino acid position 114, 128 16, 18, 37, 54, 91, 99, 106, 116, 122, or 133 of a Paenibacillus GlnR protein or at a homologous amino acid position in a homolog thereof.

89.-95. (canceled)

96. The glnR gene of claim 86, wherein the Paenibacillus glnR gene comprises a nucleic acid sequence of SEQ ID NO: 12.

97. The glnR gene of claim 86, wherein the glnR gene comprises a nucleic acid sequence selected from the group consisting of SEQ ID NO: 13-15.

98. The glnR gene of claim 88, wherein the Paenibacillus GlnR protein comprises an amino acid sequence of SEQ ID NO: 16.

99. The glnR gene of claim 88, wherein the GlnR protein comprises an amino acid selected from the group consisting of SEQ ID NO: 17-19.

100. A GlnR protein comprising at least one amino acid substitution at amino acid position 114, 16, 18, 37, 54, 91, 99, 106, 116, 122, 128 or 133 of a Paenibacillus GlnR protein or at a homologous amino acid position in a homolog thereof.

101.-107. (canceled)

108. The GlnR protein of claim 100, wherein the Paenibacillus GlnR protein comprises an amino acid sequence of SEQ ID NO: 16.

109. The GlnR protein of claim 100, wherein the GlnR protein comprises an amino acid selected from the group consisting of SEQ ID NO: 17-19.

110.-135. (canceled)

136. A method of providing fixed nitrogen to a plant comprising applying a microbial composition to a plant, a plant part, or a locus in which the plant is located, or a locus in which the plant will be grown, wherein the microbial composition comprises one or more engineered gram-positive diazotrophic bacteria capable of fixing nitrogen irrespective of exogenous nitrogen levels.

137. The method of claim 136, wherein the one or more engineered gram-positive diazotrophic bacteria comprise a heterologous promoter operably linked to a nif operon, wherein the heterologous promoter replaces at least a portion of the mf operon endogenous promoter and promotes expression of the nif operon irrespective of exogenous nitrogen levels.

138.-142. (canceled)

143. The method of claim 137, wherein the heterologous promoter has a nucleic acid sequence selected from SEQ ID NOs: 1-11.

144. The method of claim 136, wherein the one or more engineered gram-positive diazotrophic bacteria are selected from the group consisting of 41-2753, 41-2755, 41-4230, 41-4231, 41-4232, 41-4233 and 41-4236.

145. The method of claim 136, wherein the one or more engineered gram-positive diazotrophic bacteria comprise a mutant glnR gene, wherein the mutant glnR gene encodes a mutant GlnR protein that promotes expression of the nif operon irrespective of exogenous nitrogen levels.

146. The method of claim 145, wherein the mutant glnR gene comprises at least one nucleotide substitution at nucleotide position 341, 382, 384, 45, 46, 52, 111, 160, 272, 296, 316, 365, or 397 of a Paenibacillus glnR gene or at a homologous nucleotide position in a homolog thereof.

147. (canceled)

148. The method of claim 145, wherein the mutant GlnR protein comprises at least one amino acid substitution of at amino acid position 144, 16, 18, 37, 54, 91, 99, 106, 116, 122, 128 or 133 of a Paenibacillus GlnR protein or at a homologous amino acid position in a homolog thereof.

149.-155. (canceled)

156. The method of claim 146, wherein the Paenibacillus glnR gene comprises a nucleic acid sequence of SEQ ID NO: 12.

157. The method of claim 145, wherein the mutant glnR gene comprises a nucleic acid sequence selected from the group consisting of SEQ ID NO: 13-15.

158. The method of claim 148, wherein the Paenibacillus GlnR protein comprises an amino acid sequence of SEQ ID NO: 16.

159. The method of claim 145, wherein the mutant GlnR protein comprises an amino acid selected from the group consisting of SEQ ID NO: 17-19.

160.-190. (canceled)
